(19) United States
(12) Patent Application Publication
(10) Pub. No.: US 2024/0277844 A1
(43) Pub. Date: Aug. 22, 2024

(54) INDUCED NK CELLS RESPONSIVE TO CD3/TAA BISPECIFIC ANTIBODIES

(71) Applicant: Regeneron Pharmaceuticals, Inc., Tarrywotn, NY (US)

(72) Inventors: Nicolin Bloch, Elmsford, NY (US); Erica Ullman, Yorktown Heights, NY (US); Aynur Hermann, New York, NY (US); William Olson, Yorktown Heights, NY (US); George D. Yancopoulos, Yorktown Heights, NY (US)

(21) Appl. No.: 18/444,273

(22) Filed: Feb. 16, 2024

Related U.S. Application Data
(60) Provisional application No. 63/446,428, filed on Feb. 17, 2023.
(60) Provisional application No. 63/458,765, filed on Apr. 12, 2023.

(51) Int. Cl. A61K 39/00; C07K 14/725; C07K 16/28; C12N 5/0783

(52) U.S. Cl. CPC A61K 39/4631; A61K 39/4613; A61K 39/464429; C07K 14/7051; C07K 16/283; C12N 5/0646; A61K 2239/13; A61K 2239/17; A61K 2239/21; A61K 2239/22; C07K 2317/565; C07K 2317/622; C12N 2510/00

(57) ABSTRACT

In certain aspects, provided herein are compositions and methods for treating cancer. The methods of the present disclosure comprise administering to a subject in need thereof a NK cell expressing a CAR in combination with an antigen-binding molecule that binds to a tumor antigen, wherein the CAR-NK cell targets tumor cells through binding to the antigen-binding molecule.

### RELATED APPLICATIONS

This application claims the benefit of priority to U.S. Provisional patent application Ser. Nos. 63/446,428, filed Feb. 17, 2023, and 63/458,765, filed Apr. 12, 2023, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Feb. 16, 2024, is named RPB-02801_SL.xml and is 1,084,637 bytes in size.

### BACKGROUND

Cancer is the second leading cause of death in the United States. Current therapies for many cancers either fail in certain patient populations or generate toxic side-effects which greatly impact the quality of life of the patient. Adoptive immunotherapy, which involves the transfer of antigen-specific immune cells (e.g., T cells or NK cells) generated ex vivo, is a promising strategy to treat cancer. Immune cells used for adoptive immunotherapy can be generated, for example, by redirection of immune cells through genetic engineering (e.g., by engineering them to express chimeric antigen receptors, or “CARs”). CAR-NK cells provide a better safety profile, a minimal cytokine release, and less graft-vs-host disease compared to CAR-T cells. However, it is a cumbersome and time-consuming process to engineer a unique CAR-NK cells specific for every potential cancer antigen. Thus, there is a need in the art for improved adoptive immunotherapy approaches for the treatment of cancer.

### SUMMARY

The present disclosure is based in part on the discovery that co-administration of a cancer antigen-binding molecule (e.g., a cancer antigen-specific antibody) and NK cells expressing a CAR that comprises a binding domain specific for the cancer antigen-binding molecule induces cytotoxicity in tumor cells expressing that cancer antigen. Accordingly, in certain embodiments, provided herein are CAR-NK cells that are able to be used as an off-the-shelf therapeutic and that can be targeted to a broad array of different cancers by co-administering a cancer antigen-specific antibody.

Accordingly, in some aspects, provided herein is a chimeric antigen receptor (CAR) polypeptide comprising: (a) an extracellular domain comprising: (i) a CD3 extracellular domain or fragment thereof; (ii) an antigen-binding domain specific for an idiotype of an anti-CD3 antibody; or (iii) an antigen-binding domain specific for an Fc domain; (b) a hinge domain; (c) a transmembrane domain; (d) an intracellular signaling domain.

In some embodiments, the extracellular domain comprises the CD3 extracellular domain or fragment thereof. In some embodiments, the CD3 extracellular domain or fragment thereof comprises an epitope recognized by an anti-CD3 antibody. In some embodiments, the anti-CD3 antibody is selected from the anti-CD3 antibodies listed in Table 6. In some embodiments, the CD3 extracellular domain or fragment thereof comprises at least 10 consecutive amino acids of SEQ ID NO: 1959. In some embodiments, the CD3 extracellular domain or fragment thereof comprises an amino acid sequence at least 90% identical to SEQ ID NO: 1959. In some embodiments, the CD3 extracellular domain or fragment thereof comprises an amino acid sequence of SEQ ID NO: 1959.

In some embodiments, the extracellular domain comprises the antigen-binding domain specific for an idiotype of an anti-CD3 antibody. In some embodiments, the anti-CD3 antibody is selected from the anti-CD3 antibodies listed in Table 6. In some embodiments, the antigen-binding domain is a single chain fragment variable (scFv). In some embodiments, the antigen-binding domain comprises the heavy chain and light chain CDR sequences of a scFv listed in Table 1. In some embodiments, the antigen binding domain comprises the heavy chain and light chain variable region sequences of one of an scFv listed in Table 1. In some embodiments, the antigen-binding domain comprises the amino acid sequence of a scFv listed in Table 1.

In some embodiments, the extracellular domain comprises the antigen binding domain specific for an Fc domain. In some embodiments, the Fc domain is selected from a human IgG1 Fc domain, a human IgG2 Fc domain, a human IgG3 Fc domain, and a human IgG4 Fc domain. In some embodiments, the Fc domain is an IgG3 Fc domain. In some embodiments, the Fc domain comprises the amino acid sequence of an Fc shown in FIG. 3.

In some embodiments, the antigen binding domain is a single chain fragment variable (scFv).

In some embodiments, the hinge domain is a CD28 or CD8 hinge domain. In some embodiments, the hinge domain comprises the amino acid sequence selected from SEQ ID NOs: 1-5. In some embodiments, the transmembrane domain is an NKG2D transmembrane domain, an NKG2D inverted transmembrane domain, a CD28 transmembrane domain, a CD8 transmembrane domain, a CD16 transmembrane domain, or a FcgR1 (CD64) transmembrane domain. In some embodiments, the transmembrane domain comprises an amino acid sequence selected from SEQ ID NOs: 6-13. In some embodiments, the intracellular signaling comprises any combination of FcgR1 intracellular signaling domain, a CD3z intracellular signaling domain, a 4-1BB intracellular signaling domain, a 2B4 intracellular signaling domain, a CD16 intracellular signaling domain, a CD64 intracellular signaling domain, or a CD28 intracellular signaling domain. In some embodiments, the intracellular signaling domain is FcgR1 intracellular signaling domain, a 4-1BB-CD3z intracellular signaling domain, a 2B4-CD32 intracellular signaling domain, a CD16 intracellular signaling domain, a CD64 intracellular signaling domain, or a CD28-CD3z intracellular signaling domain.

In some aspects, provided herein is a vector comprising the nucleic acid described herein. In some embodiments, the vector is an expression vector. In some embodiments, the vector is a viral vector. In some embodiments, the viral vector is lentiviral vector.

In some aspects, provided herein is a natural killer (NK) cell comprising the nucleic acid described herein. In some aspects, provided herein is a natural killer (NK) cell expressing the CAR polypeptide described herein. In some embodiments, the cell is a primary NK cell or an inducible NK cells differentiated from an induced pluripotent stem cell (iPSC). In some aspects, provided herein is an immune cell (e.g., a phagocyte) comprising the nucleic acid described herein, or expressing the CAR polypeptide described herein.

In some aspects, provided herein is a method of treating cancer in a subject, the method comprising conjointly administering to the subject: (A) a natural killer (NK) cell expressing a CAR polypeptide comprising an extracellular domain; and (B) a multi-specific antigen-binding molecule comprising a first antigen-binding domain that binds to a tumor antigen and a second antigen-binding domain that binds to the extracellular domain. In certain embodiments, the method comprising conjointly administering to the subject: (A) a natural killer (NK) cell expressing a CAR polypeptide comprising an extracellular domain that comprises a CD3 extracellular domain or fragment thereof; and (B) a multi-specific antigen-binding molecule comprising a CD3-binding domain that specifically binds to the CD3 extracellular domain or fragment thereof and a tumor antigen-binding domain that specifically binds to a tumor antigen.

In some aspects, provided herein is a method of treating cancer in a subject, the method comprising conjointly administering to the subject: (A) an antigen binding molecule that binds to a tumor antigen; and (B) a natural killer (NK) cell expressing a CAR polypeptide comprising an extracellular domain that binds to the antigen-binding molecule. In some embodiments, the method comprising conjointly administering to the subject: (A) a multi-specific antigen binding molecule comprising a CD3-binding domain that specifically binds to CD3 and a tumor antigen-binding domain that specifically binds to a tumor antigen; and (B) a natural killer (NK) cell expressing a CAR polypeptide comprising an extracellular domain that comprises an antigen-binding domain specific for an idiotype of an anti-CD3 antibody, wherein the antigen binding domain of the CAR polypeptide binds to the idiotype of the CD3-binding domain of the multi-specific antigen binding molecule.

In some aspects, provided herein is a method of treating cancer in a subject, the method comprising conjointly administering to the subject: (a) an antigen binding molecule that binds to a tumor antigen and that comprises an Fc domain; and (b) a natural killer (NK) cell expressing a CAR polypeptide comprising an extracellular domain that binds to the Fc domain. In some embodiments, the method comprising conjointly administering to the subject: (A) a multi-specific antigen binding molecule comprising a CD3-binding domain that specifically binds to CD3, a tumor antigen-binding domain that specifically binds to a tumor antigen, and a Fc domain; and (B) a natural killer (NK) cell expressing a CAR polypeptide comprising an extracellular domain that comprises an antigen binding domain specific for an Fc domain, wherein the antigen binding domain of the CAR polypeptide binds to the Fc domain of the multi-specific antigen binding molecule.

In some embodiments, the antigen-binding molecule (e.g., the multi-specific antigen binding molecule) and the NK cells are administered concurrently or sequentially. In some embodiments, wherein the antigen binding molecule (e.g., the multi-specific antigen binding molecule) and the NK cells are pre-mixed and administered to the subject simultaneously. In some embodiments, the subject is lymphopenic and the antigen binding molecule (e.g., the multi-specific antigen binding molecule) and the NK cells are pre-mixed and administered to the subject simultaneously. In some embodiments, the NK cells or the premixed NK cells and antigen-binding molecule (e.g., the premixed NK cells and multi-specific antigen binding molecule) are administered after at least one does of the antigen-binding molecule (e.g., the multi-specific antigen binding molecule).

In some embodiments, the antigen binding molecule (e.g., the multi-specific antigen binding molecule) is a bispecific antigen binding molecule. In some embodiments, the tumor antigens include but are not limited to, e.g., CD19, CD123, STEAP2, CD20, SSTR2, CD38, STEAP1, 5T4, ENPP3, PSMA, MUC16, GPRC5D, BCMA, CA19.9, MSLN, CD22, SLC3A2-APIS, CLDN18.2, and CEACAM5. In some embodiments, the antigen binding molecule (e.g., the multi-specific antigen binding molecule) comprises a multi-specific antibody or antigen-binding fragment thereof. In some embodiments, the multi-specific antibody or antigen-binding fragment thereof is chimeric, humanized, or human.

In some embodiments, the antigen binding molecule (e.g., the multi-specific antigen binding molecule) is selected from a bispecific CD3xCD19 antibody, a bispecific CD3x GPRC5D antibody, a bispecific CD3xCD123 antibody, a bispecific CD3xSTEAP2 antibody, a bispecific CD3xCD20 antibody, a bispecific CD3xSSTR 2 antibody, a bispecific CD3xCD38 antibody, a bispecific CD3xSTEAP1 antibody, a bispecific CD3×5T4 antibody, a bispecific CD3xENPP3 antibody, a bispecific CD3xMUC16 antibody, a bispecific CD3xBCMA antibody, a bispecific CD3xPSMA antibody, and a trispecific CD3xCD28xCD38 antibody. In some embodiments, the antigen-binding molecule (e.g., the multi-specific antigen binding molecule) is a multi-specific antigen binding molecule listed in Table 6.

In some aspects, provided herein is a pharmaceutical composition comprising: (A) a natural killer (NK) cell expressing a CAR polypeptide comprising an extracellular domain comprising a CD3 extracellular domain or fragment thereof; and (B) a multi-specific antigen binding molecule comprising a CD3-binding domain that specifically binds to the CD3 extracellular domain or fragment thereof and a tumor antigen-binding domain that specifically binds to a tumor antigen.

In some aspects, provided herein is a pharmaceutical composition comprising: (A) a multi-specific antigen binding molecule comprising a CD3-binding domain that specifically binds to CD3 and a tumor antigen-binding domain that specifically binds to a tumor antigen; and (B) a natural killer (NK) cell expressing a CAR polypeptide comprising an extracellular domain that comprises an antigen-binding domain specific for an idiotype of an anti-CD3 antibody, wherein the antigen binding domain of the CAR polypeptide binds to the idiotype of the CD3-binding domain of the multi-specific antigen binding molecule.

In some aspects, provided herein is a pharmaceutical composition comprising: (A) a multi-specific antigen binding molecule comprising a CD3-binding domain that specifically binds to CD3, a tumor antigen-binding domain that specifically binds to a tumor antigen, and a Fc domain; and (B) a natural killer (NK) cell expressing a CAR polypeptide comprising an extracellular domain that comprises an antigen binding domain specific for an Fc domain, wherein the antigen binding domain of the CAR polypeptide binds to the Fc domain of the multi-specific antigen binding molecule.

In some aspects, provided herein is a cell bank comprising NK cells that express a CAR described herein.

### BRIEF DESCRIPTION OF THE DRAWINGS

FIGS. 1A-1B show NFAT activity of Jurkat/NFAT-Luc cl. 3C7 cells (FIG. 1A) or Jurkat/NFAT-Luc/ahFc-CD28-CD3 cells (FIG. 1B). Both cells were incubated with a titration of an Isotype control (REGN1932, gray square) or a one-arm anti-CD20 antibody (REGN2959, black) and target cells lacking CD20 expression (Jurkat, open symbol/dashed line) or positive for CD20 expression (Ramos.2G6.4C10, closed symbol/solid line). 5 hours later, NFAT activity was assessed by luminescent readout.

FIG. 2 shows the cytotoxicity of KHYG/ahFc-CD28-CD3 cells. KHYG/ahFc-CD28-CD3 cells were incubated with a titration of an Isotype control (REGN1932, gray square, gray dashed line) or a one-arm anti-CD20 antibody (REGN2959, black circle black solid line) in the presence of a fixed amount of Ramos/GFP target cells. 4 hours later, tag release was detected using an extracellular detection system.

FIG. 3 shows sequence alignment between hIgG3 and IgG4 stealth*constant regions. hIgG3, REGN2280, and REGN7075 contain 100% sequence identity in the CH3 region encompassing the star mutation: FSCSVMHEALHNRFTQKSLSLSPGK (SEQ ID NO: 14). Figure discloses SEQ ID NOS 91-94, respectively, in order of appearance.

FIGS. 4A-4F show single chain variable fragments (scFv) derived from anti-human CD3 idiotypic monoclonal antibodies (mAbs) blocking anti-hCD3 mAb binding to immobilized hCD3 ε/δ (epsilon/delta heterodimer). FIGS. 4B and 4D show that 1PN29950_²HCLC (open circles) and PN29950_² LCHC (open squares) blocked binding of 20.0 pM REGN18409 (FIG. 4B, Anti-hCD3, 7221G) or REGN18411 (FIG. 4D, Anti-hCD3, 7221G20) to immobilized hCD3 ε/δ. FIG. 4F shows PN77570_HCLC (open circles) and PN77570_LCHC (open squares) blocked binding of 20.0 pM REGN2533 (Anti-hCD3, 9F7) to immobilized hCD3 ε/δ. Additional graphs show dose-dependent binding of REGN18409 (FIG. 4A), REGN18411 (FIG. 4C), and REGN2533 (FIG. 4E) (filled inverted triangles) to immobilized hCD3 ε/δ with an EC₅₀ of 7.5 pM, 11.3 pM, and 8.1 pM, respectively. Parental bivalent mAb REGN5766 (filled circles) blocking binding of 20.0 pM REGN18409 (FIG. 4A) or REGN18411 (FIG. 4C) to hCD3 ε/δ with IC₅₀ values of 42.0 pM and 40.0 pM, respectively. FIG. 4E shows parental bivalent mAb REGN2984 (filled circles) blocking binding of 20.0 pM REGN2533 to hCD3 ε/δ with an IC₅₀ value of 32.0 pM. Isotype controls, mIgG1 (FIG. 4A and FIG. 4C) and mIgG2a (FIG. 4E) (southern biotech #0102-01 and 0103-01, respectively; filled triangles) and negative scFv control (FIG. 4A, FIG. 4C, & FIG. 4E) (REGN4393; filled squares) demonstrated no inhibition under identical assay conditions. X-axis is Log₁₀ molar concentration for inset graphs, and for outset graphs, the X-axis is the dilution factor (derived from the Log₁₀ dilution factor). Y-axis for all graphs show absorbance at 450 nm. 1PN annotates the root Protein Number. 2HCLC or 2 LCHC corresponds to the heavy chain, linker, and light chain orientation of the scFv.

FIG. 5 shows binding of antibodies to KHYG-1/NFAT-Luc/CAR1 cells.

FIGS. 6A and 6B show binding of antibodies to KHYG-1/NFAT-Luc/CAR6 and CAR15 cells.

FIG. 7 shows reporter Activation of KHYG-1/NFAT-Luc/CAR1 cells.

FIGS. 8A and 8B show reporter Activation of KHYG-1/NFAT-Luc/CAR6 and CAR15 cells.

FIG. 9 shows cytotoxic Activation of KHYG-1/NFAT-Luc/CAR1 cells.

FIGS. 10A and 10B show cytotoxic Activation of KHYG-1/NFAT-Luc/CAR6 and CAR15 cells.

FIGS. 11A-11G show cytotoxicity and cytokine release from CBNK/CAR6 cells.

### DETAILED DESCRIPTION

### General

Provided herein are methods and compositions for the treatment of cancer. As disclosed herein, administration of NK cells expressing a CAR that comprises an antigen-binding domain specific for an idiotype of an anti-CD3 antibody, or an antigen-binding domain specific for an Fc domain in combination with a bispecific antibody that binds CD3 and a tumor antigen (TAA) induced cytotoxicity in tumor cells expressing the specific tumor antigen.

Accordingly, in certain aspects, provided herein are chimeric immune receptor (CARs) comprising an extracellular domain that comprises a CD3 extracellular domain, an antigen-binding domain specific for an idiotype of an anti-CD3 antibody, or an antigen-binding domain specific for an Fc domain.

In some aspects, provided herein are methods for treating cancer using a NK cell (e.g., inducible NK cells) expressing a CAR described herein in combination with an antigen-binding molecule that binds to a tumor antigen, wherein the CAR-NK cell binds the antigen-binding molecule which then targets cancer cells expressing the tumor antigen to induce anti-tumor activities (e.g., cytotoxicity).

In some embodiment, the methods may comprise conjointly administering to the subject: (A) a natural killer (NK) cell expressing a CAR polypeptide comprising an extracellular domain (e.g., a CD3 extracellular domain or a fragment thereof); and (B) a multi-specific antigen-binding molecule comprising a first antigen-binding domain that binds to a tumor antigen and a second antigen-binding domain that binds to the extracellular domain (e.g., the CD3 extracellular domain or a fragment thereof).

In some embodiment, the methods may comprise conjointly administering to the subject: (A) an antigen-binding molecule that binds to a tumor antigen (e.g., a multi-specific antigen binding molecule comprising a CD3-binding domain that specifically binds to CD3 and a tumor antigen-binding domain that specifically binds to a tumor antigen); and (B) a natural killer (NK) cell expressing a CAR polypeptide comprising an extracellular domain that binds to the antigen-binding molecule (e.g., an extracellular domain that comprises an antigen-binding domain specific for the idiotype of the CD3 multi-specific antigen binding molecule).

In some embodiment, the methods may comprise conjointly administering to the subject: (a) an antigen binding molecule that binds to a tumor antigen and that comprises an Fc domain (e.g., a multi-specific antigen binding molecule comprising a CD3-binding domain that specifically binds to CD3, a tumor antigen-binding domain that specifically binds to a tumor antigen, and an Fc domain); and (b) a natural killer (NK) cell expressing a CAR polypeptide comprising an extracellular domain that binds to the Fc domain.

In some aspects, provided herein are pharmaceutical compositions comprising a CAR-NK cell described herein and an antigen-binding molecule described herein, wherein the CAR-NK cell binds the antigen-binding molecule. In some embodiments, the pharmaceutical compositions further comprise a pharmaceutically acceptable carrier.

CAR-NK cells provide a better safety profile, a minimal cytokine release, and less graft-vs-host disease compared to CAR-T cells. Instead of engineering NK cells each time to express a CAR specific for a particular tumor antigen, the CAR-NK cells disclosed herein can be used an off-shelf “universal” CAR-NK cell which can be used in combination of various tumor antigen-binding molecules to target different types of tumors. For example, the NK cells expressing a CAR comprising a CD3 extracellular domain or an antigen-binding domain specific for an idiotype of an anti-CD3 antibody can be used in combination with various CD3 bispecific antibodies known in the art. The NK cells expressing a CAR comprising an antigen-binding domain specific for an Fc domain can be used in combination with various CD3 bispecific antibodies with the Fc domain, or any other antibody that binds to a tumor antigen and comprises the Fc domain.

### Definitions

For convenience, certain terms employed in the specification, examples, and appended claims are collected here.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The articles “a” and “an” are used herein to refer to one or to more than one (i.e., to at least one) of the grammatical object of the article. By way of example, “an element” means one element or more than one element.

As used herein, the term “administering” or “administration” means providing a pharmaceutical agent or composition to a subject, and includes, but is not limited to, administering by a medical professional and self-administering. Such an agent can contain, for example, a CAR T cell provided herein.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies, trispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s) and” “neoplasm(s)”” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

The term “chimeric antigen receptor” (CAR) refers to molecules that combine a binding domain against a component present on the target cell, for example an antibody-based specificity for a desired antigen (e.g., a tumor antigen) with a T cell receptor-activating intracellular domain to generate a chimeric protein that exhibits a specific anti-target cellular immune activity. Generally, CARs consist of an extracellular single chain antigen-binding domain (scFv) fused to the intracellular signaling domain of the T cell antigen receptor complex zeta chain, and have the ability, when expressed in T cells, to redirect antigen recognition based on the monoclonal antibody's specificity.

As used herein, the phrase “conjoint administration” or “administered conjointly” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

A “costimulatory domain” or “costimulatory molecule” refers to the cognate binding partner on an immune cell (e.g., a B cell, a T cell, a NK cell, or a myeloid cell) that specifically binds with a costimulatory ligand, thereby mediating a costimulatory response by the cell, such as, but not limited to proliferation. The costimulatory domain may be a human costimulatory domain. Exemplary costimulatory molecules include, CD28, CD27, 4-1BB (CD137), OX40, CD30, CD40, ICOS, CD2, LIGHT, CD244 (2B4), and NKG2C.

A “costimulatory ligand” refers to a molecule on an antigen-presenting cell that specifically binds a cognate costimulatory molecule on an immune cell (e.g., a B cell, a T cell, a NK cell, or a myeloid cell), thereby providing a signal which mediates an immune cell (e.g., a B cell, a T cell, a NK cell, or a myeloid cell) response, including, but not limited to, proliferation activation, differentiation and the like. A costimulatory ligand can include but is not limited to CD7, B7-1 (CD80), B7-2 (CD86), 4-1BBL, OX40 L, inducible costimulatory ligand (ICOSLG), intercellular adhesion molecule (ICAM), CD30 L, CD40 L, CD70, MICA, MICB, and HVEM.

A “costimulatory signal” refers to a signal, which in combination with a primary signal, leads to immune cell (e.g., B cell, T cell, NK cell, or myeloid cell) proliferation and/or upregulation or downregulation of key molecules.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

“Gene construct” refers to a nucleic acid, such as a vector, plasmid, viral genome or the like which includes a “coding sequence” for a polypeptide or which can otherwise transcribe to a biologically active RNA (e.g., antisense, decoy, ribozyme, etc.), may be transfected into cells, e.g., mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The terms “ligand-binding domain” and “antigen-binding domain” are used interchangeably herein, and refer to that portion of a chimeric antigen receptor that binds specifically to a predetermined antigen.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier” means a pharmaceutically acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a natural or synthetic molecule, or some combination thereof, comprising a single nucleotide or two or more nucleotides linked by a phosphate group at the 3′ position of one nucleotide to the 5′ end of another nucleotide. The polymeric form of nucleotides is not limited by length and can comprise either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides. The polynucleotide is not necessarily associated with the cell in which the nucleic acid is found in nature, and/or operably linked to a polynucleotide to which it is linked in nature.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

A “signal transducing domain” or “signaling domain” of a CAR, as used herein, is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRζ, FcRγ, FcRβ, FcRε, CD3γ, CD3δ, CD3ε, CD3ζ, CD5, CD22, CD28, 4-1BB, CD79a, CD79b and CD66d.

A “spacer” as used herein refers to a peptide that joins the proteins (e.g., those in a fusion protein). Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

The term “specifically binds” or “specific binding”, as used herein, when referring to a polypeptide refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule. For example, in the case of the ability of a PIG-specific CAR to bind to a peptide presented on an MHC (e.g., class I MHC or class II MHC); typically, a CAR specifically binds to its peptide/MHC with an affinity of at least a K_{D} of about 10-4 M or less, and binds to the predetermined antigen/binding partner with an affinity (as expressed by KD) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “variant” refers to an amino acid or peptide sequence having conservative amino acid substitutions, non-conservative amino acid substitutions (e.g., a degenerate variant), substitutions within the wobble position of each codon (e.g., DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, to which the nucleic acid has been linked, and may or may not be able to replicate autonomously or integrate into a chromosome of a host cell. Such vectors may include any vector, (e.g., a plasmid, cosmid or phage chromosome) containing a gene construct in a form suitable for expression by a cell (e.g., linked to a transcriptional control element).

### Chimeric Antigen Receptors (CARs)

Chimeric antigen receptors (CARs) are receptors comprising a targeting moiety that is associated with one or more signaling domains and/or costimulatory domains in a single fusion molecule. In some aspects, the binding moiety of a CAR comprises an extracellular domain comprising: (i) a CD3 extracellular domain or fragment thereof; (ii) an antigen-binding domain specific for an idiotype of an anti-CD3 antibody; or (iii) an antigen-binding domain specific for an Fc domain. In some embodiments, the antigen-binding domain is a single-chain variable fragment (scFv), comprising the light and heavy chain variable fragments of a monoclonal antibody joined by a flexible linker. In certain embodiments, the CAR further comprises a transmembrane domain, a transmembrane domain, and an intracellular signaling domain.

### CD3 Extracellular Domain

In some embodiments, provided herein are CARs comprising an extracellular domain comprising a CD3 extracellular domain or fragment thereof.

The term “CD3,” as used herein, refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma. Human CD3-epsilon comprises the amino acid sequence as set forth in SEQ ID NO:116 of U.S. Patent Application Publication No. US 2020/0024356A1, the content of which is incorporated by reference herein in its entirety; human CD3-delta comprises the amino acid sequence as set forth in SEQ ID NO:117 of U.S. Patent Application Publication No. US 2020/0024356A1, the content of which is incorporated by reference herein in its entirety; human CD3-zeta comprises the amino acid sequence as set forth in SEQ ID NO: 118 of U.S. Patent Application Publication No. US 2020/0024356A1, the content of which is incorporated by reference herein in its entirety; and CD3-gamma comprises the amino acid sequence as set forth in SEQ ID NO 119 of U.S. Patent Application Publication No. US 2020/0024356A1, the content of which is incorporated by reference herein in its entirety. All references to proteins, polypeptides and protein fragments herein are intended to refer to the human version of the respective protein, polypeptide or protein fragment unless explicitly specified as being from a non-human species. Thus, the expression “CD3” means human CD3 unless specified as being from a non-human species, e.g., “mouse CD3,” “monkey CD3,” etc.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. Cell surface-expressed CD3 includes CD3 proteins contained within the context of a functional T cell receptor in the membrane of a cell. Cell surface-expressed CD3 includes CD3 protein expressed as part of a homodimer or heterodimer on the surface of a cell (e.g., gamma/epsilon, delta/epsilon, and zeta/zeta CD3 dimers). Cell surface-expressed CD3 also includes a CD3 chain (e.g., CD3-epsilon, CD3-delta or CD3-gamma) that is expressed by itself, without other CD3 chain types, on the surface of a cell. A cell surface-expressed CD3 can comprise or consist of a CD3 protein expressed on the surface of a cell which normally expresses CD3 protein. Alternatively, cell surface-expressed CD3 can comprise or consist of CD3 protein expressed on the surface of a cell that normally does not express human CD3 on its surface but has been artificially engineered to express CD3 on its surface.

In some embodiments, the CD3 extracellular domain or fragment thereof is a human CD3 extracellular domain or a fragment thereof, such as a human CD3& extracellular domain or fragment thereof, a human CD38 extracellular domain or fragment thereof, a human CD3γ extracellular domain or fragment thereof, or a human CD35 extracellular domain or fragment thereof. In some embodiments, the extracellular domain of a human CD38 is set forth in SEQ ID NO: 33, the extracellular domain of a human CD38 is set forth in SEQ ID NO: 34, and the extracellular domain of a human CD3Y is set forth in SEQ ID NO: 35 of WO 2016/085889, which is incorporated by reference herein in its entirety.

In some embodiments, the CD3 extracellular domain or fragment thereof comprises at least 10, 15, 20, 25, 30, 35, 40, 45, 50, 55, 60, 65, 70, 75, 80, 85, 90, 95, 100, 105, 110, or 113 consecutive amino acids of SEQ ID NO: 33 of WO 2016/085889 (i.e., SEQ ID NO: 1959 shown in Table 22 below). In some embodiments, the CD3 extracellular domain or fragment thereof comprises an amino acid sequence at least at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity to SEQ ID NO: 33 of WO 2016/085889 (i.e., SEQ ID NO: 1959 shown in Table 22 below). In some embodiments, the CD3 extracellular domain or fragment thereof comprises an amino acid sequence of SEQ ID NO: 33 of WO 2016/085889 (i.e., SEQ ID NO: 1959 shown in Table 22 below).

TABLE 22
Examples of CD3 extracellular domain

GVWGQDGNEEMGGITQTPYKVSISGTTVILTCPQYPGSEILWQHNDKNI
GGDEDDKNIGSDEDHLSLKEFSELEQSGYYVCYPRGSKPEDANFYLYLR
ARVCENCMEMDVMSV (SEQ ID NO: 1959)

In some embodiments, the CD3 extracellular domain or fragment thereof comprises at least 10, 15, 20, 25, 30, 35, 40, 45, 50, 55, 60, 65, 70, 75, 80, 85, or 87 consecutive amino acids of SEQ ID NO: 34 of WO 2016/085889. In some embodiments, the CD3 extracellular domain or fragment thereof comprises an amino acid sequence at least at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity to SEQ ID NO: 34 of WO 2016/085889. In some embodiments, the CD3 extracellular domain or fragment thereof comprises an amino acid sequence of SEQ ID NO: 34 of WO 2016/085889.

In some embodiments, the CD3 extracellular domain or fragment thereof comprises at least 10, 15, 20, 25, 30, 35, 40, 45, 50, 55, 60, 65, 70, 75, 80, 85, 90, 95, or 97 consecutive amino acids of SEQ ID NO: 35 of WO 2016/085889. In some embodiments, the CD3 extracellular domain or fragment thereof comprises an amino acid sequence at least at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity to SEQ ID NO: 35 of WO 2016/085889. In some embodiments, the CD3 extracellular domain or fragment thereof comprises an amino acid sequence of SEQ ID NO: 35 of WO 2016/085889.

In some embodiments, the CD3 extracellular domain or fragment thereof comprises an epitope recognized by an anti-CD3 antibody. As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). The antibodies and antigen-binding fragments disclosed herein may bind soluble CD3 and/or cell surface expressed CD3. Soluble CD3 includes natural CD3 proteins as well as recombinant CD3 protein variants such as, e.g., monomeric and dimeric CD3 constructs, which lack a transmembrane domain or are otherwise unassociated with a cell membrane. In some embodiments, the anti-CD3 antibody is selected from the anti-CD3 antibodies listed in Table 6.

### Antigen-Binding Domain Specific for an Idiotype of an Anti-CD3 Antibody

In certain embodiments, the binding domain and/or extracellular domain of a CAR provided herein provides the CAR with the ability to bind to a target antigen of interest. A binding domain (e.g., a ligand-binding domain or antigen-binding domain) can be any protein, polypeptide, oligopeptide, or peptide that possesses the ability to specifically recognize and bind to a biological molecule (e.g., a cell surface receptor or tumor protein, or a component thereof). A binding domain includes any naturally occurring, synthetic, semi-synthetic, or recombinantly produced binding partner for a biological molecule of interest. For example, and as further described herein, a binding domain may be antibody light chain and heavy chain variable regions, or the light and heavy chain variable regions can be joined together in a single chain and in either orientation (e.g., V_{L}-V_{H} or V_{H}-V_{L}). A variety of assays are known for identifying binding domains of the present disclosure that specifically bind with a particular target, including Western blot, ELISA, flow cytometry, or surface plasmon resonance analysis (e.g., using BIACORE analysis). Exemplary methods of producing anti-idiotypic antibodies are described in U.S. Pat. No. 10,150,817 B2 and in Example 1 of WO 2017/162587 A1, each of which is incorporated by reference in its entirety.

In some embodiments, the binding domain and/or extracellular domain of the CARs provided herein comprise an antigen-binding domain specific for an idiotype of an anti-CD3 antibody. In some embodiments, the anti-CD3 antibody is selected from the anti-CD3 antibodies listed in Table 6. In certain embodiment, the anti-CD3 antibody is an anti-CD3 antibody designated as CH2527 in WO 2017/162587 A1. For example, in some embodiments, the anti-CD3 antibody comprises a CDR H1 sequence, a CDR H2 sequence, and a CDR H3 sequence of SEQ ID NOs: 11, 12, and 13 disclosed in WO 2017/162587, respectively, incorporated herewith by reference in its entirety. In some embodiments, the anti-CD3 antibody comprises a CDR H1 sequence, a CDR H2 sequence, and a CDR H3 sequence of SEQ ID NOs: 44, 45, and 46 disclosed in WO 2017/162587, respectively, incorporated herewith by reference in its entirety. In some embodiment, the anti-CD3 antibody comprises a variable heavy chain (VH) sequence of SEQ ID No: 43 disclosed in WO 2017/162587, which is incorporated herewith by reference in its entirety.

In some embodiments, the antigen-binding domain described herein is a single chain fragment variable (scFv) specific for an idiotype of an anti-CD3 antibody, and may be a murine, human or humanized scFv. In some embodiments, the single chain fragment variable (scFv) specific for an idiotype of an anti-CD3 antibody is the scFvs designated as 4.15.64 or 4.32.63 disclosed in WO 2017/162587, which is incorporated herewith by reference in its entirety.

In some embodiments, the antigen-binding domain comprises the heavy chain and/or the light chain CDR sequences of a scFv listed in Table 1. In some embodiments, the antigen-binding domain comprises the heavy chain and/or the light chain variable region sequences of a scFv listed in Table 1. In some embodiments, the heavy chain variable region disclosed herein may comprise at least 50%, at least 60%, at least 70%, at least 80%, at least 90%, at least 91%, at least 92%, at least 93%, at least 94%, at least 95%, at least 96%, at least 97%, at least 98%, or at least 99% homology to a heavy chain variable region sequence of a scFv listed in Table 1. In some embodiments, the light chain variable region disclosed herein may comprise at least 50%, at least 60%, at least 70%, at least 80%, at least 90%, at least 91%, at least 92%, at least 93%, at least 94%, at least 95%, at least 96%, at least 97%, at least 98%, or at least 99% homology to a light chain variable region sequence of a scFv listed in Table 1. In some embodiments, the antigen-binding domain comprises the amino acid sequence of a scFv listed in Table 1. In some embodiments, the scFv described herein may comprise at least 50%, at least 60%, at least 70%, at least 80%, at least 90%, at least 91%, at least 92%, at least 93%, at least 94%, at least 95%, at least 96%, at least 97%, at least 98%, or at least 99% homology to a scFv sequence listed in Table 1.

TABLE 1
Examples of Antigen-binding domains specific for an idiotype of an anti-CD3 antibody

| | | SEQ |
|---|---|---|
| Anti-ID | Sequence | ID NO. |
| 4.15.64 | QIQLVQSGPELKKPGETVRISCKASGYTFTDYSIHWVKQAPGKCLKWMGWINTETGEPAY | 15 |
| anti- | ADDFKGRFAFSLETSASTAYLQINNLKNEDTATFFCAHPYDYDVLDYWGQGTSVTVSSGG | |
| idiotypic | GGSGGGGSGGGGSGGGGSDTVLTQSPASLGVSLGQRATISCRASKSVSTSNYSYIHWYQQ | |
| scFv | KPGQPPKLLIKYVSYLESGVPARFSGSGSGTDFTLNIHPVEEEDAATYYCQHSREFPWTF | |
| | GCGTKLEIK | |
| | (CDR sequences are in bold) | |
| Anti-ID | DYSIH | 16 |
| 4.15.64 | | |
| CDR | | |
| H1 Kabat | | |
| Anti-ID | WINTETGEPAYADDFKG | 17 |
| 4.15.64 | | |
| CDR | | |
| H2 Kabat | | |
| Anti-ID | PYDYDVLDY | 18 |
| 4.15.64 | | |
| CDR | | |
| H3 Kabat | | |
| Anti-ID | RASKSVSTSNYSYIH | 19 |
| 4.15.64 | | |
| CDR L 1 | | |
| Kabat | | |
| Anti-ID | YVSYLES | 20 |
| 4.15.64 | | |
| CDR L2 | | |
| Kabat | | |
| Anti-ID | QHSREFPWT | 21 |
| 4.15.64 | | |
| CDR L3 | | |
| Kabat | | |
| 4.32.63 | QVQLKESGPGLVAPSQSLSITCTVSGFSLTSYGVSWVRQPPGKCLEWLGIIWGDGSTNYH | 23 |
| anti- | SALISRLSISKDNSKSQVFLKLNSLQTDDTATYYCAKGITTVVDDYYAMDYWGQGTSVTV | |
| idiotypic | SSGGGGSGGGGSGGGGSGGGGSDIQMTQSPASLSASVGETVTITCRASENIDSYLAWYQQ | |
| scFv | KQGKSPQLLVYAATFLADDVPSRFSGSGSGTQYSLKINSLQSEDVARYYCQHYYSTPYTF | |
| | GCGTKLEIK | |
| | (CDR sequences are in bold) | |
| Anti-ID | SYGVS | 24 |
| 4.32.63 | | |
| CDR | | |
| H1 Kabat | | |
| Anti-ID | IIWGDGSTNYHSALIS | 25 |
| 4.32.63 | | |
| CDR | | |
| H2 Kabat | | |
| Anti-ID | GITTVVDDYYAMDY | 29 |
| 4.32.63 | | |
| CDR | | |
| H3 Kabat | | |
| Anti-ID | RASENIDSYLA | 30 |
| 4.32.63 | | |
| CDR L 1 | | |
| Kabat | | |
| Anti-ID | AATFLAD | 31 |
| 4.32.63 | | |
| CDR L2 | | |
| Kabat | | |
| Anti-ID | QHYYSTPYT | 32 |
| 4.32.63 | | |
| CDR L3 | | |
| Kabat | | |

In some embodiments, provided herein is a chimeric antigen receptor (CAR) polypeptide comprising (a) an extracellular domain comprising an antigen-binding domain specific for an idiotype of an anti-CD28 antibody; (b) a hinge domain; (c) a transmembrane domain; and (d) an intracellular signaling domain. Such CARs can be used in combination of an anti-CD28 x TAA to target a tumor cell expressing the TAA.

### Antigen-Binding Domain Specific for an Fc Domain

In some embodiments, the extracellular domain comprises the antigen binding domain specific for an Fc domain. In some embodiments, the Fc domain is selected from a human IgG1 Fc domain, a human IgG2 Fc domain, a human IgG3 Fc domain, and a human IgG4 Fc domain. In some embodiments, the Fc domain is an IgG3 Fc domain. In some embodiments, the Fc domain comprises the amino acid sequence of an Fc shown in FIG. 3.

In some embodiments, the antigen-binding domain is a single chain fragment variable (scFv). In some embodiments, the single chain fragment variable (scFv) specific for an idiotype of an anti-CD3 antibody or for an Fc domain may be a murine, human or humanized scFv. Single chain antibodies may be cloned from the V region genes of a hybridoma specific for a desired target. A technique which can be used for cloning the variable region heavy chain (VH) and variable region light chain (VL) has been described, for example, in Orlandi et al., PNAS, 1989; 86: 3833-3837. Thus, in certain embodiments, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In certain embodiments, the CARs of the present disclosure may comprise a linker between the various domains, added for appropriate spacing and conformation of the molecule. For example, in one embodiment, there may be a linker between the binding domain VH or V_{L} which may be between 1-10 amino acids long. In other embodiments, the linker between any of the domains of the chimeric antigen receptor may be between 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long. Ranges including the numbers described herein are also included herein, e.g., a linker 10-30 amino acids long.

In certain embodiments, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains. The ordinarily skilled artisan will recognize that design of a CAR can include linkers that are all or partially flexible, such that the linker can include a flexible linker as well as one or more portions that confer less flexible structure to provide for a desired CAR structure.

### Hinge Domain, Transmembrane Domain, and Intracellular Signaling Domain

The binding domain of the CAR may be followed by a “spacer,” or, “hinge,” which refers to the region that moves the extracellular domain or binding domain (e.g., a CD3 extracellular domain, or an antigen-binding domain specific for an idiotype of an anti-CD3 antibody or for an Fc domain as described herein) away from the effector cell surface to enable proper cell/cell contact, antigen binding, and activation (Patel et al., Gene Therapy, 1999; 6: 412-419). The hinge region in a CAR is generally between the transmembrane (TM) and the extracellular domain or binding domain (e.g., a CD3 extracellular domain, or an antigen-binding domain specific for an idiotype of an anti-CD3 antibody or for an Fc domain as described herein). In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8a, CD4, CD28 and CD7, which may be wild-type hinge regions from these molecules or may be altered.

In some embodiments, the CAR described herein further comprises a hinge domain. The hinge domain may be selected from the hinge domains of Table 2 below. In certain embodiments, the hinge domain is a CD28 or CD8 hinge domain. In some embodiments, the CAR further comprises a hinge domain comprising an amino acid sequence with at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity to an amino acid sequence set forth in Table 2. In specific embodiments, the hinge domain comprises an amino acid sequence selected from SEQ ID NOs:1-5.

TABLE 2
Example hinge domains

| | | SEQ |
|---|---|---|
| | | ID |
| Hinge domain | Amino Acid Sequence | NO. |
| IgG4(12 aa) | ESKYGPPCPSCP | 33 |
| IgG4m(S228P) | ESKYGPPCPPCP | 34 |
| IgG4m(S228P)- | ESKYGPPCPPCPAPEFLGGPSVFLFPPKPKDTLMISRTPEVTCVVVDVSQEDPEVQ | 35 |
| CH2-CH3 | FNWYVDGVEVHNAKTKPREEQFNSTYRVVSVLTVLHQDWLNGKEYKCKVSNK | |
| (229aa) | GLPSSIEKTISKAKGQPREPQVYTLPPSQEEMTKNQVSLTCLVKGFYPSDIAVEW | |
| | ESNGQPENNYKTTPPVLDSDGSFFLYSRLTVDKSRWQEGNVFSCSVMHEALHN | |
| | HYTQKSLSLSLGK | |
| IgG4m(S228P)- | ESKYGPPCPPCPGQPREPQVYTLPPSQEEMTKNQVSLTCLVKGFYPSDIAVEWES | 36 |
| CH3(119 aa) | NGQPENNYKTTPPVLDSDGSFFLYSRLTVDKSRWQEGNVFSCSVMHEALHNHY | |
| | TQKSLSLSLGK | |
| IgG4m(S228P)- | ESKYGPPCPPCPAPPVAGPSVFLFPPKPKDTLMISRTPEVTCVVVDVSQEDPEVQF | 39 |
| CH2(APEFLG | NWYVDGVEVHNAKTKPREEQFNSTYRVVSVLTVLHQDWLNGKEYKCKVSNK | |
| (SEQ ID NO: | GLPSSIEKTISKAKGQPREPQVYTLPPSQEEMTKNQVSLTCLVKGFYPSDIAVEW | |
| 37)→APPVA | ESNGQPENNYKTTPPVLDSDGSFFLYSRLTVDKSRWQEGNVFSCSVMHEALHN | |
| (SEQ ID NO: | HYTQKSLSLSLGK | |
| 38))-CH3 | | |
| IgG4m(S228P)- | ESKYGPPCPPCPAPPVAGPSVFLFPPKPKDTLMISRTPEVTCVVVDVSQEDPEVQF | 40 |
| CH2 (APEFLG | NWYVDGVEVHNAKTKPREEQFQSTYRVVSVLTVLHQDWLNGKEYKCKVSNK | |
| (SEQ ID NO: | GLPSSIEKTISKAKGQPREPQVYTLPPSQEEMTKNQVSLTCLVKGFYPSDIAVEW | |
| 37)→APPVA | ESNGQPENNYKTTPPVLDSDGSFFLYSRLTVDKSRWQEGNVFSCSVMHEALHN | |
| (SEQ ID NO: | HYTQKSLSLSLGK | |
| 38)) + N297Q)- | | |
| CH3 | | |
| IgG1 hinge | EPKSCDKTHTCPPCP | 41 |
| IgG2 hinge | ERKCCVECPPCP | 42 |
| IgG3 hinge | LKTPLGDTTHTCPRCPEPKSCDTPPPCPRCPEPKSCDTPPPCPRCPEPKSCDTPPPC | 43 |
| | PRCP | |
| IgA hinge | PVPSTPPTPSPSTPPTPSPSC | 44 |
| IgD hinge | ESPKAQASSVPTAQPQAEGSLAKATTAPATTRNTGRGGEEKKKEKEKEEQEERE | 45 |
| | TKTP | |
| human CD8α | KPTTTPAPRPPTPAPTIASQPLSLRPEACRPAAGGAVHTRGLDFACDIY | 1 |
| human CD8α | AKPTTTPAPRPPTPAPTIASQPLSLRPEACRPAAGGAVHTRGLDFACD | 2 |
| (48 aa) | | |
| human CD8α | TTTPAPRPPTPAPTIASQPLSLRPEACRPAAGGAVHTRGLDFACD | 3 |
| (45 aa) | | |
| CD8b hinge-1 | SVVDFLPTTAQPTKKSTLKKRVCRLPRPETQKGPLCSP | 46 |
| CD8b hinge-2 | VDFLPTTAQPTKKSTLKKRVCRLPRPETQKGPLCSP | 47 |
| human CD28- | IEVMYPPPYLDNERSNGTIIHVKGKHLCPSPLFPGPSKP | 4 |
| HD1 | | |
| human CD28- | IEVMYPPPYLDNEKSNGTIIHVKGKHLCPSPLFPGPSKP | 5 |
| HD2 | | |
| murine CD4 | LSEGDKVKMDSRIQVLSRGVNQT | 48 |
| hinge | | |
| CD4 hinge-1 | SGQVLLESNIKVLPTWSTPVQP | 49 |
| CD4 hinge-2 | DSGQVLLESNIKVLPTWSTPVQP | 50 |
| murine CD3ζ | QSFGLLDPK | 51 |
| hinge | | |

In some embodiments, the CAR described herein further comprises a transmembrane domain. The “transmembrane” region or domain is the portion of the CAR that anchors the extracellular binding portion to the plasma membrane of the immune effector cell, and facilitates binding of the extracellular domain or the binding domain (e.g., a CD3 extracellular domain or an antigen-binding domain specific for an idiotype of an anti-CD3 antibody or for an Fc domain as described herein) to its binding partner. In some embodiments, the transmembrane domain may be a CD28 transmembrane domain. Other transmembrane domains that may be employed in some embodiments include those obtained from CD8, CD8a, CD4, CD28, CD45, CD9, CD16, CD22, CD33, CD64, CD80, CD86, CD134, CD137, and CD154. In certain embodiments, the transmembrane domain is synthetic in which case it would comprise predominantly hydrophobic residues such as leucine and valine.

In some embodiments, the transmembrane domain is selected from a transmembrane domain of CD28, CD8a, ICOS, 4-1BB, CD4, Tim4, OX40, CD27, CD2, LFA-1, CD30, CD40, PD-1, CD7, LIGHT, NKG2C, B7-H3, NKG2D, NKp44, NKp46, DAP12, CD16, NKp30, FcRγ, DAP10, 2B4, or DNAM-1. In some embodiments, the transmembrane domain may be selected from the transmembrane domains of Table 3 below. In certain embodiments, the transmembrane domain is an NKG2D transmembrane domain, an NKG2D inverted transmembrane domain, a CD28 transmembrane domain, a CD8 transmembrane domain, a CD16 transmembrane domain, or a FcgR1 (CD64) transmembrane domain. In some embodiments, the CAR further comprises a transmembrane domain comprising an amino acid sequence with at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity to an amino acid sequence set forth in Table 3. In specific embodiments, the transmembrane domain comprises an amino acid sequence selected from SEQ ID NOs: 6-13.

TABLE 3
Example transmembrane domains

| Transmembrane | | SEQ ID |
|---|---|---|
| domain | Amino Acid Sequence | NO: |
| human CD28- | FWVLVVVGGVLACYSLLVTVAFIIFWV | 6 |
| TMD1 | | |
| human CD28- | MFWVLVVVGGVLACYSLLVTVAFIIFWV | 7 |
| TMD2 | | |
| human CD8α- | IWAPLAGTCGVLLLSLVITLY | 8 |
| TMD1 | | |
| human CD8α- | IYIWAPLAGTCGVLLLSLVITLYC | 9 |
| TMD2 | | |
| ICOS-TMD | FWLPIGCAAFVVVCILGCILI | 52 |
| 4-1BB TMD1 | IISFFLALTSTALLFLLFFLTLRFSVV | 53 |
| 4-1BB TMD2 | IISFFLALTSTALLFLLFFLTLRFSVVKRGR | 54 |
| CD4 TMD1 | TWSTPVQPMALIVLGGVAGLLLFIGLGIFFSVRS | 55 |
| CD4 TMD2 | MALIVLGGVAGLLLFIGLGIFF | 56 |
| Tim4 | LLMIIAPSLGFVLFALFVAFL | 57 |
| OX40 TMD1 | VAAILGLGLVLGLLGPLAILL | 58 |
| OX40 TMD2 | VAAILGLGLVLGLLGPLAILLALYLL | 59 |
| CD27 | ILVIFSGMFLVFTLAGALFLH | 60 |
| CD2 | IYLIIGICGGGSLLMVFVALLVFYIT | 61 |
| LFA-1 | IAAIVGGTVAGIVLIGILLLVIW | 62 |
| CD30 | PVLDAGPVLFWVILVLVVVVGSSAFLLC | 63 |
| CD40 | ALVVIPIIFGILFAILLVLVFI | 64 |
| PD-1 | VGVVGGLLGSLVLLVWVLAVI | 65 |
| CD7 | AALAVISFLLGLGLGVACVLA | 66 |
| LIGHT | VGLGLLLLLMGAGLAVQGWFL | 67 |
| NKG2C | LTAEVLGIICIVLMATVLKTIVL | 68 |
| B7-H3 | LWVTVGLSVCLIALLVALAFV | 69 |
| NKG2D-TMD1 | PFFFCCFIAVAMGIRFIIMVA | 70 |
| NKG2D-TMD2 | PFFFCCFIAVAMGIRFIIMVT | 71 |
| NKp44 | LVPVFCGLLVAKSLVLSALLVWWG | 72 |
| NKp46 | MGLAFLVLVALVWFLVEDWLSRKR | 73 |
| DAP12 | GVLAGIVMGDLVLTVLIALAV | 74 |
| FcγRIIIa | VSFCLVMVLLFAVDTGLYFSV | 10 |
| (CD16)-TMD1 | | |
| NKp30 | AGTVLLLRAGFYAVSFLSVAVGST | 75 |
| FcγRIIIa | VSFCLVMVLLFAVDTGLYFSVKTN | 11 |
| (CD16)-TMD2 | | |
| DAP10-TMD1 | LLAGLVAADAVASLLIVGAVF | 76 |
| DAP10-TMD2 | ILLAGLVAADAVASLLIVGAVFLCARR | 77 |
| 2B4 | FLVIIVILSALFLGTLACFCV | 78 |
| DNAM-1 | YTLFVAGGTVLLLLFVISITTIIVIFLN | 79 |
| CD148 | AVFGCIFGALVIVTVGGFIFW | 80 |
| CD3 zeta- | LCYLLDGILFIYGVILTALFLRV | 81 |
| TMD1 | | |
| CD3 zeta- | LCYLLDGILFIYGVILTALFL | 82 |
| TMD2 | | |
| CD62L | PLFIPVAVMVTAFSGLAFIIWLA | 83 |
| CD8 | MALPVTALLLPLALLLHAARP | 84 |
| LAG-3 | LLFLILGVLSLLLLVTGAFGF | 85 |
| Her2 | SIISAVVGILLVVVLGVVFGILII | 86 |
| GITR | PLGWLTVVLLAVAACVLLLTSAQLGLHIWQL | 87 |
| NKG2D | VASWITVMIIFRIGMAVAIFCCFFF | 12 |
| inverted TM | | |
| FcgR1 Tm | VLFYLAVGIMFLVNTVLWVTI | 13 |

In certain embodiments, the CARs provided herein further comprise an intracellular signaling domain. The intracellular signaling domain (also referred to herein as the “signaling domain”) comprises the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

In certain embodiments, the CARs provided herein comprise one or more immunoreceptor tyrosine-based activation motifs or ITAMs. Examples of ITAM containing primary cytoplasmic signaling sequences that are of use include those derived from TCRζ, FcRgamma, FcRβ, CD3γ, CD3δ, CD3ε, CD3ζ, CD5, CD22, CD28, 4-1BB, CD79a, CD79b and CD66d. In one embodiment, the intracellular signaling domain of the CARs described herein are derived from CD3ζ.

In certain embodiments, the CARs provided herein further comprise a costimulatory domain. Costimulatory molecules are cell surface molecules other than antigen receptors or Fc receptors that provide a second signal required for efficient activation and function of immune cells (e.g., T lymphocytes or NK cells) upon binding to antigen. Examples of such co-stimulatory molecules include CD27, CD28, 4-1BB (CD137), OX40 (CD134), CD30, CD40, ICOS (CD278), CD2, LIGHT, and NKD2C. Accordingly, while the present disclosure provides exemplary costimulatory domains derived from CD28. The inclusion of one or more co-stimulatory signaling domains may enhance the efficacy and expansion of T cells expressing CAR receptors. Also disclosed herein are CAR polypeptides, wherein the intracytoplasmic/costimulatory region of the CAR polypeptide further comprises a 4-1BB domain (e.g., in addition to a CD28/domain). The costimulatory region of such a CAR polypeptide may comprise a complete 4-1BB domain or fragment thereof, and/or a complete CD28/ζ domain or fragment thereof. The intracellular signaling and costimulatory signaling domains may be linked in any order in tandem to the carboxyl terminus of the transmembrane domain.

In some embodiments, the intracellular signaling domain is FcgR1 intracellular signaling domain, a 4-1BB-CD3z intracellular signaling domain, a 2B4-CD3z intracellular signaling domain, a CD16 intracellular signaling domain, a CD64 intracellular signaling domain, or a CD28-CD3z intracellular signaling domain.

In certain aspects, the CAR polypeptides provided herein comprise at least one intracytoplasmic/costimulatory region comprising a cluster of differentiation 28 zeta (CD28/ζ) domain. Additionally, the hinge/spacer region and/or the transmembrane region of the CAR or the transmembrane region of the CAR may comprise a CD28/ζ domain. The CAR may comprise at least one cluster of differentiation 28 zeta (CD28/ζ) amino acid sequence selected from the amino acid sequences set forth in SEQ ID NOs. 5, 6, and 22. A CAR polypeptide disclosed herein may comprise at least one cluster of differentiation 28 zeta (CD28/ζ) amino acid sequence with at least 50%, at least 60%, at least 70%, at least 80%, at least 90%, at least 91%, at least 92%, at least 93%, at least 94%, at least 95%, at least 96%, at least 97%, at least 98%, or at least 99% homology to an amino acid sequence set forth in SEQ ID NOs. 5, 6, and 22. The CAR polypeptide may comprise all three sequences set forth in SEQ ID NOs 5, 6, and 22. For example, the CAR hinge domain may comprise SEQ ID NO: 5, the transmembrane domain may comprise SEQ ID NO: 6, and the co-stimulatory domain may comprise SEQ ID NO: 22.

TABLE 4
Exemplary CD28/ζ Sequences.

| SEQ ID | | |
|---|---|---|
| NO: | Domain | Sequence |
| 5 | Hinge | IEVMYPPPYLDNEKSNGTIIHVKGKHLCPSPLFPGPSKP |
| 6 | Transmembrane | FWVLVVVGGVLACYSLLVTVAFIIFWV |
| 22 | Co-Stimulatory | RSKRSRLLHSDYMNMTPRRPGPTRKHYQPYAPPRDFAAYRS |

The CAR polypeptide sequence disclosed herein may comprise any one of the amino acid sequences set forth in SEQ ID NO 26 to 28. A CAR polypeptide disclosed herein may comprise at least amino acid sequence with at least 50%, at least 60%, at least 70%, at least 80%, at least 90%, at least 91%, at least 92%, at least 93%, at least 94%, at least 95%, at least 96%, at least 97%, at least 98%, or at least 99% homology to an amino acid sequence set forth in SEQ ID NO: 26 to 28.

TABLE 5
Additional Exemplary CAR Sequences:

| | | |
|---|---|---|
| SEQ ID NO: 26 | CD3ζ | RVKFSRSADAPAYQQGQNQLYNELNLGRREEYD |
| | | VLDKRRGRDPEMGGKPQRRKNPQEGLYNELQK |
| | | DKMAEAYSEIGMKGERRRGKGHDGLYQGLSTA |
| | | TKDTYDALHMQALPPR |
| SEQ ID NO: 27 | CD8 | TTTPAPRPPTPAPTIASQPLSLRPEACRPAAGGAV |
| | | HTRGLDFACDIYIWAPLAGTCGVLLLSLVITLYC |
| SEQ ID NO: 28 | 4-1BB | KRGRKKLLYIFKQPFMRPVQTTQEEDGCSCRFPE |
| | | EEEGGCEL |

### Nucleic Acids and Vectors

In certain aspects, also disclosed are nucleic acids and polynucleotide vectors encoding the CAR polypeptides disclosed herein.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

In certain embodiments, the polynucleotide encoding the CAR described herein is inserted into a vector. The vector is a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra. An expression vector may comprise additional elements, for example, the expression vector may have two replication systems, thus allowing it to be maintained in two organisms, for example in human cells for expression and in a prokaryotic host for cloning and amplification.

The expression vector may have the necessary 5′ upstream and 3′ downstream regulatory elements such as promoter sequences such as CMV, PGK and EF1alpha. promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector may have additional sequence such as 6×-histidine (SEQ ID NO: 88), c-Myc, and FLAG tags which are incorporated into the expressed CARs. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector may also be engineered for replication and/or expression functionality (e.g., transcription and translation) in a particular cell type, cell location, or tissue type. Expression vectors may include a selectable marker for maintenance of the vector in the host or recipient cell.

In various embodiments, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontech), pCl-neo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the CARs disclosed herein can be ligated into such expression vectors for the expression of the chimeric protein in mammalian cells.

In certain embodiments, the nucleic acids encoding the CAR are provided in a viral vector. A viral vector can be that derived from, for example, a retrovirus (e.g., a foamy virus) or lentivirus. As used herein, the term, “viral vector,” refers to a nucleic acid vector construct that includes at least one element of viral origin and has the capacity to be packaged into a viral vector particle. The viral vector can contain the coding sequence for the various chimeric proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the viral vector containing the coding sequence for a CAR described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Murine Stem Cell Virus (MSCV) and Rous Sarcoma Virus (RSV)). Retroviruses” also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72: 8463-8471 and Zufferey et al., J. Virol. 1998; 72:9873-9880).

Retroviral vectors (i.e., both lentiviral and non-lentiviral) for use can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of a CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vectors provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CARs are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding a CAR carried by a retroviral vector can be transduced into a cell through infection and pro virus integration.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al. (2001, Molecular Cloning: A Laboratory Manual, Cold Spring Harbor Laboratory, New York).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Induced Natural Killer (NK) Cells

In certain aspects, also disclosed herein are Natural Killer (NK) cells that are engineered to express the disclosed CAR polypeptides. Natural-killer (NK) cells are CD56+CD3 large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

In some embodiments, the NK cells are obtained from the subject to be treated (i.e., are autologous). However, in certain embodiments, NK cell lines or donor effector cells (allogeneic) are used. In some embodiments, the NK cell is an inducible NK cells differentiated from an induced pluripotent stem cell (iPSC).

NK cells can be obtained from a number of sources, including peripheral blood mononuclear cells (PBMC), unstimulated leukapheresis products (PBSC), bone marrow, cord blood, human embryonic stem cells (hESCs), induced pluripotent stem cells (iPSCs) by methods well known in the art. NK cells can be detected by specific surface markers, such as CD16, CD56, and CD8 in humans with absence of CD3 expression.

In some embodiment, NK cells may be obtained from blood collected from a subject using any number of techniques known to the skilled artisan. For example, the starting population of NK cells may be obtained by isolating mononuclear cells using ficoll density gradient centrifugation. A specific subpopulation of NK cells can be further isolated by positive or negative selection techniques. For example, NK cells can be isolated using a combination of antibodies directed to surface markers unique to the positively selected cells, e.g., by incubation with antibody-conjugated beads for a time period sufficient for positive selection of the NK cells. Alternatively, enrichment of NK cells population can be accomplished by negative selection using a combination of antibodies directed to surface markers unique to the negatively selected cells. For example, the cell culture may be depleted of any cells expressing CD3, CD14, and/or CD19 cells and may be characterized to determine the percentage of CD56⁺/CD3⁻ cells or NK cells.

In some embodiments, umbilical cord blood (CB) is used to derive NK cells. In some embodiments, the NK cells are isolated and expanded by the previously described method of ex vivo expansion of NK cells. For example, CB mononuclear cells may be isolated by ficoll density gradient centrifugation and cultured in a bioreactor with IL-2 and artificial antigen presenting cells (aAPCs). After a few days, the cell culture is depleted of any cells expressing CD3 and re-cultured for additional days. The cells are again CD3-depleted and characterized to determine the percentage of CD56⁺/CD3⁻ cells or NK cells. In other embodiments, umbilical CB is used to derive NK cells by isolating CD34⁺ cells, and differentiating them into CD56⁺/CD3⁻ cells by culturing the isolated CD34+ cells in a medium containing SCF, IL-7, IL-15, and IL-2.

In some embodiments, NK cells are generated from pluripotent stem cells. The pluripotent stem cells, either human embryonic stem cells (hESCs) or iPSCs, can grow indefinitely in an undifferentiated state via self-renewal. Therefore, the ability to routinely derive NK cells from hESCs and iPSCs allows for an unlimited number of uniform NK cells to be produced from the starting pluripotent stem cell population to provide a standardized, off-the-shelf approach. It has also been reported that iPSC-derived NK (INK) cells can produce inflammatory cytokines and exert strong cytotoxicity against an array of hematologic and solid tumors. hESCs and iPSCs can be engineered to express a CAR described herein using genetic engineering approaches such as transposons and lentiviral delivery which ensure efficient transgene insertion and stable expression in iPSCs. TALENS and CRISPR/Cas9 may also be used for more precision in knocking in or deleting specific genes. The engineered and undifferentiated hESCs or iPSCs may be then used to differentiate into NK cells expressing the CAR described herein. Methods of differentiating hESCs or iPSCs into NK cells are well known in the art, including those described in Cichocki et al, Sci Transl Med. 2020; 12(568):eaaz5618; Goldenson et al. Front Immunol. 2022; 13:841107; Li et al. Cell Stem Cell 2018; 23:181-192; Maddineni S, et al. J Immunother Cancer 2022; 10:e004693, each of which is incorporated herein by this reference in its entirety.

The present disclosure provides methods for making the NK cells which express the CARs described herein. In one embodiment, the method comprises transfecting or transducing NK cells isolated from a subject such that the NK cells express one or more CAR as described herein. In certain embodiments, the NK cells are isolated from an individual and genetically modified without further manipulation in vitro. Such cells can then be directly re-administered into the individual. In further embodiments, the NK cells are expanded in vitro prior to being genetically modified to express a CAR. In this regard, the NK cells may be cultured before or after being genetically modified (i.e., transduced or transfected to express a CAR as described herein). The NK cells may be expanded in the presence of artificial antigen presenting cells (aAPCs). The expansion culture may further comprise cytokines to promote expansion, such as IL-2, IL-21, and/or IL-18. The cytokines may be replenished in the expansion culture, such as every 2-3 days. The APCs may be added to the culture at least a second time, such as after CAR transduction.

In some aspects, also disclosed herein are other immune cells (e.g., phagocytes) that are engineered to express the disclosed CAR polypeptides.

### Binding Properties of the Chimeric Antigen Receptors

As used herein, the term “binding” in the context of the binding of a chimeric antigen receptor comprising an extracellular domain described herein to, e.g., an antigen-binding molecule (e.g., a multi-specific antigen-binding molecule that binds to CD3 and a tumor antigen). Binding typically refers to an interaction or association between a minimum of two entities or molecular structures, such as an antigen-binding domain:antigen interaction. For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the chimeric antigen receptor as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods. 1997, 201(2):223-31; Geuijen, C A, et al. J Immunol Methods. 2005, 302(1-2):68-77).

Accordingly, in some embodiments, a chimeric antigen receptor of the present disclosure binds to an antigen-binding molecule (e.g., a multi-specific antigen-binding molecule that binds to CD3 and a tumor antigen) having an affinity corresponding to a K_{D} value that is at least ten-fold lower than its affinity for binding to a non-specific antigen (e.g., BSA, casein). According to the present disclosure, in some embodiments, the affinity of a chimeric antigen receptor with a K_{D} value that is equal to or less than ten-fold lower than a non-specific binding partner may be considered non-detectable binding.

The term “K_{D}” (M) refers to the dissociation equilibrium constant of a particular antigen-binding domain:antigen interaction. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g., a chimeric antigen receptor) to its interactive partner molecule (e.g. antigen X) compared to the binding affinity of the molecule (e.g., chimeric antigen receptor) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be

The term “k_{d}” (sec-1 or 1/s) refers to the dissociation rate constant of a particular antigen-binding domain:antigen interaction, or the dissociation rate constant of a chimeric antigen receptor. Said value is also referred to as the k_{off} value.

The term “kₐ” (M−1 x sec-1 or 1/M) refers to the association rate constant of a particular antigen-binding domain:antigen interaction, or the association rate constant of a chimeric antigen receptor.

The term “K_{A}” (M−1 or 1/M) refers to the association equilibrium constant of a particular antigen-binding domain:antigen interaction, or the association equilibrium constant of a chimeric antigen receptor. The association equilibrium constant is obtained by dividing the kₐ by the k_{d}.

The term “EC50” or “EC₅₀” refers to the half maximal effective concentration, which includes the concentration of a chimeric antigen receptor that induces a response halfway between the baseline and maximum after a specified exposure time. The EC₅₀ essentially represents the concentration of a chimeric antigen receptor where 50% of its maximal effect is observed. In certain embodiments, the EC₅₀ value equals the concentration of a chimeric antigen receptor of the present disclosure that gives half-maximal binding to cells expressing an antigen (e.g., a tumor-associated antigen), as determined by e.g. a FACS binding assay. Thus, reduced or weaker binding is observed with an increased EC₅₀, or half maximal effective concentration value.

In one embodiment, decreased binding can be defined as an increased EC₅₀ chimeric antigen receptor concentration that enables binding to the half-maximal amount of target cells.

The present disclosure provides chimeric antigen receptors with antigen-binding domains derived from antibodies that bind a human antigen with high affinity (e.g., nanomolar or sub-nanomolar K_{D} values).

According to certain embodiments, the present disclosure provides chimeric antigen receptors with antigen-binding domains derived from corresponding antibodies that bind an antigen-binding molecule (e.g., a multi-specific antigen-binding molecule that binds to CD3 and a tumor antigen) (e.g., at 25° C.) with a K_{D} of less than about 5 nM as measured by surface plasmon resonance. In certain embodiments, the corresponding antibodies bind an antigen-binding molecule (e.g., a multi-specific antigen-binding molecule that binds to CD3 and a tumor antigen) with a K_{D} of less than about 20 nM, less than about 10 nM, less than about 8 nM, less than about 7 nM, less than about 6 nM, less than about 5 nM, less than about 4 nM, less than about 3 nM, less than about 2 nM, less than about 1 nM, less than about 800 pM, less than about 700 pM, less than about 500 pM, less than about 400 pM, less than about 300 pM, less than about 200 pM, less than about 100 pM, less than about 50 pM, or less than about 25 pM as measured by surface plasmon resonance.

The present disclosure also provides chimeric antigen receptors with antigen-binding domains derived from corresponding antibodies that bind an antigen-binding molecule (e.g., a multi-specific antigen-binding molecule that binds to CD3 and a tumor antigen) with a dissociative half-life (t_{1/2}) of greater than about 10 minutes or greater than about 125 minutes as measured by surface plasmon resonance at 25° C. In certain embodiments, the corresponding antibodies bind an antigen-binding molecule (e.g., a multi-specific antigen-binding molecule that binds to CD3 and a tumor antigen) with a t_{1/2} of greater than about 3 minutes, greater than about 4 minutes, greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 110 minutes, or greater than about 120 minutes, as measured by surface plasmon resonance at 25° C.

### Therapeutic Antibodies

### General

In certain aspects, the methods and compositions provided herein relate to the use of therapeutic antibodies (e.g., bispecific CD3-binding antibodies).

As set forth above, as used herein, the term “antibody” encompasses both full antibody molecules and antigen-binding fragments of full antibody molecules. Non-limiting examples of antigen-binding fragments include: (i) Fab fragments; (ii) F(ab′)2 fragments; (iii) heavy chain of the Fab (Fd) fragments; (iv) Fv fragments; (v) single-chain Fv (scFv) molecules; (vi) domain antibody (dAb) fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} Or V_{L}-V_{L} dimers. Alternatively, the antigen-binding fragment of an antibody may contain a monomeric V_{H} Or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody disclosed herein include: (i) V_{H}-C_{H}1; (ii) V_{H}-C_{H}2; (iii) V_{H}-C_{H}3; (iv) V_{H}-C_{H}1-C_{H}2; (v) V_{H}—C_{H}1-C_{H}2-C_{H}3; (vi) V_{H}-C_{H}2-C_{H}3; (vii) V_{H}-C_{L}; (viii) V_{H}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody disclosed herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody disclosed herein using routine techniques available in the art. In certain embodiments provided herein, at least one variable domain of a multispecific antibody is capable of specifically binding to CD3.

In some embodiments, the antibodies provided herein may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody disclosed herein in the presence of complement. “Antibody-dependent cell-mediated cytotoxicity” (ADCC) refers to a cell-mediated reaction in which nonspecific cytotoxic cells that express Fc receptors (FCRS) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments provided herein, the CD3 multispecific (e.g., bispecific or trispecific) antibodies provided herein are human antibodies. The term “human antibody,” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies provided herein, in some embodiments, may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. In certain embodiments, the disclosure encompasses antibodies having one or more mutations in the hinge, C_{H}2 or C_{H}3 region which may be desirable, for example, in production, to improve the yield of the desired antibody form.

The antibodies disclosed herein may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes disclosed herein. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

In certain embodiments, the methods and compositions provided herein include one-arm antibodies that bind a tumor antigen (TAA). As used herein, a “one-arm antibody” means an antigen-binding molecule comprising a single antibody heavy chain and a single antibody light chain.

### Sequence Variants

In some embodiments, the CD3 multispecific (e.g., bispecific or trispecific) antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. In certain embodiments, the disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies disclosed herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding (e.g., as measured by cell binding titration or FACS binding) or binding affinity (e.g., K_{D}), improved or enhanced antagonistic or agonistic biological properties (as the case may be), reduced immunogenicity, etc. . . .

In some embodiments, the CD3 multispecific (e.g., bispecific or trispecific) antibodies provided herein comprise variants of any of the HCVR, LCVR, and/or CDR amino acid sequences disclosed herein having one or more conservative substitutions. For example, in certain embodiments the CD3 multispecific (e.g., bispecific or trispecific) antibodies provided herein have HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

### Fc Variants

According to certain embodiments provided herein, antibodies and multispecific antigen-binding molecules are provided comprising an Fc domain comprising one or more mutations which enhance or diminish antibody binding to the FcRn receptor, e.g., at acidic pH as compared to neutral pH. In certain embodiments, the disclosure includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428 L (e.g., M428 L) and 434S (e.g., N434S) modification; a 428 L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428 L modification (e.g., T250Q and M428 L); and a 307 and/or 308 modification (e.g., 308F or 308P).

In certain embodiments, the disclosure includes CD3 multispecific antigen-binding molecules (e.g., anti-CD3/anti-MUC16 bispecific, anti-BCMA x anti-CD3, or anti-CD3/anti-CD20 bispecific antibodies), comprising an Fc domain comprising one or more pairs or groups of mutations selected from the group consisting of: 250Q and 248 L (e.g., T250Q and M248 L); 252Y, 254T and 256E (e.g., M252Y, S254T and T256E); 428 L and 434S (e.g., M428 L and N434S); and 433K and 434F (e.g., H433K and N434F). All possible combinations of the foregoing Fc domain mutations, and other mutations within the antibody variable domains disclosed herein, are contemplated.

In some embodiments, the CD3xTAA bispecific antibodies of the present disclosure comprise an IgG Fc sequence with amino acid substitutions, e.g., using two residues derived from IgG3. For example, the CD3xTAA bispecific antibodies of the present disclosure may comprise IgG1 Fc sequence with amino acid substitutions H365R and Y366F in the CH3 region. Exemplary Fc sequences are also shown in FIG. 3.

### Bioequivalents

Provided herein antigen-binding molecules having amino acid sequences that vary from those of the exemplary molecules disclosed herein but that retain the ability to bind the same antigen or antigens. Such variant molecules may comprise one or more additions, deletions, or substitutions of amino acids when compared to parent sequence, but exhibit biological activity that is essentially equivalent to that of the described bispecific antigen-binding molecules.

In certain embodiments, the disclosure includes antigen-binding molecules that are bioequivalent to any of the exemplary antigen-binding molecules set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein. Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Antibody Binding

As used herein, the term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods. 1997, 201(2):223-31; Geuijen, C A, et al. J Immunol Methods. 2005, 302(1-2):68-77).

Accordingly, the antibody or antigen-binding protein provided herein binds to the predetermined antigen or cell surface molecule (receptor) having an affinity corresponding to a K_{D} value that is at least ten-fold lower than its affinity for binding to a non-specific antigen (e.g., BSA, casein). In some embodiments, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody disclosed herein.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D}) (higher, or stronger, affinity), for example expressed as 5-fold or 10-fold greater binding affinity, as the case may be.

The term “k_{d}” (sec-1 or 1/s) refers to the dissociation rate constant of a particular antibody-antigen interaction, or the dissociation rate constant of an antibody or antibody-binding fragment. Said value is also referred to as the k_{off} value.

The term “kₐ” (M−1 x sec-1 or 1/M) refers to the association rate constant of a particular antibody-antigen interaction, or the association rate constant of an antibody or antibody-binding fragment.

The term “K_{A}” (M−1 or 1/M) refers to the association equilibrium constant of a particular antibody-antigen interaction, or the association equilibrium constant of an antibody or antibody-binding fragment. The association equilibrium constant is obtained by dividing the kₐ by the k_{d}.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody disclosed herein that gives half-maximal binding to cells expressing CD3 or tumor-associated antigen (e.g., CD123, STEAP2, CD20, PSMA, SSTR2, CD38, STEAP1, 5T4, ENPP3, MUC16, or BCMA), as determined by e.g. a FACS binding assay. Thus, reduced or weaker binding is observed with an increased EC₅₀, or half maximal effective concentration value.

In one embodiment, decreased binding of an antibody (e.g., CD3 multispecific antibodies) can be defined as an increased EC₅₀ antibody concentration which enables binding to the half-maximal amount of target cells.

In another embodiment, the EC₅₀ value represents the concentration of an antibody (e.g., a CD3 multispecific antibody disclosed herein) that elicits half-maximal depletion of target cells by effector cell (e.g., T cell or NK cell) cytotoxic activity. Thus, increased cytotoxic activity (e.g. T cell or NK cell-mediated tumor cell killing) is observed with a decreased EC₅₀, or half maximal effective concentration value.

In still another embodiment, the EC₅₀ value represents the concentration of an antibody (e.g., a CD3 multispecific antibody disclosed herein) that elicits half-maximal activation of target cells by effector cell (e.g., T cell or NK cell) activation. For example, T cell activation can be measured by Jurkat NFAT reporter bioassay (e.g., the Jurkat/NFAT-Luc bioassay described in the Example 1). Thus, increased effector cell activation (e.g. T cell or NK cell activation) is observed with a decreased EC₅₀, or half-maximal effective concentration value.

pH-Dependent Binding

In certain embodiments, the disclosure includes antibodies and multispecific antigen-binding molecules with pH-dependent binding characteristics. For example, a CD3 multispecific antibody disclosed herein may exhibit reduced binding to CD3 at acidic pH as compared to neutral pH. Alternatively, CD3 multispecific antibodies disclosed herein may exhibit enhanced binding to CD3 at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, a CD3 multispecific antibody or antigen-binding fragment thereof may be regarded as exhibiting “reduced binding to CD3 at acidic pH as compared to neutral pH” for purposes disclosed herein if the CD3 multispecific antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment disclosed herein can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Antigen-Binding Domains and Construction of Multispecific Molecules

Antigen-binding domains specific for particular antigens can be prepared by any antibody generating technology known in the art. Once obtained, two different antigen-binding domains, specific for two different antigens (e.g., CD3 and a human tumor antigen (e.g., MUC16, BCMA, CD20, etc.)), can be appropriately arranged relative to one another to produce a bispecific antigen-binding molecule disclosed herein using routine methods. In certain embodiments, one or more of the individual components (e.g., heavy and light chains) of the multispecific antigen-binding molecules disclosed herein are derived from chimeric, humanized or fully human antibodies. Methods for making such antibodies are well known in the art. For example, one or more of the heavy and/or light chains of the bispecific antigen-binding molecules disclosed herein can be prepared using VELOCIMMUNE™ technology. Using VELOCIMMUNE™ technology (or any other human antibody generating technology), high affinity chimeric antibodies to a particular antigen (e.g., CD3 or human tumor antigen (e.g., MUC16, BCMA, CD20, etc.)) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules disclosed herein.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

### CD3 Multispecific Antigen-Binding Molecules

In certain embodiments, the methods and compositions provided herein relate to CD3 antigen-binding molecules (i.e., antigen binding molecules that comprise at least one antigen binding domain that binds to CD3). In certain embodiments, the CD3 multispecific antigen-binding molecules provided herein further comprise an antigen binding domain that binds to a cancer antigen (i.e., an antigen expressed on a cancer cell). In certain embodiments, the CD3 multispecific antigen-binding molecules provided herein further comprise an antigen binding domain that binds to a costimulatory receptor (e.g., CD28).

In some embodiments, the CD3 antibody is any one of the CD3 antibodies listed in Table 6.

TABLE 6
CD3 antibodies

| Name | Targets |
|---|---|
| CH2527 | CD3 |
| Odronextamab (REGN1979) | CD20 × CD3 |
| REGN2281 | CD20 × CD3 |
| REGN5458 | BCMA × CD3 |
| AMV-564 | CD33 × CD3 |
| GBR 1342 | CD38 × CD3 |
| XmAb18968 | CD3 × CD38 |
| SAR443216 | CD3 × CD28 × HER2 |
| A-319 | CD19 × CD3 |
| AMG 330/eluvixtamab | CD33/CD3 |
| emerfetamab | CD33/CD3 |
| ertumaxomab | CD3-HER2neu |
| REGN4018 | CD3[7221G] × MUC16 |
| H4tH7221G | Anti-hCD3[7221G] |
| H4tH7221G20 | Anti-hCD3[7221G20] |
| REGN4019 | CD3[7221G20] × MUC16 |
| REGN1453 | CD3[9F07] × CD20 |
| REGN5375 | CD20 × CD3[7221P Goldi2] |
| REGN5951 | CD20 × CD3[7221G Goldi] |
| REGN4336 | PSMA × CD3[7221G20] |
| REGN2533 | CD3[09F7] |
| REGN 2280 | CD20 × CD3[09F7] |
| REGN 5949 | CD20 × CD3[7221G] |
| REGN 5950 | CD20 × CD3[7221G20] |
| H4tH7221G5 | CD3[7221G5] |
| H4sH17400D | CD20 × CD3[7221G5] |

As used herein, the expression “multispecific antigen-binding molecule” refers to a protein, polypeptide or molecular complex comprising at least a first antigen-binding domain and a second antigen-binding domain. In some embodiments, each antigen-binding domain within the multispecific antigen-binding molecule may comprises at least one CDR that alone, or in combination with one or more additional CDRs and/or FRs, specifically binds to a particular antigen. In the context disclosed herein, the first antigen-binding domain specifically binds a first antigen (e.g., CD3), and the second antigen-binding domain specifically binds a second, distinct antigen (e.g., a tumor antigen).

In some embodiments, the CD3 multispecific antigen-binding molecule is a CD3 multispecific antibody. The CD3 multispecific antibodies of provided herein may be, for example, bi-specific, or tri-specific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The CD3 bispecific antibodies provided herein can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

In certain embodiments, the disclosure includes bispecific antibodies wherein one arm of an immunoglobulin binds CD3, and the other arm of the immunoglobulin is specific for a cancer antigen (also referred to herein as a tumor antigen, or “TAA”). In certain embodiments, the disclosure includes trispecific antibodies wherein a first arm of an immunoglobulin binds CD3, a second arm of the immunoglobulin is specific for a tumor antigen, and a third arm of the immunoglobulin binds an additional T cell antigen (e.g., CD28) or an additional tumor antigen.

In some embodiments, the CD3-binding arm may comprise any of the HCVR/LCVR or CDR amino acid sequences as disclosed in WO 2014/047231 or WO 2017/053856. In certain embodiments, the CD3-binding arm binds to human CD3 and induces human T cell activation. In certain embodiments, the CD3-binding arm binds weakly to human CD3 and induces human T cell activation. In other embodiments, the CD3-binding arm binds weakly to human CD3 and induces tumor-associated antigen-expressing cell killing in the context of a bispecific or multispecific antibody. In other embodiments, the CD3-binding arm binds or associated weakly with human and cynomolgus (monkey) CD3, yet the binding interaction is not detectable by in vitro assays known in the art.

In certain embodiments, the multispecific antibodies or antigen-binding fragments comprise an antigen-binding arm that binds to CD28, ICOS, HVEM, CD27, 4-1BB, OX40, DR3, GITR, CD30, SLAM, CD2, 2B4, CD226, TIM1, or TIM2 to induce T cell activation.

In certain embodiments, the CD3 multispecific antigen-binding molecule comprises an antigen-binding domain specific for a cancer antigen. In certain embodiments, the cancer antigen is selected from AIM-2, ALDH1A1, alpha-actinin-4, alpha-fetoprotein (“AFP”), ARTC1, B-RAF, BAGE-1, BCLX (L), BCMA, BCR-ABL fusion protein b3a2, beta-catenin, BING-4, CA-125, CALCA, carcinoembryonic antigen (“CEA”), CASP-5, CASP-8, CD19, CD20, CD22, CD38, CD45, CD123, CD274, Cdc27, CDK12, CDK4, CDKN2A, CEA, CLPP, CLDN18.2, CEACAM5, COA-1, CPSF, CSNK1A1, CTAG1, CTAG2, cyclin DI, Cyclin-A1, dek-can fusion protein, DKK1, EFTUD2, Elongation factor 2, ENPP3, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GPRC5D, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MSLN, MUC1, MUCSAC, MUC16, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pm1-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SLC3A2-APIS, SNRPD1, SOX10, Sp17, SPA17, SSX-2, SSX-4, STEAP1, STEAP2, survivin, SSTR2, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, 5T4, and XAGE-1b/GAGED2a.

In some embodiments, the cancer antigen is include ADAM 17, BCMA, CA-IX, CD19, CD20, CD22, CD30, CD33, CD38, CD52, CD56, CD70, CD74, CD79b, CD123, CD138, CDH3, CEA, EphA2, EpCAM, ERBB2, ENPP3, EGFR, EGFR-VIII, FLT3, FOLR1, GD-2, glypican-3, gpA33, GPNMB, GPRC5D, HER2, HER3, LMP1, LMP2A, MUC16, Mesothelin, PSMA, PSCA, RON, ROR1, ROR2, STEAP1, STEAP2, SSTR2, SSTR5, 5T4, and Trop-2. In some embodiments, the tumore antigen may be CD19, CD123, STEAP2, CD20, SSTR2, CD38, STEAP1, 5T4, ENPP3, PSMA, MUC16, GPRC5D, BCMA, CA19.9, MSLN, CD22, SLC3A2-APIS, CLDN18.2, or CEACAM5.

In some embodiments, the tumore antigen may be CD19, CD123, STEAP2, CD20, SSTR2, CD38, STEAP1, 5T4, ENPP3, PSMA, MUC16, GPRC5D, BCMA, CA19.9, MSLN, CD22, SLC3A2-APIS, CLDN18.2, or CEACAM5.

In some embodiments, the cancer antigen is CD20, MUC16, BCMA, PSMA, or STEAP2.

CD20 is a non-glycosylated phosphoprotein expressed on the cell membranes of mature B cells. CD20 is considered a B cell tumor-associated antigen because it is expressed by more than 95% of B-cell non-Hodgkin lymphomas (NHLs) and other B-cell malignancies, but it is absent on precursor B-cells, dendritic cells and plasma cells. The human CD20 protein has the amino acid sequence shown in SEQ ID NO: 5 of U.S. Patent Application Publication No. US 2020/0129617, the content of which is incorporated by reference herein in its entirety.

MUC16 refers to mucin 16. MUC16 is a single transmembrane domain highly glycosylated integral membrane glycoprotein that is highly expressed in ovarian cancer. The amino acid sequence of human MUC16 is set forth in SEQ ID NO:1899 of U.S. Patent Application Publication No. US 2018/0118848A1, the content of which is incorporated by reference herein in its entirety.

BCMA refers to B-cell maturation antigen. BCMA (also known as TNFRSF17 and CD269) is a cell surface protein expressed on malignant plasma cells, and plays a central role in regulating B cell maturation and differentiation into immunoglobulin-producing plasma cells. The amino acid sequence of human BCMA is shown in SEQ ID NO: 115 of U.S. Patent Application Publication No. US 2020/0024356, the content of which is incorporated by reference herein in its entirety. It can also be found in GenBank accession number NP_001183.2.

PSMA refers to prostate-specific membrane antigen, also known as folate hydrolase 1 (FOLH1). PSMA is an integral, non-shed membrane glycoprotein that is highly expressed in prostate epithelial cells and is a cell-surface marker for prostate cancer. The amino acid sequence of human PSMA is set forth in SEQ ID NO: 7 of U.S. Patent Application Publication No. US 2020/0129617, the content of which is incorporated by reference herein in its entirety.

STEAP2 refers to six-transmembrane epithelial antigen of prostate 2. STEAP2 is an integral, six-transmembrane-spanning protein that is highly expressed in prostate epithelial cells and is a cell-surface marker for prostate cancer. STEAP2 is a 490-amino acid protein encoded by STEAP2 gene located at the chromosomal region 7q21 in humans. The amino acid sequence of human STEAP2 is set forth in SEQ ID NO: 9 of U.S. Patent Application Publication No. US 2020/0129617, the content of which is incorporated by reference herein in its entirety.

In some embodiments, the CD3 multispecific antibody may be a bispecific CD3xCD19 antibody, a bispecific CD3x GPRC5D antibody, a bispecific CD3xCD123 antibody, a bispecific CD3xSTEAP2 antibody, a bispecific CD3xCD20 antibody, a bispecific CD3xSSTR 2 antibody, a bispecific CD3xCD38 antibody, a bispecific CD3xSTEAP1 antibody, a bispecific CD3×5T4 antibody, a bispecific CD3xENPP3 antibody, a bispecific CD3xMUC16 antibody, a bispecific CD3xBCMA antibody, a bispecific CD3xPSMA antibody, or a trispecific CD3xCD28xCD38 antibody.

In certain embodiments, the disclosure includes antibodies having the HCVR, LCVR and/or CDR amino acid sequences of the antibodies set forth herein, the anti-CD3 antibodies disclosed in WO 2014/047231 or WO 2017/053856, the bispecific anti-CD20 x anti-CD3 antibodies disclosed in WO 2014/047231, the bispecific anti-PSMA x anti-CD3 antibodies disclosed in WO 2017/023761, the bispecific anti-MUC16 x anti-CD3 antibodies disclosed in WO 2018/067331, the bispecific anti-STEAP2 x anti-CD3 antibodies disclosed in WO 2018/058001, or the bispecific anti-BCMA x anti-CD3 antibodies disclosed in WO 2020/018820, each of which is incorporated herein by reference.

In certain embodiments, the multispecific antigen-binding molecule is a multispecific antibody or antigen-binding fragment thereof. Each antigen-binding domain of a multispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3. In the context of a trispecific antigen-binding molecule comprising a first, a second, and a third antigen-binding domain (e.g., a trispecific antibody), the CDRs of the first antigen-binding domain may be designated with the prefix “A1”, the CDRs of the second antigen-binding domain may be designated with the prefix “A2”, and the CDRs of the third antigen-binding domain may be designated with the prefix “A3”. Thus, the CDRs of the first antigen-binding domain may be referred to herein as A1-HCDR1, A1-HCDR2, and A1-HCDR3; the CDRs of the second antigen-binding domain may be referred to herein as A2-HCDR1, A2-HCDR2, and A2-HCDR3; and the CDRs of the third antigen-binding domain may be referred to herein as A3-HCDR1, A3-HCDR2, and A3-HCDR3.

The bispecific antigen-binding molecules discussed above or herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4. In various embodiments, the bispecific antibody comprises a chimeric hinge that reduces Fcγ receptor binding relative to a wild-type hinge of the same isotype.

The first antigen-binding domain and the second antigen-binding domain may be directly or indirectly connected to one another to form a bispecific antigen-binding molecule disclosed herein. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules disclosed herein will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules disclosed herein. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context disclosed herein include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antigen-binding molecules provided herein, the multimerizing domains, e.g., Fc domains, may comprise one or more amino acid changes (e.g., insertions, deletions or substitutions) as compared to the wild-type, naturally occurring version of the Fc domain. In certain embodiments, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428 L (e.g., M428 L) and 434S (e.g., N434S) modification; a 428 L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428 L modification (e.g., T250Q and M428 L); and a 307 and/or 308 modification (e.g., 308F or 308P).

Tin certain embodiments, provided herein are bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antigen-binding molecules disclosed herein are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

The CD3 multispecific (e.g., bispecific or trispecific) antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. In certain embodiments, the disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies disclosed herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding (e.g., as measured by cell binding titration or FACS binding) or binding affinity (e.g., K_{D}), improved or enhanced antagonistic or agonistic biological properties (as the case may be), reduced immunogenicity, etc. Antibodies and antigen-binding fragments obtained in this general manner are encompassed within the present disclosure.

Provided herein are also CD3 multispecific (e.g., bispecific or trispecific) antibodies comprising variants of any of the HCVR, LCVR, and/or CDR amino acid sequences disclosed herein having one or more conservative substitutions. In certain embodiments, the disclosure includes CD3 multispecific (e.g., bispecific or trispecific) antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

### Exemplary CD3xMUC16 Antibodies

In some embodiments, the methods and compositions provided herein include bispecific antibodies wherein one arm of an immunoglobulin binds human CD3, and the other arm of the immunoglobulin is specific for human MUC16. The term “MUC16,” as used herein, refers to the human MUC16 protein unless specified as being from a non-human species (e.g., “mouse MUC16,” “monkey MUC16,” etc.). The human MUC16 protein has the amino acid sequence shown in SEQ ID NO:1899 of U.S. Patent Application Publication No. US 2018/0118848A1, the content of which is incorporated by reference herein in its entirety. Such molecules may be referred to herein as, e.g., “anti-CD3/anti-MUC16,” or “anti-CD3xMUC16” or “CD3xMUC16” bispecific molecules, or other similar terminology (e.g., anti-MUC16/anti-CD3). Such bispecific antigen-binding molecules are constructed with a first antigen-binding arm that binds MUC16 and a second antigen-binding arm that binds CD3. The MUC16-binding arm can comprise any of the HCVR/LCVR or CDR amino acid sequences as set forth in Table 7 herein. The CD3-binding arm can comprise any of the HCVR/LCVR or CDR amino acid sequences as set forth in Tables 8-12 herein. Sequences in Tables 7-12 were disclosed in U.S. Patent Application Publication No. US 2018/0118848A1, the content of which is incorporated by reference herein in its entirety.

Table 7 sets forth the amino acid sequence identifiers of the heavy and light chain variable regions and CDRs of selected anti-MUC16 antibodies disclosed herein.

TABLE 7
Amino Acid Sequence Identifiers

| | HCVR | HCDR1 | HCDR2 | HCDR3 | LCVR | LCDR1 | LCDR2 | LCDR3 |
|---|---|---|---|---|---|---|---|---|
| | (SEQ | (SEQ | (SEQ | (SEQ | (SEQ | (SEQ | (amino | (SEQ |
| Antibody | ID | ID | ID | ID | ID | ID | acid | ID |
| Designation | NOs) | NOs) | NOs) | NOs) | NOs) | NOs) | sequence) | NOs) |
| H1H8755P | 95 | 97 | 99 | 101 | 103 | 105 | AAS | 107 |
| H1H8767P | 109 | 111 | 113 | 115 | 117 | 119 | TAS | 312 |
| H1H8770P | 121 | 123 | 127 | 129 | 131 | 324 | TAS | 312 |
| H1H8783P | 133 | 135 | 137 | 139 | 141 | 324 | TAS | 312 |
| H1H8790P | 143 | 145 | 147 | 149 | 151 | 396 | AAS | 312 |
| H1H8794P | 153 | 155 | 157 | 159 | 161 | 163 | AAS | 96 |
| H1H8794P2 | 153 | 155 | 157 | 159 | 858 | 860 | AAS | 864 |
| H1H8799P | 98 | 100 | 102 | 104 | 106 | 108 | GAS | 112 |
| H1H8799P2 | 98 | 100 | 102 | 104 | 170 | 172 | GAS | 176 |
| H1H8804P | 114 | 116 | 118 | 120 | 122 | 124 | GAS | 128 |
| H1H8808P | 130 | 132 | 134 | 136 | 138 | 140 | GIS | 144 |
| H1H8810P | 146 | 148 | 150 | 152 | 154 | 156 | GAS | 160 |
| H1H8813P | 162 | 164 | 166 | 168 | 170 | 172 | GAS | 176 |
| H1M7129N | 178 | 180 | 182 | 184 | 186 | 188 | GAS | 192 |
| H1M7137N | 194 | 196 | 198 | 200 | 394 | 396 | AAS | 400 |
| H1M9519N | 202 | 204 | 206 | 208 | 210 | 212 | DAS | 216 |
| H1M9521N | 218 | 220 | 222 | 224 | 226 | 228 | GAS | 232 |
| H1M9528N | 234 | 236 | 238 | 240 | 242 | 244 | GAS | 248 |
| H2M7128N | 250 | 252 | 254 | 256 | 1936 | 1938 | GAS | 1942 |
| H1M7130N | 1944 | 1946 | 1948 | 1950 | 1952 | 1954 | GAS | 1958 |
| H2M7131N | 258 | 260 | 262 | 264 | 266 | 268 | GAS | 272 |
| H2M7133N | 274 | 276 | 278 | 280 | 1936 | 1938 | GAS | 1942 |
| H2M7134N | 282 | 284 | 286 | 288 | 290 | 292 | GAS | 296 |
| H2M7135N | 298 | 300 | 302 | 304 | 306 | 308 | AAS | 312 |
| H2M7138N | 314 | 316 | 318 | 320 | 322 | 324 | AAS | 328 |
| H2M9538N | 330 | 332 | 334 | 336 | 338 | 340 | GAS | 344 |
| H3M9524N | 346 | 348 | 350 | 352 | 354 | 356 | WAS | 360 |
| H3M9525N | 362 | 364 | 366 | 368 | 370 | 372 | WAS | 376 |
| H3M9529N | 378 | 380 | 382 | 384 | 386 | 388 | WAS | 392 |

Table 8 sets forth the amino acid sequence identifiers of the heavy and light chain variable regions and CDRs of selected anti-CD3 antibodies disclosed herein. Methods of making the anti-CD3 antibodies disclosed herein can also be found in US publication 2014/0088295.

TABLE 8
Amino Acid Sequence Identifiers

| | HCVR | HCDR1 | HCDR2 | HCDR3 | LCVR | LCDR1 | LCDR2 | LCDR3 |
|---|---|---|---|---|---|---|---|---|
| | (SEQ | (SEQ | (SEQ | (SEQ | (SEQ | (SEQ | (amino | (SEQ |
| Antibody | ID | ID | ID | ID | ID | ID | acid | ID |
| Designation | NOs) | NOs) | NOs) | NOs) | NOs) | NOs) | sequences) | NOs) |
| H1H2712N | 402 | 404 | 406 | 408 | 410 | 412 | GAS | 416 |
| H1M2692N | 418 | 420 | 422 | 424 | 426 | 428 | GAS | 432 |
| H1M3542N | 434 | 436 | 438 | 440 | 442 | 444 | AAS | 448 |
| H1M3544N | 450 | 452 | 454 | 456 | 458 | 460 | GAS | 464 |
| H1M3549N | 466 | 468 | 470 | 472 | 474 | 476 | AAS | 480 |
| H1M3613N | 482 | 484 | 486 | 488 | 490 | 492 | AAS | 496 |
| H2M2689N | 498 | 500 | 502 | 504 | 506 | 508 | DAS | 512 |
| H2M2690N | 514 | 516 | 518 | 520 | 522 | 524 | GAS | 528 |
| H2M2691N | 530 | 532 | 534 | 536 | 538 | 540 | GAS | 544 |
| H2M2704N | 546 | 548 | 550 | 552 | 554 | 556 | GAS | 560 |
| H2M2705N | 562 | 564 | 566 | 568 | 570 | 572 | GAS | 576 |
| H2M2706N | 578 | 580 | 582 | 584 | 586 | 588 | GAS | 592 |
| H2M2707N | 594 | 596 | 598 | 600 | 602 | 604 | GAS | 608 |
| H2M2708N | 610 | 612 | 614 | 616 | 618 | 620 | GAS | 624 |
| H2M2709N | 626 | 628 | 630 | 632 | 634 | 636 | GAS | 640 |
| H2M2710N | 642 | 644 | 646 | 648 | 650 | 652 | GAS | 656 |
| H2M2711N | 658 | 660 | 662 | 664 | 666 | 668 | GAS | 672 |
| H2M2774N | 674 | 676 | 678 | 680 | 682 | 684 | GAS | 688 |
| H2M2775N | 690 | 692 | 694 | 696 | 698 | 700 | GAT | 704 |
| H2M2776N | 706 | 708 | 710 | 712 | 714 | 716 | GAT | 720 |
| H2M2777N | 722 | 724 | 726 | 728 | 730 | 732 | GAS | 736 |
| H2M2778N | 738 | 740 | 742 | 744 | 746 | 748 | GAS | 752 |
| H2M2779N | 754 | 756 | 758 | 760 | 762 | 764 | WAS | 768 |
| H2M2789N | 770 | 772 | 774 | 776 | 778 | 780 | AAS | 784 |
| H2M2862N | 786 | 788 | 790 | 792 | 794 | 796 | GAS | 800 |
| H2M2885N | 802 | 804 | 806 | 808 | 810 | 812 | GAT | 816 |
| H2M2886N | 818 | 820 | 822 | 824 | 826 | 828 | GAT | 832 |
| H2M3540N | 834 | 836 | 838 | 840 | 842 | 844 | AAS | 848 |
| H2M3541N | 850 | 852 | 854 | 856 | 858 | 860 | AAS | 864 |
| H2M3543N | 866 | 868 | 870 | 872 | 874 | 876 | AAS | 880 |
| H2M3547N | 882 | 884 | 886 | 888 | 890 | 892 | AAS | 896 |
| H2M3548N | 898 | 900 | 902 | 904 | 906 | 908 | AAS | 912 |
| H2M3563N | 914 | 916 | 918 | 920 | 922 | 924 | AAS | 928 |
| H1H5751P | 930 | 932 | 934 | 936 | 938 | 940 | AAS | 944 |
| H1H5752P | 946 | 948 | 950 | 952 | 954 | 956 | EAS | 960 |
| H1H5753B | 962 | 964 | 966 | 968 | 970 | 972 | AVS | 976 |
| H1H5754B | 978 | 980 | 982 | 984 | 986 | 988 | DAS | 992 |
| H1H5755B | 994 | 996 | 998 | 1000 | 1002 | 1004 | AAS | 1008 |
| H1H5756B | 1010 | 1012 | 1014 | 1016 | 1018 | 1020 | AAS | 1024 |
| H1H5757B | 1026 | 1028 | 1030 | 1032 | 1034 | 1036 | AAS | 1040 |
| H1H5758B | 1042 | 1044 | 1046 | 1048 | 1050 | 1052 | AAS | 1056 |
| H1H5761P | 1058 | 1060 | 1062 | 1064 | 1066 | 1068 | AAS | 1072 |
| H1H5763P | 1074 | 1076 | 1078 | 1080 | 1082 | 1084 | AAS | 1088 |
| H1H5764P | 1090 | 1092 | 1094 | 1096 | 1098 | 1100 | AAS | 1104 |
| H1H5769P | 1106 | 1108 | 1110 | 1112 | 1114 | 1116 | GAS | 1120 |
| H1H5771P | 1122 | 1124 | 1126 | 1128 | 1130 | 1132 | GAS | 1136 |
| H1H5772P | 1138 | 1140 | 1142 | 1144 | 1146 | 1148 | GAS | 1152 |
| H1H5777P | 1154 | 1156 | 1158 | 1160 | 1162 | 1164 | GAS | 1168 |
| H1H5778P | 1170 | 1172 | 1174 | 1176 | 1178 | 1180 | GAS | 1184 |
| H1H5780P | 1186 | 1188 | 1190 | 1192 | 1194 | 1196 | GAS | 1200 |
| H1H5781P | 1202 | 1204 | 1206 | 1208 | 1210 | 1212 | GAS | 1216 |
| H1H5782P | 1218 | 1220 | 1222 | 1224 | 1226 | 1228 | KAS | 1232 |
| H1H5785B | 1234 | 1236 | 1238 | 1240 | 1242 | 1244 | WAS | 1248 |
| H1H5786B | 1250 | 1252 | 1254 | 1256 | 1258 | 1260 | TAS | 1264 |
| H1H5788P | 1266 | 1268 | 1270 | 1272 | 1274 | 1276 | GAS | 1280 |
| H1H5790B | 1282 | 1284 | 1286 | 1288 | 1290 | 1292 | TAS | 1296 |
| H1H5791B | 1298 | 1300 | 1302 | 1304 | 1306 | 1308 | TAS | 1312 |
| H1H5792B | 1314 | 1316 | 1318 | 1320 | 1322 | 1324 | TAS | 1328 |
| H1H5793B | 1330 | 1332 | 1334 | 1336 | 1338 | 1340 | VAS | 1344 |
| H1H5795B | 1346 | 1348 | 1350 | 1352 | 1354 | 1356 | TAS | 1360 |
| H1H5796B | 1362 | 1364 | 1366 | 1368 | 1370 | 1372 | AAS | 1376 |
| H1H5797B | 1378 | 1380 | 1382 | 1384 | 1386 | 1388 | VAS | 1392 |
| H1H5798B | 1394 | 1396 | 1398 | 1400 | 1402 | 1404 | TAS | 1408 |
| H1H5799P | 1410 | 1412 | 1414 | 1416 | 1418 | 1420 | TAS | 1424 |
| H1H5801B | 1426 | 1428 | 1430 | 1432 | 1434 | 1436 | VAS | 1440 |
| H1H7194B | 1442 | 1444 | 1446 | 1448 | 1634 | 1636 | AAS | 1640 |
| H1H7195B | 1450 | 1452 | 1454 | 1456 | 1634 | 1636 | AAS | 1640 |
| H1H7196B | 1458 | 1460 | 1462 | 1464 | 1634 | 1636 | AAS | 1640 |
| H1H7198B | 1466 | 1468 | 1470 | 1472 | 1634 | 1636 | AAS | 1640 |
| H1H7203B | 1474 | 1476 | 1478 | 1480 | 1634 | 1636 | AAS | 1640 |
| H1H7204B | 1482 | 1484 | 1486 | 1488 | 1634 | 1636 | AAS | 1640 |
| H1H7208B | 1490 | 1492 | 1494 | 1496 | 1634 | 1636 | AAS | 1640 |
| H1H7211B | 1498 | 1500 | 1502 | 1504 | 1634 | 1636 | AAS | 1640 |
| H1H7221B | 1506 | 1508 | 1510 | 1512 | 1634 | 1636 | AAS | 1640 |
| H1H7223B | 1514 | 1516 | 1518 | 1520 | 1634 | 1636 | AAS | 1640 |
| H1H7226B | 1522 | 1524 | 1526 | 1528 | 1634 | 1636 | AAS | 1640 |
| H1H7232B | 1530 | 1532 | 1534 | 1536 | 1634 | 1636 | AAS | 1640 |
| H1H7233B | 1538 | 1540 | 1542 | 1544 | 1634 | 1636 | AAS | 1640 |
| H1H7241B | 1546 | 1548 | 1550 | 1552 | 1634 | 1636 | AAS | 1640 |
| H1H7242B | 1554 | 1556 | 1558 | 1560 | 1634 | 1636 | AAS | 1640 |
| H1H7250B | 1562 | 1564 | 1566 | 1568 | 1634 | 1636 | AAS | 1640 |
| H1H7251B | 1570 | 1572 | 1574 | 1576 | 1634 | 1636 | AAS | 1640 |
| H1H7254B | 1578 | 1580 | 1582 | 1584 | 1634 | 1636 | AAS | 1640 |
| H1H7258B | 1586 | 1588 | 1590 | 1592 | 1634 | 1636 | AAS | 1640 |
| H1H7269B | 1594 | 1596 | 1598 | 1600 | 1634 | 1636 | AAS | 1640 |
| H1H7279B | 1602 | 1604 | 1606 | 1608 | 1634 | 1636 | AAS | 1640 |
| H1xH7221G | 1610 | 1612 | 1614 | 1616 | 1634 | 1636 | AAS | 1640 |
| H1xH7221G3 | 1618 | 1620 | 1622 | 1624 | 1634 | 1636 | AAS | 1640 |
| H1xH7221G5 | 1626 | 1628 | 1630 | 1632 | 1634 | 1636 | AAS | 1640 |

Tables 9 and 10 set out the amino acid sequence identifiers for heavy chain variable regions (Table 9) and light chain variable regions (Table 10), and their corresponding CDRs, of additional anti-CD3 HCVRs and LCVRs useful in anti-MUC16 x anti-CD3 bispecific antibodies disclosed herein.

TABLE 9
(Heavy Chain Variable Region Amino Acid Sequences)

| Heavy Chain | SEQ ID NOs | | | |
|---|---|---|---|---|
| Identifier | HCVR | HCDR1 | HCDR2 | HCDR3 |
| CD3-VH-AA | 1642 | 1644 | 1646 | 1648 |
| CD3-VH-B | 1658 | 1660 | 1662 | 1664 |
| CD3-VH-C | 1674 | 1676 | 1678 | 1680 |
| CD3-VH-D | 1690 | 1692 | 1694 | 1696 |
| CD3-VH-E | 1706 | 1708 | 1710 | 1712 |
| CD3-VH-F^{#} | 1721 | 1722 | 1723 | 1724 |

TABLE 10
(Light Chain Variable Region Amino Acid Sequences)

| | LCVR | LCDR1 | LCDR2 | LCDR3 |
|---|---|---|---|---|
| Light Chain | (SEQ | (SEQ | (amino acid | (SEQ |
| Identifier | ID NOs) | ID NOs) | sequences) | ID NOs) |
| CD3-VL-AA | 1650 | 1652 | GAS | 1656 |
| CD3-VL-B | 1666 | 1668 | GAS | 1672 |
| CD3-VL-C | 1682 | 1684 | GAS | 1688 |
| CD3-VL-D | 1698 | 1700 | GAS | 1704 |
| CD3-VL-E | 1714 | 1716 | GAS | 1720 |
| CD3-VL-F^{#} | 1725 | 1726 | DTS | 1728 |

Table 11 sets forth the amino acid sequence identifiers of the heavy chain variable regions and CDRs of engineered anti-CD3 antibodies disclosed herein. The amino acid sequence identifiers of the light chain variable region and CDRs are also identified below in Table 12.

TABLE 11
Heavy Chain Amino Acid Sequence Identifiers

| Antibody | | | | |
|---|---|---|---|---|
| CD3-VH | SEQ ID NOs: | | | |
| Designation | HCVR | CDR1 | CDR2 | CDR3 |
| CD3-VH-G | 1730 | 1732 | 1734 | 1736 |
| CD3-VH-G2 | 1738 | 1740 | 1742 | 1744 |
| CD3-VH-G3 | 1746 | 1748 | 1750 | 1752 |
| CD3-VH-G4 | 1754 | 1756 | 1758 | 1760 |
| CD3-VH-G5 | 1762 | 1764 | 1766 | 1768 |
| CD3-VH-G8 | 1770 | 1772 | 1774 | 1776 |
| CD3-VH-G9 | 1778 | 1780 | 1782 | 1784 |
| CD3-VH-G10 | 1786 | 1788 | 1790 | 1792 |
| CD3-VH-G11 | 1794 | 1796 | 1798 | 1800 |
| CD3-VH-G12 | 1802 | 1804 | 1806 | 1808 |
| CD3-VH-G13 | 1810 | 1812 | 1814 | 1816 |
| CD3-VH-G14 | 1818 | 1820 | 1822 | 1824 |
| CD3-VH-G15 | 1826 | 1828 | 1830 | 1832 |
| CD3-VH-G16 | 1834 | 1836 | 1838 | 1840 |
| CD3-VH-G17 | 1842 | 1844 | 1846 | 1848 |
| CD3-VH-G18 | 1850 | 1852 | 1854 | 1856 |
| CD3-VH-G19 | 1858 | 1860 | 1862 | 1864 |
| CD3-VH-G20 | 1866 | 1868 | 1870 | 1872 |
| CD3-VH-G21 | 1874 | 1876 | 1878 | 1880 |
| CD3-VH-P | 1882 | 1884 | 1886 | 1888 |

TABLE 12
Light Chain Amino Acid Sequence Identifiers

| | LCVR | CDR1 | CDR2 | CDR3 |
|---|---|---|---|---|
| Antibody | (SEQ | (SEQ | (amino acid | (SEQ |
| Designation | ID NO) | ID NO) | sequence) | ID NO) |
| VK1-39JK5 | 1890 | 1892 | TAS | 1896 |

In certain exemplary embodiments, the first antigen-binding domain that specifically binds human CD3 comprises heavy chain complementarity determining regions (HCDR1, HCDR2 and HCDR3) from a heavy chain variable region (HCVR) selected from the group consisting of SEQ ID NOs: 1730, 1762, 1778, 1786, and 1866, and light chain complementarity determining regions (LCDR1, LCDR2 and LCDR3) from a light chain variable region (LCVR) comprising an amino acid sequence of SEQ ID NO: 117.

In certain exemplary embodiments, the first antigen-binding domain that specifically binds human CD3 comprises three heavy chain complementarity determining regions (A1-HCDR1, A1-HCDR2 and A1-HCDR3) and three light chain complementarity determining regions (A1-LCDR1, A1-LCDR2 and A1-LCDR3), wherein A1-HCDR1 comprises an amino acid sequence selected from the group consisting of SEQ ID NOs: 1732, 1764, 1780, 1788, and 1868; A1-HCDR2 comprises an amino acid sequence selected from the group consisting of SEQ ID NOs: 1734, 1766, 1782, 1790, and 1870; A1-HCDR3 comprises an amino acid sequence selected from the group consisting of SEQ ID NOs:1736, 1768, 1784, 1792, and 1872; A1-LCDR1 comprises an amino acid sequence of SEQ ID NO:119; A1-LCDR2 comprises an amino acid sequence TAS; and A1-LCDR3 comprises an amino acid sequence of SEQ ID NO:312.

In certain exemplary embodiments, the first antigen-binding domain that specifically binds human CD3 comprises the heavy and light chain CDRs of a HCVR/LCVR amino acid sequence pair selected from the group consisting of: SEQ ID NOs: 1730/117, 1762/117, 1778/117, 1786/117, and 1866/117.

In certain exemplary embodiments, the first antigen-binding domain that specifically binds human CD3 comprises three heavy chain complementarity determining regions (A1-HCDR1, A1-HCDR2 and A1-HCDR3) and three light chain complementarity determining regions (A1-LCDR1, A1-LCDR2 and A1-LCDR3), and the second antigen-binding domain that specifically binds human MUC16 comprises three heavy chain complementarity determining regions (A2-HCDR1, A2-HCDR2 and A2-HCDR3) and three light chain complementarity determining regions (A2-LCDR1, A2-LCDR2 and A2-LCDR3); wherein A1-HCDR1 comprises an amino acid sequence selected from the group consisting of SEQ ID NOs: 1732, 1764, 1780, 1788, and 1868; A1-HCDR2 comprises an amino acid sequence selected from the group consisting of SEQ ID NOs: 1734, 1766, 1782, 1790, and 1870; A1-HCDR3 comprises an amino acid sequence selected from the group consisting of SEQ ID NOs: 1736, 1768, 1784, 1792, and 1872; A1-LCDR1 comprises an amino acid sequence of SEQ ID NO: 119; A1-LCDR2 comprises an amino acid sequence TAS; and A1-LCDR3 comprises an amino acid sequence of SEQ ID NO:312; and wherein A2-HCDR1 comprises an amino acid sequence of SEQ ID NO:111; A2-HCDR2 comprises an amino acid sequence of SEQ ID NO:113; A2-HCDR3 comprises an amino acid sequence of SEQ ID NO: 115; A2-LCDR1 comprises an amino acid sequence of SEQ ID NO:119; A2-LCDR2 comprises an amino acid sequence TAS; and A2-LCDR3 comprises an amino acid sequence of SEQ ID NO:312.

Additional bispecific anti-MUC16 x anti-CD3 antibodies disclosed in, e.g., WO 2018/067331, which is hereby incorporated by reference.

### Exemplary CD3xBCMA Antibodies

In some embodiments, the methods and compositions provided herein include bispecific antibodies wherein one arm of an immunoglobulin binds human CD3, and the other arm of the immunoglobulin is specific for human BCMA. The term “BCMA,” as used herein, refers to the human BCMA protein unless specified as being from a non-human species (e.g., “mouse BCMA,” “monkey BCMA,” etc.). The human BCMA protein has the amino acid sequence shown in SEQ ID NO: 115 of U.S. Patent Application Publication No. US 2020/0024356A1, the content of which is incorporated herein by reference in its entirety. Such molecules may be referred to herein as, e.g., “anti-BCMA x anti-CD3” or “anti-CD3/anti-BCMA,” or “anti-CD3xBCMA” or “CD3xBCMA” bispecific molecules, or other similar terminology (e.g., anti-BCMA/anti-CD3). The BCMA-binding arm can comprise any of the HCVR/LCVR or CDR amino acid sequences as set forth in Table 13 herein. The CD3-binding arm can comprise any of the HCVR/LCVR or CDR amino acid sequences as set forth in Table 14 herein, or the anti-CD3 antibodies disclosed in WO 2014/047231 or WO 2017/053856. Sequences in Tables 13 and 14 were disclosed in U.S. Patent Application Publication No. US 2020/0024356A1, the content of which is incorporated herein by reference in its entirety.

Table 13 sets forth the amino acid sequence identifiers of the heavy and light chain variable regions and CDRs of selected anti-BCMA antibodies disclosed herein.

TABLE 13
Amino Acid Sequence Identifiers

| | HCVR | HCDR1 | HCDR2 | HCDR3 | LCVR | LCDR1 | LCDR2 | LCDR3 |
|---|---|---|---|---|---|---|---|---|
| | (SEQ | (SEQ | (SEQ | (SEQ | (SEQ | (SEQ | (amino | (SEQ |
| Antibody | ID | ID | ID | ID | ID | ID | acid | ID |
| Designation | NOs) | NOs) | NOs) | NOs) | NOs) | NOs) | sequences) | NOs) |
| mAb16711 | 165 | 167 | 169 | 171 | 173 | 175 | AAS | 177 |
| mAb16716 | 179 | 181 | 183 | 185 | 187 | 972 | AAS | 189 |
| mAb16732 | 191 | 1740 | 193 | 195 | 197 | 199 | TAS | 201 |
| mAb16747 | 203 | 205 | 207 | 209 | 211 | 213 | AAT | 215 |
| mAb21581 | 217 | 219 | 221 | 223 | 225 | 396 | AAS | 1640 |
| mAb21587 | 227 | | | | 229 | | | |
| mAb21589 | 231 | | | | 125 | | | |

Table 14 sets forth the amino acid sequence identifiers of the heavy and light chain variable regions and CDRs of selected anti-CD3 antibodies. Other anti-CD3 antibodies for use in preparing bispecific antibodies in accordance with the present disclosure can be found in, e.g., WO 2014/047231.

TABLE 14
Amino Acid Sequence Identifiers

| | HCVR | HCDR1 | HCDR2 | HCDR3 | LCVR | LCDR1 | LCDR2 | LCDR3 |
|---|---|---|---|---|---|---|---|---|
| | (SEQ | (SEQ | (SEQ | (SEQ | (SEQ | (SEQ | (amino | (SEQ |
| Antibody | ID | ID | ID | ID | ID | ID | acid | ID |
| Designation | NOs) | NOs) | NOs) | NOs) | NOs) | NOs) | sequences) | NOs) |
| mAb7221G | 1610 | 740 | 438 | 1512 | 394 | 396 | AAS | 312 |
| mAb7221G20 | 1866 | 740 | 406 | 1848 | 394 | 396 | AAS | 312 |

In certain exemplary embodiments, the isolated anti-BCMA x anti-CD3 bispecific antigen binding molecule comprises a first antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 217; and (b) three light chain complementarity determining regions (LCDR1, LCDR2 and LCDR3) contained within a light chain variable region (LCVR) comprising the amino acid sequence of SEQ ID NO:394. In some cases, the isolated bispecific antigen binding molecule comprises a HCDR1 comprising the amino acid sequence of SEQ ID NO:219, a HCDR2 comprising the amino acid sequence of SEQ ID NO:221, and a HCDR3 comprising the amino acid sequence of SEQ ID NO:223. In some cases, the isolated bispecific antigen-binding molecule comprises a LCDR1 comprising the amino acid sequence of SEQ ID NO:396, a LCDR2 comprising the amino acid sequence AAS, and a LCDR3 comprising the amino acid sequence of SEQ ID NO:312. In some cases, the first antigen-binding domain comprises a HCVR comprising the amino acid sequence of SEQ ID NO: 217, and a LCVR comprising the amino acid sequence of SEQ ID NO: 394.

In certain exemplary embodiments, the isolated anti-BCMA x anti-CD3 bispecific antigen-binding molecule comprises a second antigen-binding domain that comprises: (a) three heavy chain complementarity determining regions (HCDR1, HCDR2 and HCDR3) contained within a heavy chain variable region (HCVR) comprising the amino acid sequence of SEQ ID NO: 1610 or SEQ ID NO: 1866; and (b) three light chain complementarity determining regions (LCDR1, LCDR2 and LCDR3) contained within a light chain variable region (LCVR) comprising the amino acid sequence of SEQ ID NO:394. In some cases, the second antigen-binding domain comprises: (a) a HCDR1 comprising the amino acid sequence of SEQ ID NO: 740; (b) a HCDR2 comprising the amino acid sequence of SEQ ID NO: 438 or SEQ ID NO: 406; and (c) a HCDR3 comprising the amino acid sequence of SEQ ID NO: 1512 or SEQ ID NO: 1848. In some cases, the second antigen-binding domain comprises a LCDR1 comprising the amino acid sequence of SEQ ID NO: 396, a LCDR2 comprising the amino acid sequence AAS, and a LCDR3 comprising the amino acid sequence of SEQ ID NO:312. In some cases, the second antigen-binding domain comprises: (a) HCDR1, HCDR2, HCDR3 domains, respectively, comprising the amino acid sequences of SEQ ID NOs: 740, 438, 1512; and LCDR1, LCDR2, LCDR3 domains, respectively, comprising the amino acid sequences of SEQ ID NO: 396, AAS, SEQ ID NO: 312; or (b) HCDR1, HCDR2, HCDR3 domains, respectively, comprising the amino acid sequences of SEQ ID NOs: 740, 406, 1848; and LCDR1, LCDR2, LCDR3 domains, respectively, comprising the amino acid sequences of SEQ ID NO: 396, AAS, SEQ ID NO: 312. In some cases, the second antigen-binding domain comprises: (a) a HCVR comprising the amino acid sequence of SEQ ID NO: 1610, and a LCVR comprising the amino acid sequence of SEQ ID NO: 394; or (b) a HCVR comprising the amino acid sequence of SEQ ID NO: 1866, and a LCVR comprising the amino acid sequence of SEQ ID NO: 394.

In certain exemplary embodiments, the isolated anti-BCMA x anti-CD3 bispecific antigen-binding molecule comprises: (a) a first antigen-binding domain that comprises HCDR1, HCDR2, HCDR3 domains, respectively, comprising the amino acid sequences of SEQ ID NOs: 219, 221, 223, and LCDR1, LCDR2, LCDR3 domains, respectively, comprising the amino acid sequences of SEQ ID NO: 396, AAS, SEQ ID NO: 312; and (b) a second antigen binding domain that comprises HCDR1, HCDR2, HCDR3 domains, respectively, comprising the amino acid sequences of SEQ ID NOs: 740, 438, 1512, and LCDR1, LCDR2, LCDR3 domains, respectively, comprising the amino acid sequences of SEQ ID NO: 396, AAS, SEQ ID NO: 312. In some cases, the isolated bispecific antigen-binding molecule comprises: (a) a first antigen binding domain that comprises a HCVR comprising the amino acid sequence of SEQ ID NO: 217, and a LCVR comprising the amino acid sequence of SEQ ID NO: 394; and (b) a second antigen binding domain that comprises a HCVR comprising the amino acid sequence of SEQ ID NO: 1610, and a LCVR comprising the amino acid sequence of SEQ ID NO: 394.

In certain exemplary embodiments, the isolated anti-BCMA x anti-CD3 bispecific antigen-binding molecule comprises: (a) a first antigen-binding domain that comprises HCDR1, HCDR2, HCDR3 domains, respectively, comprising the amino acid sequences of SEQ ID NOS: 219, 221, 223, and LCDR1, LCDR2, LCDR3 domains, respectively, comprising the amino acid sequences of SEQ ID NO: 396, AAS, SEQ ID NO: 312; and (b) a second antigen binding domain that comprises HCDR1, HCDR2, HCDR3 domains, respectively, comprising the amino acid sequences of SEQ ID NOs: 740, 406, 1848, and LCDR1, LCDR2, LCDR3 domains, respectively, comprising the amino acid sequences of SEQ ID NO: 396, AAS, SEQ ID NO: 312. In some cases, the isolated bispecific antigen-binding molecule comprises: (a) a first antigen binding domain that comprises a HCVR comprising the amino acid sequence of SEQ ID NO: 217, and a LCVR comprising the amino acid sequence of SEQ ID NO: 394; and (b) a second antigen binding domain that comprises a HCVR comprising the amino acid sequence of SEQ ID NO: 1866, and a LCVR comprising the amino acid sequence of SEQ ID NO: 394.

In certain exemplary embodiments, the isolated anti-BCMA x anti-CD3 bispecific antigen-binding molecule comprises: (a) a first antigen-binding domain that specifically binds human BCMA, and comprises the CDRs of a HCVR comprising an amino acid sequence selected from the group consisting of SEQ ID NOs: 165, 179, 191, 203, 217, 227, and 231, and the CDRs of a LCVR comprising an amino acid sequence selected from the group consisting of SEQ ID NO: 173, 187, 197, 211, 225, 394, 229, and 125; and (b) a second antigen-binding domain that specifically binds human CD3. In some cases, the first antigen-binding domain comprises the CDRs from a HCVR/LCVR amino acid sequence pair selected from the group consisting of SEQ ID NOs: 165/173, 179/187, 191/197, 203/211, 217/225, 227/229, 231/125, 165/394, 179/394, 191/394, 203/394, 217/394, 227/394, and 231/394. In some cases, the first antigen-binding domain comprises HCDR1-HCDR2-HCDR3-LCDR1-LCDR2-LCDR3 domains, respectively, selected from the group consisting of SEQ ID NOs: 167-169-171-175-AAS-177, 181-183-185-972-AAS-189, 1740-193-195-199-TAS-201, 205-207-209-213-AAT-215, 219-221-223-396-AAS-1640, 167-169-171-396-AAS-312, 181-183-185-396-AAS-312, 1740-193-195-396-AAS-312, 205-207-209-396-AAS-312, and 219-221-223-396-AAS-312. In some cases, the first antigen-binding domain comprises the HCVR/LCVR amino acid sequence pair selected from the group consisting of SEQ ID NOs: 165/173, 179/187, 191/197, 203/211, 217/225, 227/229, 231/125, 165/394, 179/394, 191/394, 203/394, 217/394, 227/394, and 231/394. In some cases, the second antigen-binding domain comprises the CDRs of a HCVR/LCVR amino acid sequence pair selected from the group consisting of SEQ ID NOs: 1610/394 and 1866/394.

In certain exemplary embodiments, the isolated anti-BCMA x anti-CD3 bispecific antigen binding molecule competes for binding to BCMA, or binds to the same epitope on BCMA as a reference antibody, wherein the reference antibody comprises a first antigen-binding domain comprising an HCVR/LCVR pair comprising the amino acid sequences of SEQ ID NOS: 217/394 and a second antigen-binding domain comprising an HCVR/LCVR pair comprising the amino acid sequences of either SEQ ID NOs: 1610/394 or SEQ ID NOs: 1866/394.

In certain exemplary embodiments, the isolated anti-BCMA x anti-CD3 bispecific antigen binding molecule competes for binding to human CD3, or binds to the same epitope on human CD3 as a reference antibody, wherein the reference antibody comprises a first antigen-binding domain comprising an HCVR/LCVR pair comprising the amino acid sequences of SEQ ID NOs: 217/394 and a second antigen-binding domain comprising an HCVR/LCVR pair comprising the amino acid sequences of either SEQ ID NOs: 1610/394 or SEQ ID NOS: 1866/394.

Additional bispecific anti-BCMA x anti-CD3 antibodies are disclosed in, e.g., WO 2020/018820.

### CD3xCD20 Antibodies

In some embodiments, provided herein are bispecific antibodies wherein one arm of an immunoglobulin binds human CD3, and the other arm of the immunoglobulin is specific for human CD20. The term “CD20,” as used herein, refers to the human CD20 protein unless specified as being from a non-human species (e.g., “mouse CD20,” “monkey CD20,” etc.). The human CD20 protein has the amino acid sequence shown in SEQ ID NO:1369 of U.S. Pat. No. 9,657,102B2, the content of which is incorporated by reference herein in its entirety. Such molecules may be referred to herein as, e.g., “anti-CD3/anti-CD20,” or “anti-CD3xCD20” or “CD3xCD20” bispecific molecules, or other similar terminology.

In certain embodiments, the first antigen-binding domain that specifically binds CD3 comprises a heavy chain variable region (HCVR) having an amino acid sequence selected from the group consisting of SEQ ID NOs: 1250, 1266, 1282, 1298, 1314 and 1329 or a substantially similar sequence thereof having at least 90%, at least 95%, at least 98% or at least 99% sequence identity. All sequences disclosed in this section (i.e., “CD3xCD20 antibodies” section) for antigen-binding domains that specifically bind CD3 or CD20 and the corresponding SEQ ID NOs. are from U.S. Pat. No. 9,657,102B2, the content of which is incorporated by reference herein in its entirety.

In certain embodiments, the first antigen-binding domain that specifically binds CD3 comprises a light chain variable region (LCVR) having an amino acid sequence selected from the group consisting of SEQ ID NOs: 1258, 1274, 1290, 1306, 1322 and 1333, or a substantially similar sequence thereof having at least 90%, at least 95%, at least 98% or at least 99% sequence identity.

In certain embodiments, the first antigen-binding domain that specifically binds CD3 comprises a HCVR and LCVR (HCVR/LCVR) amino acid sequence pair selected from the group consisting of SEQ ID NOs: 1250/1258, 1266/1274, 1282/1290, 1298/1306, 1314/1322, and 1329/1333.

In certain embodiments, the first antigen-binding domain that specifically binds CD3 comprises a heavy chain CDR1 (HCDR1) domain having an amino acid sequence selected from the group consisting of SEQ ID NOs:1252, 1268, 1284, 1300, 1316 and 1330, or a substantially similar sequence thereof having at least 90%, at least 95%, at least 98% or at least 99% sequence identity; a heavy chain CDR2 (HCDR2) domain having an amino acid sequence selected from the group consisting of SEQ ID NOs: 1254, 1270, 1286, 1302, 1318 and 1331, or a substantially similar sequence thereof having at least 90%, at least 95%, at least 98% or at least 99% sequence identity; a heavy chain CDR3 (HCDR3) domain having an amino acid sequence selected from the group consisting of SEQ ID NOs: 1256, 1272, 1288, 1304, 1320 and 1332, or a substantially similar sequence thereto having at least 90%, at least 95%, at least 98% or at least 99% sequence identity; a light chain CDR1 (LCDR1) domain having an amino acid sequence selected from the group consisting of SEQ ID NOs: 1260, 1276, 1292, 1308, 1324 and 1334, or a substantially similar sequence thereof having at least 90%, at least 95%, at least 98% or at least 99% sequence identity; a light chain CDR2 (LCDR2) domain having an amino acid sequence selected from the group consisting of SEQ ID NOs: 1262, 1278, 1294, 1310, 1326 and 1335, or a substantially similar sequence thereof having at least 90%, at least 95%, at least 98% or at least 99% sequence identity, and a light chain CDR3 (LCDR3) domain having an amino acid sequence selected from the group consisting of SEQ ID NOs: 1264, 1280, 1296, 1312, 1328 and 1336, or a substantially similar sequence thereof having at least 90%, at least 95%, at least 98% or at least 99% sequence identity.

In certain embodiments, the first antigen-binding domain that specifically binds CD3 comprises HCDR1-HCDR2-HCDR3-LCDR1-LCDR2-LCDR3 domains, respectively, having the amino acid sequences selected from the group consisting of: SEQ ID NOs: 1252-1254-1256-1260-1262-1264; 1268-1270-1272-1276-1278-1280; 1284-1286-1288-1292-1294-1296; 1300-1302-1304-1308-1310-1312; 1316-1318-1320-1324-1326-1328; and 1330-1331-1332-1334-1335-1336.

In certain embodiments, the second antigen-binding domain that specifically binds CD20 comprises a heavy chain variable region (HCVR) having the amino acid sequence of SEQ ID NO: 1242, or a substantially similar sequence thereof having at least 90%, at least 95%, at least 98% or at least 99% sequence identity.

In certain embodiments, the second antigen-binding domain that specifically binds CD20 comprises a light chain variable region (LCVR) having the amino acid sequence selected from the group consisting of SEQ ID NOs:1258, 1274, 1290, 1306, 1322 and 1333, or a substantially similar sequence thereof having at least 90%, at least 95%, at least 98% or at least 99% sequence identity.

In certain embodiments, the second antigen-binding domain that specifically binds CD20 comprises a HCVR and LCVR (HCVR/LCVR) amino acid sequence pair selected from the group consisting of SEQ ID NOs: 1242/1258, 1242/1274, 1242/1290, 1242/1306, 1242/1322 and 1242/1333.

In certain embodiments, the second antigen-binding domain that specifically binds CD20 comprises a heavy chain CDR1 (HCDR1) domain having the amino acid sequence of SEQ ID NO:1244, or a substantially similar sequence thereof having at least 90%, at least 95%, at least 98% or at least 99% sequence identity; a heavy chain CDR2 (HCDR2) domain having the amino acid sequence of SEQ ID NO: 1246, or a substantially similar sequence thereof having at least 90%, at least 95%, at least 98% or at least 99% sequence identity; a heavy chain CDR3 (HCDR3) domain having the amino acid sequence of SEQ ID NO: 1248, or a substantially similar sequence thereto having at least 90%, at least 95%, at least 98% or at least 99% sequence identity; a light chain CDR1 (LCDR1) domain having an amino acid sequence selected from the group consisting of SEQ ID NOs: 1260, 1276, 1292, 1308, 1324 and 1334, or a substantially similar sequence thereof having at least 90%, at least 95%, at least 98% or at least 99% sequence identity; a light chain CDR2 (LCDR2) domain having an amino acid sequence selected from the group consisting of SEQ ID NOs: 1262, 1278, 1294, 1310, 1326 and 1335, or a substantially similar sequence thereof having at least 90%, at least 95%, at least 98% or at least 99% sequence identity; and a light chain CDR3 (LCDR3) domain having an amino acid sequence selected from the group consisting of SEQ ID NOs: 1264, 1280, 1296, 1312, 1328 and 1336, or a substantially similar sequence thereof having at least 90%, at least 95%, at least 98% or at least 99% sequence identity.

In certain embodiments, the second antigen-binding domain that specifically binds CD20 comprises HCDR1-HCDR2-HCDR3-LCDR1-LCDR2-LCDR3 domains, respectively, having the amino acid sequences selected from the group consisting of: SEQ ID NOs: 1244-1246-1248-1260-1262-1264; 1244-1246-1248-1276-1278-1280; 1244-1246-1248-1292-1294-1296; 1244-1246-1248-1308-1310-1312; 1244-1246-1248-1324-1326-1328; and 1244-1246-1248-1334-1335-1336.

Additional bispecific anti-CD20/anti-CD3 antibodies are disclosed in e.g., U.S. Pat. No. 9,657,102, which is incorporated by reference herein in its entirety.

### Other Exemplary CD3 Multispecific Antibodies

Additional exemplary CD3 multispecific antibodies that can be used in the compositions and methods disclosed herein include but are not limited to, e.g., bispecific CD3xCD123 antibodies disclosed in U.S. Pat. No. 10,787,521B2, U.S. Patent Application Publication Nos. 2018/0222987A1 and US 2019/0241657A1, and International Application Publication Nos. WO 2016/036937A1, WO 2017/210443A1, WO 2019/050521A1, WO 2019/210147A1, WO 2019/232528A1, and WO 2020/092404A1; bispecific CD3xSTEAP2 antibodies disclosed in International Application Publication Nos. WO 2018/058001A1; bispecific CD3xCD20 antibodies disclosed in WO 2014/047231A1, WO 2015/143079A1, WO 2016/081490A1, WO 2017/112775A1, WO 2017/210485A1, WO 2018/114748A1, WO 2018/093821A8, WO 2018/223004A1, WO 2018/188612A1, WO 2019/155008A1, WO 2019/228406A1, WO 2020/088608A1, WO 2020/156405A1, and U.S. Patent Application Publication Nos. US 2020/0199231A1, and US 2020/0172627A1; bispecific CD3xSSTR 2 antibodies disclosed in International Application Publication No. WO 2018/005706A1; bispecific CD3xCD38 antibodies disclosed in International Application Nos. WO 2015/149077A1 and WO 2020/018556A1, and U.S. Patent Application Publication Nos. US 2018/0305465A1 and US 2020/0102403A1; bispecific CD3xSTEAP1 antibodies disclosed in Olivier Nolan-Stevaux (2020) Abstract at Proceedings of the Annual Meeting of the American Association for Cancer Research 2020; bispecific CD3×5T4 antibodies disclosed in International Application Publication No. WO 2013/041687A1, U.S. Patent Application Publication Nos. US 2017/0342160A1, U.S. 20200277397A1; bispecific CD3xENPP3 antibodies as descried in International Application Publication No. WO 2020/180726A1; bispecific CD3xMUC16 antibodies disclosed in International Application Publication Nos. WO 2018/067331A9 and WO 2019/246356A1; bispecific CD3xBCMA antibodies disclosed in International Application Publication Nos. WO 2013/072406A1, WO 2014/140248A1, WO 2016/166629A1, WO 2017/031104A1, WO 2017/134134A1, WO 2017/095267A1, WO 2019/220369A3, WO 2019/075359A1, WO 2019/226761A1, WO 2020/025596A1, WO 2020/191346A1, WO 2020018820A1, U.S. Patent Application Publication Nos. US 2013/0273055A1, US 2019/0263920A1; bispecific CD3xCD19 antibodies disclosed in International Application Publication Nos. WO 2012/055961A1, WO 2016/048938A1, WO 2017/087603A1, WO 2017/096368A1, WO 2018/188612A1, WO 2019/237081A1, WO 2020/048525A1, WO 2020/135335A1, U.S. Patent Application Publication Nos. US 2016/0326249A1, US 2020/0283523A1, US 2019/0284279A1, U.S. Pat. No. 9,315,567B2, U.S. Pat. No. 7,575,923B2, U.S. Pat. No. 7,635,472B2; bispecific CD3xGPRC5D antibodies disclosed in International Application Publication Nos. WO 2018/017786A3, WO 2019/220369A3; bispecific CD3xPSMA antibodies disclosed in U.S. Patent Application Publication No. US 2017/0320947A1; trispecific CD3xCD28xCD38 antibodies disclosed in U.S. Patent Application Publication No. US 2020/0140552A1; or other CD3 multispecific antibodies disclosed in International Application Publication Nos. WO 2016/086189A2, WO 2020/088608A1, WO2019191120A1, and WO 2016/105450A3, the contents of each of which is incorporated by reference herein in its entirety.

In some embodiments, the aforementioned multispecific (e.g., bispecific or trispecific) antigen-binding molecules that specifically bind CD3 and a tumor antigen may comprise an anti-CD3 antigen-binding molecule which binds to CD3 with a weak binding affinity such as exhibiting a K_{D} of greater than about 40 nM, as measured by an in vitro affinity binding assay. The aforementioned bispecific antigen-binding molecules may comprise an anti-CD3 antigen-binding molecule which binds to CD3 and exhibits an EC50 of greater than about 100 nM, as measured by a FACS titration assay. The aforementioned bispecific antigen-binding molecules may comprise an anti-CD3 antigen-binding molecule which exhibits no measurable or observable binding to CD3, as measured by an in vitro affinity binding assay or a FACS titration assay, yet retains ability to activate human PBMC cells and/or induce cytotoxic activity on tumor antigen-expressing cell lines.

### Therapeutic Formulation and Administration

In some aspects, provided herein are pharmaceutical compositions comprising NK cells that express a CAR as described herein. In some aspects, provided herein are pharmaceutical compositions comprising a CD3 multispecific antigen-binding molecule as described herein. In some aspects, provided herein are pharmaceutical compositions in which a CD3 multispecific antigen-binding molecule described herein is co-formulated with NK cells that express a CAR as described elsewhere herein.

The pharmaceutical compositions provided herein can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like.

Various delivery systems are known and can be used to administer a pharmaceutical composition provided herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In some embodiments, a pharmaceutical composition provided herein can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition disclosed herein. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition disclosed herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In some aspects, provided herein are pharmaceutical compositions comprising a NK cell (e.g., an inducible NK cell) expressing a CAR as described herein.

In certain embodiments, the CAR-NK cell populations may be administered either alone, or as a pharmaceutical composition in combination with pharmaceutically or physiologically acceptable carriers, diluents, excipients and/or with other components or cell populations. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions disclosed herein may be formulated for intravenous administration.

The administration of the CAR-NK cells may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, or lymph node.

### Methods for Treating Cancers

In certain embodiments, the disclosure includes methods for treating in a subject. In some embodiments, the methods may comprise conjointly (e.g., concurrently or sequentially) administering to a subject in need thereof: (1) a natural killer (NK) cell expressing a CAR polypeptide comprising an extracellular domain; and (2) a multi-specific antigen-binding molecule comprising a first antigen-binding domain that binds to a tumor antigen and a second antigen-binding domain that binds to the extracellular domain. In certain embodiments, the methods may comprise conjointly (e.g., concurrently or sequentially) administering to a subject in need thereof: (1) a natural killer (NK) cell expressing a CAR polypeptide comprising an extracellular domain that comprises a CD3 extracellular domain or fragment thereof; and (2) a multi-specific antigen-binding molecule comprising a first antigen-binding domain that binds to a tumor antigen and a second antigen-binding domain that binds to the CD3 extracellular domain or fragment thereof. In some embodiments, the CD3 extracellular domain or fragment thereof comprises an epitope recognized by an anti-CD3 antibody. In some embodiments, the anti-CD3 antibody is selected from the anti-CD3 antibodies listed in Table 6. In some embodiments, the CD3 extracellular domain or fragment thereof comprises at least 10 consecutive amino acids of SEQ ID NO: 1959. In some embodiments, the CD3 extracellular domain or fragment thereof comprises an amino acid sequence at least 90% identical to SEQ ID NO: 1959. In certain embodiments, the CD3 extracellular domain or fragment thereof comprises an amino acid sequence of SEQ ID NO: 1959.

In some embodiments, the methods may comprise administering to a subject in need thereof a pharmaceutical composition comprising (1) a natural killer (NK) cell expressing a CAR polypeptide comprising an extracellular domain that comprises a CD3 extracellular domain or fragment thereof; and (2) a multi-specific antigen-binding molecule comprising a first antigen-binding domain that binds to a tumor antigen and a second antigen-binding domain that binds to the CD3 extracellular domain or fragment thereof. The therapeutic composition may further comprise a pharmaceutically acceptable carrier or diluent.

In some aspects, the methods may comprise conjointly (e.g., concurrently or sequentially) administering to a subject in need thereof: (1) an antigen binding molecule that binds to a tumor antigen; and (2) a natural killer (NK) cell expressing a CAR polypeptide comprising an extracellular domain that binds to the antigen-binding molecule.

In certain embodiments, the methods may comprise conjointly (e.g., concurrently or sequentially) administering to a subject in need thereof: (1) a multi-specific antigen binding molecule comprising a CD3-binding domain that specifically binds to CD3 and a tumor antigen-binding domain that specifically binds to a tumor antigen; and (2) a natural killer (NK) cell expressing a CAR polypeptide comprising an extracellular domain that comprises an antigen-binding domain specific for an idiotype of an anti-CD3 antibody, wherein the antigen-binding domain of the CAR polypeptide binds to the idiotype of the CD3-binding domain of the multi-specific antigen binding molecule. In some embodiments, the anti-CD3 antibody is selected from the anti-CD3 antibodies listed in Table 6. In some embodiments, the antigen-binding domain is a single chain fragment variable (scFv). In some embodiments, the antigen-binding domain comprises the heavy chain and light chain CDR sequences of a scFv listed in Table 1. In some embodiments, the antigen-binding domain comprises the heavy chain and light chain variable region sequences of one of a scFv listed in Table 1. In some embodiments, the antigen-binding domain comprises the amino acid sequence of a scFv listed in Table 1.

In some embodiments, the methods may comprise administering to a subject in need thereof a pharmaceutical composition comprising (1) a multi-specific antigen binding molecule comprising a CD3-binding domain that specifically binds to CD3 and a tumor antigen-binding domain that specifically binds to a tumor antigen; and (2) a natural killer (NK) cell expressing a CAR polypeptide comprising an extracellular domain that comprises an antigen-binding domain specific for an idiotype of an anti-CD3 antibody, wherein the antigen-binding domain of the CAR polypeptide binds to the idiotype of the CD3-binding domain of the multi-specific antigen binding molecule. The therapeutic composition may further comprise a pharmaceutically acceptable carrier or diluent.

In some aspects, the methods may comprise conjointly (e.g., concurrently or sequentially) administering to a subject in need thereof: (a) an antigen binding molecule that binds to a tumor antigen and that comprises an Fc domain; and (b) a natural killer (NK) cell expressing a CAR polypeptide comprising an extracellular domain that binds to the Fc domain.

In certain embodiments, the methods may comprise conjointly (e.g., concurrently or sequentially) administering to a subject in need thereof: (1) a multi-specific antigen binding molecule comprising a CD3-binding domain that specifically binds to CD3, a tumor antigen-binding domain that specifically binds to a tumor antigen, and a Fc domain; and (2) a natural killer (NK) cell expressing a CAR polypeptide comprising an extracellular domain that comprises an antigen binding domain specific for an Fc domain, wherein the antigen binding domain of the CAR polypeptide binds to the Fc domain of the multi-specific antigen binding molecule.

In some embodiments, the anti-CD3 antibody is selected from the anti-CD3 antibodies listed in Table 6. In some embodiments, the antigen-binding domain is a single chain fragment variable (scFv). In some embodiments, the antigen-binding domain comprises the heavy chain and light chain CDR sequences of a scFv listed in Table 1. In some embodiments, the antigen-binding domain comprises the heavy chain and light chain variable region sequences of one of a scFv listed in Table 1. In some embodiments, the antigen-binding domain comprises the amino acid sequence of a scFv listed in Table 1.

In some embodiments, the methods may comprise administering to a subject in need thereof a pharmaceutical composition comprising (1) a multi-specific antigen binding molecule comprising a CD3-binding domain that specifically binds to CD3, a tumor antigen-binding domain that specifically binds to a tumor antigen, and a Fc domain; and (2) a natural killer (NK) cell expressing a CAR polypeptide comprising an extracellular domain that comprises an antigen binding domain specific for an Fc domain, wherein the antigen binding domain of the CAR polypeptide binds to the Fc domain of the multi-specific antigen binding molecule. The therapeutic composition may further comprise a pharmaceutically acceptable carrier or diluent.

As used herein, the terms “treat”, “treating”, or the like, mean to alleviate symptoms, or eliminate the causation of symptoms either on a temporary or permanent basis. For example, “treating cancer” may mean to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with cancer, and who needs treatment for the same. In many embodiments, the term “subject” may be interchangeably used with the term “patient”.

In some embodiments, cancers that may be treated by methods and compositions provided herein include, but are not limited to, cancer from the cervix, anus, vagina, vulva, penis, tongue base, larynx, tonsil, bladder, blood, bone, bone marrow, brain, breast, colon, esophagus, gastrointestine, gum, head, kidney, liver, lung, nasopharynx, neck, ovary, prostate, skin, non-melanoma skin cancer (NMSC), cutaneous squamous cell carcinoma (SCC), stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, cancers that may be treated by methods and compositions provided herein express the tumor antigen targeted by the antigen binding molecule or the multi-specific antigen-binding molecule (e.g., the CD3 multispecific antigen-binding molecules). In some embodiments, cancers treated by methods and compositions provided herein may be a tumor with an expression of the tumor antigen as determined by flow cytometry on ≥20% of the tumor cells. In particular, the compositions and methods disclosed herein may be used for the treatment, prevention and/or amelioration of any disease or disorder associated with or mediated by, e.g., CD20, PSMA, MUC16, STEAP2 or BCMA expression or activity or the proliferation of CD20⁺, PSMA⁺, MUC16⁺, STEAP2⁺, or BCMA⁺ cells. The mechanism of action by which the therapeutic methods disclosed herein are achieved include killing of the cells expressing such antigens in the presence of effector cells, for example, by CDC, apoptosis, ADCC, phagocytosis, or by a combination of two or more of these mechanisms.

In some embodiments, the CD3 multispecific antigen binding molecule used in the present compositions or methods is a bispecific anti-CD3 x anti-PSMA antibody. The compositions or methods are useful for treating a PSMA-expressing cancer including prostate cancer, kidney cancer, bladder cancer, colorectal cancer, and gastric cancer. In some embodiments, the cancer is prostate cancer (e.g., castrate-resistant prostate cancer).

In some embodiments, the CD3 multispecific antigen binding molecule used in the present compositions or methods is a bispecific anti-CD3 x anti-MUC16 antibody. The compositions or methods are useful for treating a MUC16-expressing cancer including ovarian cancer, breast cancer, pancreatic cancer, non-small-cell lung cancer, intrahepatic cholangiocarcinoma-mass forming type, adenocarcinoma of the uterine cervix, and adenocarcinoma of the gastric tract. In some embodiments, the cancer is ovarian cancer.

In some embodiments, the CD3 multispecific antigen binding molecule used in the present compositions or methods is a bispecific anti-CD3 x anti-STEAP2 antibody. The compositions or methods are useful for treating a STEAP2-expressing cancer including prostate cancer, bladder cancer, cervical cancer, lung cancer, colon cancer, kidney cancer, breast cancer, pancreatic cancer, stomach cancer, uterine cancer, and ovarian cancer. In some embodiments, the cancer is prostate cancer (e.g., castrate-resistant prostate cancer).

In some embodiments, the CD3 multispecific antigen binding molecule used in the present compositions or methods is a bispecific anti-CD3 x anti-BCMA antibody. The compositions or methods are useful for treating a BCMA-expressing cancer including multiple myeloma or other B-cell or plasma cell cancers, such as Waldenstrom's macroglobulinemia, Burkitt lymphoma, and diffuse large B-Cell lymphoma, Non-Hodgkin's lymphoma, chronic lymphocytic leukemia, follicular lymphoma, mantle cell lymphoma, marginal zone lymphoma, lymphoplasmacytic lymphoma, and Hodgkin's lymphoma. In some embodiments, the cancer is multiple myeloma.

In some embodiments, the CD3 multispecific antigen binding molecule used in the present compositions or methods is a bispecific anti-CD3 x anti-CD20 antibody. The compositions or methods are useful for treating a CD20-expressing cancer including non-Hodgkin lymphoma, Hodgkin lymphoma, chronic lymphocytic leukemia, acute lymphoblastic leukemia, small lymphocytic lymphoma, diffuse large B-cell lymphoma, follicular lymphoma, mantle cell lymphoma, marginal zone lymphoma, Waldenstrom macroglobulinemia, primary mediastinal B-cell lymphoma, lymphoblastic lymphoma, or Burkitt lymphoma. In some embodiments, the cancer is follicular lymphoma. In some embodiments, the cancer is diffuse large B-cell lymphoma (DLBCL).

In certain embodiments, the methods disclosed herein are used in a subject that has been treated with certain cancer drugs (e.g., cancer immunotherapy, CAR-T cell therapy, or CD3 multispecific antigen binding molecules such as those described herein).

In some embodiments, for any of the methods disclosed herein, the subject treated, or the subject evaluated, is a subject to be treated or who has been treated with a cancer immunotherapy, e.g., a CD3 multispecific antigen binding molecule as described herein.

In some embodiments, the methods provided herein treat, delay, or inhibit the growth of a tumor, or induce tumor cell death. In certain embodiments, the methods provided herein promote tumor regression. In certain embodiments, the methods provided herein reduce tumor cell load or to reduce tumor burden. In certain embodiments, the methods provided herein prevent tumor recurrence.

In certain embodiments, the disclosed natural killer (NK) cell expressing a CAR polypeptide, and/or antigen-binding molecule (e.g., a CD3 multispecific antigen binding molecule) are administered to a patient in conjunction with (e.g., before, simultaneously or following) any number of relevant treatment modalities, including but not limited to additional cancer treatments.

The additional therapeutically active component(s) may be administered just prior to, concurrent with, or shortly after the administration of the disclosed natural killer (NK) cell expressing a CAR polypeptide, and/or antigen-binding molecule (e.g., a CD3 multispecific antigen binding molecule); (for purposes of the present disclosure, such administration regimens are considered the administration of the disclosed natural killer (NK) cell expressing a CAR polypeptide, and/or antigen-binding molecule (e.g., a CD3 multispecific antigen binding molecule) “in combination with” an additional therapeutically active component).

Combined administration, as described above, may be simultaneous, separate, or sequential. For simultaneous administration, the agents may be administered as one composition or as separate compositions, as appropriate.

### Administration Regimens

In certain embodiments, provided herein are methods comprising administering to a subject a NK cell expressing a CAR described herein at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In certain embodiments, provided herein are methods comprising administering to a subject a CD3 multispecific antigen binding molecule at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In certain embodiments, the methods involve the administration of a NK cell expressing a CAR described herein in combination with a CD3 multispecific antigen binding molecule at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

According to certain embodiments, multiple doses of a NK cell expressing a CAR described herein in combination with a CD3 multispecific antigen binding molecule may be administered to a subject over a defined time course. The methods according to this aspect disclosed herein may comprise sequentially administering to a subject multiple doses of a NK cell expressing a CAR described herein in combination with a CD3 multispecific antigen binding molecule. As used herein, “sequentially administering” means that each dose of a CAR-NK cell or an antigen-binding molecule is administered to the subject at a different point in time, e.g., on different days separated by a predetermined interval (e.g., hours, days, weeks or months). In certain embodiments, the disclosure includes methods which comprise sequentially administering to the patient a single initial dose of a NK cell expressing a CAR described herein, followed by one or more secondary doses of the NK cell expressing a CAR described herein, and optionally followed by one or more tertiary doses of the NK cell expressing a CAR described herein. In certain embodiments, the present disclosure further comprises sequentially administering to the patient a single initial dose of a CD3 multispecific antigen binding molecule, followed by one or more secondary doses of the CD3 multispecific antigen binding molecule, and optionally followed by one or more tertiary doses of the CD3 multispecific antigen binding molecule.

The terms “initial dose,” “secondary doses,” and “tertiary doses,” refer to the temporal sequence of administration of the antigen-binding molecule disclosed herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the therapeutic agents described herein, but generally may differ from one another in terms of frequency of administration. In certain embodiments, however, the amount of an antigen-binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment disclosed herein, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26) or more weeks after the immediately preceding dose. The phrase “the immediately preceding dose,” as used herein, means, in a sequence of multiple administrations, the dose of the therapeutic agents described herein which is administered to a patient prior to the administration of the very next dose in the sequence with no intervening doses.

The methods according to this aspect disclosed herein may comprise administering to a patient any number of secondary and/or tertiary doses of the therapeutic agents described herein. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXEMPLIFICATION

### Example 1: Evaluation of the Ability of Anti-CD20 One-Arm Antibody to Induce Target-Dependent AhFc-CAR-Dependent Signaling Using Jurkat/NFAT-Luc/ahFc-CD28-CD3z, and ahFc-CAR to Induce AhFc-CAR Ramos Cytolysis Using KHYG1/ahFc-CD28-CD3z

### Experimental Procedure

### Cell Line Engineering

Engineering of Luciferase based reporter cell line: The human acute T cell leukemia derived Jurkat E6 cell line was transduced with an NFAT response element driven luciferase reporter construct. Puromycin resistant cells were maintained in RPMI1640 supplemented with 10% FBS, L-glutamine, Penicillin and Streptomycin and 1 mg/mL Puromycin. The cell line was single cell-sorted, and a single clone identified and renamed Jurkat/NFAT-Luc cl. 3C7 (ACL8722). Jurkat/NFAT-Luc c1. 3C7 were transduced with a vector encoding a chimeric construct comprised of an mROR signal sequence, an anti-human Fc scFv moiety, a G4S linker (SEQ ID NO: 89), CD28 hinge, transmembrane, and cytoplasmic domain, and CD32 cytoplasmic domain. Blasticidin resistant cells were maintained in RPMI1640 supplemented with 10% FBS, L-glutamine, Penicillin and Streptomycin, 1 mg/mL Puromycin and 10 mg/mL Blasticidin. The cell line was renamed Jurkat/NFAT-Luc/ahFc-CD28-CD3z (ACL21770).

Engineering of a B cell line reporter cell for evaluating cytolytic activity: The human B lymphocyte cell line Ramos.2G6.4C10 was transduced with a vector encoding a chimeric construct comprised of enhanced GFP (eGFP) (WP_031943942.1 M1-K239), a GSGGSG linker (SEQ ID NO: 90) and a HiBIT tag (VSGWRLFKKIS) (SEQ ID NO: 1960). eGFP⁺ cells were sorted and maintained in RPMI1640 supplemented with 10% FBS, L-glutamine, Penicillin and Streptomycin. The cell line was renamed Ramos/GFP (ACL21777).

Engineering of an NK cell line with cytolytic activity: The human natural killer cell leukemia derived KHYG-1 cell line was transduced with a vector encoding a chimeric construct comprised of an mROR signal sequence, an scFv moiety targeting human Fc, a G4S linker (SEQ ID NO: 89), CD28 hinge, transmembrane, and cytoplasmic domain, and CD3z cytoplasmic domain. Blasticidin resistant cells were maintained in RPMI1640 supplemented with 10% FBS, L-glutamine, Penicillin and Streptomycin, 10 ng/ml IL2 and 5 mg/mL Blasticidin. The cell line was renamed KHYG/ahFc-CD28-CD3z (ACL21772).

### Jurkat/NFAT-Luc/ahFc-CD28-CD3z Signaling Bioassay:

To assess target and antibody dependent agonistic activity of the anti-Fc chimeric construct, a cell-based reporter assay was established where an antibody is co-incubated with target cells and Jurkat/NFAT-Luc cells expressing the anti-hFc CAR construct, which, upon clustering of the CAR construct, leads to the activation of Nuclear Factor of Activated T cells (NFAT) response element driven luciferase expression. Assessment of the impact of an anti-CD20 antibody (REGN2959: anti-CD20 (10F2) one arm antibody IgG1) and a non-targeting isotype control (REGN1932: isotype control IgG1) was performed in the presence of cells either positive (Ramos.2G6.4C10) or negative (Jurkat) for CD20 expression.

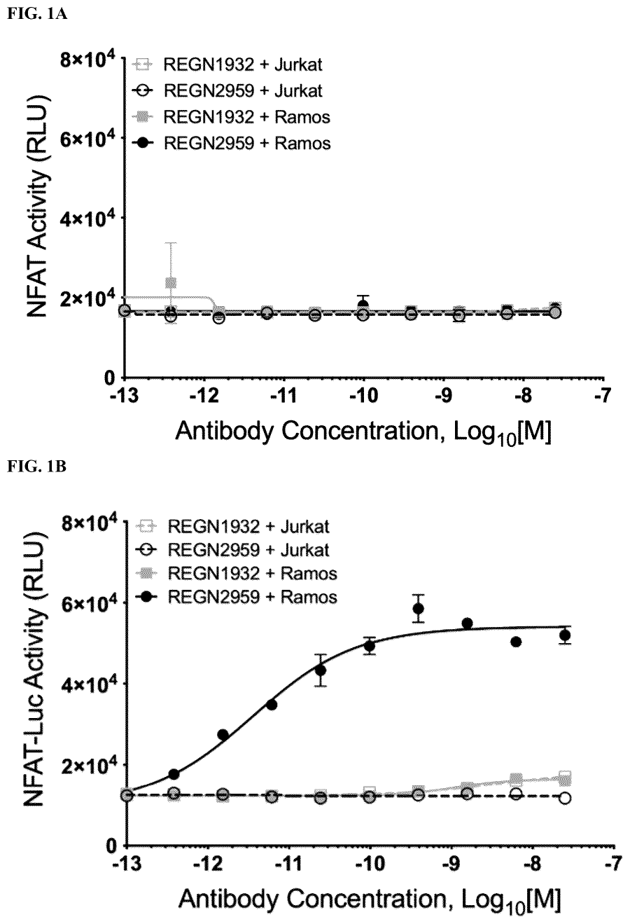

RPMI1640 supplemented with 10% FBS, L-glutamine, Penicillin and Streptomycin, was used as assay medium to prepare cell suspensions and antibody dilutions. A day prior to screening, all reporter and target cells were resuspended at 3×10⁵ cells/mL. Day of assay, Jurkat/NFAT-Luc cl.3C7 or Jurkat/NFAT-Luc/ahFc-CD28-CD3z reporter cells were plated at 2.5×10⁴ reporter cells/well in 96 well white flat bottom plates. Anti-CD20 one-arm antibody [REGN2959] or an isotype control [REGN1932] were serially diluted (1:4) over a 9-point titration range (25 nM to 0.38 PM) (FIGS. 1A and 1B) with a 10th point containing no antibody (represented as 0.10 pM), and added to cells prior to the addition of 2.5×10⁴ either Ramos.2G6.4C10 or Jurkat target cells/well. Plates were incubated for 5 hours at 37° C./5% CO₂ and then 100 μL detection reagent was added to the wells to lyse the cells and detect luciferase activity. The emitted light was measured in RLU on a multilabel plate reader Envision (PerkinElmer).

Fold induction was calculated using the following equation:

$$Maximum Fold Induction = \frac{Highest mean RLU value within tested dose range}{Mean RLU values of no protein control}$$

### KHYG/ahFc-CD28-CD3z Target Cell Cytolysis Assay:

To assess antibody dependent NK cell activation through the anti-Fc chimeric construct, a NK-cell line based cytolytic assay was established where an antibody is co-incubated with target cells and KHYG cells expressing the anti-hFc CAR construct. Clustering of the CAR construct leads to the cytolysis of target cells. Detection of the tag released in the supernatant is used as surrogate of target lysis.

RPMI1640 supplemented with 10% FBS, L-glutamine, Penicillin and Streptomycin, was used as assay medium to prepare cell suspensions and antibody dilutions. A day prior to screening, the transduced NK cell line and target cell were resuspended at 3×10⁵ cells/mL. Day of assay, KHYG/ahFc-CD28-CD3z cells were plated at 2.5×10⁴ reporter cells/well in 96 well white flat bottom plates. 5×10³ Ramos/GFP were added. Anti-CD20 one-arm antibody [REGN2959] or an isotype control [REGN1932] were serially diluted (1:4) over a 9-point titration range (25 nM to 0.38 pM) (FIGS. 1A and 1B) with a 10^{th} point containing no antibody (represented as 0.10 pM), and added to cells, and plates were incubated for 5 hours at 37° C./5% CO₂ prior addition of 100 μL detection reagent to detect extracellular tag. The emitted light was measured in RLU on a multilabel plate reader Envision (PerkinElmer).

Percent cytotoxicity was calculated using the following equation:

$$Percent cytotoxicity = \frac{( sample RLU ) - ( mean RLU in absence of antibody )}{\begin{matrix}( mean RLU in presence of 100 ug / mL of digitonin ) - \\ ( mean RLU in absence of antibody )\end{matrix}} \times 100$$

Maximum percent cytotoxicity was highest replicates mean cytotoxicity across the antibody dose range.

### Results:

### Jurkat/NFAT-Luc/ahFc-CD28-CD3z Signaling Bioassay:

As shown in FIGS. 1A and 1B, in the presence of CAR expressing Jurkat reporter cells, target expressing cells (Ramos) and anti-CD20 (REGN2959), a dose-dependent increase in NFAT-driven luciferase reporter expression was detected (maximum signal: 4.54 fold). In contrast, the absence of either CAR expression on Jurkat reporter cells, target expression (using Jurkat as target cells), or antibody targeting (using the isotype control REGN1932), did not lead to an increase in luciferase reporter expression (maximum signal: 1.33 fold) (FIGS. 1A and 1B, Table 15).

Table 15 shows the maximum fold induction of signal across the antibody dose range from Jurkat/NFAT-Luc cl.3C7 or Jurkat/NFAT-Luc/ahFc-CD28-CD3 incubated with Jurkat or Ramos and anti-CD20 (REGN2959) or an isotype control (REGN1932).

TABLE 15
Maximum Fold Induction from Jurkat reporter cells,
in the presence or absence of ahFc-CD28-CD3 receptor,
Ramos target cells and CD20 antibody.

| | Max fold induction* | |
|---|---|---|
| | Jurkat/NFAT-Luc | Jurkat/NFAT-Luc/ |
| | cl.3C7 | ahFc-CD28-CD3 |
| Jurkat + REGN1932 | 1.04 | 1.33 |
| Jurkat + REGN2959 | 1.00 | 1.05 |
| Ramos + REGN1932 | 1.43 | 1.29 |
| Ramos + REGN2959 | 1.05 | 4.54 |

*Max fold induction was defined as the highest mean RLU across the ab dose range divided by the mean RLU in the absence of protein.

### KHYG/ahFc-CD28-CD3z Target Cell Cytolysis Assay:

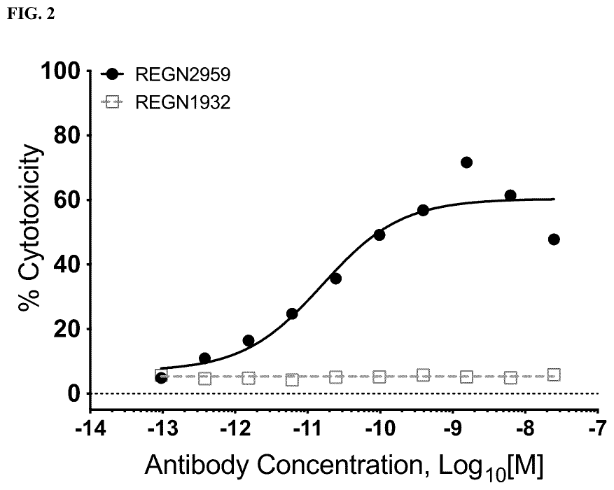

As shown in FIG. 2, in the presence of the CAR expressing KHYG-1 NK cell line and target cells (Ramos/GFP), addition of anti-CD20 (REGN2959) led to a dose-dependent increase in target cytolysis (maximum cytolysis detected: 71.65%). In contrast, a non-targeted antibody fails to induce Ramos cytolysis (maximum cytolysis detected: 5.86%) (FIG. 2, Table 16).

Table 16 shows the maximum percent cytolysis induction across the antibody dose range from KHYG-1/ahFc-CD28-CD3 incubated with Ramos and anti-CD20 (REGN2959) or an isotype control (REGN1932).

TABLE 16
Maximum Percent Cytolysis Induction from KHYG-1
cells expressing ahFc-CD28-CD3 incubated with
Ramos/GFP cells and CD20 antibody.

| | Max percent |
|---|---|
| | cytolysis* |
| Ramos + REGN1932 | 5.86 |
| Ramos + REGN2959 | 71.65 |

*Max percent cytolysis was defined as the highest percent cytolysis across the ab dose range.

### Example 2: Biacore Binding Data for scFvs Directed at CD3 Antibody Idiotypes (09F7 and 7221G) or a Modified Fc (Fc*)

VelocImmune (humanized) mice were immunized with either CD3 bivalent or CD3 bispecific antibodies, to generate anti-idiotype antibodies. Similarly, antibodies recognizing defined features of a modified antibody Fc domain were generated by immunizing VelocImmune mice with said Fc-modified antibodies. Anti-drugs with desired binding properties, as determined by ELISA, were reformatted into single chain variable fragments (ScFv's). Surface plasmon resonance (SPR) technology was used to evaluate the binding of ScFv's to antibody immunogens and antibodies with similar target specificity but different binding properties.

Biacore kinetics for binding of ScFv supernatants directed at CD3 antibody idiotypes (09F7 and 7221G) or a modified Fc (Fc*) to a panel of human antibodies were determined in an scFv capture format at 25° C. Anti-09F7 scFv (also referred to as “PN29950_LCHC”) was derived from anti-idiotype antibodies generated from mice immunized with REGN1453 (Anti-hCD20 x Anti-hCD3-9F07). Anti-7221G scFv (also referred to as “PN77570_HCLC”) were derived from anti-idiotype antibodies generated from mice immunized with H4tH7221G (Anti-hCD3-7221G). Anti-Fc*scFv (also referred to as “PN78216_HCLC”) was derived from an antibody generated from mice immunized with Fc-modified antibodies.

### Experimental Procedure

Equilibrium dissociation constants (KD values) of anti-idiotype (09F7, 7221G, Fc*) scFv fused to an HA-tag supernatants binding to a panel of human antibodies were determined using real-time surface plasmon resonance biosensor technology on a Biacore T-200 or 8k instrument. Briefly, the CM5 Biacore sensor surface was derivatized by amine coupling with a monoclonal mouse anti-HA antibody (Abcam, Cat #ab18181, Clone HA.C5). All Biacore binding studies were performed in a buffer composed of 10 mM HEPES pH 7.4, 150 mM NaCl, 0.05% v/v Surfactant P20 (HBS-EP running buffer). scFv supernatants (targeting 09F7, 7221G, or Fc*antibodies) were captured onto the anti-HA surface by injecting at a flow rate of 5 or 10 μL/min for 90 or 120 seconds. A single concentration (50 or 100 nM) of antibodies were injected over the captured scFvs at a flow rate of 30 μL/minute. scFv-antibody association was monitored for 90 or 120 seconds, and dissociation was monitored for 120 seconds. At the end of each cycle, the scFv capture surface was regenerated using two 10-second injections of 50 mM NaOH. All binding kinetics experiments were performed at 25° C.

### Data Analysis:

The specific SPR-Biacore sensorgrams were obtained by a double referencing procedure. This was performed by first subtracting the signal of each injection over a reference surface (anti-HA) from the signal over the experimental surface (anti-HA-captured scFvs) thereby removing contributions from refractive index changes. In addition, running buffer injections were performed to allow subtraction of the signal changes resulting from the dissociation of captured scFv from the coupled anti-HA surface. Kinetic association (kₐ) and dissociation (k_{d}) rate constants were determined by fitting the real-time sensorgrams to a 1:1 binding model using Scrubber v2.0c curve fitting software or Cytiva Insight v4.0 software. Binding dissociation equilibrium constants (K_{D}) and dissociative half-lives (t1/2) were calculated from the kinetic rate constants as:

$${K}_{D} ( M ) = \frac{kd}{ka} , and {t}^{1 / 2} ( min ) = \frac{ln ( 2 )}{60 * kd} \qquad ( 2 )$$

### Results:

Kinetics results are presented in Table 17 for PN29950_LCHC, an anti-7221G scFv; Table 18 for PN77570_HCLC, an anti-09F7 scFv; and Table 19 for PN78216_HCLC, an anti-Fc*scFv. All kinetics results were determined at 25° C.

TABLE 17
Summary of kinetic and equilibrium binding parameters of various format of anti-CD3,
Fc*, and control antibodies to surface captured anti-7221G scFv (PN29950_LCHC).

| scFv | | | Ab | | | | |
|---|---|---|---|---|---|---|---|
| Capture | REGN # / Ab | | Bind | | | | t½ |
| (RU) | PID # / Cat # | Ab Common Name | (RU) | ka (1/Ms) | kd (1/s) | KD (M) | (min) |
| 780.8 | REGN2533 | (CD3-09F7) [Fc*/Fc*, IgG4s] | 3.2 | NB* | NB* | NB* | NB* |
| 2081.6 | REGN1453 | (CD3-9F07 × CD20) [Fc*/Fc, hIgG1/hIgG4 hybrid] | 6.9 | NB* | NB* | NB* | NB* |
| 734.1 | REGN2280 | (CD20 × CD3-09F7) [Fc*/Fc, IgG1 → only IgG1*] | 2.4 | NB* | NB* | NB* | NB* |
| 1527.2 | H4tH7221G | (CD3-7221G) [Fc*/Fc*, IgG4t] | 2175.8 | 6.01E+05 | ≤1E−4 | 1.66E−10 | ≥115.5 |
| 684.3 | REGN5949 | (CD20 × CD3-7221G) [Fc*/Fc, IgG4s] | 1430.0 | 2.25E+05 | 3.61E−04 | 1.60E−09 | 32.0 |
| 580.3 | H4tH7221G5 | (CD3-7221G5) [Fc*/Fc*, IgG4t] | 418.3 | 1.27E+05 | 1.84E−03 | 1.44E−08 | 6.3 |
| 557.3 | H4sH17400D | (CD20 × CD3-7221G5) [Fc*/Fc, IgG4s] | 47.4 | 5.02E+05 | 1.31E−01 | 2.60E−07 | 0.1 |
| 642.2 | H4tH7221G20 | (CD3-7221G20) [Fc*/Fc*, IgG4t] | 1131.5 | 3.71E+05 | ≤1E−4 | 2.70E−10 | ≥115.5 |
| 618.2 | REGN5950 | (CD20 × CD3-7221G20) [Fc*/Fc, IgG4s] | 1030.4 | 1.98E+05 | 5.16E−04 | 2.61E−09 | 22.4 |
| 1488.2 | REGN4336 | (PSMA × CD3-7221G20) [Fc*/Fc, IgG4s] | 1517.1 | 2.69E+05 | 5.45E−04 | 2.03E−09 | 21.2 |
| 516.8 | REGN5951 | (CD20 × CD3-7221G Goldi) [Fc*/Fc, IgG4s] | 1267.8 | 2.86E+05 | 5.68E−04 | 1.99E−09 | 20.3 |
| 500.6 | REGN5375 | (CD20 × CD3-7221P Goldi2) [Fc*/Fc, IgG4s] | 472.2 | 4.43E+05 | 3.07E−02 | 6.93E−08 | 0.4 |
| 1221.1 | REGN7075 | (EGFR × CD28) [Fc*/Fc, IgG4s] | 0.0 | NB* | NB* | NB* | NB* |
| 886.1 | REGN5856 | (4-1BB) [Fc/Fc, IgG4s] for VDJ cross-reactivity | 0.7 | NB* | NB* | NB* | NB* |
| 832.4 | REGN10453 | (SLAMF7) [Fc/Fc, IgG4s] for VDJ cross-reactivity | −0.1 | NB* | NB* | NB* | NB* |
| 436.1 | REGN1932 | (FelD1) [Fc/Fc, IgG1] | 0.3 | NB* | NB* | NB* | NB* |
| 419.3 | REGN1945 | (FelD1) [Fc/Fc, IgG4] | 0.1 | NB* | NB* | NB* | NB* |
| 408.3 | REGN7540 | (FelD1) [Fc/Fc, IgG4s] | −0.3 | NB* | NB* | NB* | NB* |
| 493.4 | HCA194 | hIgG3 Kappa | −0.2 | NB* | NB* | NB* | NB* |
| 461.1 | HCA178 | hIgG3 Lambda | 0.1 | NB* | NB* | NB* | NB* |

*NB = Non-Binding

TABLE 18
Summary of kinetic and equilibrium binding parameters of various format of anti-CD3,
Fc*, and control antibodies to surface captured anti-09F7 scFv (PN77570_HCLC).

| scFv | | | Ab | | | | |
|---|---|---|---|---|---|---|---|
| Capture | REGN # / Ab | | Bind | | | | t½ |
| (RU) | PID # / Cat # | Ab Common Name | (RU) | ka (1/Ms) | kd (1/s) | KD (M) | (min) |
| 87.5 | REGN2533 | (CD3-09F7) [Fc*/Fc*, IgG4s] | 208.8 | 4.99E+05 | ≤1E−4 | 2.00E−10 | ≥115.5 |
| 523.1 | REGN1453 | (CD3-9F07 × CD20) [Fc*/Fc, hIgG1/hIgG4 hybrid] | 1163.0 | 3.65E+05 | 1.33E−04 | 3.64E−10 | 86.9 |
| 85.0 | REGN2280 | (CD20 × CD3-09F7) [Fc*/Fc, IgG1 → only IgG1*] | 289.8 | 2.65E+05 | ≤1E−4 | 3.79E−10 | ≥115.5 |
| 448.2 | H4tH7221G | (CD3-7221G) [Fc*/Fc*, IgG4t] | 4.4 | IC** | IC** | IC** | IC** |
| 81.8 | REGN5949 | (CD20 × CD3-7221G) [Fc*/Fc, IgG4s] | 0.3 | NB* | NB* | NB* | NB* |
| 75.5 | H4tH7221G5 | (CD3-7221G5) [Fc*/Fc*, IgG4t] | 0.6 | NB* | NB* | NB* | NB* |
| 73.6 | H4sH17400D | (CD20 × CD3-7221G5) [Fc*/Fc, IgG4s] | 0.4 | NB* | NB* | NB* | NB* |
| 79.0 | H4tH7221G20 | (CD3-7221G20) [Fc*/Fc*, IgG4t] | 0.3 | NB* | NB* | NB* | NB* |
| 78.8 | REGN5950 | (CD20 × CD3-7221G20) [Fc*/Fc, IgG4s] | 0.5 | NB* | NB* | NB* | NB* |
| 399.5 | REGN4336 | (PSMA × CD3-7221G20) [Fc*/Fc, IgG4s] | 1.6 | NB* | NB* | NB* | NB* |
| 69.8 | REGN5951 | (CD20 × CD3-7221G Goldi) [Fc*/Fc, IgG4s] | 0.5 | NB* | NB* | NB* | NB* |
| 68.0 | REGN5375 | (CD20 × CD3-7221P Goldi2) [Fc*/Fc, IgG4s] | 0.5 | NB* | NB* | NB* | NB* |
| 313.3 | REGN7075 | (EGFR × CD28) [Fc*/Fc, IgG4s] | 0.0 | NB* | NB* | NB* | NB* |
| 90.4 | REGN5856 | (4-1BB) [Fc/Fc, IgG4s] for VDJ cross-reactivity | −0.3 | NB* | NB* | NB* | NB* |
| 89.9 | REGN10453 | (SLAMF7) [Fc/Fc, IgG4s] for VDJ cross-reactivity | 0.0 | NB* | NB* | NB* | NB* |
| 59.7 | REGN1932 | (FelD1) [Fc/Fc, IgG1] | −0.1 | NB* | NB* | NB* | NB* |
| 57.6 | REGN1945 | (FelD1) [Fc/Fc, IgG4] | −0.1 | NB* | NB* | NB* | NB* |
| 56.3 | REGN7540 | (FelD1) [Fc/Fc, IgG4s] | −0.4 | NB* | NB* | NB* | NB* |
| 67.1 | HCA194 | hIgG3 Kappa | 0.5 | NB* | NB* | NB* | NB* |
| 62.4 | HCA178 | hIgG3 Lambda | −0.1 | NB* | NB* | NB* | NB* |

*NB = Non-Binding
**IC = Inconclusive

TABLE 19
Summary of kinetic and equilibrium binding parameters of various format of anti-CD3,
Fc*, and control antibodies to surface captured anti-Fc* scFv (PN78216_HCLC).

| scFv | | | Ab | | | | |
|---|---|---|---|---|---|---|---|
| Capture | REGN # / Ab | | Bind | | | | t½ |
| (RU) | PID # / Cat # | Ab Common Name | (RU) | ka (1/Ms) | kd (1/s) | KD (M) | (min) |
| 106.9 | REGN2533 | (CD3-09F7) [Fc*/Fc*, IgG4s] | 214.5 | 2.60E+05 | ≤1E−4 | 3.85E−10 | ≥115.5 |
| 736.2 | REGN1453 | (CD3-9F07 × CD20) [Fc*/Fc, hIgG1/hIgG4 hybrid] | 792.3 | 2.02E+05 | 2.53E−04 | 1.25E−09 | 45.7 |
| 102.2 | REGN2280 | (CD20 × CD3-09F7) [Fc*/Fc, IgG1 → only IgG1*] | 230.8 | 1.43E+05 | 4.59E−04 | 3.22E−09 | 25.2 |
| 610.1 | H4tH7221G | (CD3-7221G) [Fc*/Fc*, IgG4t] | 965.9 | 4.04E+05 | ≤1E−4 | 2.48E−10 | ≥115.5 |
| 97.0 | REGN5949 | (CD20 × CD3-7221G) [Fc*/Fc, IgG4s] | 182.2 | 1.14E+05 | 6.40E−04 | 5.61E−09 | 18.1 |
| 86.8 | H4tH7221G5 | (CD3-7221G5) [Fc*/Fc*, IgG4t] | 143.8 | 2.09E+05 | ≤1E−4 | 4.78E−10 | ≥115.5 |
| 83.6 | H4sH17400D | (CD20 × CD3-7221G5) [Fc*/Fc, IgG4s] | 98.8 | 8.98E+04 | 7.77E−04 | 8.65E−09 | 14.9 |
| 92.2 | H4tH7221G20 | (CD3-7221G20) [Fc*/Fc*, IgG4t] | 193.4 | 2.59E+05 | ≤1E−4 | 3.87E−10 | ≥115.5 |
| 91.6 | REGN5950 | (CD20 × CD3-7221G20) [Fc*/Fc, IgG4s] | 144.4 | 1.08E+05 | 6.87E−04 | 6.38E−09 | 16.8 |
| 603.5 | REGN4336 | (PSMA × CD3-7221G20) [Fc*/Fc, IgG4s] | 511.8 | 1.58E+05 | 3.18E−04 | 2.01E−09 | 36.3 |
| 77.9 | REGN5951 | (CD20 × CD3-7221G Goldi) [Fc*/Fc, IgG4s] | 138.3 | 1.18E+05 | 7.33E−04 | 6.23E−09 | 15.8 |
| 75.3 | REGN5375 | (CD20 × CD3-7221P Goldi2) [Fc*/Fc, IgG4s] | 136.4 | 1.41E+05 | 7.75E−04 | 5.50E−09 | 14.9 |
| 563.7 | REGN7075 | (EGFR × CD28) [Fc*/Fc, IgG4s] | 332.2 | 1.30E+05 | 3.47E−04 | 2.67E−09 | 33.2 |
| 113.8 | REGN5856 | (4-1BB) [Fc/Fc, IgG4s] for VDJ cross-reactivity | 235.0 | 2.66E+05 | ≤1E−4 | 3.76E−10 | ≥115.5 |
| 110.8 | REGN10453 | (SLAMF7) [Fc/Fc, IgG4s] for VDJ cross-reactivity | 0.3 | NB* | NB* | NB* | NB* |
| 64.0 | REGN1932 | (FelD1) [Fc/Fc, IgG1] | 0.4 | NB* | NB* | NB* | NB* |
| 61.2 | REGN1945 | (FelD1) [Fc/Fc, IgG4] | 0.1 | NB* | NB* | NB* | NB* |
| 59.4 | REGN7540 | (FelD1) [Fc/Fc, IgG4s] | 1.1 | NB* | NB* | NB* | NB* |
| 74.1 | HCA194 | hIgG3 Kappa | 91.2 | 1.63E+05 | 1.22E−04 | 7.51E−10 | 94.3 |
| 67.4 | HCA178 | hIgG3 Lambda | 48.9 | 7.88E+04 | ≤1E−4 | 1.27E−09 | ≥115.5 |

*NB = Non-Binding

### Example 3: Blocking ELISA Data Testing Human CD3 Anti-Idiotype scFvs

ELISA-based methods were used to assess the blocking of anti-hCD3 mAbs binding to ELISA plate coated with hCD3 ε/δ protein in presence of dilutions of hCD3 anti-idiotype scFv.

### Reagents

TABLE 20
ID of scFv derived from human CD3 anti-idiotypic mAbs:

| PN# | CAR # | Description |
|---|---|---|
| PN29950_VL-VH | CAR 6 | Derived from H4tH7221G (Anti- |
| PN29950_VH-VL | | hCD3-7221G) immunization |
| PN77570_VL-VH | CAR 15 | Derived from REGN1453 (Anti- |
| PN77570_VH-VL | | hCD20 x Anti-hCD3-9F07) |
| | | immunization |

VH—Variable Heavy chain;
VL—Variable Light chain;
CAR—Chimeric Antigen Receptor

TABLE 21
Parental mAbs, ligands, and controls:

| Reagent | Description |
|---|---|
| REGN18409 | Anti-hCD3-7221G mAb |
| REGN18411 | Anti-hCD3-7221G20 mAb |
| REGN2533 | Anti-hCD3-9F07 mAb |
| REGN5766 | Parental control |
| | (mouse mAb binding |
| | Anti-hCD3-7221G/G20) |
| REGN2984 | Parental control |
| | (mouse mAb binding |
| | Anti-hCD3-9F07) |
| REGN4393 | Negative control |
| | (Anti-hFc scFv) |
| Mouse IgG1-unlabelled (Southern | Isotype control (mIgG1) |
| Biotech#0102-01) | |
| Mouse IgG2a-unlabelled (Southern | Isotype control (mIgG2) |
| Biotech#0103-01) | |
| Biotinylated hCD3 Epsilon/Delta | Ligand |
| (ε/δ) (Acro Biosystems#CDD-H82W6) | |

mAb—monoclonal antibody

### Experimental Procedure

Avidin (Thermo Scientific) at 5.0 μg/mL in PBS, was coated on 96-well microtiter plates and incubated overnight at 4° C. Nonspecific binding sites were subsequently blocked using a 0.5% (w/v) solution of BSA in PBS (assay buffer) and incubated at room temperature (RT) for approximately 1 hour. 3.0 μg/mL hCD3 ε/δ (Acro Biosystems) was then captured on the avidin coated ELISA plates by incubating for 1 hour at RT.

In 96-well dilution plates, pre-bind blocking reaction using the hCD3 anti-idiotypic scFv and control mAbs with constant amount of Anti-human CD3 mAbs was set up. For the pre-bind reaction, human CD3 anti-idiotype scFv expressed and purified from Chinese Hamster Ovary (CHO) cells were three-fold serially diluted in assay buffer, starting at neat supernatant; parental control mAbs, scFv negative control, and isotype control mAbs, were three-fold serially diluted from 5.0 nM to 84.6 fM in assay buffer; and biotin-hCD3 ε/δ (used as positive control) was three-fold serially diluted from 4.17 μM to 70.5 pM in assay buffer. Serially diluted scFv-PN29950 and controls mAbs was mixed with 20 pM of each anti-hCD3 mAb, REGN18409 and REGN18411 and serially diluted scFv-PN277570 and control mAbs was mixed with 20 pM of REGN2533. Table 23 summarizes the concentrations of each hCD3 anti-idiotype scFv and control mAbs used.

The pre-bind reaction mix was incubated at RT for 1 hour and then transferred to hCD3 ε/δ coated ELISA plate and incubated at RT for 1 hour. Binding of each anti-hCD3 mAb (REGN18409, REGN18411, and REGN2533) in presence of the respective scFv and control mAbs was detected using HRP conjugated anti-human Fc polyclonal antibody (Jackson Immunoresearch) by incubating for 1 hour at RT. The assay plates were developed using TMB colorimetric substrates according to the manufacturer's recommended procedure.

The absorbance at 450 nm for each well was recorded and plotted as the function of the dilution of each hCD3 anti-idiotype scFv tested. Data was analyzed in GraphPad Prism software using a four-parameter logistic equation over an 11-point inhibition curve. Since the CHO supernatants did not have a measured concentration, there were no calculated IC₅₀ values for the scFv molecules (reported as n/a is table 23). Instead, the results showing hCD3 anti-idiotype scFv blocking each anti-hCD3 mAb was reported as percent blocking as shown in Table 23.

Percent blocking at the lowest hCD3 anti-idiotype scFv dilution (i.e., highest concentration) was calculated as an indicator of the ability of the molecules to block binding of each anti-hCD3 mAb to hCD3 ε/δ relative to the baseline of the assay. The baseline signal of the assay, defined as 0% binding to hCD3 ε/δ, was determined from OD450 nm readings from anti-hFc detection in wells with assay buffer alone. Binding signal of 20 pM of each anti-hCD3 mAb (REGN18409, REGN18411 or REGN2533) in absence of the hCD3 anti-idiotype was defined as 100% binding or 0% blocking.

### Results Summary and Conclusions:

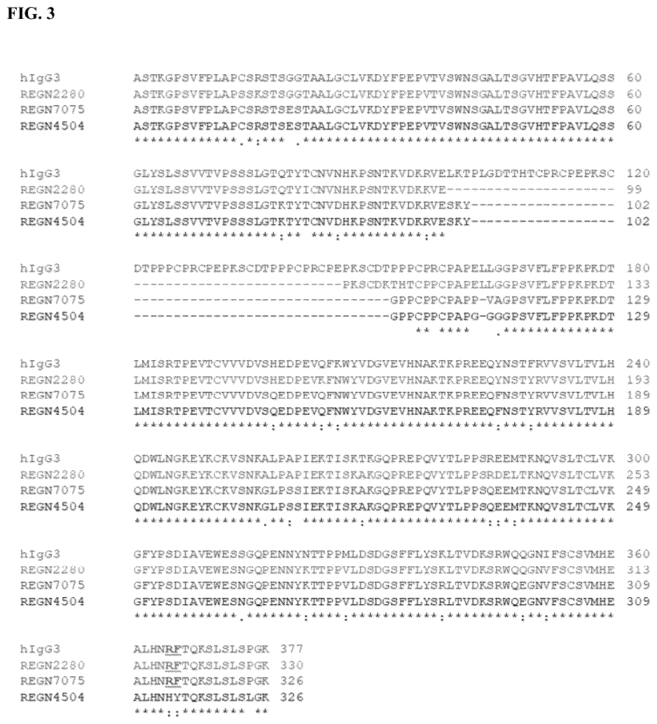

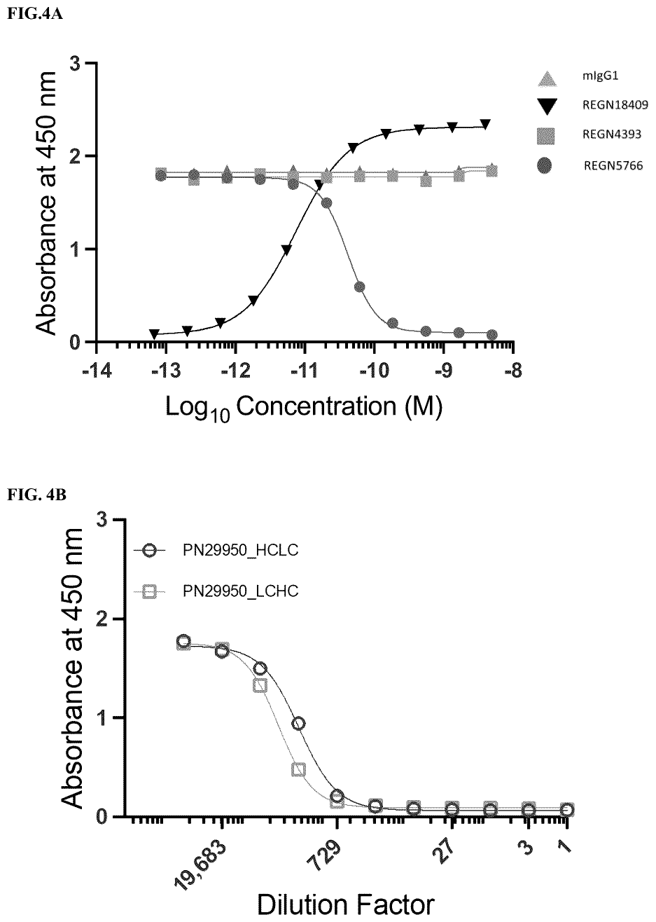

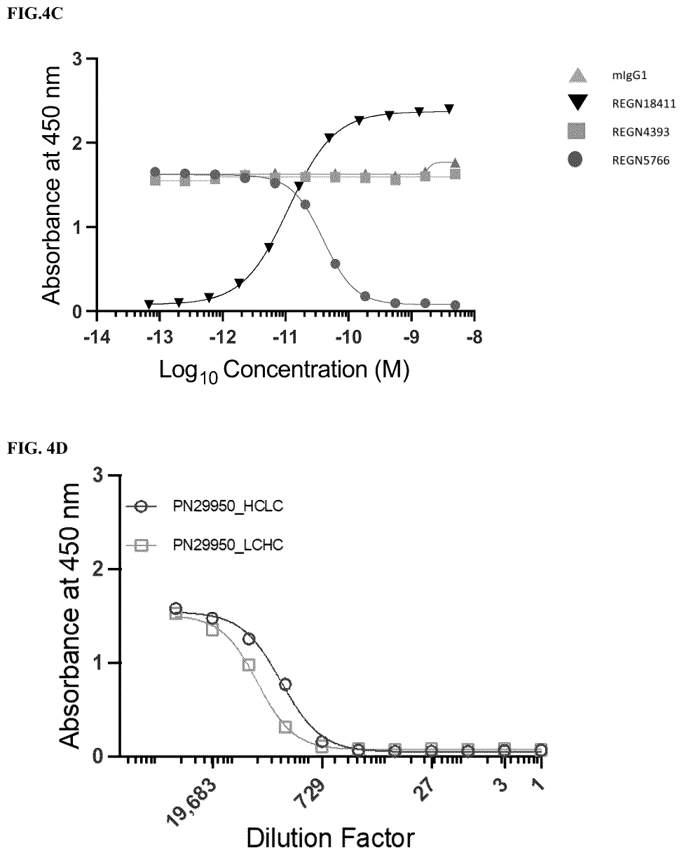

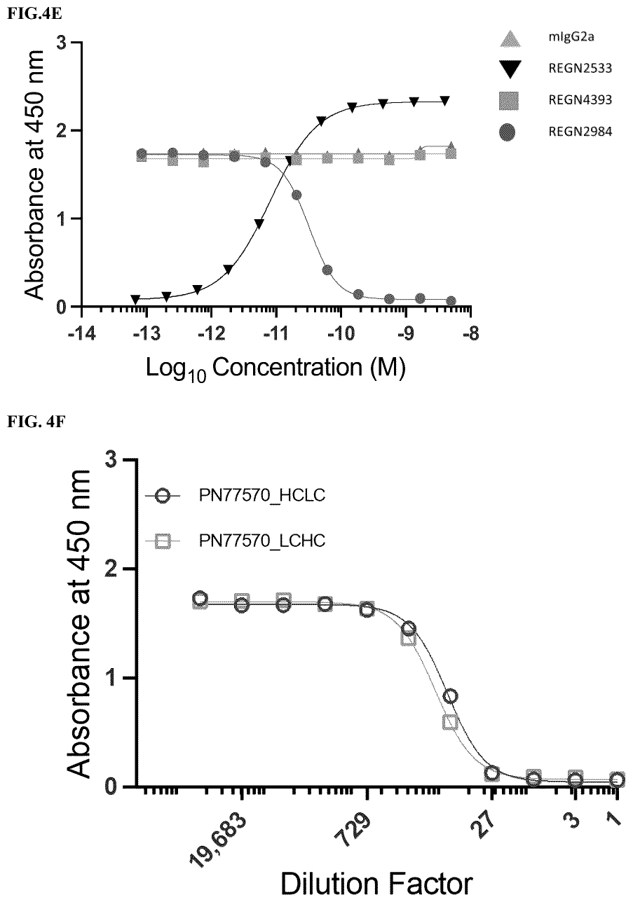

The ability of hCD3 anti-idiotype scFv molecules PN29950, to block REGN18409 and REGN18411, and PN77570 to block REGN2533 binding to plate-coated hCD3 ε/δ protein was assessed using blocking ELISA. The blocking results are summarized in Table 23 and shown in FIGS. 4A-4F. The percent blockade calculated at highest scFv concentration (undiluted CHO supernatant) is reported. All hCD3 anti-idiotype scFv tested blocked binding of 20 pM of respective anti-hCD3 mAb to baseline.

Parental control mAbs (REGN5766 and REGN2984), blocked 20 PM anti-hCD3 mAbs (REGN18408/REGN18411 and REGN2533) with IC₅₀ [M] values of 42 pM/39 pM, and 32 pM respectively and demonstrated approximately 100% blockade at highest mAb concentration. hCD3 ε/δ (ligand control) demonstrated blocking at or close to baseline for each anti-hCD3 mAb. The corresponding isotype control mAbs (Southern Biotech) and scFv negative control (REGN4393), did not show any blocking of anti-hCD3 mAbs, under identical assay conditions.

TABLE 23
Summary of percent blocking (%) and IC₅₀[M] values for hCD3 anti-idiotype scFv
and controls blocking 20 pM anti-hCD3 mAbs binding to immobilized hCD3 ε/δ.

| | | | | | |
|---|---|---|---|---|---|
| | | | | Percent Blocking | |
| | | | Blocking at | at highest | |
| | | 20 pM Anti- | highest | hCD3 Anti- | Blocking |
| | | hCD3 mAb | hCD3 | idiotype CHO | IC₅₀ |
| | Buffer | binding to | anti-idiotype | sup/control | [M] |
| | Alone | plate | concentration | antibody | at highest |
| | (O.D. | coated hCD3 | (O.D. | concentration | mAb |
| ID | 450 nm) | (O.D. 450 nm) | 450 nm) | (%) | concentration |

Blocking Anti-hCD3, 7221G

| PN29950_HCLC | 0.07 | 1.88 | 0.07 | 100.1 | n/a |
|---|---|---|---|---|---|
| PN29950_LCHC | 0.07 | 1.82 | 0.07 | 99.8 | n/a |
| REGN5766 (7221G Parental Ctrl) | 0.07 | 1.81 | 0.08 | 99.6 | 42.1 pM |
| REGN4393 (scFv Neg. Ctrl) | 0.07 | 1.88 | 1.84 | Non-Blocking | |
| mIgG1 (Isotype Ctrl) | 0.07 | 1.86 | 1.88 | Non-Blocking | |
| Ligand (Positive Ctrl) | 0.07 | 1.89 | 0.21 | 92.2 | n/a |

Blocking Anti-hCD3, 7221G20

| PN29950_HCLC | 0.07 | 1.73 | 0.07 | 100.2 | n/a |
|---|---|---|---|---|---|
| PN29950_LCHC | 0.07 | 1.67 | 0.07 | 99.9 | n/a |
| REGN5766 (7221G Parental Ctrl) | 0.07 | 1.68 | 0.07 | 100.1 | 39.1 pM |
| REGN4393 (scFv Neg. Ctrl) | 0.07 | 1.72 | 1.63 | Non-Blocking | |
| mIgG1 (Isotype Ctrl) | 0.07 | 1.67 | 1.77 | Non-Blocking | |
| Ligand (Positive Ctrl) | 0.07 | 1.73 | 0.54 | 71.6 | n/a |

Blocking Anti-hCD3, 9F07

| PN77570_HCLC | 0.06 | 1.80 | 0.06 | 100.0 | n/a |
|---|---|---|---|---|---|
| PN77570_LCHC | 0.06 | 1.75 | 0.07 | 99.7 | n/a |
| REGN2984 (9F07 Parental Ctrl) | 0.06 | 1.76 | 0.06 | 99.9 | 32.2 pM |
| REGN4393 (scFv Neg. Ctrl) | 0.06 | 1.81 | 1.74 | Non-Blocking | |
| mIgG2a (Isotype Ctrl) | 0.06 | 1.77 | 1.82 | Non-Blocking | |
| Ligand (Positive Ctrl) | 0.06 | 1.82 | 0.11 | 97.1 | n/a |

100% non-blocking = OD_{450 nm} value of wells with HRP-conjugated secondary protein in assay buffer alone

$$Maximum \% Blocking = 100 - \frac{\begin{matrix}[ Experimental {Signal}_{( {hCD}_{3} anti - idiotype + anti - {hCD}_{3} )} - \\ Background {Signal}_{( bufferonly )} ]\end{matrix}}{[ Maximum {Signal}_{( anti - {hCD}_{3} alone )} - Background {Signal}_{( bufferonly )} ]} \times 100$$

Experimental Signal=OD₄₅₀ of anti-hCD3 mAbs binding observed in presence of hCD3 anti-idiotype scFv or control mAbs
Maximum Signal=OD₄₅₀ of 20 pM anti-hCD3 mAb binding in absence of hCD3 anti-idiotype scFv or control mAbs
Background Signal=OD₄₅₀ of anti-human Fc HRP binding in assay buffer only control n/a: no calculated IC₅₀ values for ScFv's, as concentration unknown.

### Example 4: Evaluation of Antibody Binding to KHYG1 Cell Lines Engineered to Express Chimeric Antigen Receptors with Anti-Idiotype scFvs

Anti-idiotype scFv's with desired binding strength and specificity were reformatted into chimeric antigen receptors (CAR's) and expressed in KHYG1 cells, a natural killer leukemic cell line. KHYG1 cells engineered to express a CAR targeting an antibody with a modified Fc, KHYG1/NFAT-Luc/CAR1, or a CAR targeting the CD3 binding arm of a CD3 bispecific antibody, KHYG1/NFAT-Luc/CAR6 and KHYG1/NFAT-Luc/CAR15, were evaluated for target binding in flow cytometry assays. KHYG1/NFAT-Luc/CAR1 cells were evaluated for their ability to bind antibodies containing a modified Fc, while KHYG1/NFAT-Luc/CAR6 and KHYG1/NFAT-Luc/CAR15 cell lines were evaluated for binding to CD3 bispecific antibodies. Detection of antibody binding to KHYG1/NFAT-Luc/CAR6 and KHYG1/NFAT-Luc/CAR15 cell lines was assessed with an Alexa 647-conjugated secondary antibody, while antibodies tested for binding to KHYG1/NFAT-Luc/CAR1 cells were directly conjugated with Alexa 647.

TABLE 24
Reagent/Antibody Information/Materials

| AbPID/REGN# | Description |
|---|---|
| REGN5949 | CD20 × CD3-A647, (IgG4s, Fc*) |
| REGN7540 | Isotype Control-A647, (IgG4s) |
| REGN7540 | Isotype Control, (IgG4s) |
| REGN5949 | CD20 × CD3, (IgG4s, Fc*) |
| REGN5950 | CD20 × CD3, (IgG4s, Fc*) |
| REGN1979 | CD20 × CD3, (IgG4s, Fc*) |

### Experimental Procedure

### Description of Cell Lines Expressing Chimeric Antigen Receptors Targeting Antibody Domains:

Engineering of a NK reporter cell line with chimeric antigen receptors directed at antibody domains: The human natural killer cell leukemia derived KHYG-1 cell line was stably transduced with a Nuclear Factor of Activated T cells (NFAT)-luciferase reporter construct. A puromycin-resistant clone (ACL20834) was isolated and subsequently transduced with a chimeric construct comprised of an mROR signal sequence, an scFv moiety targeting specific antibody domains, such as a modified human Fc (PN78216) or a CD3 anti-idiotype (PN29950 and PN77570), a G4S linker (SEQ ID NO: 89), CD28 hinge, transmembrane, and cytoplasmic domain, a CD3z cytoplasmic domain, and cytoplasmic eGFP. Blasticidin resistant cells were maintained in RPMI1640 supplemented with 10% FBS, L-glutamine, Penicillin and Streptomycin, 10 ng/mL IL2, 1 μg/ml puromycin, and 5 μg/mL Blasticidin. The cell line engineered to recognize the modified Fc domain is named as KHYG1/NFAT-Luc/PN78216_VH-VL-CD28 bridge-TM-cyto-CD3z-eGFP (ACL22442) and also referred to as KHYG1/NFAT-Luc/CAR1. The cell lines engineered to recognize the antigenic determinants of specific CD3 antibodies (otherwise known as CD3 anti-idiotypes) are named as KHYG1/NFAT-Luc/PN29950_VL-VH-CD28 bridge-TM-cyto-CD3z-eGFP High Sort (ACL22550) and KHYG1/NFAT-Luc/PN77570_VH-VL-CD28 bridge-TM-cyto-CD3z-eGFP (ACL22594) and also referred to as KHYG1/NFAT-Luc/CAR6 and KHYG1/NFAT-Luc/CAR15.

### ASSAY Set-Up:

For flow binding experiments, KHYG1/NFAT-Luc/CAR1, KHYG1/NFAT-Luc/CAR6 and KHYG1/NFAT-Luc/CAR15 cells were washed and resuspended in stain buffer (2% FBS in PBS). 3×10⁵ cells/well were added to the wells of a 96-well, V-bottom plate. A 10-point 1:4 dose titration of antibodies ranging from 400 nM to 6.1 pM were added to cells, with the final point of the titration containing no antibody, plotted at 1.5 pM. Antibodies tested for binding to KHYG1/NFAT-Luc/CAR1 cells were directly conjugated with Alexa 647 fluorophore and consisted of an antibody with the modified Fc or a control antibody that does not harbor the modified Fc (REGN5949-A647 and REGN7540-A647, respectively). Antibodies tested for binding to KHYG1/NFAT-Luc/CAR6 and KHYG1/NFAT-Luc/CAR15 cell lines, consisted of non-fluorophore conjugated CD3 bispecific antibodies (REGN5949, REGN5950, REGN1979) or a matched isotype control (REGN7540). Cells and antibodies were incubated for 30 min at 4° C. and then washed in stain buffer. Subsequently, for KHYG1/NFAT-Luc/CAR6 and CAR15 cell lines, 2 μg/ml Alexa 647 conjugated goat-anti human Fcg fragment specific secondary antibody, diluted in stain buffer, were incubated with cells for 30 min at 4° C. After all cells were washed in stain buffer, they were resuspended in viability dye (reconstituted in DMSO according to the manufacturer's protocol and diluted 1:1000 in PBS). The mixture was incubated for 30 min at 4° C. and then washed in stain buffer. Cells were resuspended in PFA (2% diluted in stain buffer) for 30 min at 4° C. After washing, cells were resuspended in stain buffer and analyzed by flow cytometry. EC₅₀ values of the antibodies were determined from a 4-parameter logistic equation over a 10-point dose response curve (including secondary only control) using GraphPad Prism software. In Prism, the 0 nM concentration was plotted as 1.5 pM.

### Summary of Results:

### KHYG1/NFAT-Luc/CAR1 Binding Results:

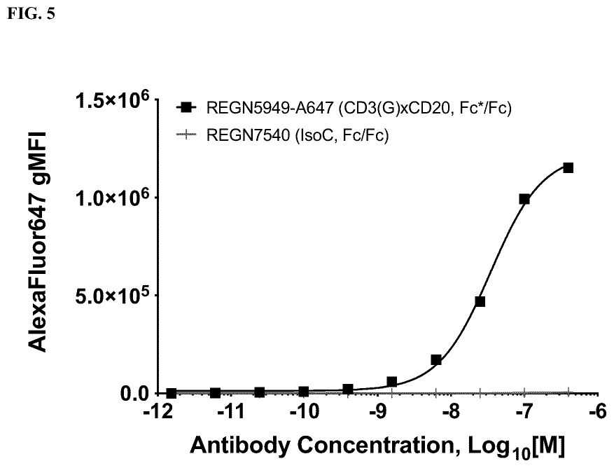

KHYG-1 cells, which express a CAR directed at an antibody containing a modified Fc (Fc*), bound A647-labelled REGN5949, but were not able to bind a control antibody (REGN7540) that had a similar Fc (IgG4s) but without the modification (Table 25 and FIG. 5).

### KHYG1/NFAT-Luc/CAR6 and CAR15 Binding Results:

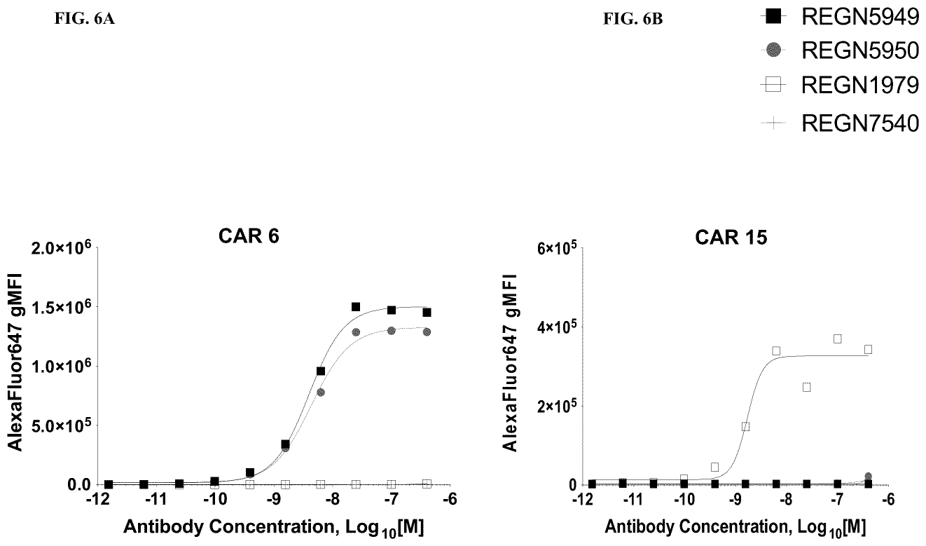

KHYG-1 Cells, which express a CAR (CAR6 or CAR15) directed at an anti-idiotype CD3 antibody, were evaluated for their ability to bind a variety of CD3xCD20 bispecific antibodies (to note, the CD3 arms used in antibodies REGN5949, REGN5950, and REGN1979 are not identical). Antibodies, REGN5949 and REGN5950 bound to KHYG-1 cells expressing CAR6, while no binding of REGN1979 was observed (Table 26 and FIG. 6A). Conversely, antibodies REGN5949 and REGN5950 did not bind to KHYG-1 cells expressing CAR15, while REGN1979 binding was observed (Table 26 and FIG. 6B). The isotype control antibody (REGN7540) did not bind to either CAR6 or CAR15-expressing KHYG-1 cells.

TABLE 25
Potency values, EC₅₀ [M] and Max Fold
Binding to KHYG-1/NFAT/CAR1 cells:

| | EC₅₀ (M) | | Max gMFI | |
|---|---|---|---|---|
| | REGN5949 | REGN7540 | REGN5949 | REGN7540 |
| CAR | (Fc*) | (Fc) | (Fc*) | (Fc) |
| 1 | 3.47E−09 | ND | 1.15E+06 | 6.20E+03 |

Abbreviations:
ND: Not Determined because no dose dependent response was observed
Max (gMFI) is the highest gMFI value within tested dose-range.

TABLE 26
Potency values, EC₅₀ [M] and Max Fold Binding to KHYG-1/NFAT/CAR6 and CAR15 cells:

| | EC₅₀ (M) | | | | Max gMFI | | | |
|---|---|---|---|---|---|---|---|---|
| CAR | REGN5949 | REGN5950 | REGN1979 | REGN7540 | REGN5949 | REGN5950 | REGN1979 | REGN7540 |
| 6 | 3.95E−09 | 4.36E−09 | ND | ND | 1.50E+06 | 1.30E+06 | 7.17E+03 | 5.95E+03 |
| 15 | ND | ND | 1.70E−09 | ND | 4.23E+03 | 2.19E+04 | 3.70E+05 | 1.33E+04 |

Abbreviations:
ND: Not Determined because no dose dependent response was observed
Max (gMFI) is the highest gMFI value within tested dose-range.

### Example 5: KHYG1/NFAT-Luc/CAR Reporter Signaling Bioassay

ScFv's with desired binding strength and specificity were reformatted into chimeric antigen receptors (CAR's) and expressed in KHYG1 cells, a natural killer leukemic cell line. A series of functional assays were performed to identify ideal candidates, including an engineered reporter assay, where activation of CAR's on KHYG1 cells lead to a luminescent signal.

TABLE 27
Reagent/Antibody Information/Materials:

| AbPID/REGN# | Description |
|---|---|
| REGN5949 | Anti-CD20 × CD3, hIgG4s (Fc*) |
| REGN5950 | Anti-CD20 × CD3, hIgG4s (Fc*) |
| H4sH17400D | Anti-CD20 × CD3, hIgG4s (Fc*) |
| H4sH14303N2 | Anti-CD20, hIgG4s (Fc) |
| REGN1979 | Anti-CD20 × CD3, hIgG4s (Fc*) |
| REGN5951 | Anti-CD20 × CD3, hIgG4s (Fc*) |
| REGN5375 | Anti-CD20 × CD3, hIgG4s (Fc*) |
| REGN7540 | Isotype, hIgG4s (Fc) |

### Experimental Procedure

KHYG1/NFAT-Luc/CAR1 (ahFc*-CD28-CD3z) Signaling Bioassay:

To assess target and antibody dependent agonistic activity of the CAR construct directed at the modified Fc, a cell-based reporter assay was established where an Fc-modified antibody directed at CD20 (either a CD20 bivalent antibody or CD20xCD3 bispecific antibody, H4H14303N2 and REGN5949, respectively) was co-incubated with Ramos target cells (which express CD20) and KHYG1/NFAT-Luc/CAR1 effector cells at a 1:1 (target:effector cell) ratio. Binding of the antibody to CD20 on target cells and subsequent engagement of the modified Fc by the CAR1-expressing KHYG1 cells leads to clustering of the CAR construct and subsequent activation of Nuclear Factor of Activated T cells (NFAT) response element driven luciferase expression.

### Assay Set-Up:

The experiment was carried out in assay medium containing, RPMI1640 supplemented with 10% FBS, L-glutamine, Penicillin and Streptomycin. KHYG1/NFAT-Luc/CAR1 reporter cells were plated at 2.5×10⁴ reporter cells/well in 96 well white flat bottom plates. Subsequently, 2.5×10⁴ Ramos.2G6.4C10 (CD20+) or HEK293 (CD20⁻) target cells/well were added to plates. CD20 bivalent (H4H14303N2), bispecific (REGN5949) or isotype control (REGN7540) antibodies were serially diluted (1:4) over an 11-point titration range (100 nM to 95 fM) (FIGURES and) with a 12th point containing no antibody (represented as 24 fM) and added to wells, for a final volume of 100 μl in wells. Plates were incubated for 5 hours at 37° C./5% CO₂ and then 100 μL detection reagent was added to the wells to lyse the cells and detect luciferase activity. The emitted light was measured in RLU on a multilabel plate reader Envision (PerkinElmer). EC₅₀ values were determined from a 4-parameter logistic equation over a 12-point dose response curve using GraphPad Prism software. In Prism, the 0 nM concentration was plotted as 24 fM.

### KHYG1/NFAT-Luc/CAR6 and CAR15 (CD3 Anti-Idiotype-CD28-CD3z) Signaling Bioassay:

To assess target and antibody dependent agonistic activity of the CD3 anti-idiotype CAR chimeric constructs (CAR6 and CAR15), a cell-based reporter assay was established where CD3 x CD20 bispecific antibodies (containing different CD3 binding arms, REGN5949, REGN5950, H4sH17400D, REGN1979, REGN5951, REGN5375) or a matched isotype control (REGN7540) were co-incubated with Ramos target cells (which express CD20) and KHYG1/NFAT-Luc/CAR6 or KHYG1/NFAT-Luc/CAR15 effector cells at a 1:1 (target:effector cell) ratio. Binding of the bispecific antibody to CD20 on target cells and subsequent engagement of the CD3 binding arm by the anti-idiotype CAR-expressing KHYG1 cells, leads to clustering of the CAR construct and subsequent activation of Nuclear Factor of Activated T cells (NFAT) response element driven luciferase expression.

### Assay Set-Up:

The experiment was carried out in assay medium containing, RPMI1640 supplemented with 10% FBS, L-glutamine, Penicillin and Streptomycin. KHYG1/NFAT-Luc/CAR6 or KHYG1/NFAT-Luc/CAR15 reporter cells were plated at 2.5×10⁴ cells/well in 96 well white flat bottom plates. Subsequently, 2.5×10⁴ Ramos.2G6.4C10 target cells/well were added to plates. CD20xCD3 bispecific antibodies containing a variety of CD3 binding arms (REGN5949, REGN5950, H4sH17400D, REGN1979, REGN5951, REGN5375) or an isotype control antibody (REGN7540) were serially diluted (1:5) over a 9-point titration range (100 nM to 256 fM) (FIGURES and) with a 10th point containing no antibody (represented as 51 fM) and added to wells, for a final volume of 100 μl in wells. Plates were incubated for 4 hours at 37° C./5% CO₂ and then 100 μL detection reagent was added to the wells to lyse the cells and detect luciferase activity. The emitted light was measured in RLU on a multilabel plate reader Envision (PerkinElmer). EC50 values were determined from a 4-parameter logistic equation over a 10-point dose response curve using GraphPad Prism software. In Prism, the 0 nM concentration was plotted as 51 fM.

Fold induction was calculated using the following equation:

$$Maximum Fold Induction = \frac{Highest mean RLU value within tested dose range}{Mean RLU values of no protein control}$$

### Summary of Results:

### KHYG/NFAT-Luc/CAR1 Reporter Activation Results:

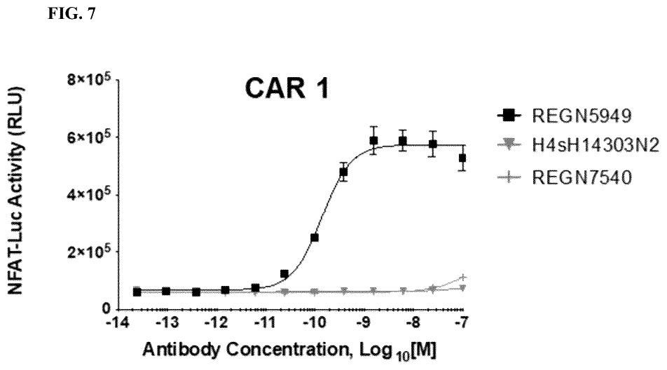

KHYG-1 Cells, expressing a CAR directed at an antibody containing a modified Fc (Fc*), were activated in the presence of target cells expressing CD20, and an antibody against CD20 that had a modified Fc (Fc*), recognized by the CAR (Table 28 and FIG. 7). A control antibody, H4sH14303N2, that targets CD20 but does not contain a modified Fc, as well as an additional non-targeting control antibody, REGN7540, did not activate KHYG-1/NFAT-Luc/CAR1 cells (Table 28 and FIG. 7). No activation was observed in the presence of a target cell not expressing CD20 (data not shown).

### KHYG1/NFAT-Luc/CAR6 and CAR15 Reporter Activation Results:

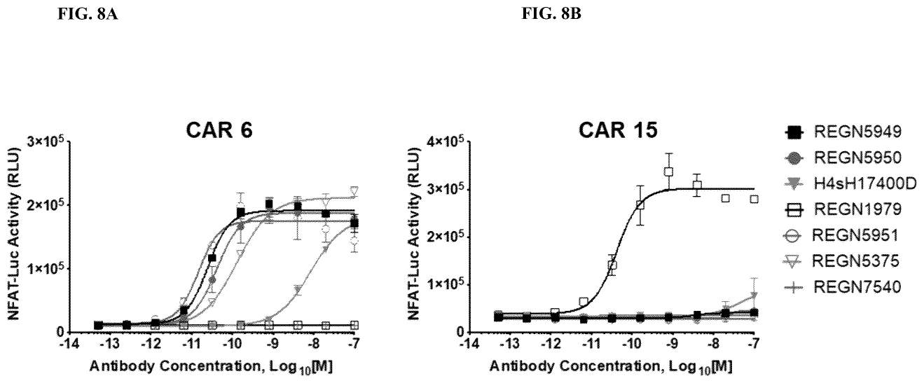

KHYG-1/NFAT-Luc cells, expressing a CAR (CAR6 or CAR15) directed at an anti-idiotype CD3 antibody, were activated in the presence of target cells expressing CD20, and specific CD20xCD3 bispecific antibodies. Namely, KHYG-1/NFAT-Luc cells expressing CAR6 were activated in the presence of Ramos cells (CD20⁺) and antibodies, REGN5951, REGN5375, REGN5949, REGN5950, and H4sH17400D, in a dose-dependent manner (Table 29, Table 30, and FIG. 8A). However, antibody REGN1979 did not activate KHYG-1/NFAT-Luc/CAR6 reporter activity (Table 29, Table 30, and FIG. 8A). Conversely, only REGN1979 was able to activate KHYG-1/NFAT-Luc/CAR15 reporter activity (Table 29, Table 30, and FIG. 8B). The isotype control antibody, REGN7540, did not activate KHYG-1/NFAT-Luc/CAR6 or CAR15 cells (Table 29, Table 30, and FIGS. 8A and 8B).

TABLE 28
Potency values, EC₅₀ [M] and Max Fold Activation of KHYG-1/NFAT/CAR1 cells:

| | EC₅₀ (M) | | | Max Fold Activation | | |
|---|---|---|---|---|---|---|
| | REGN5949 | H4sH14303N2 | REGN7540 | REGN5949 | H4sH14303N2 | REGN7540 |
| CAR | (Fc*) | (Fc) | (Fc) | (Fc*) | (Fc) | (Fc) |
| 1 | 1.39E−10 | ND | ND | 9.9 | 1.2 | 1.9 |

Abbreviations:
ND: Not Determined because no dose dependent response was observed
Max fold activation is the highest value within tested dose-range over the no antibody control value.

TABLE 29
Potency values, EC₅₀ of KHYG-1/NFAT/CAR6 and CAR15 cells:

| | EC₅₀ (M) | | | | | | |
|---|---|---|---|---|---|---|---|
| | REGN5949 | REGN5950 | H4sH17400D | REGN1979 | REGN5951 | REGN5375 | REGN7540 |
| CAR 6 | 2.48E−11 | 4.16E−11 | 8.50E−09 | ND | 1.46E−11 | 1.31E−10 | ND |
| CAR 15 | ND | ND | ND | 4.20E−11 | ND | ND | ND |

Abbreviations:
ND: Not Determined because no dose dependent response was observed

TABLE 30
Max Fold Activation of KHYG-1/NFAT/CAR6 and CAR15 cells:

| | Max Fold Activation | | | | | | |
|---|---|---|---|---|---|---|---|
| | REGN5949 | REGN5950 | H4sH17400D | REGN1979 | REGN5951 | REGN5375 | REGN7540 |
| CAR 6 | 18.5 | 16.7 | 13.4 | 1.0 | 18.8 | 19.7 | 1.2 |
| CAR 15 | 1.3 | 1.2 | 1.9 | 9.9 | 1.0 | 1.0 | 1.0 |

Max fold activation is the highest value within tested dose-range over the no antibody control value.

### Example 6: KHYG1/NFAT-Luc/CAR Cytotoxic NK Bioassay

As discussed in Example 5 above, ScFv's with desired binding strength and specificity were reformatted into chimeric antigen receptors (CAR's) and expressed in KHYG1 cells, a natural killer leukemic cell line. A series of functional assays were performed to identify ideal candidates, including a cytotoxic NK-killing assay with engineered KHYG-1 effector cells and Ramos/GFP-HiBiT target cells.

To assess antibody-dependent NK cell activation, a NK-cell line based cytolytic assay was established where an antibody was co-incubated with target cells (Ramos/HiBit) and KHYG-1 cells expressing a CAR construct. Clustering of the CAR construct leads to the cytolysis of target cells. Target cell lysis leads to the release of intracellular HiBit into supernatant. A detection reagent is added that contains the complementary polypeptide LgBiT, which spontaneously interacts with the HiBiT tag to reconstitute the bright, luminescent NanoBiT® enzyme (Promega).

### Engineering of a B-Cell Reporter for Cytolytic Activity:

From the prior described Ramos/GFP cell line (ACL21777), a clone was isolated by single-cell sorting and maintained in RPMI1640 supplemented with 10% FBS, L-glutamine, Penicillin, and streptomycin. This cell line was renamed Ramos/GFP-HiBiT cl 1B10 (ACL22182).

Same antibodies shown in Table 27 in Example 5 above were used for this study.

### Experimental Procedure

KHYG1/NFAT-Luc/CAR1 (ahFc*-CD28-CD3z) Cytotoxic Bioassay:

To assess target and antibody dependent cytotoxic activity of the CAR construct directed at the modified Fc, a NK cell-based cytotoxic assay was established where an Fc-modified antibody directed at CD20 (either a CD20 bivalent antibody or CD20xCD3 bispecific antibody, H4H14303N2 and REGN5949, respectively) was co-incubated with Ramos/HiBiT target cells (which express CD20) and KHYG1/NFAT-Luc/CAR1 effector cells at a 1:5 (target:effector) ratio. Binding of the antibody to CD20 on target cells and subsequent engagement of the modified Fc by the CAR1-expressing KHYG1 cells, led to clustering of the CAR construct and subsequent lysis of target cells, which was measured via release of HiBiT into the supernatant where it complemented LgBiT to form a highly luminescent NanoBiT enzyme.

### Assay Set-Up:

The experiment was carried out in assay medium containing, RPMI1640 supplemented with 10% FBS, L-glutamine, Penicillin and Streptomycin. KHYG1/NFAT-Luc/CAR1 cells were plated at 2.5×10⁴ cells/well in 96 well white flat bottom plates. Subsequently, 5.0×10³ Ramos/HiBit target cells/well were added to plates. CD20 bivalent (H4H14303N2), bispecific (REGN5949) or isotype control (REGN7540) antibodies were serially diluted (1:4) over a 12-point titration range (100 nM to 95 fM) (FIGURES and) with an 12th point containing no antibody (represented as 24 fM) and added to wells, for a final volume of 100 μl in wells. Plates were incubated for 5 hours at 37° C./5% CO₂ and then 100 μL non-lytic Nano-Glo extracellular detection reagent was added, according to manufacturer's specifications. The emitted light was measured in RLU on a multilabel plate reader Envision (PerkinElmer). EC₅₀ values were determined from a 4-parameter logistic equation over an 11-point dose response curve using GraphPad Prism software. In Prism, the 0 nM concentration was plotted as 24 fM.

$$Percent Cytotoxicity = \frac{( sample RLU ) - ( Spontaneous Signal )}{( maximum signal ) - ( Spontaneous signal )} \times 100$$

Spontaneous signal=target cells alone (absence of antibody)
Maximum signal=target cells alone lysed at the end of assay incubation.

### KHYG1/NFAT-Luc/CAR6 and CAR15 (CD3 Anti-Idiotype-CD28-CD3z) Cytotoxic Bioassay:

To assess target and antibody dependent cytotoxic activity of the CD3 anti-idiotype CAR chimeric constructs (CAR6 and CAR15), an assay was established where CD3 x CD20 bispecific antibodies (with a range of different CD3 binding arms, REGN5949, REGN5950, H4sH17400D, REGN1979, REGN5951, REGN5375) or a matched isotype control (REGN7540) were co-incubated with Ramos/HiBit target cells (which express CD20) and KHYG1/NFAT-Luc/CAR6 or KHYG1/NFAT-Luc/CAR15 effector cells at a 1:5 (target:effector) ratio. Binding of the bispecific antibody to CD20 on target cells and subsequent engagement of the CD3 binding arm by the anti-idiotype CAR-expressing KHYG1 cells, leads to clustering of the CAR construct and subsequent lysis of target cells, which is measured via release of HiBiT into the supernatant where it complements LgBiT to form a highly luminescent NanoBiT enzyme.

### Assay Set-Up:

The experiment was carried out in assay medium containing, RPMI1640 supplemented with 10% FBS, L-glutamine, Penicillin and Streptomycin. KHYG1/NFAT-Luc/CAR6 or KHYG1/NFAT-Luc/CAR15 cells were plated at 2.5×10⁴ cells/well in 96 well white flat bottom plates. Subsequently, 5×10³ Ramos/HiBiT target cells/well were added to plates. CD20xCD3 bispecific antibodies (REGN5949, REGN5950, H4sH17400D, REGN1979, REGN5951, REGN5375) or an isotype control antibody (REGN7540) were serially diluted (1:5) over a 9-point titration range (100 nM to 256 fM) (FIGURES and) with a 10th point containing no antibody (represented as 51 fM) and added to wells, for a final volume of 100 μl in wells. Plates were incubated for 4 hours at 37° C./5% CO₂ and then 100 μL of non-lytic Nano-Glo extracellular detection reagent was added, according to manufacturer's specifications. The emitted light was measured in RLU on a multilabel plate reader Envision (PerkinElmer). EC₅₀ values were determined from a 4-parameter logistic equation over a 10-point dose response curve using GraphPad Prism software. In Prism, the 0 nM concentration was plotted as 51 fM.

Percent cytotoxicity was calculated using the following equation:

$$\% Cytotoxicity = \frac{( sample RLU ) - ( Spontaneous Signal )}{( maximum signal ) - ( Spontaneous signal )} \times 100$$

Spontaneous signal=target cells alone (absence of antibody)
Maximum signal=target cells alone lysed at the end of assay incubation.

### Summary of Results:

### KHYG/NFAT-Luc/CAR1 Cytotoxic Activation Results:

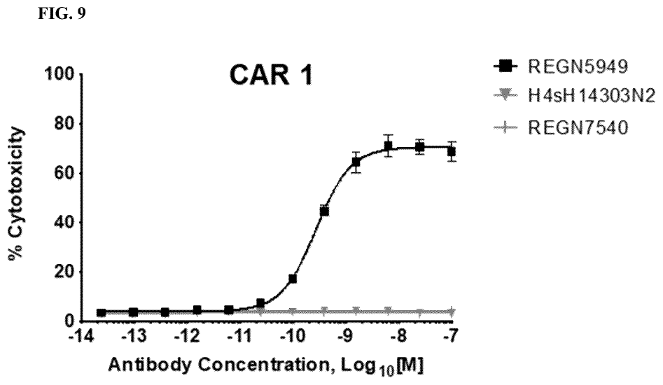

KHYG-1 Cells, expressing a CAR directed at an antibody containing a modified Fc (Fc*), induced killing of CD20⁺ Ramos/GFP-HiBiT target cells, in the presence of an antibody against CD20 that had a modified Fc (Fc*), recognized by the CAR (Table 31 and FIG. 9). A control antibody, H4sH14303N, that targets CD20 but does not contain a modified Fc, as well as a non-targeting control antibody, REGN7540, did not activate KHYG-1/NFAT-Luc/CAR1 cells to kill Ramos target cells (Table 31 and FIG. 9).

### KHYG1/NFAT-Luc/CAR6 and CAR15 Reporter Activation Results:

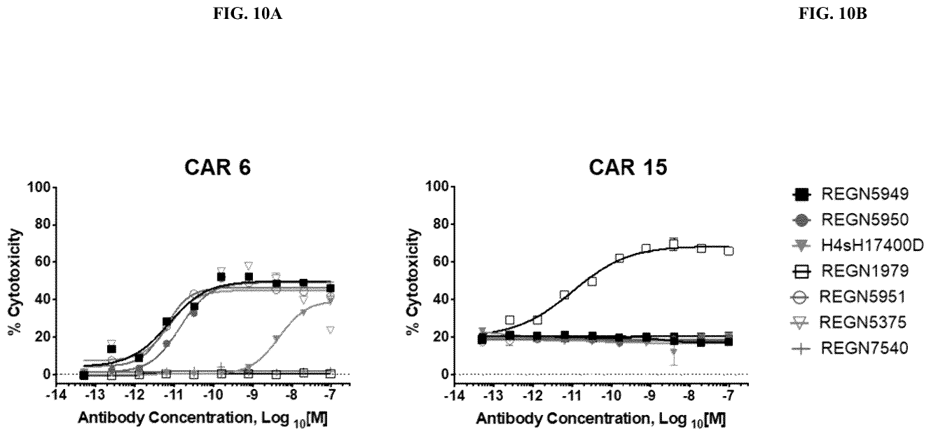

KHYG-1/NFAT-Luc Cells, expressing a CAR (CAR6 or CAR15) directed at an anti-idiotype CD3 antibody, led to killing of CD20⁺ Ramos/GFP-HiBiT target cells in the presence of specific CD20xCD3 bispecific antibodies. Namely, KHYG-1/NFAT-Luc cells expressing CAR6 led to target cell killing in the presence of antibodies, REGN5951, REGN5375, REGN5949, REGN5950, and H4sH17400D, in a dose-dependent manner (Table 32 and FIG. 10A). However, antibody REGN1979 did not induce KHYG-1/NFAT-Luc/CAR6 cytotoxic activity (Table 32 and FIG. 10A). Conversely, only REGN1979 was able to induce KHYG-1/NFAT-Luc/CAR15 target cell killing (Table 32 and FIG. 10B). The isotype control antibody, REGN7540, did not induce cytotoxic activity of KHYG-1/NFAT-Luc/CAR6 or CAR15 cells (Table 32 and FIGS. 10A and 10B).

TABLE 31
Potency values, EC₅₀ [M] and Max Lysis of
Target cells using KHYG-1/NFAT/CAR1 cells:

| | EC₅₀ (M) | | | Max Lysis (%) | | |
|---|---|---|---|---|---|---|
| | REGN5949 | H4sH14303N2 | REGN7540 | REGN5949 | H4sH14303N2 | REGN7540 |
| CAR | (Fc*) | (Fc) | (Fc) | (Fc*) | (Fc) | (Fc) |
| 1 | 2.74E−10 | ND | ND | 71.1 | 4.5 | 4.8 |

Abbreviations:
ND: Not Determined because no dose dependent response was observed
Max Lysis is the highest calculated % cytotoxicity value within tested dose-range.

TABLE 32
Potency values, EC₅₀ [M] and Max Cytotoxicity of KHYG-1/NFAT/CAR6 and CAR15 cells:

| | EC₅₀ (M) | | | | | | |
|---|---|---|---|---|---|---|---|
| | REGN5949 | REGN5950 | H4sH17400D | REGN1979 | REGN5951 | REGN5375 | REGN7540 |
| CAR 6 | 7.16E−12 | 1.40E−11 | 4.67E−09 | ND | 5.97E−12 | 7.51E−12 | ND |
| CAR 15 | ND | ND | ND | 1.01E−11 | ND | ND | ND |
| | Max Cytotoxic Activity (%) | | | | | | |
| | REGN5949 | REGN5950 | H4sH17400D | REGN1979 | REGN5951 | REGN5375 | REGN7540 |
| CAR 6 | 52.5 | 51.8 | 39.1 | 1.0 | 51.2 | 57.9 | 4.2 |
| CAR 15 | 21.3 | 21.7 | 22.9 | 69.6 | 19.7 | 19.8 | 21.2 |

Abbreviations: ND: Not Determined because no dose dependent response was observed
Max Lysis is the highest calculated % cytotoxicity value within tested dose-range.

### Example 7: CBNK Cells Engineered with an Anti-CD3 Idiotype Chimeric Antigen Receptor (CAR) Mediate Target Cell Cytotoxicity

Similar to experiments described in Example 6, antibody-dependent NK cell activation, was evaluated using NK cells derived from primary human cord blood (CBNK) that were engineered to express a CAR construct and co-incubated with target cells (Ramos/HiBit).

### Engineering of Cord Blood (CB)-Derived NK Cells (CBNK), Expressing CAR:

Human CD34⁺-HSPC Cord Blood (CB)-derived NK cells (CBNK) were generated using standard Synthetic Biology & Cell Engineering (SBCE) protocols. Briefly, a two-step, serum-free, cytokine-based ex vivo protocol was used to promote the generation of NK cells from HSPCs, using the StemSpan™ NK Cell Generation Kit (Catalog #09960). In the first step, CD34⁺ HSPCs were cultured for 14 days in medium containing expansion supplement (mainly SCF, IL7, FLT3, TPO) to stimulate their proliferation and differentiation into lymphoid progenitor cells. At the end of this initial phase, (days 8-10) the cells were engineered with a lentiviral vector (LVV) containing the anti-CD3 idiotype ScFv (CAR6), fused to the 28z-CAR-membrane-bound IL-15 construct. 48-72 hours after transduction, armored CBNK cells were sorted. In the second step, these lymphoid progenitor cells were cultured for another 14 days in medium containing the differentiation supplement (mainly IL15, IL7, SCF, FLT3 and UM729) to promote their expansion and differentiation into CD56⁺ NK cells. These primary aCD3-ID-28z-CAR-mb15-CBNK cells (referred to as CBNK/CAR6) were finally expanded by culturing with engineered 41BBL-mbIL21-K562 feeder cells for a week, before the cells were used in functional assays.

TABLE 33
Reagent/Antibody Information/Materials:

| AbPID/REGN# | Description |
|---|---|
| REGN5949 | Anti-CD20 × CD3, hIgG4s (Fc*) |
| REGN5950 | Anti-CD20 × CD3, hIgG4s (Fc*) |
| REGN1979 | Anti-CD20 × CD3, hIgG4s (Fc*) |
| REGN4018 | Control × CD3, hIgG4s (Fc*) |

### Experimental Procedure

### CBNK Cytotoxic Bioassay:

To assess target and antibody dependent cytotoxic activity of CBNK's expressing the CD3 anti-idiotype CAR chimeric construct (CAR6), an assay was established where CD3 x CD20 bispecific antibodies, or an isotype matched non-targeting x CD3 control were co-incubated with Ramos/HiBit target cells (which express CD20) and CBNK/CAR6 effector cells at a 1:4 (target:effector) ratio. Binding of the bispecific antibody to CD20 on target cells and subsequent engagement of the CD3 binding arm by the anti-idiotype CAR-expressing CBNK cells, led to clustering of the CAR construct and subsequent lysis of target cells, which was measured via release of HiBiT into the supernatant where it complemented LgBiT to form a highly luminescent NanoBiT enzyme.

### Assay Set-Up:

The experiment was carried out in assay medium containing, RPMI1640 supplemented with 10% FBS, L-glutamine, Penicillin and Streptomycin. CBNK/CAR6 cells were plated at 2.0×10⁴ cells/well in 96 well white flat bottom plates. Subsequently, 5×10³ Ramos/HiBiT target cells/well were added to plates. CD20xCD3 bispecific antibodies (REGN5949, REGN5950, REGN1979) or an isotype matched non-targeting Control x CD3 (REGN4018), were serially diluted (1:5) over a 9-point titration range (25 nM to 64 fM) (FIGURES and) with a 10th point containing no antibody (represented as 13 fM) and added to wells, for a final well volume of 100 μL. Plates were incubated for 4 hours at 37° C./5% CO₂ and then 25 μl removed for assessment of cytokine. Subsequently, 75 μL of non-lytic Nano-Glo extracellular detection reagent was added to wells, according to manufacturer's specifications. The emitted light was measured in RLU on a multilabel plate reader Envision (PerkinElmer). EC₅₀ values were determined from a 4-parameter logistic equation over a 10-point dose response curve using GraphPad Prism software. In Prism, the 0 nM concentration was plotted as 13 fM.

$$\% Cytotoxicity = \frac{( sample RLU ) - ( Spontaneous Signal )}{( maximum signal ) - ( Spontaneous signal )} \times 100$$

Spontaneous signal=target cells alone (absence of antibody)
Maximum signal=target cells alone lysed at the end of assay incubation.

For assessing the presence of cytokine in supernatant, iQue Qbeads Assay Builder kit, from Sartorius, was used. Cytokine capture beads and standards were prepared according to manufacturer's instruction. Sample preparation was also according to manufacturer's recommendation. Briefly, 10 μl of supernatant, from the 25 μl collected from assay wells, was added to 96 well v-bottom plates, followed by the addition of 10 μl capture beads. After a short centrifugation, plates were incubated for 60 min. in the dark, followed by the addition of 10 μl detection cocktail. After a short centrifugation, plates were incubated for 90 min in the dark, followed by 2 rounds of washing with stain buffer (2% FBS in PBS). Samples were resuspended in 25 μl of stain buffer and transferred to 96 well U-bottom plates. Samples and standards were run on the iQue flow cytometer and quantitation of cytokine performed according to manufacturer's instruction.

### Summary of Results:

### CBNK/CAR6 Activation Results:

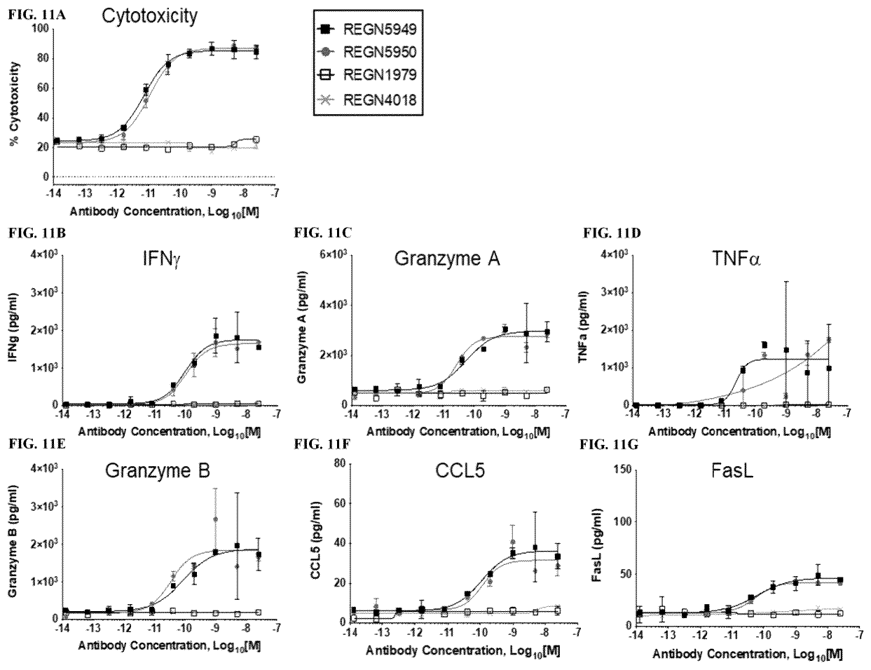

CBNK Cells, expressing a CAR directed at an anti-idiotype CD3 antibody (CAR6), led to killing of CD20⁺ Ramos/GFP-HiBiT target cells in the presence of specific CD20xCD3 bispecific antibodies. Namely, CBNK/CAR6 led to target cell killing in the presence of antibodies, REGN5949 and REGN5950, however not REGN1979 (Table 34, Table 35, and FIG. 11A). The non-targeting control x CD3 antibody, REGN4018, also did not lead to target cell killing (Table 34, Table 35, and FIG. 11A).

CBNK Cells, expressing a CAR directed at an anti-idiotype CD3 antibody (CAR6), led to release of cytokines in the presence of CD20⁺ Ramos/GFP-HiBiT target cells and specific CD20xCD3 bispecific antibodies. Namely, CBNK/CAR6 led to release of IFNγ, TNFα, Granzyme A, Granzyme B, CCL5 and FasL in the presence of antibodies, REGN5949 and REGN5950, however not REGN1979 (Table 34, Table 35, and FIGS. 11B-11G). The non-targeting control x CD3 antibody, REGN4018, also did not lead to cytokine release (Table 34, Table 35, and FIGS. 11B-11G).

TABLE 34
Potency values, EC₅₀ [M] from CBNK/CAR6 cells

| Assay | EC₅₀ (M) | | | |
|---|---|---|---|---|
| Read-out | REGN5949 | REGN5950 | REGN1979 | REGN4018 |
| Cytotoxicity | 6.87E−12 | 1.06E−11 | ND | ND |
| IFNγ release | 9.94E−11 | 1.08E−10 | ND | ND |
| Granzyme A | 5.42E−11 | 2.61E−11 | ND | ND |
| release | | | | |
| TNFα release | 2.36E−11 | NC | ND | ND |
| Granzyme B | 8.64E−11 | 3.29E−11 | ND | ND |
| release | | | | |
| CCL5 release | 1.09E−10 | 1.17E−10 | ND | ND |
| FasL release | 6.32E−11 | 5.53E−11 | ND | ND |

Abbreviations:
ND: Not Determined because no dose dependent response was observed
NC: Not Calculated because although a dose dependent response was observed, a curve could not be fit

TABLE 35
Max Activity from CBNK/CAR6 cells

| | Max Activity | | | |
|---|---|---|---|---|
| Assay Read-out | REGN5949 | REGN5950 | REGN1979 | REGN4018 |
| Cytotoxicity | 86.9 | 88.7 | 25.5 | 24.8 |
| (%) | | | | |
| IFNγ release | 1.83E+03 | 1.68E+03 | 45.6 | 70.3 |
| (pg/ml) | | | | |
| Granzyme A | 3.04E+03 | 3.06E+03 | 6.43E+02 | 6.41E+02 |
| release (pg/ml) | | | | |
| TNFα release | 1.62E+03 | 1.74E+03 | 11.9 | 14.2 |
| (pg/ml) | | | | |
| Granzyme | 1.96E+03 | 2.66E+03 | 2.39E+02 | 2.04E+02 |
| B release | | | | |
| (pg/ml) | | | | |
| CCL5 release | 38.1 | 40.8 | 6.9 | 8.4 |
| (pg/ml) | | | | |
| FasL release | 49.2 | 43.7 | 16.4 | 18.1 |
| (pg/ml) | | | | |

Max activity is the highest % cytotoxicity or cytokine release value (pg/ml) within tested dose-range.

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

A number of embodiments disclosed herein have been described. Nevertheless, it will be understood that various modifications may be made without departing from the spirit and scope disclosed herein. Accordingly, other embodiments are within the scope of the following claims. Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments disclosed herein described herein. Such equivalents are intended to be encompassed by the following claims.

We claim:

1. A chimeric antigen receptor (CAR) polypeptide comprising:
(a) an extracellular domain comprising:
(i) a CD3 extracellular domain or fragment thereof;
(ii) an antigen-binding domain specific for an idiotype of an anti-CD3 antibody; or
(iii) an antigen-binding domain specific for an Fc domain;
(b) a hinge domain;
(c) a transmembrane domain; and
(d) an intracellular signaling domain.

2. The CAR polypeptide of claim 1, wherein the extracellular domain comprises the CD3 extracellular domain or fragment thereof.

3. The CAR polypeptide of claim 2, wherein the CD3 extracellular domain or fragment thereof comprises an epitope recognized by an anti-CD3 antibody.

4. (canceled)

5. The CAR polypeptide of claim 2, wherein the CD3 extracellular domain or fragment thereof comprises (1) at least 10 consecutive amino acids of SEQ ID NO: 1959 and/or (2) an amino acid sequence at least 90% identical to SEQ ID NO: 1959.

6.-7. (canceled)

8. The CAR polypeptide of claim 1, wherein the extracellular domain comprises the antigen-binding domain specific for an idiotype of an anti-CD3 antibody.

9. (canceled)

10. The CAR polypeptide of claim 8, wherein the antigen-binding domain is a single chain fragment variable (scFv).

11. The CAR polypeptide of claim 10, wherein the antigen-binding domain comprises:
(1) the heavy chain CDR 1 (CDR-H1) of SEQ ID NO: 16, the heavy chain CDR 2 (CDR-H2) of SEQ ID NO: 17, the heavy chain CDR 3 (CDR-H3) of SEQ ID NO: 18, the light chain CDR 1 (CDR-L1) of SEQ ID NO: 19, the light chain CDR 2 (CDR-L2) of SEQ ID NO: 20, and the light chain CDR 3 (CDR-L3) of SEQ ID NO: 21; or
(2) CDR-H1 of SEQ ID NO: 24, CDR-H2 of SEQ ID NO: 25, CDR-H3 of SEQ ID NO: 29, CDR-L1 of SEQ ID NO: 30, CDR-L2 of SEQ ID NO: 31, and CDR-L3 of SEQ ID NO: 32.

12.-13. (canceled)

14. The CAR polypeptide of claim 1, wherein the extracellular domain comprises the antigen binding domain specific for an Fc domain.

15.-16. (canceled)

17. The CAR polypeptide of claim 14, wherein the Fc domain comprises the amino acid sequence of an Fc selected from SEQ ID NOs. 91-94.

18. The CAR polypeptide of claim 14, wherein the antigen binding domain is a single chain fragment variable (scFv).

19. The CAR polypeptide of claim 1, wherein the hinge domain is a CD28 or CD8 hinge domain.

20. (canceled)

21. The CAR polypeptide of claim 1, wherein the transmembrane domain is an NKG2D transmembrane domain, an NKG2D inverted transmembrane domain, a CD28 transmembrane domain, a CD8 transmembrane domain, a CD16 transmembrane domain, or a FcgR1 (CD64) transmembrane domain.

22. (canceled)

23. The CAR polypeptide of claim 1, wherein the intracellular signaling domain is FcgR1 intracellular signaling domain, a 4-1BB-CD3z intracellular signaling domain, a 2B4-CD3z intracellular signaling domain, a CD16 intracellular signaling domain, a CD64 intracellular signaling domain, or a CD28-CD3z intracellular signaling domain.

24. A nucleic acid encoding the CAR polypeptide of claim 1.

25. A vector comprising the nucleic acid of claim 24.

26.-28. (canceled)

29. A natural killer (NK) cell comprising the nucleic acid of claim 24 and/or expressing the CAR polypeptide of claim 1.

30.-31. (canceled)

32. A method of treating cancer in a subject, the method comprising conjointly administering to the subject:
(A) a natural killer (NK) cell expressing a CAR polypeptide comprising an extracellular domain; and
(B) a multi-specific antigen-binding molecule comprising a first antigen-binding domain that binds to a tumor antigen and a second antigen-binding domain that binds to the extracellular domain.

33. The method of claim 32, the method comprising conjointly administering to the subject:
(A) a natural killer (NK) cell expressing a CAR polypeptide of claim 2; and
(B) a multi-specific antigen-binding molecule comprising a CD3-binding domain that specifically binds to the CD3 extracellular domain or fragment thereof and a tumor antigen-binding domain that specifically binds to a tumor antigen.

34. A method of treating cancer in a subject, the method comprising conjointly administering to the subject:
(A) an antigen binding molecule that binds to a tumor antigen; and
(B) a natural killer (NK) cell expressing a CAR polypeptide comprising an extracellular domain that binds to the antigen-binding molecule.

35. The method of claim 34, the method comprising conjointly administering to the subject:
(A) a multi-specific antigen binding molecule comprising a CD3-binding domain that specifically binds to CD3 and a tumor antigen-binding domain that specifically binds to a tumor antigen; and
(B) a natural killer (NK) cell expressing a CAR polypeptide of claim 8, wherein the antigen binding domain of the CAR polypeptide binds to the idiotype of the CD3-binding domain of the multi-specific antigen binding molecule.

36. A method of treating cancer in a subject, the method comprising conjointly administering to the subject:
(a) an antigen binding molecule that binds to a tumor antigen and that comprises an Fc domain; and
(b) a natural killer (NK) cell expressing a CAR polypeptide comprising an extracellular domain that binds to the Fc domain.

37. The method of claim 36, the method comprising conjointly administering to the subject:
(A) a multi-specific antigen binding molecule comprising a CD3-binding domain that specifically binds to CD3, a tumor antigen-binding domain that specifically binds to a tumor antigen, and a Fc domain; and
(B) a natural killer (NK) cell expressing a CAR polypeptide of claim 14, wherein the antigen binding domain of the CAR polypeptide binds to the Fc domain of the multi-specific antigen binding molecule.

38.-47. (canceled)

48. The method of claim 33, wherein the tumor antigen is selected from CD19, CD123, STEAP2, CD20, SSTR2, CD38, STEAP1, 5T4, ENPP3, PSMA, MUC16, GPRC5D, BCMA, CA19.9, MSLN, CD22, SLC3A2-APIS, CLDN18.2, and CEACAM5.

49.-52. (canceled)

53. A pharmaceutical composition comprising:
(A) a natural killer (NK) cell expressing a CAR polypeptide of claim 2; and
(B) a multi-specific antigen binding molecule comprising a CD3-binding domain that specifically binds to the CD3 extracellular domain or fragment thereof and a tumor antigen-binding domain that specifically binds to a tumor antigen.

54. A pharmaceutical composition comprising:
(A) a multi-specific antigen binding molecule comprising a CD3-binding domain that specifically binds to CD3 and a tumor antigen-binding domain that specifically binds to a tumor antigen; and
(B) a natural killer (NK) cell expressing a CAR polypeptide of claim 8, wherein the antigen binding domain of the CAR polypeptide binds to the idiotype of the CD3-binding domain of the multi-specific antigen binding molecule.

55. A pharmaceutical composition comprising:
(A) a multi-specific antigen binding molecule comprising a CD3-binding domain that specifically binds to CD3, a tumor antigen-binding domain that specifically binds to a tumor antigen, and a Fc domain; and
(B) a natural killer (NK) cell expressing a CAR polypeptide of claim 14, wherein the antigen binding domain of the CAR polypeptide binds to the Fc domain of the multi-specific antigen binding molecule.

56.-66. (canceled)

67. A cell bank comprising NK cells that express a CAR of claim 1.

68. The method of claim 35, wherein the tumor antigen is selected from CD19, CD123, STEAP2, CD20, SSTR2, CD38, STEAP1, 5T4, ENPP3, PSMA, MUC16, GPRC5D, BCMA, CA19.9, MSLN, CD22, SLC3A2-APIS, CLDN18.2, and CEACAM5.

69. The method of claim 37, wherein the tumor antigen is selected from CD19, CD123, STEAP2, CD20, SSTR2, CD38, STEAP1, 5T4, ENPP3, PSMA, MUC16, GPRC5D, BCMA, CA19.9, MSLN, CD22, SLC3A2-APIS, CLDN18.2, and CEACAM5.


SEQUENCE LISTING (1960 sequences)

SEQ ID NO: 1 - protein, 49 aa, synthetic construct
KPTTTPAPRPPTPAPTIASQPLSLRPEACRPAAGGAVHTRGLDFACDIY

SEQ ID NO: 2 - protein, 48 aa, synthetic construct
AKPTTTPAPRPPTPAPTIASQPLSLRPEACRPAAGGAVHTRGLDFACD

SEQ ID NO: 3 - protein, 45 aa, synthetic construct
TTTPAPRPPTPAPTIASQPLSLRPEACRPAAGGAVHTRGLDFACD

SEQ ID NO: 4 - protein, 39 aa, synthetic construct
IEVMYPPPYLDNERSNGTIIHVKGKHLCPSPLFPGPSKP

SEQ ID NO: 5 - protein, 39 aa, synthetic construct
IEVMYPPPYLDNEKSNGTIIHVKGKHLCPSPLFPGPSKP

SEQ ID NO: 6 - protein, 27 aa, synthetic construct
FWVLVVVGGVLACYSLLVTVAFIIFWV

SEQ ID NO: 7 - protein, 28 aa, synthetic construct
MFWVLVVVGGVLACYSLLVTVAFIIFWV

SEQ ID NO: 8 - protein, 21 aa, synthetic construct
IWAPLAGTCGVLLLSLVITLY

SEQ ID NO: 9 - protein, 24 aa, synthetic construct
IYIWAPLAGTCGVLLLSLVITLYC

SEQ ID NO: 10 - protein, 21 aa, synthetic construct
VSFCLVMVLLFAVDTGLYFSV

SEQ ID NO: 11 - protein, 24 aa, synthetic construct
VSFCLVMVLLFAVDTGLYFSVKTN

SEQ ID NO: 12 - protein, 25 aa, synthetic construct
VASWITVMIIFRIGMAVAIFCCFFF

SEQ ID NO: 13 - protein, 21 aa, synthetic construct
VLFYLAVGIMFLVNTVLWVTI

SEQ ID NO: 14 - protein, 25 aa, synthetic construct
FSCSVMHEALHNRFTQKSLSLSPGK

SEQ ID NO: 15 - protein, 249 aa, synthetic construct
QIQLVQSGPELKKPGETVRISCKASGYTFTDYSIHWVKQAPGKCLKWMGWINTETGEPAYADDFKGRFAFSLETSASTAYLQINNLKNEDTATFFCAHPYDYDVLDYWGQGTSVTVSSGGGGSGGGGSGGGGSGGGGSDTVLTQSPASLGVSLGQRATISCRASKSVSTSNYSYIHWYQQKPGQPPKLLIKYVSYLESGVPARFSGSGSGTDFTLNIHPVEEEDAATYYCQHSREFPWTFGCGTKLEIK

SEQ ID NO: 16 - protein, 5 aa, synthetic construct
DYSIH

SEQ ID NO: 17 - protein, 17 aa, synthetic construct
WINTETGEPAYADDFKG

SEQ ID NO: 18 - protein, 9 aa, synthetic construct
PYDYDVLDY

SEQ ID NO: 19 - protein, 15 aa, synthetic construct
RASKSVSTSNYSYIH

SEQ ID NO: 20 - protein, 7 aa, synthetic construct
YVSYLES

SEQ ID NO: 21 - protein, 9 aa, synthetic construct
QHSREFPWT

SEQ ID NO: 22 - protein, 41 aa, synthetic construct
RSKRSRLLHSDYMNMTPRRPGPTRKHYQPYAPPRDFAAYRS

SEQ ID NO: 23 - protein, 249 aa, synthetic construct
QVQLKESGPGLVAPSQSLSITCTVSGFSLTSYGVSWVRQPPGKCLEWLGIIWGDGSTNYHSALISRLSISKDNSKSQVFLKLNSLQTDDTATYYCAKGITTVVDDYYAMDYWGQGTSVTVSSGGGGSGGGGSGGGGSGGGGSDIQMTQSPASLSASVGETVTITCRASENIDSYLAWYQQKQGKSPQLLVYAATFLADDVPSRFSGSGSGTQYSLKINSLQSEDVARYYCQHYYSTPYTFGCGTKLEIK

SEQ ID NO: 24 - protein, 5 aa, synthetic construct
SYGVS

SEQ ID NO: 25 - protein, 16 aa, synthetic construct
LLWGDGSTNYHSALIS

SEQ ID NO: 26 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 28 - protein, 42 aa, synthetic construct
KRGRKKLLYIFKQPFMRPVQTTQEEDGCSCRFPEEEEGGCEL

SEQ ID NO: 29 - protein, 14 aa, synthetic construct
GITTVVDDYYAMDY

SEQ ID NO: 30 - protein, 11 aa, synthetic construct
RASENIDSYLA

SEQ ID NO: 31 - protein, 7 aa, synthetic construct
AATFLAD

SEQ ID NO: 32 - protein, 9 aa, synthetic construct
QHYYSTPYT

SEQ ID NO: 33 - protein, 12 aa, synthetic construct
ESKYGPPCPSCP

SEQ ID NO: 34 - protein, 12 aa, synthetic construct
ESKYGPPCPPCP

SEQ ID NO: 35 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 119 aa, synthetic construct
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 6 aa, synthetic construct
APEFLG

SEQ ID NO: 38 - protein, 5 aa, synthetic construct
APPVA

SEQ ID NO: 39 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 15 aa, synthetic construct
EPKSCDKTHTCPPCP

SEQ ID NO: 42 - protein, 12 aa, synthetic construct
ERKCCVECPPCP

SEQ ID NO: 43 - protein, 61 aa, synthetic construct
LKTPLGDTTHTCPRCPEPKSCDTPPPCPRCPEPKSCDTPPPCPRCPEPKSCDTPPPCPRCP

SEQ ID NO: 44 - protein, 21 aa, synthetic construct
PVPSTPPTPSPSTPPTPSPSC

SEQ ID NO: 45 - protein, 58 aa, synthetic construct
ESPKAQASSVPTAQPQAEGSLAKATTAPATTRNTGRGGEEKKKEKEKEEQEERETKTP

SEQ ID NO: 46 - protein, 38 aa, synthetic construct
SVVDFLPTTAQPTKKSTLKKRVCRLPRPETQKGPLCSP

SEQ ID NO: 47 - protein, 36 aa, synthetic construct
VDFLPTTAQPTKKSTLKKRVCRLPRPETQKGPLCSP

SEQ ID NO: 48 - protein, 23 aa, synthetic construct
LSEGDKVKMDSRIQVLSRGVNQT

SEQ ID NO: 49 - protein, 22 aa, synthetic construct
SGQVLLESNIKVLPTWSTPVQP

SEQ ID NO: 50 - protein, 23 aa, synthetic construct
DSGQVLLESNIKVLPTWSTPVQP

SEQ ID NO: 51 - protein, 9 aa, synthetic construct
QSFGLLDPK

SEQ ID NO: 52 - protein, 21 aa, synthetic construct
FWLPIGCAAFVVVCILGCILI

SEQ ID NO: 53 - protein, 27 aa, synthetic construct
IISFFLALTSTALLFLLFFLTLRFSVV

SEQ ID NO: 54 - protein, 31 aa, synthetic construct
IISFFLALTSTALLFLLFFLTLRFSVVKRGR

SEQ ID NO: 55 - protein, 34 aa, synthetic construct
TWSTPVQPMALIVLGGVAGLLLFIGLGIFFSVRS

SEQ ID NO: 56 - protein, 22 aa, synthetic construct
MALIVLGGVAGLLLFIGLGIFF

SEQ ID NO: 57 - protein, 21 aa, synthetic construct
LLMIIAPSLGFVLFALFVAFL

SEQ ID NO: 58 - protein, 21 aa, synthetic construct
VAAILGLGLVLGLLGPLAILL

SEQ ID NO: 59 - protein, 26 aa, synthetic construct
VAAILGLGLVLGLLGPLAILLALYLL

SEQ ID NO: 60 - protein, 21 aa, synthetic construct
ILVIFSGMFLVFTLAGALFLH

SEQ ID NO: 61 - protein, 26 aa, synthetic construct
IYLIIGICGGGSLLMVFVALLVFYIT

SEQ ID NO: 62 - protein, 23 aa, synthetic construct
IAAIVGGTVAGIVLIGILLLVIW

SEQ ID NO: 63 - protein, 28 aa, synthetic construct
PVLDAGPVLFWVILVLVVVVGSSAFLLC

SEQ ID NO: 64 - protein, 22 aa, synthetic construct
ALVVIPIIFGILFAILLVLVFI

SEQ ID NO: 65 - protein, 21 aa, synthetic construct
VGVVGGLLGSLVLLVWVLAVI

SEQ ID NO: 66 - protein, 21 aa, synthetic construct
AALAVISFLLGLGLGVACVLA

SEQ ID NO: 67 - protein, 21 aa, synthetic construct
VGLGLLLLLMGAGLAVQGWFL

SEQ ID NO: 68 - protein, 23 aa, synthetic construct
LTAEVLGIICIVLMATVLKTIVL

SEQ ID NO: 69 - protein, 21 aa, synthetic construct
LWVTVGLSVCLIALLVALAFV

SEQ ID NO: 70 - protein, 21 aa, synthetic construct
PFFFCCFIAVAMGIRFIIMVA

SEQ ID NO: 71 - protein, 21 aa, synthetic construct
PFFFCCFIAVAMGIRFIIMVT

SEQ ID NO: 72 - protein, 24 aa, synthetic construct
LVPVFCGLLVAKSLVLSALLVWWG

SEQ ID NO: 73 - protein, 24 aa, synthetic construct
MGLAFLVLVALVWFLVEDWLSRKR

SEQ ID NO: 74 - protein, 21 aa, synthetic construct
GVLAGIVMGDLVLTVLIALAV

SEQ ID NO: 75 - protein, 24 aa, synthetic construct
AGTVLLLRAGFYAVSFLSVAVGST

SEQ ID NO: 76 - protein, 21 aa, synthetic construct
LLAGLVAADAVASLLIVGAVF

SEQ ID NO: 77 - protein, 27 aa, synthetic construct
ILLAGLVAADAVASLLIVGAVFLCARR

SEQ ID NO: 78 - protein, 21 aa, synthetic construct
FLVIIVILSALFLGTLACFCV

SEQ ID NO: 79 - protein, 28 aa, synthetic construct
YTLFVAGGTVLLLLFVISITTIIVIFLN

SEQ ID NO: 80 - protein, 21 aa, synthetic construct
AVFGCIFGALVIVTVGGFIFW

SEQ ID NO: 81 - protein, 23 aa, synthetic construct
LCYLLDGILFIYGVILTALFLRV

SEQ ID NO: 82 - protein, 21 aa, synthetic construct
LCYLLDGILFIYGVILTALFL

SEQ ID NO: 83 - protein, 23 aa, synthetic construct
PLFIPVAVMVTAFSGLAFIIWLA

SEQ ID NO: 84 - protein, 21 aa, synthetic construct
MALPVTALLLPLALLLHAARP

SEQ ID NO: 85 - protein, 21 aa, synthetic construct
LLFLILGVLSLLLLVTGAFGF

SEQ ID NO: 86 - protein, 24 aa, synthetic construct
SIISAVVGILLVVVLGVVFGILII

SEQ ID NO: 87 - protein, 31 aa, synthetic construct
PLGWLTVVLLAVAACVLLLTSAQLGLHIWQL

SEQ ID NO: 88 - protein, 6 aa, synthetic construct
HHHHHH

SEQ ID NO: 89 - protein, 5 aa, synthetic construct
GGGGS

SEQ ID NO: 90 - protein, 6 aa, synthetic construct
GSGGSG

SEQ ID NO: 91 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 92 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 93 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 94 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 122 aa, synthetic construct
EVQLVESGGGLVQPGRSLRLSCAASGFTFNDYAMHWVRQSPGKGLEWVSGIDWNGGSIDYADSVKGRFTISRDTAKNSLYLQMNSLKVEDTALYYCAKDIGHWNYIGGMDVWGQGTTVTVSS

SEQ ID NO: 96 - protein, 10 aa, synthetic construct
QQSYSSPPIT

SEQ ID NO: 97 - protein, 8 aa, synthetic construct
GFTFNDYA

SEQ ID NO: 98 - protein, 129 aa, synthetic construct
EVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSS

SEQ ID NO: 99 - protein, 8 aa, synthetic construct
IDWNGGSI

SEQ ID NO: 100 - protein, 8 aa, synthetic construct
GFAFGDHT

SEQ ID NO: 101 - protein, 15 aa, synthetic construct
AKDIGHWNYIGGMDV

SEQ ID NO: 102 - protein, 10 aa, synthetic construct
IRSRAYGGTT

SEQ ID NO: 103 - protein, 108 aa, synthetic construct
DIQMTQSPSSLSASVGDRVTITCRASQSINIYLNWYQQKPGKAPKFLIYAASSLQTGVPSRFSGSGSGTDFTLTISSLEPEDFATYYCQHSYSTPPITFGQGTRLEIK

SEQ ID NO: 104 - protein, 20 aa, synthetic construct
TSGGYDSSLHYYYYYHGMDV

SEQ ID NO: 105 - protein, 6 aa, synthetic construct
QSINIY

SEQ ID NO: 106 - protein, 108 aa, synthetic construct
EIVLTQSPGTLSLSPGERATLSCRASQSITSSYLAWYQQRPGQAPRLLIYGASSRATGIPDRFSGSGSGTDFTLTISRLEPEDFAVYYCQQYGSSPWTFGQGTKVEIK

SEQ ID NO: 107 - protein, 10 aa, synthetic construct
QHSYSTPPIT

SEQ ID NO: 108 - protein, 7 aa, synthetic construct
QSITSSY

SEQ ID NO: 109 - protein, 117 aa, synthetic construct
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 110 - skipped

SEQ ID NO: 111 - protein, 8 aa, synthetic construct
GFTFSNYY

SEQ ID NO: 112 - protein, 9 aa, synthetic construct
QQYGSSPWT

SEQ ID NO: 113 - protein, 8 aa, synthetic construct
ISGRGSTI

SEQ ID NO: 114 - protein, 129 aa, synthetic construct
EVQLVESGGGLVQPGRSLRLSCTGSGLTFGDYGMSWVRQAPGKGLEWVGFIRSKGYGGTTEYAASVKGRFTISRDDSKSTAYLQMNSLKTEDTAVYYCTSGGYDSSVHYYYYYYAMDVWGQGTTVTVSS

SEQ ID NO: 115 - protein, 10 aa, synthetic construct
VKDRGGYSPY

SEQ ID NO: 116 - protein, 8 aa, synthetic construct
GLTFGDYG

SEQ ID NO: 117 - protein, 108 aa, synthetic construct
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 118 - protein, 10 aa, synthetic construct
IRSKGYGGTT

SEQ ID NO: 119 - protein, 6 aa, synthetic construct
QSISTY

SEQ ID NO: 120 - protein, 20 aa, synthetic construct
TSGGYDSSVHYYYYYYAMDV

SEQ ID NO: 121 - protein, 123 aa, synthetic construct
QVQLVESGGGVVQPGRSLRLSCAASGFTFSDHGMHWVRQAPGKGLEWVAVISYHGSNKHYADSVKGRFTISRDNSKNALYLQMNSLRPEDTAMYYCAKDGYDFWNGYSLFDYWGQGTLVTVSS

SEQ ID NO: 122 - protein, 108 aa, synthetic construct
EIVLTQSPGTLSLSPGERATLSCRASQTVSSSYLAWYQQKPGQAPRLLIYGASSRATGIPDRFSGSGSGTDFTLTISRLEPEDFAVYYCQQYGSSPWTFGQGTKVEIK

SEQ ID NO: 123 - protein, 8 aa, synthetic construct
GFTFSDHG

SEQ ID NO: 124 - protein, 7 aa, synthetic construct
QTVSSSY

SEQ ID NO: 125 - protein, 114 aa, synthetic construct
DIVMTQTPLSLPVTPGEPASISCRSSQSLLDSDDGNTYLDWYLQKPGQSPQLLIYTLSYRASGVPDRFSGSGSGTDFTLKISRVEAEDVGIYYCMQRIEFPSLTFGGGTKVEIK

SEQ ID NO: 126 - skipped

SEQ ID NO: 127 - protein, 8 aa, synthetic construct
ISYHGSNK

SEQ ID NO: 128 - protein, 9 aa, synthetic construct
QQYGSSPWT

SEQ ID NO: 129 - protein, 16 aa, synthetic construct
AKDGYDFWNGYSLFDY

SEQ ID NO: 130 - protein, 121 aa, synthetic construct
QVQLVESGGGVVQPGRSLRLSCVASGFTFSNYGIHWVRQAPGKGLEWVAVISDDGSFKFYADSVKGRFTISRDNSKNTLYLQMNSLRVEDSAVYHCAKWQHNWNDGGFDYWGQGTLVTVSS

SEQ ID NO: 131 - protein, 108 aa, synthetic construct
DIQMTQSPSSLSASVGDRVTITCRASQSISNYLNWYQQKPGKAPKLLIYTASNLLSGVPSRFSGSGSGTDFTLTISSLQPEDFATYYCQQSYSTPPITFGQGTKLEIK

SEQ ID NO: 132 - protein, 8 aa, synthetic construct
GFTFSNYG

SEQ ID NO: 133 - protein, 123 aa, synthetic construct
QVQLVESGGGVVQPGRSLRLSCAASGFTFSDHAMHWVRQAPGKGLEWVAVISYHGTDKQYGDSVKGRFTISRDNSKNALYLQMNSLRPEDTAMYYCAKDGYDFWNAYSLFDYWGQGTLVTVSS

SEQ ID NO: 134 - protein, 8 aa, synthetic construct
ISDDGSFK

SEQ ID NO: 135 - protein, 8 aa, synthetic construct
GFTFSDHA

SEQ ID NO: 136 - protein, 14 aa, synthetic construct
AKWQHNWNDGGFDY

SEQ ID NO: 137 - protein, 8 aa, synthetic construct
ISYHGTDK

SEQ ID NO: 138 - protein, 108 aa, synthetic construct
EIVLTQSPDTLSLSPGERATLSCRASQSLSSNYLAWYRQKPGQAPRLLIYGISSRATGIPDRFSGSGSGTDFTLTISRLEPEDFAVYYCQQYGSSPWTFGQGTKVEIK

SEQ ID NO: 139 - protein, 16 aa, synthetic construct
AKDGYDFWNAYSLFDY

SEQ ID NO: 140 - protein, 7 aa, synthetic construct
QSLSSNY

SEQ ID NO: 141 - protein, 108 aa, synthetic construct
DIQMTQSPSSLSASVGDRVTITCRASQSISNYLNWYQQKPGKAPKLLIYTASSLLSGVPSRFSGSGSGTDFTLTISSLQPEDFATYYCQQSYSTPPITFGQGTRLEIK

SEQ ID NO: 142 - skipped

SEQ ID NO: 143 - protein, 123 aa, synthetic construct
EVQLVESGGGLVQPGGSLRLSCEASGFTFSNYAMSWVRQAPGKGLEWVSGMSGSGTYPYYADSVKGRFTISRDNSKNMLYLQMNSLRVKDTAVYYCAKDGVDTAMILDTFDLWGQGTMVTVSS

SEQ ID NO: 144 - protein, 9 aa, synthetic construct
QQYGSSPWT

SEQ ID NO: 145 - protein, 8 aa, synthetic construct
GFTFSNYA

SEQ ID NO: 146 - protein, 113 aa, synthetic construct
EVQLVESGGALVQPGGSLRLSCAASGFTFSSYAMHWVRQAPGKGLEFVSTISYNGGTTYYADSVKGRFTVSRDNSKNTVFLRMGSLRTEDMAVYYCARAAAPWGQGTLVTVSS

SEQ ID NO: 147 - protein, 8 aa, synthetic construct
MSGSGTYP

SEQ ID NO: 148 - protein, 8 aa, synthetic construct
GFTFSSYA

SEQ ID NO: 149 - protein, 16 aa, synthetic construct
AKDGVDTAMILDTFDL

SEQ ID NO: 150 - protein, 8 aa, synthetic construct
ISYNGGTT

SEQ ID NO: 151 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTKLEIK

SEQ ID NO: 152 - protein, 6 aa, synthetic construct
ARAAAP

SEQ ID NO: 153 - protein, 120 aa, synthetic construct
QVQLVESGGGLVKPGGSLRLSCAASGFTFRDYSMSWIRQAPGKGLEWVSYVTFFNSAIYYADSVKGRFTISRDNAKNSLYLQMNSLRAEDTAVYYCAREREPIVGGFDYWGQGTLVTVSS

SEQ ID NO: 154 - protein, 108 aa, synthetic construct
EIVLTQSPDTLSLSPGERATLSCRASQSVSNRYLAWYQQKPGQAPRLLIYGASSRATGIPDRFSGSGSGTDFTLTISRLEPEDFAVYYCHQYGSSPWTFGQGTKVEIK

SEQ ID NO: 155 - protein, 8 aa, synthetic construct
GFTFRDYS

SEQ ID NO: 156 - protein, 7 aa, synthetic construct
QSVSNRY

SEQ ID NO: 157 - protein, 8 aa, synthetic construct
VTFFNSAI

SEQ ID NO: 158 - skipped

SEQ ID NO: 159 - protein, 13 aa, synthetic construct
AREREPIVGGFDY

SEQ ID NO: 160 - protein, 9 aa, synthetic construct
HQYGSSPWT

SEQ ID NO: 161 - protein, 108 aa, synthetic construct
DIQMTQSPSSLSASVGDRVTITCRASQSINSYLNWYQQKPGKAPKLLIYAASSLQGGVPSRFSGGGSGTDFTLTITSLQPEDFATFYCQQSYSSPPITFGQGTRLEIK

SEQ ID NO: 162 - protein, 120 aa, synthetic construct
QVQLVQSGAEVKKPGASVKVSCKAFGYTLTGYYIHWVRQAPGQGLEWMGWINPNNGGTNYAQKFQGRVTMTRDKSISTAYMELSRLRSDDTAVYYCARSLELLPDGMDVWGQGTTVTVSS

SEQ ID NO: 163 - protein, 6 aa, synthetic construct
QSINSY

SEQ ID NO: 164 - protein, 8 aa, synthetic construct
GYTLTGYY

SEQ ID NO: 165 - protein, 122 aa, synthetic construct
QVQLVESGGGVVQPGRSLRLSCAASGFTFSTYGIHWVRQAPGKGLEWVAVILHDGSSNYYAESVKGRFIISRDNSKNTLYLQMNSLRAEDTALYYCTKRYSEAAGPNWFDPWGQGTLVTVSS

SEQ ID NO: 166 - protein, 8 aa, synthetic construct
INPNNGGT

SEQ ID NO: 167 - protein, 8 aa, synthetic construct
GFTFSTYG

SEQ ID NO: 168 - protein, 13 aa, synthetic construct
ARSLELLPDGMDV

SEQ ID NO: 169 - protein, 8 aa, synthetic construct
ILHDGSSN

SEQ ID NO: 170 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 171 - protein, 15 aa, synthetic construct
TKRYSEAAGPNWFDP

SEQ ID NO: 172 - protein, 7 aa, synthetic construct
QSVSSSY

SEQ ID NO: 173 - protein, 107 aa, synthetic construct
DIQMTQSPSSVSASVGDRVTITCRASRGISSWLAWYQQKPGKAPKLLIHAASSLQSGVPSRFSGSGSGTDFTLTISSLQPEDFATYYCQQAISFPFTFGPGTKVDIK

SEQ ID NO: 174 - skipped

SEQ ID NO: 175 - protein, 6 aa, synthetic construct
RGISSW

SEQ ID NO: 176 - protein, 9 aa, synthetic construct
QQYGSSPWT

SEQ ID NO: 177 - protein, 9 aa, synthetic construct
QQAISFPFT

SEQ ID NO: 178 - protein, 114 aa, synthetic construct
QVQLVESGGGVVQPGRSLKLSCAASGFTFSSYSLHWVRQAPGKGLEWVALISYDGSNTYYADSVKGRFTISRDNSNNTLFLQMTSLRAEDTAIYYCARSIATSWGQGVLVTVSS

SEQ ID NO: 179 - protein, 120 aa, synthetic construct
EVQLVESGGGLVQPGGSLRLSCAASGFTFSSYVMSWVRQAPGKGLEWVSAIIGSGGSTYYADSVKGRFTISRDNSKNTLYLQMNSLRAEDTAVYYCAKRAGDNWNWFDPWGQGTLVTVSS

SEQ ID NO: 180 - protein, 8 aa, synthetic construct
GFTFSSYS

SEQ ID NO: 181 - protein, 8 aa, synthetic construct
GFTFSSYV

SEQ ID NO: 182 - protein, 8 aa, synthetic construct
ISYDGSNT

SEQ ID NO: 183 - protein, 8 aa, synthetic construct
IIGSGGST

SEQ ID NO: 184 - protein, 7 aa, synthetic construct
ARSIATS

SEQ ID NO: 185 - protein, 13 aa, synthetic construct
AKRAGDNWNWFDP

SEQ ID NO: 186 - protein, 108 aa, synthetic construct
EIVLTQSPGTLSLSPGERATLSCRASQSVSNRYLAWHQQKPGQAPRLLIYGASSRATGIPDRFSGSGSGTDFTLTISRLEPEDFAVYYCQQYGSSPWAFGQGTKVEIK

SEQ ID NO: 187 - protein, 107 aa, synthetic construct
DIQMTQSPSSVSASLGDRVTITCRASQGISSWLAWYQRKPGKAPKLLIYAASSLQSGVPSRFSGSGSGADFTLTISSLQPEDFATYYCQQAKSVPFTFGPGTKVDIK

SEQ ID NO: 188 - protein, 7 aa, synthetic construct
QSVSNRY

SEQ ID NO: 189 - protein, 9 aa, synthetic construct
QQAKSVPFT

SEQ ID NO: 190 - skipped

SEQ ID NO: 191 - protein, 117 aa, synthetic construct
EVQLVESGGGLVQPGRSLRLSCAASGFTFDDYAMHWVRQAPGKGLEWVSGISWNSGNMGYADSVKGRFTISRDNAKNSLFLQMHSLRAEDTAFYYCAKVRLTAFDFWGQGTLVTVSS

SEQ ID NO: 192 - protein, 9 aa, synthetic construct
QQYGSSPWA

SEQ ID NO: 193 - protein, 8 aa, synthetic construct
ISWNSGNM

SEQ ID NO: 194 - protein, 119 aa, synthetic construct
QVQLQESGPGLVKPSETLSLTCTVSGGAINTYYWSWIRQPPGKGLEWIGYIYYRGNTNYNPSLRSRVTISVDTSKNQFFLKLRSVTVADSAVYFCARGWNYRAEYFQHWGQGTVVTVSS

SEQ ID NO: 195 - protein, 10 aa, synthetic construct
AKVRLTAFDF

SEQ ID NO: 196 - protein, 8 aa, synthetic construct
GGAINTYY

SEQ ID NO: 197 - protein, 107 aa, synthetic construct
DIQMTQSPSSLSASVGDRVTITCRTSQSIGNYLNWFQQKPGKAPKLLIYTASSLQNGVPSRFTGSGSGTDFTLTISSLQPEDFVIYYCQQSFSTPYTFGQGTKLEIK

SEQ ID NO: 198 - protein, 7 aa, synthetic construct
IYYRGNT

SEQ ID NO: 199 - protein, 6 aa, synthetic construct
QSIGNY

SEQ ID NO: 200 - protein, 13 aa, synthetic construct
ARGWNYRAEYFQH

SEQ ID NO: 201 - protein, 9 aa, synthetic construct
QQSFSTPYT

SEQ ID NO: 202 - protein, 118 aa, synthetic construct
QVQLQESGPGLVKPSETLSLTCTVSGGSISSHYWSWIRQPPGKGLEWIGYIYYSGSTNYNPSLKSRVTISVDRSKNRFSLKLSSVTAADTAVYYCARTFYDDYAFDYWGQGTLVTVSS

SEQ ID NO: 203 - protein, 118 aa, synthetic construct
QVQLVESGGGLVKPGGSLRLSCAASGFTFSDYYISWIRQAPGKGLEWVSYISSSGSSIKYADSVKGRFTISRDNAKNSLYLQMNSLRAEDTAVYYCAREGGNYGMDVWGQGTTVTVSS

SEQ ID NO: 204 - protein, 8 aa, synthetic construct
GGSISSHY

SEQ ID NO: 205 - protein, 8 aa, synthetic construct
GFTFSDYY

SEQ ID NO: 206 - protein, 7 aa, synthetic construct
IYYSGST

SEQ ID NO: 207 - protein, 8 aa, synthetic construct
ISSSGSSI

SEQ ID NO: 208 - protein, 12 aa, synthetic construct
ARTFYDDYAFDY

SEQ ID NO: 209 - protein, 11 aa, synthetic construct
AREGGNYGMDV

SEQ ID NO: 210 - protein, 108 aa, synthetic construct
EIVLTQSPGTLSLSPGERATLSCRASQSVDTTYLAWYQQKPGQAPRLLIFDASSRATGIPDRFSGSGSGTDFTLTISRLEPEDFAVYYCQQYGSSPFTFGGGTKVEIK

SEQ ID NO: 211 - protein, 107 aa, synthetic construct
DIQMTQSPSSVSASVGDRVTITCRASQGINNWLVWYQQKPGKAPKLLIYAATSLQSGVPSRFSGSGSGTDFTLTISSLQPEDFATYYCQQANSFPPTFGQGTKLEIK

SEQ ID NO: 212 - protein, 7 aa, synthetic construct
QSVDTTY

SEQ ID NO: 213 - protein, 6 aa, synthetic construct
QGINNW

SEQ ID NO: 214 - skipped

SEQ ID NO: 215 - protein, 9 aa, synthetic construct
QQANSFPPT

SEQ ID NO: 216 - protein, 9 aa, synthetic construct
QQYGSSPFT

SEQ ID NO: 217 - protein, 125 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 218 - protein, 118 aa, synthetic construct
QVQLQESGPGLVKPSETLSLTCTVSGGSISSYYWSWIRQPPGKGLEWIGYVDYSGSTDYNPSLKSRVTISVDTSKNQFSLRLNSVTAADTAVYYCARGFNWNYEFDYWGQGTLVTVSS

SEQ ID NO: 219 - protein, 8 aa, synthetic construct
GFTFSNFW

SEQ ID NO: 220 - protein, 8 aa, synthetic construct
GGSISSYY

SEQ ID NO: 221 - protein, 8 aa, synthetic construct
MNQDGSEK

SEQ ID NO: 222 - protein, 7 aa, synthetic construct
VDYSGST

SEQ ID NO: 223 - protein, 18 aa, synthetic construct
ARDREYCISTSCYDDFDY

SEQ ID NO: 224 - protein, 12 aa, synthetic construct
ARGFNWNYEFDY

SEQ ID NO: 225 - protein, 108 aa, synthetic construct
DIQMTQSPSSLSASVGDRVTITCRASQSISSYLNWYQQKPGKAPKLLIYAASSLHSGVPSRFSGSGSGTDFTLTISSLQPEDFATYYCQQSYSTPPITFGQGTRLEIK

SEQ ID NO: 226 - protein, 108 aa, synthetic construct
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPYTFGQGTTLEIK

SEQ ID NO: 227 - protein, 125 aa, synthetic construct
QVQLQESGPGLVKPSETLSLTCTVSGGSINYYYWNWIRQPPGKGLEWIGYISYSGNTNYNPSLKSRVTISVATSRNQFSLTLSSVTAADTAVYYCARFAEYCGGNICYYYGMDVWGQGTTVTVSS

SEQ ID NO: 228 - protein, 7 aa, synthetic construct
QSVSSTY

SEQ ID NO: 229 - protein, 108 aa, synthetic construct
EIVLTQSPGTLSLSPGERATFSCRASQSVGSSFLAWYQQKPGQAPRRLMYGASNRATGIPDRFSGSGSGTDFTLTISRLEPEDFAVYYCQQCGGSPWTFGQGTKVEIK

SEQ ID NO: 230 - skipped

SEQ ID NO: 231 - protein, 127 aa, synthetic construct
QVQLVQSGAEVKRPGSSVKVSCKASGGTFSTYAINWVRQAPGQGLEWMGGIIPIFGTTNYAQKFQGRVTITTDGSTTTTYMELSSLRSEDTAVYYCARTVYDLNSKGYYYYYGLDVWGQGTTVTVSS

SEQ ID NO: 232 - protein, 9 aa, synthetic construct
QQYGSSPYT

SEQ ID NO: 233 - skipped

SEQ ID NO: 234 - protein, 117 aa, synthetic construct
QMQLQESGPGLVKPSETLSLTCTVSGGSISSYYWNWIRQPPGKGLEWIGYIYYSGNNFNPSLKSRVTISVDTSKNQFSLKLSSVTAADTAVYYCARAFHWNYEFDYWGQGTLVTVSS

SEQ ID NO: 235 - skipped

SEQ ID NO: 236 - protein, 8 aa, synthetic construct
GGSISSYY

SEQ ID NO: 237 - skipped

SEQ ID NO: 238 - protein, 6 aa, synthetic construct
IYYSGN

SEQ ID NO: 239 - skipped

SEQ ID NO: 240 - protein, 12 aa, synthetic construct
ARAFHWNYEFDY

SEQ ID NO: 241 - skipped

SEQ ID NO: 242 - protein, 108 aa, synthetic construct
EIVLTQSPGTLSLSPGERATLSCRASQSVSNNYLAWYQQKPGQAPRLLLYGASSRTTDIPDRFSGSGSGTDFTLTISRLEPEDFAVYYCQQYDNSPLTFGGGTKVEIR

SEQ ID NO: 243 - skipped

SEQ ID NO: 244 - protein, 7 aa, synthetic construct
QSVSNNY

SEQ ID NO: 245-247 - skipped (3 entries)

SEQ ID NO: 248 - protein, 9 aa, synthetic construct
QQYDNSPLT

SEQ ID NO: 249 - skipped

SEQ ID NO: 250 - protein, 122 aa, synthetic construct
EVQLVESGGGLVQPGRSLRLSCAASGFTFDEYAMHWVRQAPGKGLEWVSSISWNQGNIDYADSVKGRFTISRDNAKGSLYLQMNSLRAEDTALYYCVKDRSSHYYYYGMDVWGQGTTVTVSS

SEQ ID NO: 251 - skipped

SEQ ID NO: 252 - protein, 8 aa, synthetic construct
GFTFDEYA

SEQ ID NO: 253 - skipped

SEQ ID NO: 254 - protein, 8 aa, synthetic construct
ISWNQGNI

SEQ ID NO: 255 - skipped

SEQ ID NO: 256 - protein, 15 aa, synthetic construct
VKDRSSHYYYYGMDV

SEQ ID NO: 257 - skipped

SEQ ID NO: 258 - protein, 119 aa, synthetic construct
QMQLQQWGAGLLKPSETLSLTCAVYGGSLSGYYWNWIRQSPGKGLEWIGKINHSGSTNYNPSLKSRVTISVDRSRIQFSLKLSSVTAADTAVYYCARALELLTDGMDVWGQGTTVTVSS

SEQ ID NO: 259 - skipped

SEQ ID NO: 260 - protein, 8 aa, synthetic construct
GGSLSGYY

SEQ ID NO: 261 - skipped

SEQ ID NO: 262 - protein, 7 aa, synthetic construct
INHSGST

SEQ ID NO: 263 - skipped

SEQ ID NO: 264 - protein, 13 aa, synthetic construct
ARALELLTDGMDV

SEQ ID NO: 265 - skipped

SEQ ID NO: 266 - protein, 108 aa, synthetic construct
EIVLTQSPGTLSLSPGERATLSCRASQSVSSNYLAWYQQTPGQAPRLLIYGASSRATGIPDRFSGSGSGTDFTLTISRLEPEDFAVYYCQQYGSSPWTFGQGTKVEIK

SEQ ID NO: 267 - skipped

SEQ ID NO: 268 - protein, 7 aa, synthetic construct
QSVSSNY

SEQ ID NO: 269-271 - skipped (3 entries)

SEQ ID NO: 272 - protein, 9 aa, synthetic construct
QQYGSSPWT

SEQ ID NO: 273 - skipped

SEQ ID NO: 274 - protein, 122 aa, synthetic construct
EVQLVESGGGLVQPGRSLRLSCAASGFTFDDYAMHWVRQAPGRGLEWVSSISWNSDNIDYADSVKGRFTIARDSAKNSLYLQMNSLRAEDTALYYCVKDRSSHYYYYGMDVWGQGTTVTVSS

SEQ ID NO: 275 - skipped

SEQ ID NO: 276 - protein, 8 aa, synthetic construct
GFTFDDYA

SEQ ID NO: 277 - skipped

SEQ ID NO: 278 - protein, 8 aa, synthetic construct
ISWNSDNI

SEQ ID NO: 279 - skipped

SEQ ID NO: 280 - protein, 15 aa, synthetic construct
VKDRSSHYYYYGMDV

SEQ ID NO: 281 - skipped

SEQ ID NO: 282 - protein, 119 aa, synthetic construct; identical to SEQ ID NO: 258

SEQ ID NO: 283 - skipped

SEQ ID NO: 284 - protein, 8 aa, synthetic construct
GGSLSGYY

SEQ ID NO: 285 - skipped

SEQ ID NO: 286 - protein, 7 aa, synthetic construct
INHSGST

SEQ ID NO: 287 - skipped

SEQ ID NO: 288 - protein, 13 aa, synthetic construct
ARALELLTDGMDV

SEQ ID NO: 289 - skipped

SEQ ID NO: 290 - protein, 108 aa, synthetic construct; identical to SEQ ID NO: 266

SEQ ID NO: 291 - skipped

SEQ ID NO: 292 - protein, 7 aa, synthetic construct
QSVSSNY

SEQ ID NO: 293-295 - skipped (3 entries)

SEQ ID NO: 296 - protein, 9 aa, synthetic construct
QQYGSSPWT

SEQ ID NO: 297 - skipped

SEQ ID NO: 298 - protein, 121 aa, synthetic construct
EVQLVESGGGLVQAGGSLRLSCAASGFTFSYHEMNWVRQAPGKGLEWVSYIGSRGTTKHYADPVKGRFTISRDNAKNSLYLQMNSLRAEDTAVYYCASEVGSILGYSMDVWGQGTTVTVSS

SEQ ID NO: 299 - skipped

SEQ ID NO: 300 - protein, 8 aa, synthetic construct
GFTFSYHE

SEQ ID NO: 301 - skipped

SEQ ID NO: 302 - protein, 8 aa, synthetic construct
IGSRGTTK

SEQ ID NO: 303 - skipped

SEQ ID NO: 304 - protein, 14 aa, synthetic construct
ASEVGSILGYSMDV

SEQ ID NO: 305 - skipped

SEQ ID NO: 306 - protein, 108 aa, synthetic construct
DIQMTQSPSSLSASVGDRVTITCRASQSVSRYLNWCQQKPGKAPKLLTYAASSLQSGVPSRFSGSGSGTDFTLTISSLQPEDFAAYFCQQSYSTPPITFGQGTRLEIK

SEQ ID NO: 307 - skipped

SEQ ID NO: 308 - protein, 6 aa, synthetic construct
QSVSRY

SEQ ID NO: 309-311 - skipped (3 entries)

SEQ ID NO: 312 - protein, 10 aa, synthetic construct
QQSYSTPPIT

SEQ ID NO: 313 - skipped

SEQ ID NO: 314 - protein, 125 aa, synthetic construct
QVQLVQSGAEVKKPGASVKVSCKASGYTFTSSDINWVRQATGQGLEWVGWMNPNYGYTGSARKFQDRVTMTRNTSLSTAYLELHSLRSEDTAVYYCARVTYCSSVTSCFRPFTYWGQGTLVTVSA

SEQ ID NO: 315 - skipped

SEQ ID NO: 316 - protein, 8 aa, synthetic construct
GYTFTSSD

SEQ ID NO: 317 - skipped

SEQ ID NO: 318 - protein, 8 aa, synthetic construct
MNPNYGYT

SEQ ID NO: 319 - skipped

SEQ ID NO: 320 - protein, 18 aa, synthetic construct
ARVTYCSSVTSCFRPFTY

SEQ ID NO: 321 - skipped

SEQ ID NO: 322 - protein, 108 aa, synthetic construct
DIQMTQSPSSLSASVGDRVTITCRASQSISNYLNWYQQKPGKAPKLLIYAASSLQSGVPSRFSGSGSGTDFTLTISSLQPEDFATYYCQQSYSTPPITFGQGTRLEIK

SEQ ID NO: 323 - skipped

SEQ ID NO: 324 - protein, 6 aa, synthetic construct
QSISNY

SEQ ID NO: 325-327 - skipped (3 entries)

SEQ ID NO: 328 - protein, 10 aa, synthetic construct
QQSYSTPPIT

SEQ ID NO: 329 - skipped

SEQ ID NO: 330 - protein, 115 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARKKLVFDYWGQGTLVTVSS

SEQ ID NO: 331 - skipped

SEQ ID NO: 332 - protein, 8 aa, synthetic construct
GGSISSYY

SEQ ID NO: 333 - skipped

SEQ ID NO: 334 - protein, 7 aa, synthetic construct
IYYSGST

SEQ ID NO: 335 - skipped

SEQ ID NO: 336 - protein, 9 aa, synthetic construct
ARKKLVFDY

SEQ ID NO: 337 - skipped

SEQ ID NO: 338 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPWTFGQGTKVEIK

SEQ ID NO: 339 - skipped

SEQ ID NO: 340 - protein, 7 aa, synthetic construct
QSVSSSY

SEQ ID NO: 341-343 - skipped (3 entries)

SEQ ID NO: 344 - protein, 9 aa, synthetic construct
QQYGNSPWT

SEQ ID NO: 345 - skipped

SEQ ID NO: 346 - protein, 118 aa, synthetic construct
QVQLQQWGAGLLKPSETLSLTCAVYGGSFSDYYWSWIRQPPGKGLEWIGEINHRGKTNYNPSLKSRVSISVDPSKNQFSLKLSSVTAADTAVYYCARQGWNYAIFDYWGQGILVPVSS

SEQ ID NO: 347 - skipped

SEQ ID NO: 348 - protein, 8 aa, synthetic construct
GGSFSDYY

SEQ ID NO: 349 - skipped

SEQ ID NO: 350 - protein, 7 aa, synthetic construct
INHRGKT

SEQ ID NO: 351 - skipped

SEQ ID NO: 352 - protein, 12 aa, synthetic construct
ARQGWNYAIFDY

SEQ ID NO: 353 - skipped

SEQ ID NO: 354 - protein, 113 aa, synthetic construct
DIVMTQSPDSLAVSLGERATINCKSSQSVLYRSNNKNYLGWYQQKPGQPPKLLIYWASSREAGVPDRFSGSGSGTEFTLTISSLQAEDEAVYYCQQYYSSPPTFGQGTKVEIK

SEQ ID NO: 355 - skipped

SEQ ID NO: 356 - protein, 12 aa, synthetic construct
QSVLYRSNNKNY

SEQ ID NO: 357-359 - skipped (3 entries)

SEQ ID NO: 360 - protein, 9 aa, synthetic construct
QQYYSSPPT

SEQ ID NO: 361 - skipped

SEQ ID NO: 362 - protein, 118 aa, synthetic construct
QVQLQQWGAGLLKPSETLSLTCAVYGGSFSGYYWSWIRQPPGKGLEWIGEINHSGKTTYSPSLKSRVTISVDTSKSQFSLKLSSVTAADTAVYYCARQGLNYAIFDYWGQGTLVTVSS

SEQ ID NO: 363 - skipped

SEQ ID NO: 364 - protein, 8 aa, synthetic construct
GGSFSGYY

SEQ ID NO: 365 - skipped

SEQ ID NO: 366 - protein, 7 aa, synthetic construct
INHSGKT

SEQ ID NO: 367 - skipped

SEQ ID NO: 368 - protein, 12 aa, synthetic construct
ARQGLNYAIFDY

SEQ ID NO: 369 - skipped

SEQ ID NO: 370 - protein, 113 aa, synthetic construct
YIVMTQSPDSLAVSLGERATINCKSSRSILYTSNNKNYLAWYQQRPGQPPKLLIYWASTRESGVPDRFSGSGSGTDFTLTISSLQAEDVAVYYCQQYFSSPPTFGQGTKVEIK

SEQ ID NO: 371 - skipped

SEQ ID NO: 372 - protein, 12 aa, synthetic construct
RSILYTSNNKNY

SEQ ID NO: 373-375 - skipped (3 entries)

SEQ ID NO: 376 - protein, 9 aa, synthetic construct
QQYFSSPPT

SEQ ID NO: 377 - skipped

SEQ ID NO: 378 - protein, 118 aa, synthetic construct
QVQLQQWGAGLLKPSETLSLTCAVYGGSFSGFYWSWIRQPPGKGLEWIGEINHSGSAKYNPSLKNRVIISIDTSKNQFSLKLDSVTAADTAVYNCARQGWNFAIFDYWGQGTLVDVSS

SEQ ID NO: 379 - skipped

SEQ ID NO: 380 - protein, 8 aa, synthetic construct
GGSFSGFY

SEQ ID NO: 381 - skipped

SEQ ID NO: 382 - protein, 7 aa, synthetic construct
INHSGSA

SEQ ID NO: 383 - skipped

SEQ ID NO: 384 - protein, 12 aa, synthetic construct
ARQGWNFAIFDY

SEQ ID NO: 385 - skipped

SEQ ID NO: 386 - protein, 113 aa, synthetic construct
DIVMTQSPDSLAVSLGGRATINCKSSQSVLHSSYNKDHLAWYQQRPGQPPKLLIYWASTRESGVPDRFSGSGSGTDFTLTISSLQAEDVAVYYCQQYNILPPTFGQGTRVEIK

SEQ ID NO: 387 - skipped

SEQ ID NO: 388 - protein, 12 aa, synthetic construct
QSVLHSSYNKDH

SEQ ID NO: 389-391 - skipped (3 entries)

SEQ ID NO: 392 - protein, 9 aa, synthetic construct
QQYNILPPT

SEQ ID NO: 393 - skipped

SEQ ID NO: 394 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 395 - skipped

SEQ ID NO: 396 - protein, 6 aa, synthetic construct
QSISSY

SEQ ID NO: 397-399 - skipped (3 entries)

SEQ ID NO: 400 - protein, 10 aa, synthetic construct
QQSYSTPPIT

SEQ ID NO: 401 - skipped

SEQ ID NO: 402 - protein, 123 aa, synthetic construct
EVQLVESGGGLVQPGGSLRLSCAATGFTFDDFTMHWVRQAPGKGLEWVSGISWNSGSIGYVDSVKGRFTISRDNAKNSLYLQMNSLRAEDTALYYCAKDNSGYGYYYYGMDVWGQGTTVTVSS

SEQ ID NO: 403 - skipped

SEQ ID NO: 404 - protein, 8 aa, synthetic construct
GFTFDDFT

SEQ ID NO: 405 - skipped

SEQ ID NO: 406 - protein, 8 aa, synthetic construct
ISWNSGSI

SEQ ID NO: 407 - skipped

SEQ ID NO: 408 - protein, 16 aa, synthetic construct
AKDNSGYGYYYYGMDV

SEQ ID NO: 409 - skipped

SEQ ID NO: 410 - protein, 107 aa, synthetic construct
EIVMTQSPATLSVSPGERATLSCRASHSVSRNSAWYQQKPGQAPRLLIYGASTRATGIPARFSGSGSGTEFTLTISSLQSEDFAIYYCQQYNNWPLTFGGGTKVEIK

SEQ ID NO: 411 - skipped

SEQ ID NO: 412 - protein, 6 aa, synthetic construct
HSVSRN

SEQ ID NO: 413-415 - skipped (3 entries)

SEQ ID NO: 416 - protein, 9 aa, synthetic construct
QQYNNWPLT

SEQ ID NO: 417 - skipped

SEQ ID NO: 418 - protein, 124 aa, synthetic construct
EVQLVESGGGLVQPGGSLRLSCAASGFSFDDYTMHWVRQPPGKGLEWVSGISWNSGSIGYADSVKGRFTISRDNAKNSLYLQMNSLRAGDTALYYCAKDNSGYGYYYYYGMDVWGQGTTVTVSS

SEQ ID NO: 419 - skipped

SEQ ID NO: 420 - protein, 8 aa, synthetic construct
GFSFDDYT

SEQ ID NO: 421 - skipped

SEQ ID NO: 422 - protein, 8 aa, synthetic construct
ISWNSGSI

SEQ ID NO: 423 - skipped

SEQ ID NO: 424 - protein, 17 aa, synthetic construct
AKDNSGYGYYYYYGMDV

SEQ ID NO: 425 - skipped

SEQ ID NO: 426 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYYNWPLTFGGGTKVEIK

SEQ ID NO: 427 - skipped

SEQ ID NO: 428 - protein, 6 aa, synthetic construct
QSVSSN

SEQ ID NO: 429-431 - skipped (3 entries)

SEQ ID NO: 432 - protein, 9 aa, synthetic construct
QQYYNWPLT

SEQ ID NO: 433 - skipped

SEQ ID NO: 434 - protein, 124 aa, synthetic construct
EVQLVESGGGLVQPGRSLRLSCVASGFPFADYTMHWVRQAPGKGLEWVSDISWNSGSKGYADSVKGRFTISRDNAKNSLYLQMNSLRTEDTAFYYCAKDMSGYAHYFYYGMDVWGQGTTVTVSS

SEQ ID NO: 435 - skipped

SEQ ID NO: 436 - protein, 8 aa, synthetic construct
GFPFADYT

SEQ ID NO: 437 - skipped

SEQ ID NO: 438 - protein, 8 aa, synthetic construct
ISWNSGSK

SEQ ID NO: 439 - skipped

SEQ ID NO: 440 - protein, 17 aa, synthetic construct
AKDMSGYAHYFYYGMDV

SEQ ID NO: 441 - skipped

SEQ ID NO: 442 - protein, 108 aa, synthetic construct
DIQMTQSPSSLSASVGDRVTITCRPSQSISSYLNWFQQKPGKAPKLLIYAASSLQSGVPSRFSGSGSGTDFTLTISSLQPEDFATYYCQQSYSTPPITFGQGTRLEIK

SEQ ID NO: 443 - skipped

SEQ ID NO: 444 - protein, 6 aa, synthetic construct
QSISSY

SEQ ID NO: 445-447 - skipped (3 entries)

SEQ ID NO: 448 - protein, 10 aa, synthetic construct
QQSYSTPPIT

SEQ ID NO: 449 - skipped

SEQ ID NO: 450 - protein, 124 aa, synthetic construct
EVQLVESGGGLVQPGRSLRLSCAASGFTFDDYTMHWVRQVPGKGLEWVSGISWNSGSLAYADSVKGRFTISRDNAKNSLYLQMNSLHPEDTALYYCVKDGSGYGHYSYYGLDVWGQGTTVTVSS

SEQ ID NO: 451 - skipped

SEQ ID NO: 452 - protein, 8 aa, synthetic construct
GFTFDDYT

SEQ ID NO: 453 - skipped

SEQ ID NO: 454 - protein, 8 aa, synthetic construct
ISWNSGSL

SEQ ID NO: 455 - skipped

SEQ ID NO: 456 - protein, 17 aa, synthetic construct
VKDGSGYGHYSYYGLDV

SEQ ID NO: 457 - skipped

SEQ ID NO: 458 - protein, 108 aa, synthetic construct; identical to SEQ ID NO: 170

SEQ ID NO: 459 - skipped

SEQ ID NO: 460 - protein, 7 aa, synthetic construct
QSVSSSY

SEQ ID NO: 461-463 - skipped (3 entries)

SEQ ID NO: 464 - protein, 9 aa, synthetic construct
QQYGSSPWT

SEQ ID NO: 465 - skipped

SEQ ID NO: 466 - protein, 124 aa, synthetic construct
EVQLVESGGGLVQPGRSLRLSCVASGFPFADYTMHWVRQAPGKGLEWVSDISWNSGSIGYADSVKGRFTISRDNAKNSLYLQMNSLRTEDTALYYCAKDMSGYAHYFYYGMDVWGQGTTVTVSS

SEQ ID NO: 467 - skipped

SEQ ID NO: 468 - protein, 8 aa, synthetic construct
GFPFADYT

SEQ ID NO: 469 - skipped

SEQ ID NO: 470 - protein, 8 aa, synthetic construct
ISWNSGSI

SEQ ID NO: 471 - skipped

SEQ ID NO: 472 - protein, 17 aa, synthetic construct
AKDMSGYAHYFYYGMDV

SEQ ID NO: 473 - skipped

SEQ ID NO: 474 - protein, 108 aa, synthetic construct
DIQMTQSPSSLSASVGDRVTITCRASQSISSYLNWYQQKPGKAPKLLIFAASSLQSGVPSRFSGSGSGTDFTLTISSLQPEDFATYYCQQSYSTPPITFGQGTRLEIK

SEQ ID NO: 475 - skipped

SEQ ID NO: 476 - protein, 6 aa, synthetic construct
QSISSY

SEQ ID NO: 477-479 - skipped (3 entries)

SEQ ID NO: 480 - protein, 10 aa, synthetic construct
QQSYSTPPIT

SEQ ID NO: 481 - skipped

SEQ ID NO: 482 - protein, 124 aa, synthetic construct
EVQLVESGGGLVQPGRSLKLSCTASGFTFADYTMHWVRQGPGKGLEWVSDISWNSGSKGYADSVKGRFTISRDNAKNSLYLQMNSLRTEDTALYYCAKDMSGYAHYYYYALDVWGQGTTVTVSS

SEQ ID NO: 483 - skipped

SEQ ID NO: 484 - protein, 8 aa, synthetic construct
GFTFADYT

SEQ ID NO: 485 - skipped

SEQ ID NO: 486 - protein, 8 aa, synthetic construct
ISWNSGSK

SEQ ID NO: 487 - skipped

SEQ ID NO: 488 - protein, 17 aa, synthetic construct
AKDMSGYAHYYYYALDV

SEQ ID NO: 489 - skipped

SEQ ID NO: 490 - protein, 108 aa, synthetic construct
DIQMTQSPSSLSASVGDRVTITCRASQSISNYLNWYQQKPGKAPKLLIYAASSLQSGVPSRFSGSGSGTDFTLTISSLQPEDFATYYCQQSYSNPPITFGQGTRLEIK

SEQ ID NO: 491 - skipped

SEQ ID NO: 492 - protein, 6 aa, synthetic construct
QSISNY

SEQ ID NO: 493-495 - skipped (3 entries)

SEQ ID NO: 496 - protein, 10 aa, synthetic construct
QQSYSNPPIT

SEQ ID NO: 497 - skipped

SEQ ID NO: 498 - protein, 121 aa, synthetic construct
QVQLVESGGGVVQPGRSLRLSCAASGFTFSRKGMHWVRQAPVKGLEWVAVISYDGSNKYYADSVKGRFTISRDNSKNTLYLQMNSLTAEDTAVYYCAKEGGHDYGGTFDYWGQGTLVTVSS

SEQ ID NO: 499 - skipped

SEQ ID NO: 500 - protein, 8 aa, synthetic construct
GFTFSRKG

SEQ ID NO: 501 - skipped

SEQ ID NO: 502 - protein, 8 aa, synthetic construct
ISYDGSNK

SEQ ID NO: 503 - skipped

SEQ ID NO: 504 - protein, 14 aa, synthetic construct
AKEGGHDYGGTFDY

SEQ ID NO: 505 - skipped

SEQ ID NO: 506 - protein, 107 aa, synthetic construct
DIQMTQSPSSLSASVGDRVTITCQASQDINNYLNWYQQKPGKAPKFLIYDASNLETGVPSRFSGSGSGTDFTFTISSLQPEDIATYYCQQYDDLPFTFGPGTKVDIK

SEQ ID NO: 507 - skipped

SEQ ID NO: 508 - protein, 6 aa, synthetic construct
QDINNY

SEQ ID NO: 509-511 - skipped (3 entries)

SEQ ID NO: 512 - protein, 9 aa, synthetic construct
QQYDDLPFT

SEQ ID NO: 513 - skipped

SEQ ID NO: 514 - protein, 123 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 515 - skipped

SEQ ID NO: 516 - protein, 8 aa, synthetic construct
GFTFDDYT

SEQ ID NO: 517 - skipped

SEQ ID NO: 518 - protein, 8 aa, synthetic construct
ISWNSGSI

SEQ ID NO: 519 - skipped

SEQ ID NO: 520 - protein, 16 aa, synthetic construct
AKDNSGYGHYYYGMDV

SEQ ID NO: 521 - skipped

SEQ ID NO: 522 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 523 - skipped

SEQ ID NO: 524 - protein, 6 aa, synthetic construct
QSVSSN

SEQ ID NO: 525-527 - skipped (3 entries)

SEQ ID NO: 528 - protein, 9 aa, synthetic construct
QHYINWPLT

SEQ ID NO: 529 - skipped

SEQ ID NO: 530 - protein, 123 aa, synthetic construct; identical to SEQ ID NO: 514

SEQ ID NO: 531 - skipped

SEQ ID NO: 532 - protein, 8 aa, synthetic construct
GFTFDDYT

SEQ ID NO: 533 - skipped

SEQ ID NO: 534 - protein, 8 aa, synthetic construct
ISWNSGSI

SEQ ID NO: 535 - skipped

SEQ ID NO: 536 - protein, 16 aa, synthetic construct
AKDNSGYGHYYYGMDV

SEQ ID NO: 537 - skipped

SEQ ID NO: 538 - protein, 107 aa, synthetic construct; identical to SEQ ID NO: 522

SEQ ID NO: 539 - skipped

SEQ ID NO: 540 - protein, 6 aa, synthetic construct
QSVSSN

SEQ ID NO: 541-543 - skipped (3 entries)

SEQ ID NO: 544 - protein, 9 aa, synthetic construct
QHYINWPLT

SEQ ID NO: 545 - skipped

SEQ ID NO: 546 - protein, 118 aa, synthetic construct
QVQLVESGGGVVQPGRSLRLSCGASGFTFRSYGMHWVRQTPGRGLEWVAMIYYDGNNKYYADSVRGRFTVSRDNSKNTLYLQMSSLRAEDTALYFCARGPGYNWLDPWGQGTLVTVSS

SEQ ID NO: 547 - skipped

SEQ ID NO: 548 - protein, 8 aa, synthetic construct
GFTFRSYG

SEQ ID NO: 549 - skipped

SEQ ID NO: 550 - protein, 8 aa, synthetic construct
IYYDGNNK

SEQ ID NO: 551 - skipped

SEQ ID NO: 552 - protein, 11 aa, synthetic construct
ARGPGYNWLDP

SEQ ID NO: 553 - skipped

SEQ ID NO: 554 - protein, 107 aa, synthetic construct
  VARIANT 38: note = Any amino acid
EIVMTQSPATLSVSPGERATLSCRASQSISRNLAWYQXKPGQAPRLLIYGASTRATGIPARFSGSGSGTEFTLTISSLQSEDFAVYYCQQYNNRPLTFGGGTEVEIK

SEQ ID NO: 555 - skipped

SEQ ID NO: 556 - protein, 6 aa, synthetic construct
QSISRN

SEQ ID NO: 557-559 - skipped (3 entries)

SEQ ID NO: 560 - protein, 9 aa, synthetic construct
QQYNNRPLT

SEQ ID NO: 561 - skipped

SEQ ID NO: 562 - protein, 118 aa, synthetic construct
QVQLVESGGGVVQPGRSLRLACVASGFTFRSYGMHWVRQAPGKGLQWVAMIYYDGKNKYYADSVRGRFTISRDNSKNTLYLQMNNLRVEDTAMYFCARGPGYNWLDPWGQGTLVTVSS

SEQ ID NO: 563 - skipped

SEQ ID NO: 564 - protein, 8 aa, synthetic construct
GFTFRSYG

SEQ ID NO: 565 - skipped

SEQ ID NO: 566 - protein, 8 aa, synthetic construct
IYYDGKNK

SEQ ID NO: 567 - skipped

SEQ ID NO: 568 - protein, 11 aa, synthetic construct
ARGPGYNWLDP

SEQ ID NO: 569 - skipped

SEQ ID NO: 570 - protein, 107 aa, synthetic construct
EIVMTQSPATLSVSPGERATLSCRASQRISSNLAWYQQKPGQAPRLLIYGASTRATGSPARFSGSGSGTDFTLTISSLQSEDVAVYYCQQHHNWPLTFGGGTKVEIK

SEQ ID NO: 571 - skipped

SEQ ID NO: 572 - protein, 6 aa, synthetic construct
QRISSN

SEQ ID NO: 573-575 - skipped (3 entries)

SEQ ID NO: 576 - protein, 9 aa, synthetic construct
QQHHNWPLT

SEQ ID NO: 577 - skipped

SEQ ID NO: 578 - protein, 118 aa, synthetic construct
QVQLVESGGGVVQPGRSLRLSCAASGFTFRSYAMHWVRQAPGKGLEWVAMVYYDGNNQYYADSVRGRFTISRDNSKNTLYLQMNSLRADDTAVYFCARGPGYNWLDPWGQGTLVTVSS

SEQ ID NO: 579 - skipped

SEQ ID NO: 580 - protein, 8 aa, synthetic construct
GFTFRSYA

SEQ ID NO: 581 - skipped

SEQ ID NO: 582 - protein, 8 aa, synthetic construct
VYYDGNNQ

SEQ ID NO: 583 - skipped

SEQ ID NO: 584 - protein, 11 aa, synthetic construct
ARGPGYNWLDP

SEQ ID NO: 585 - skipped

SEQ ID NO: 586 - protein, 107 aa, synthetic construct
EIVMTQSPATLSVSPGERATLSCRASQSVSRNLAWYQQKPGQAPRLLIYGASTRATGIPARFSGSGSGTDFTLTISSLQSEDFAVYYCQQYNNWPLTFGGGTKVVIK

SEQ ID NO: 587 - skipped

SEQ ID NO: 588 - protein, 6 aa, synthetic construct
QSVSRN

SEQ ID NO: 589-591 - skipped (3 entries)

SEQ ID NO: 592 - protein, 9 aa, synthetic construct
QQYNNWPLT

SEQ ID NO: 593 - skipped

SEQ ID NO: 594 - protein, 118 aa, synthetic construct
QVQLVESGGGVVQPGRSLRLSCIASGFTFRSYGMHWVRQAPGKGLEWVAMIYYDGNNKYYADSVRGRFTISRDNSKNTLYLQMNSLRADDTAVYFCARGPGYNWLDPWGQGTLVTVSS

SEQ ID NO: 595 - skipped

SEQ ID NO: 596 - protein, 8 aa, synthetic construct
GFTFRSYG

SEQ ID NO: 597 - skipped

SEQ ID NO: 598 - protein, 8 aa, synthetic construct
IYYDGNNK

SEQ ID NO: 599 - skipped

SEQ ID NO: 600 - protein, 11 aa, synthetic construct
ARGPGYNWLDP

SEQ ID NO: 601 - skipped

SEQ ID NO: 602 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNRPLTFGGGTKVEIK

SEQ ID NO: 603 - skipped

SEQ ID NO: 604 - protein, 6 aa, synthetic construct
QSVSSN

SEQ ID NO: 605-607 - skipped (3 entries)

SEQ ID NO: 608 - protein, 9 aa, synthetic construct
QQYNNRPLT

SEQ ID NO: 609 - skipped

SEQ ID NO: 610 - protein, 118 aa, synthetic construct
QVQLVESGGGVVQPGRSLRLSCAASGFTFRSFGMHWVRQAPGRGLEWVAMIYFDGKNKYYADSVRGRFTISRDNSKNTLYLEMSSLRAEDTAVYFCARGPGYNWLDPWGQGTLVTVSS

SEQ ID NO: 611 - skipped

SEQ ID NO: 612 - protein, 8 aa, synthetic construct
GFTFRSFG

SEQ ID NO: 613 - skipped

SEQ ID NO: 614 - protein, 8 aa, synthetic construct
IYFDGKNK

SEQ ID NO: 615 - skipped

SEQ ID NO: 616 - protein, 11 aa, synthetic construct
ARGPGYNWLDP

SEQ ID NO: 617 - skipped

SEQ ID NO: 618 - protein, 107 aa, synthetic construct
EIVMTQSPATLSVSPGERVTLSCRASQSISRNLAWYQQKPGQAPRLLIYGASTRATGVPARFSGSGSGTEFTLTISSLQSEDFAVFHCQQYNNRPLTFGGGTEVEIK

SEQ ID NO: 619 - skipped

SEQ ID NO: 620 - protein, 6 aa, synthetic construct
QSISRN

SEQ ID NO: 621-623 - skipped (3 entries)

SEQ ID NO: 624 - protein, 9 aa, synthetic construct
QQYNNRPLT

SEQ ID NO: 625 - skipped

SEQ ID NO: 626 - protein, 118 aa, synthetic construct; identical to SEQ ID NO: 610

SEQ ID NO: 627 - skipped

SEQ ID NO: 628 - protein, 8 aa, synthetic construct
GFTFRSFG

SEQ ID NO: 629 - skipped

SEQ ID NO: 630 - protein, 8 aa, synthetic construct
IYFDGKNK

SEQ ID NO: 631 - skipped

SEQ ID NO: 632 - protein, 11 aa, synthetic construct
ARGPGYNWLDP

SEQ ID NO: 633 - skipped

SEQ ID NO: 634 - protein, 107 aa, synthetic construct
  VARIANT 38: note = Any amino acid
EIVMTQSPATLSVSPGERVTLSCRASQSISRNLAWYQXKPGQAPRLLIYGASTRATGVPARFSGSGSGTEFTLTISSLQSEDFAVFHCQQYNNRPLTFGGGTEVEIK

SEQ ID NO: 635 - skipped

SEQ ID NO: 636 - protein, 6 aa, synthetic construct
QSISRN

SEQ ID NO: 637-639 - skipped (3 entries)

SEQ ID NO: 640 - protein, 9 aa, synthetic construct
QQYNNRPLT

SEQ ID NO: 641 - skipped

SEQ ID NO: 642 - protein, 118 aa, synthetic construct; identical to SEQ ID NO: 610

SEQ ID NO: 643 - skipped

SEQ ID NO: 644 - protein, 8 aa, synthetic construct
GFTFRSFG

SEQ ID NO: 645 - skipped

SEQ ID NO: 646 - protein, 8 aa, synthetic construct
IYFDGKNK

SEQ ID NO: 647 - skipped

SEQ ID NO: 648 - protein, 11 aa, synthetic construct
ARGPGYNWLDP

SEQ ID NO: 649 - skipped

SEQ ID NO: 650 - protein, 107 aa, synthetic construct
EIVMTQSPATLSVSPGERATLSCRASQSVSRNLAWYQQKPGQAPRLLIYGASTRATGVPARFSGSGSGTEFTLTISSLQSEDFAVFHCQQYNNRPLTFGGGTEVEIK

SEQ ID NO: 651 - skipped

SEQ ID NO: 652 - protein, 6 aa, synthetic construct
QSVSRN

SEQ ID NO: 653-655 - skipped (3 entries)

SEQ ID NO: 656 - protein, 9 aa, synthetic construct
QQYNNRPLT

SEQ ID NO: 657 - skipped

SEQ ID NO: 658 - protein, 118 aa, synthetic construct; identical to SEQ ID NO: 610

SEQ ID NO: 659 - skipped

SEQ ID NO: 660 - protein, 8 aa, synthetic construct
GFTFRSFG

SEQ ID NO: 661 - skipped

SEQ ID NO: 662 - protein, 8 aa, synthetic construct
IYFDGKNK

SEQ ID NO: 663 - skipped

SEQ ID NO: 664 - protein, 11 aa, synthetic construct
ARGPGYNWLDP

SEQ ID NO: 665 - skipped

SEQ ID NO: 666 - protein, 107 aa, synthetic construct; identical to SEQ ID NO: 650

SEQ ID NO: 667 - skipped

SEQ ID NO: 668 - protein, 6 aa, synthetic construct
QSVSRN

SEQ ID NO: 669-671 - skipped (3 entries)

SEQ ID NO: 672 - protein, 9 aa, synthetic construct
QQYNNRPLT

SEQ ID NO: 673 - skipped

SEQ ID NO: 674 - protein, 118 aa, synthetic construct
QVQLVESGGGVVQPGRSLRLSCVASGFTFRSYGMHWVRQAPGKGLQWVAMIYYDGKNKYYADSVRGRFTISRDNSKNTLYLQMNSLRAEDTAMYFCARGPGYNWLDPWGQGTLVTVSS

SEQ ID NO: 675 - skipped

SEQ ID NO: 676 - protein, 8 aa, synthetic construct
GFTFRSYG

SEQ ID NO: 677 - skipped

SEQ ID NO: 678 - protein, 8 aa, synthetic construct
IYYDGKNK

SEQ ID NO: 679 - skipped

SEQ ID NO: 680 - protein, 11 aa, synthetic construct
ARGPGYNWLDP

SEQ ID NO: 681 - skipped

SEQ ID NO: 682 - protein, 107 aa, synthetic construct
EIVMTQSPATLSVSPGERATLSCRASQRISSNLAWYQQKPGQAPRLLIYGASTRATGSPARFSGSGSGTEFTLTISSLQSEDVAVYYCQQHNNWPLTFGGGTKVEIK

SEQ ID NO: 683 - skipped

SEQ ID NO: 684 - protein, 6 aa, synthetic construct
QRISSN

SEQ ID NO: 685-687 - skipped (3 entries)

SEQ ID NO: 688 - protein, 9 aa, synthetic construct
QQHNNWPLT

SEQ ID NO: 689 - skipped

SEQ ID NO: 690 - protein, 118 aa, synthetic construct; identical to SEQ ID NO: 610

SEQ ID NO: 691 - skipped

SEQ ID NO: 692 - protein, 8 aa, synthetic construct
GFTFRSFG

SEQ ID NO: 693 - skipped

SEQ ID NO: 694 - protein, 8 aa, synthetic construct
IYFDGKNK

SEQ ID NO: 695 - skipped

SEQ ID NO: 696 - protein, 11 aa, synthetic construct
ARGPGYNWLDP

SEQ ID NO: 697 - skipped

SEQ ID NO: 698 - protein, 107 aa, synthetic construct
EIVMTQSPATLSVSPGERVILSCRASQSISRNLAWYQQKPGQAPRLLIYGATTRATGVPARFSGSGSGTEFTLTISSLQSEDFAVFYCQQYNNRPLTFGGGTEVEIK

SEQ ID NO: 699 - skipped

SEQ ID NO: 700 - protein, 6 aa, synthetic construct
QSISRN

SEQ ID NO: 701-703 - skipped (3 entries)

SEQ ID NO: 704 - protein, 9 aa, synthetic construct
QQYNNRPLT

SEQ ID NO: 705 - skipped

SEQ ID NO: 706 - protein, 118 aa, synthetic construct; identical to SEQ ID NO: 610

SEQ ID NO: 707 - skipped

SEQ ID NO: 708 - protein, 8 aa, synthetic construct
GFTFRSFG

SEQ ID NO: 709 - skipped

SEQ ID NO: 710 - protein, 8 aa, synthetic construct
IYFDGKNK

SEQ ID NO: 711 - skipped

SEQ ID NO: 712 - protein, 11 aa, synthetic construct
ARGPGYNWLDP

SEQ ID NO: 713 - skipped

SEQ ID NO: 714 - protein, 107 aa, synthetic construct
  VARIANT 38: note = Any amino acid
EIVMTQSPATLSVSPGERVILSCRASQSISRNLAWYQXKPGQAPRLLIYGATTRATGVPARFSGSGSGTEFTLTISSLQSEDFAVFYCQQYNNRPLTFGGGTEVEIK

SEQ ID NO: 715 - skipped

SEQ ID NO: 716 - protein, 6 aa, synthetic construct
QSISRN

SEQ ID NO: 717-719 - skipped (3 entries)

SEQ ID NO: 720 - protein, 9 aa, synthetic construct
QQYNNRPLT

SEQ ID NO: 721 - skipped

SEQ ID NO: 722 - protein, 118 aa, synthetic construct; identical to SEQ ID NO: 594

SEQ ID NO: 723 - skipped

SEQ ID NO: 724 - protein, 8 aa, synthetic construct
GFTFRSYG

SEQ ID NO: 725 - skipped

SEQ ID NO: 726 - protein, 8 aa, synthetic construct
IYYDGNNK

SEQ ID NO: 727 - skipped

SEQ ID NO: 728 - protein, 11 aa, synthetic construct
ARGPGYNWLDP

SEQ ID NO: 729 - skipped

SEQ ID NO: 730 - protein, 107 aa, synthetic construct; identical to SEQ ID NO: 602

SEQ ID NO: 731 - skipped

SEQ ID NO: 732 - protein, 6 aa, synthetic construct
QSVSSN

SEQ ID NO: 733-735 - skipped (3 entries)

SEQ ID NO: 736 - protein, 9 aa, synthetic construct
QQYNNRPLT

SEQ ID NO: 737 - skipped

SEQ ID NO: 738 - protein, 124 aa, synthetic construct
EVQLVESGGGLVQPGRSLRLSCAASGFTFDDYSMHWVRQAPGKGLEWVSGISWNSRSIDYADSVKGRFTISRDNAKNSLYLQMNSLRAEDTALYYCVKDNSGYGRYYYYGMDVWGQGTTVSVSS

SEQ ID NO: 739 - skipped

SEQ ID NO: 740 - protein, 8 aa, synthetic construct
GFTFDDYS

SEQ ID NO: 741 - skipped

SEQ ID NO: 742 - protein, 8 aa, synthetic construct
ISWNSRSI

SEQ ID NO: 743 - skipped

SEQ ID NO: 744 - protein, 17 aa, synthetic construct
VKDNSGYGRYYYYGMDV

SEQ ID NO: 745 - skipped

SEQ ID NO: 746 - protein, 107 aa, synthetic construct
KIVMTQSPATLSVSPGERATLSCRASQSVSGNLAWYQQKPGQAPRLLIYGASTRATSIPARFSGSGSGTEFTLTISSLQSEDFAVYFCQHYYNWPLTFGGGTKVEIR

SEQ ID NO: 747 - skipped

SEQ ID NO: 748 - protein, 6 aa, synthetic construct
QSVSGN

SEQ ID NO: 749-751 - skipped (3 entries)

SEQ ID NO: 752 - protein, 9 aa, synthetic construct
QHYYNWPLT

SEQ ID NO: 753 - skipped

SEQ ID NO: 754 - protein, 123 aa, synthetic construct; identical to SEQ ID NO: 402

SEQ ID NO: 755 - skipped

SEQ ID NO: 756 - protein, 8 aa, synthetic construct
GFTFDDFT

SEQ ID NO: 757 - skipped

SEQ ID NO: 758 - protein, 8 aa, synthetic construct
ISWNSGSI

SEQ ID NO: 759 - skipped

SEQ ID NO: 760 - protein, 16 aa, synthetic construct
AKDNSGYGYYYYGMDV

SEQ ID NO: 761 - skipped

SEQ ID NO: 762 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 763 - skipped

SEQ ID NO: 764 - protein, 12 aa, synthetic construct
QSVLYSSNNKNY

SEQ ID NO: 765-767 - skipped (3 entries)

SEQ ID NO: 768 - protein, 9 aa, synthetic construct
QQYYSTPYT

SEQ ID NO: 769 - skipped

SEQ ID NO: 770 - protein, 123 aa, synthetic construct
QVTLKESGPALVKPTQTLTLTCTFSGFSLSTSGMCVSWIRQPPGKALEWLARIDWDDDKYYSTSLKTRLTISKDTSKNQVVLTMTNMDPVDTATYYCARMDIVGARGGWFDPWGQGTLVTVSS

SEQ ID NO: 771 - skipped

SEQ ID NO: 772 - protein, 10 aa, synthetic construct
GFSLSTSGMC

SEQ ID NO: 773 - skipped

SEQ ID NO: 774 - protein, 7 aa, synthetic construct
IDWDDDK

SEQ ID NO: 775 - skipped

SEQ ID NO: 776 - protein, 15 aa, synthetic construct
ARMDIVGARGGWFDP

SEQ ID NO: 777 - skipped

SEQ ID NO: 778 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 779 - skipped

SEQ ID NO: 780 - protein, 6 aa, synthetic construct
QGISNY

SEQ ID NO: 781-783 - skipped (3 entries)

SEQ ID NO: 784 - protein, 9 aa, synthetic construct
QQYNSYPLT

SEQ ID NO: 785 - skipped

SEQ ID NO: 786 - protein, 134 aa, synthetic construct
QVQLVESGGGVVQPGRSLRLSCAASGFTFRSYGMHWVRQAPGKGLEWVAMIYYDGNNKKYADSVRGRFTISRDNSKNTLYLQMNSLRVEDTAVYFCARGPGYNWLDPWGQGTLVTVSSAKTTAPPVYPLAPGSL

SEQ ID NO: 787 - skipped

SEQ ID NO: 788 - protein, 8 aa, synthetic construct
GFTFRSYG

SEQ ID NO: 789 - skipped

SEQ ID NO: 790 - protein, 8 aa, synthetic construct
IYYDGNNK

SEQ ID NO: 791 - skipped

SEQ ID NO: 792 - protein, 11 aa, synthetic construct
ARGPGYNWLDP

SEQ ID NO: 793 - skipped

SEQ ID NO: 794 - protein, 107 aa, synthetic construct
EIVMTQSPATLSVSPGERATLSCRASQSVSRNLAWYQQKPGQAPRLLIYGASTRATDIPARFSGSGSGTEFILNISSLQSEDFALYYCQQYSNWPLTFGGGTEVEIK

SEQ ID NO: 795 - skipped

SEQ ID NO: 796 - protein, 6 aa, synthetic construct
QSVSRN

SEQ ID NO: 797-799 - skipped (3 entries)

SEQ ID NO: 800 - protein, 9 aa, synthetic construct
QQYSNWPLT

SEQ ID NO: 801 - skipped

SEQ ID NO: 802 - protein, 118 aa, synthetic construct; identical to SEQ ID NO: 610

SEQ ID NO: 803 - skipped

SEQ ID NO: 804 - protein, 8 aa, synthetic construct
GFTFRSFG

SEQ ID NO: 805 - skipped

SEQ ID NO: 806 - protein, 8 aa, synthetic construct
IYFDGKNK

SEQ ID NO: 807 - skipped

SEQ ID NO: 808 - protein, 11 aa, synthetic construct
ARGPGYNWLDP

SEQ ID NO: 809 - skipped

SEQ ID NO: 810 - protein, 107 aa, synthetic construct; identical to SEQ ID NO: 698

SEQ ID NO: 811 - skipped

SEQ ID NO: 812 - protein, 6 aa, synthetic construct
QSISRN

SEQ ID NO: 813-815 - skipped (3 entries)

SEQ ID NO: 816 - protein, 9 aa, synthetic construct
QQYNNRPLT

SEQ ID NO: 817 - skipped

SEQ ID NO: 818 - protein, 118 aa, synthetic construct; identical to SEQ ID NO: 610

SEQ ID NO: 819 - skipped

SEQ ID NO: 820 - protein, 8 aa, synthetic construct
GFTFRSFG

SEQ ID NO: 821 - skipped

SEQ ID NO: 822 - protein, 8 aa, synthetic construct
IYFDGKNK

SEQ ID NO: 823 - skipped

SEQ ID NO: 824 - protein, 11 aa, synthetic construct
ARGPGYNWLDP

SEQ ID NO: 825 - skipped

SEQ ID NO: 826 - protein, 107 aa, synthetic construct; identical to SEQ ID NO: 698

SEQ ID NO: 827 - skipped

SEQ ID NO: 828 - protein, 6 aa, synthetic construct
QSISRN

SEQ ID NO: 829-831 - skipped (3 entries)

SEQ ID NO: 832 - protein, 9 aa, synthetic construct
QQYNNRPLT

SEQ ID NO: 833 - skipped

SEQ ID NO: 834 - protein, 117 aa, synthetic construct
QVHLEESGGGVVQPGRSLRLSCSASGFTFSSYAMHWVRQAPGKGLEWVAVIWYDGTNKYYLDSVKGRFTISRDNSKNTLFLQMNSLRAEDTAVYYCARDRGSIITHWGQGTLVTVSS

SEQ ID NO: 835 - skipped

SEQ ID NO: 836 - protein, 8 aa, synthetic construct
GFTFSSYA

SEQ ID NO: 837 - skipped

SEQ ID NO: 838 - protein, 8 aa, synthetic construct
IWYDGTNK

SEQ ID NO: 839 - skipped

SEQ ID NO: 840 - protein, 10 aa, synthetic construct
ARDRGSIITH

SEQ ID NO: 841 - skipped

SEQ ID NO: 842 - protein, 108 aa, synthetic construct
DIQMTQSPSSLSASIGDRVTITCRASQNISSYLNWYQQKPGKAPKLLIYAASSLQSGVPSRFSGSGSGTDFTLTISSLQPEDFATYYCQQTYSTPPITFGQGTRLEIK

SEQ ID NO: 843 - skipped

SEQ ID NO: 844 - protein, 6 aa, synthetic construct
QNISSY

SEQ ID NO: 845-847 - skipped (3 entries)

SEQ ID NO: 848 - protein, 10 aa, synthetic construct
QQTYSTPPIT

SEQ ID NO: 849 - skipped

SEQ ID NO: 850 - protein, 124 aa, synthetic construct
EVQLVESGGGLVRPGRSLRLSCAASGFTFDDYTMHWVRQAPGKGLEWVSDISWNSGTIGYADSVKGRFTISRDNAKNSLYLQMNSLRPEDTALYYCAKDMSGYAHYYYYGMDVWGQGTTVTVSS

SEQ ID NO: 851 - skipped

SEQ ID NO: 852 - protein, 8 aa, synthetic construct
GFTFDDYT

SEQ ID NO: 853 - skipped

SEQ ID NO: 854 - protein, 8 aa, synthetic construct
ISWNSGTI

SEQ ID NO: 855 - skipped

SEQ ID NO: 856 - protein, 17 aa, synthetic construct
AKDMSGYAHYYYYGMDV

SEQ ID NO: 857 - skipped

SEQ ID NO: 858 - protein, 108 aa, synthetic construct; identical to SEQ ID NO: 394

SEQ ID NO: 859 - skipped

SEQ ID NO: 860 - protein, 6 aa, synthetic construct
QSISSY

SEQ ID NO: 861-863 - skipped (3 entries)

SEQ ID NO: 864 - protein, 10 aa, synthetic construct
QQSYSTPPIT

SEQ ID NO: 865 - skipped

SEQ ID NO: 866 - protein, 124 aa, synthetic construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTALYYCAKDMSGYAHYGYYGMDVWGQGTTVTVSS

SEQ ID NO: 867 - skipped

SEQ ID NO: 868 - protein, 8 aa, synthetic construct
GFTFDDYT

SEQ ID NO: 869 - skipped

SEQ ID NO: 870 - protein, 8 aa, synthetic construct
ISWNSGSI

SEQ ID NO: 871 - skipped

SEQ ID NO: 872 - protein, 17 aa, synthetic construct
AKDMSGYAHYGYYGMDV

SEQ ID NO: 873 - skipped

SEQ ID NO: 874 - protein, 108 aa, synthetic construct
DIQMTQSPSSLSASVGDRVTITCRASQSIRNYLNWYQQKPGKVPKLLIYAASSLQSGVPSRFSGSGSGTDFTLTISSLQPEDFATYYCQQSYSNPPITFGQGTRLEIK

SEQ ID NO: 875 - skipped

SEQ ID NO: 876 - protein, 6 aa, synthetic construct
QSIRNY

SEQ ID NO: 877-879 - skipped (3 entries)

SEQ ID NO: 880 - protein, 10 aa, synthetic construct
QQSYSNPPIT

SEQ ID NO: 881 - skipped

SEQ ID NO: 882 - protein, 123 aa, synthetic construct
EAQLVESGGGLVQPGRSLRLSCTTSGFTFDDYTMHWVRQAPGKGLEWISDISWNGGTKGYADSVKGRFTISRDNAKNSLYLQMDSLRGEDTALYYCVKDKSGYGHFYFGLDVWGQGTTVTVSS

SEQ ID NO: 883 - skipped

SEQ ID NO: 884 - protein, 8 aa, synthetic construct
GFTFDDYT

SEQ ID NO: 885 - skipped

SEQ ID NO: 886 - protein, 8 aa, synthetic construct
ISWNGGTK

SEQ ID NO: 887 - skipped

SEQ ID NO: 888 - protein, 16 aa, synthetic construct
VKDKSGYGHFYFGLDV

SEQ ID NO: 889 - skipped

SEQ ID NO: 890 - protein, 108 aa, synthetic construct
DIQMTQSPSSLTASVGDRVTFTCRASQSISRHLSWYQQKPGKAPKLLIYAASSLESGVPSRFSGSGSGTDFTLTISSLHPEDFATYYCQQSYSNPPITFGQGTRLEIK

SEQ ID NO: 891 - skipped

SEQ ID NO: 892 - protein, 6 aa, synthetic construct
QSISRH

SEQ ID NO: 893-895 - skipped (3 entries)

SEQ ID NO: 896 - protein, 10 aa, synthetic construct
QQSYSNPPIT

SEQ ID NO: 897 - skipped

SEQ ID NO: 898 - protein, 123 aa, synthetic construct
EVQLVESGGGLVQPGRSLRLSCAASGFKFADYAMHWVRQAPGKGLEWVSEISWNSGSIGYVDSVKGRFTISRDNAKNSLYLQMNSLRAEDTALYYCVKDKSGYGHYYIGMDVWGQGTTVIVSS

SEQ ID NO: 899 - skipped

SEQ ID NO: 900 - protein, 8 aa, synthetic construct
GFKFADYA

SEQ ID NO: 901 - skipped

SEQ ID NO: 902 - protein, 8 aa, synthetic construct
ISWNSGSI

SEQ ID NO: 903 - skipped

SEQ ID NO: 904 - protein, 16 aa, synthetic construct
VKDKSGYGHYYIGMDV

SEQ ID NO: 905 - skipped

SEQ ID NO: 906 - protein, 108 aa, synthetic construct; identical to SEQ ID NO: 394

SEQ ID NO: 907 - skipped

SEQ ID NO: 908 - protein, 6 aa, synthetic construct
QSISSY

SEQ ID NO: 909-911 - skipped (3 entries)

SEQ ID NO: 912 - protein, 10 aa, synthetic construct
QQSYSTPPIT

SEQ ID NO: 913 - skipped

SEQ ID NO: 914 - protein, 124 aa, synthetic construct
EVHLVESGGGLVQPGRSLRLSCAASGFTFDDYTMHWVRQAPGKGLEWVSDISWNSGSIGYADSVKGRFTVSRDNAKNSLYLQMNSLRGEDTALYYCAKDMSGYGHYGKYGMDVWGQGTTVTVSS

SEQ ID NO: 915 - skipped

SEQ ID NO: 916 - protein, 8 aa, synthetic construct
GFTFDDYT

SEQ ID NO: 917 - skipped

SEQ ID NO: 918 - protein, 8 aa, synthetic construct
ISWNSGSI

SEQ ID NO: 919 - skipped

SEQ ID NO: 920 - protein, 17 aa, synthetic construct
AKDMSGYGHYGKYGMDV

SEQ ID NO: 921 - skipped

SEQ ID NO: 922 - protein, 108 aa, synthetic construct
DIQMTQSPSSLSASVGDRVTITCRASQSIRSYLNWYQQKPGKVPKLLIYAASSLQSGVPSRFSGSGSGTDFTLTINSLQPDDFATYYCQQTYSNPPITFGQGTRLEIK

SEQ ID NO: 923 - skipped

SEQ ID NO: 924 - protein, 6 aa, synthetic construct
QSIRSY

SEQ ID NO: 925-927 - skipped (3 entries)

SEQ ID NO: 928 - protein, 10 aa, synthetic construct
QQTYSNPPIT

SEQ ID NO: 929 - skipped

SEQ ID NO: 930 - protein, 124 aa, synthetic construct
EVQLVESGGDLVQPGRSLRLSCAASGFTFDDYTMHWVRQAPGKGLEWVSIISWNGNTIDYADSVKGRFTISRDNAKNSLYLQMNSLRAEDTALYYCAKDKSGYGHFYYYVLDVWGQGTTVTVSS

SEQ ID NO: 931 - skipped

SEQ ID NO: 932 - protein, 8 aa, synthetic construct
GFTFDDYT

SEQ ID NO: 933 - skipped

SEQ ID NO: 934 - protein, 8 aa, synthetic construct
ISWNGNTI

SEQ ID NO: 935 - skipped

SEQ ID NO: 936 - protein, 17 aa, synthetic construct
AKDKSGYGHFYYYVLDV

SEQ ID NO: 937 - skipped

SEQ ID NO: 938 - protein, 107 aa, synthetic construct
DIQMTQSPSSLSASVGDRVTITCRASQSINNYLNWYQQKPGKAPKLLIYAASSLQSGVPSRFSGSGSGTDFTLTISSLQPEDFATYYSQQSYSFPWTFGQGTKVEIK

SEQ ID NO: 939 - skipped

SEQ ID NO: 940 - protein, 6 aa, synthetic construct
QSINNY

SEQ ID NO: 941-943 - skipped (3 entries)

SEQ ID NO: 944 - protein, 9 aa, synthetic construct
QQSYSFPWT

SEQ ID NO: 945 - skipped

SEQ ID NO: 946 - protein, 121 aa, synthetic construct
QVQLVESGGGVVQPGRSLRPSCAASGFSFRDYGMHWVRQAPGKGLEWMAHIWYNGKNKYYADSVKGRFTISRDNSKNTLYLQMNSLRPEDTAVYYCARDGVSARGTPFDYWGQGTLVTVSS

SEQ ID NO: 947 - skipped

SEQ ID NO: 948 - protein, 8 aa, synthetic construct
GFSFRDYG

SEQ ID NO: 949 - skipped

SEQ ID NO: 950 - protein, 8 aa, synthetic construct
IWYNGKNK

SEQ ID NO: 951 - skipped

SEQ ID NO: 952 - protein, 14 aa, synthetic construct
ARDGVSARGTPFDY

SEQ ID NO: 953 - skipped

SEQ ID NO: 954 - protein, 107 aa, synthetic construct
ETTLTQSPAFMSAAPGDKVSISCIASQYIDDDVNWYQQKPGETAIFIIQEASTLVPGISPRFSGSGYGTHFTLTINNIDSEDAAFYFCLQHDNFPYTFGQGTKLEIK

SEQ ID NO: 955 - skipped

SEQ ID NO: 956 - protein, 6 aa, synthetic construct
QYIDDD

SEQ ID NO: 957-959 - skipped (3 entries)

SEQ ID NO: 960 - protein, 9 aa, synthetic construct
LQHDNFPYT

SEQ ID NO: 961 - skipped

SEQ ID NO: 962 - protein, 128 aa, synthetic construct
QVQLVESGGGLVQPGGSLRLSCAASYFTFSSFAMNWVRQAPGQGLEWVSAISGRVSAISGSGGITYYADSVKGRFIISRDNSKNTLYLQMSGLRAEDTAVYYCAKGPYLTTVTPFDYWGQGTLVTVSS

SEQ ID NO: 963 - skipped

SEQ ID NO: 964 - protein, 8 aa, synthetic construct
YFTFSSFA

SEQ ID NO: 965 - skipped

SEQ ID NO: 966 - protein, 8 aa, synthetic construct
ISGSGGIT

SEQ ID NO: 967 - skipped

SEQ ID NO: 968 - protein, 14 aa, synthetic construct
AKGPYLTTVTPFDY

SEQ ID NO: 969 - skipped

SEQ ID NO: 970 - protein, 108 aa, synthetic construct
DIQLTQSPSSVSASVGDRVTITCRASQGISSWLAWYQQKPGKAPKLLIYAVSSLQNGVPSRFSGSGSGTDFTLTISSLQPEDFATYYCQQANSFPFTFGPGTKLEIKR

SEQ ID NO: 971 - skipped

SEQ ID NO: 972 - protein, 6 aa, synthetic construct
QGISSW

SEQ ID NO: 973-975 - skipped (3 entries)

SEQ ID NO: 976 - protein, 9 aa, synthetic construct
QQANSFPFT

SEQ ID NO: 977 - skipped

SEQ ID NO: 978 - protein, 123 aa, synthetic construct
EVQLVESGGGLVQPGRSLRLSCAASGFTFADYAMHWVRQAPGKGLEWVSGISWNSGSIGYADSVKGRFTISRDNAKNSLYLQMNSLRAEDTALYYCVKDNSGYASYYYGMDVWGQGTTVTVSS

SEQ ID NO: 979 - skipped

SEQ ID NO: 980 - protein, 8 aa, synthetic construct
GFTFADYA

SEQ ID NO: 981 - skipped

SEQ ID NO: 982 - protein, 8 aa, synthetic construct
ISWNSGSI

SEQ ID NO: 983 - skipped

SEQ ID NO: 984 - protein, 16 aa, synthetic construct
VKDNSGYASYYYGMDV

SEQ ID NO: 985 - skipped

SEQ ID NO: 986 - protein, 108 aa, synthetic construct
DIQLTQSPSSLSASVGDRVTITCRASQSISSYLNWYQQKPGKAPKLLIYDASNLETGVPSRFSGSGSGTDFTFTISSLQPEDIATYYCQQYDNLPFTFGPGTKVDIKR

SEQ ID NO: 987 - skipped

SEQ ID NO: 988 - protein, 6 aa, synthetic construct
QSISSY

SEQ ID NO: 989-991 - skipped (3 entries)

SEQ ID NO: 992 - protein, 9 aa, synthetic construct
QQYDNLPFT

SEQ ID NO: 993 - skipped

SEQ ID NO: 994 - protein, 126 aa, synthetic construct
EVQLVESGGGVVQPGRSLRLSCAASGFTFSNYGMHWVRQAPGKGLEWVTVILHDGSYKYYADSVKGRFTISRDNSKNTLHLQMNSLRTEDTAVYYCAKGPMFRGVPYNHYYGMDVWGQGTTVTVSS

SEQ ID NO: 995 - skipped

SEQ ID NO: 996 - protein, 8 aa, synthetic construct
GFTFSNYG

SEQ ID NO: 997 - skipped

SEQ ID NO: 998 - protein, 8 aa, synthetic construct
ILHDGSYK

SEQ ID NO: 999 - skipped

SEQ ID NO: 1000 - protein, 19 aa, synthetic construct
AKGPMFRGVPYNHYYGMDV

SEQ ID NO: 1001 - skipped

SEQ ID NO: 1002 - protein, 108 aa, synthetic construct
DIQMTQSPSSLSASVGDRVTITCRASQGIRNDLGWYQQKPGKAPKRLIYAASSLQSGVSSRFSGSGSGTEFTLTISSLQPEDFATYYCLQHNSYPYTFGQGTKVEIKR

SEQ ID NO: 1003 - skipped

SEQ ID NO: 1004 - protein, 6 aa, synthetic construct
QGIRND

SEQ ID NO: 1005-1007 - skipped (3 entries)

SEQ ID NO: 1008 - protein, 9 aa, synthetic construct
LQHNSYPYT

SEQ ID NO: 1009 - skipped

SEQ ID NO: 1010 - protein, 119 aa, synthetic construct
EVQLVESGGGVVQPGRSLRLSCTASGFPFSRYGMHWVRQAPGKGLEWVTFIWYDGSNKYYADSAKGRFTITRDNSKNTVYLQMDSLRADDTAVYYCVRDQAALYYFDSWGQGTLVTVSS

SEQ ID NO: 1011 - skipped

SEQ ID NO: 1012 - protein, 8 aa, synthetic construct
GFPFSRYG

SEQ ID NO: 1013 - skipped

SEQ ID NO: 1014 - protein, 8 aa, synthetic construct
IWYDGSNK

SEQ ID NO: 1015 - skipped

SEQ ID NO: 1016 - protein, 12 aa, synthetic construct
VRDQAALYYFDS

SEQ ID NO: 1017 - skipped

SEQ ID NO: 1018 - protein, 108 aa, synthetic construct
DIQMTQSPSSVSASVGDRVTITCRASQGISRWLAWYQQKPGKAPKLLISAASSLQSGVPSRFSGSGSGTDFTLTISGLQPEDFATYYCQKANSFPFTFGPGTKLEIKR

SEQ ID NO: 1019 - skipped

SEQ ID NO: 1020 - protein, 6 aa, synthetic construct
QGISRW

SEQ ID NO: 1021-1023 - skipped (3 entries)

SEQ ID NO: 1024 - protein, 9 aa, synthetic construct
QKANSFPFT

SEQ ID NO: 1025 - skipped

SEQ ID NO: 1026 - protein, 126 aa, synthetic construct
EVQLVESGGGVVQPGRSLRLSCAASGFTFSSYGMHWVRQAPGKGLEWVTVILHDGSNRYSADSVKGRFTISRDNSKNTLYLQMNILRVEDTAVYYCTKGAMVRGVPYNHYYGMDVWGQGTTVTVSS

SEQ ID NO: 1027 - skipped

SEQ ID NO: 1028 - protein, 8 aa, synthetic construct
GFTFSSYG

SEQ ID NO: 1029 - skipped

SEQ ID NO: 1030 - protein, 8 aa, synthetic construct
ILHDGSNR

SEQ ID NO: 1031 - skipped

SEQ ID NO: 1032 - protein, 19 aa, synthetic construct
TKGAMVRGVPYNHYYGMDV

SEQ ID NO: 1033 - skipped

SEQ ID NO: 1034 - protein, 108 aa, synthetic construct
DIQMTQSPSSLSASVGDRVTITCRASQGIRNDLGWYQQKPGKAPKRLIYAASILQSGVPSRFSGSGSGTEFTLTISSLQPEDFATYYCLQHNSYPYTFGQGTKLEIKR

SEQ ID NO: 1035 - skipped

SEQ ID NO: 1036 - protein, 6 aa, synthetic construct
QGIRND

SEQ ID NO: 1037-1039 - skipped (3 entries)

SEQ ID NO: 1040 - protein, 9 aa, synthetic construct
LQHNSYPYT

SEQ ID NO: 1041 - skipped

SEQ ID NO: 1042 - protein, 126 aa, synthetic construct
EVQLVESGGGVVQPGRSLRLSCAASGFTFSSYGMHWVRQAPGKGLEWVTVILHDGSNRYYADSVKGRFTISRDNSKNTLYLQMNILRAEDTAVYYCTKGAMVRGVPYNHYYGMDVWGQGTTVTVSS

SEQ ID NO: 1043 - skipped

SEQ ID NO: 1044 - protein, 8 aa, synthetic construct
GFTFSSYG

SEQ ID NO: 1045 - skipped

SEQ ID NO: 1046 - protein, 8 aa, synthetic construct
ILHDGSNR

SEQ ID NO: 1047 - skipped

SEQ ID NO: 1048 - protein, 19 aa, synthetic construct
TKGAMVRGVPYNHYYGMDV

SEQ ID NO: 1049 - skipped

SEQ ID NO: 1050 - protein, 108 aa, synthetic construct
DIVMTQSPSSLSASVGDRVTITCRASQGIRNDLGWYQQKPGKAPKRLIYAASNLQSGVPSRFSGSGSGTEFTLTISSLQPEDFATYYCLQHNSYPYTFGQGTKLEIKR

SEQ ID NO: 1051 - skipped

SEQ ID NO: 1052 - protein, 6 aa, synthetic construct
QGIRND

SEQ ID NO: 1053-1055 - skipped (3 entries)

SEQ ID NO: 1056 - protein, 9 aa, synthetic construct
LQHNSYPYT

SEQ ID NO: 1057 - skipped

SEQ ID NO: 1058 - protein, 126 aa, synthetic construct
QVQLVESGGGVVQPGRSLRLSCAASGFTFSSYGMHWVRQAPGKGLEWVAVISYDGSNKYYADSVKGRFTISRDYSKNTLYLQMNSLRAEDTAVFYCAKGAMVRGVPYNYYYGMDVWGQGTTVTVSS

SEQ ID NO: 1059 - skipped

SEQ ID NO: 1060 - protein, 8 aa, synthetic construct
GFTFSSYG

SEQ ID NO: 1061 - skipped

SEQ ID NO: 1062 - protein, 8 aa, synthetic construct
ISYDGSNK

SEQ ID NO: 1063 - skipped

SEQ ID NO: 1064 - protein, 19 aa, synthetic construct
AKGAMVRGVPYNYYYGMDV

SEQ ID NO: 1065 - skipped

SEQ ID NO: 1066 - protein, 107 aa, synthetic construct
DIQMTQSPSSLSASVGDRVTITCRASQGIRNDLGWFQQKPGKAPKRLIYAASSLQSGVPSRFSGSGSGTEFTLTISSLQPEDFATYYCLQHNSYPYTFGQGTKLEIK

SEQ ID NO: 1067 - skipped

SEQ ID NO: 1068 - protein, 6 aa, synthetic construct
QGIRND

SEQ ID NO: 1069-1071 - skipped (3 entries)

SEQ ID NO: 1072 - protein, 9 aa, synthetic construct
LQHNSYPYT

SEQ ID NO: 1073 - skipped

SEQ ID NO: 1074 - protein, 126 aa, synthetic construct
QVQLVESGGGVVQPGRSLRLSCAASGLTFSSYGMHWVRQAPGKGLEWVAVISYDGSNKYYTDSVKGRFTISRDNSKNTLYLQMNSLRAEDTAVYYCAKGAMVRGVPYNYYYGMDVWGQGTTVTVSS

SEQ ID NO: 1075 - skipped

SEQ ID NO: 1076 - protein, 8 aa, synthetic construct
GLTFSSYG

SEQ ID NO: 1077 - skipped

SEQ ID NO: 1078 - protein, 8 aa, synthetic construct
ISYDGSNK

SEQ ID NO: 1079 - skipped

SEQ ID NO: 1080 - protein, 19 aa, synthetic construct
AKGAMVRGVPYNYYYGMDV

SEQ ID NO: 1081 - skipped

SEQ ID NO: 1082 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 1083 - skipped

SEQ ID NO: 1084 - protein, 6 aa, synthetic construct
QGIRND

SEQ ID NO: 1085-1087 - skipped (3 entries)

SEQ ID NO: 1088 - protein, 9 aa, synthetic construct
LQHNSYPYT

SEQ ID NO: 1089 - skipped

SEQ ID NO: 1090 - protein, 126 aa, synthetic construct
QVQLVESGGGVVQPGRSLRLSCAASGFTFRSFGMHWVRQAPGKGLEWVAVISYDGNYKYYADSVKGRFTISRDNSKNTLYLQMNSLRAEDTAVHYCAKGAMVRGVPYNFYYGMDVWGQGTTVTVSS

SEQ ID NO: 1091 - skipped

SEQ ID NO: 1092 - protein, 8 aa, synthetic construct
GFTFRSFG

SEQ ID NO: 1093 - skipped

SEQ ID NO: 1094 - protein, 8 aa, synthetic construct
ISYDGNYK

SEQ ID NO: 1095 - skipped

SEQ ID NO: 1096 - protein, 19 aa, synthetic construct
AKGAMVRGVPYNFYYGMDV

SEQ ID NO: 1097 - skipped

SEQ ID NO: 1098 - protein, 107 aa, synthetic construct
DIQMTQSPSSLSASVGDRVTITCRASQVIRNDLGWYQQKPGKAPKRLIYAASSLQSGIPSRFSGSGSGTEFTLTISSLQPEDFATYYCLQHNSYPYTFGQGTKLEIK

SEQ ID NO: 1099 - skipped

SEQ ID NO: 1100 - protein, 6 aa, synthetic construct
QVIRND

SEQ ID NO: 1101-1103 - skipped (3 entries)

SEQ ID NO: 1104 - protein, 9 aa, synthetic construct
LQHNSYPYT

SEQ ID NO: 1105 - skipped

SEQ ID NO: 1106 - protein, 123 aa, synthetic construct
EVQLVESGGGLVQPGRSLRLSCAASGFTFDDYTMHWVRQVPGKGLEWISGISWNSGSMDYADSVKGRFTISRDNARNSLFLQMNSVRTEDTALYYCAKDKSGYGSFYYGMDVWGQGTTVTVSS

SEQ ID NO: 1107 - skipped

SEQ ID NO: 1108 - protein, 8 aa, synthetic construct
GFTFDDYT

SEQ ID NO: 1109 - skipped

SEQ ID NO: 1110 - protein, 8 aa, synthetic construct
ISWNSGSM

SEQ ID NO: 1111 - skipped

SEQ ID NO: 1112 - protein, 16 aa, synthetic construct
AKDKSGYGSFYYGMDV

SEQ ID NO: 1113 - skipped

SEQ ID NO: 1114 - protein, 108 aa, synthetic construct
EIVLTQSPGTLSLSPGDRATLSCRASQSVSSIYLAWYQQKPGQAPRLLIHGASTRATGIPDRFSGSGSGTDFTLTISRLEPEDFAVYYCQQRAHSPYTFGQGTKLEIK

SEQ ID NO: 1115 - skipped

SEQ ID NO: 1116 - protein, 7 aa, synthetic construct
QSVSSIY

SEQ ID NO: 1117-1119 - skipped (3 entries)

SEQ ID NO: 1120 - protein, 9 aa, synthetic construct
QQRAHSPYT

SEQ ID NO: 1121 - skipped

SEQ ID NO: 1122 - protein, 123 aa, synthetic construct
EVQLVESGGGLVQPGRSLRLSCAASGFSFDNYAMHWVRQAPGQGLEWVSGISWNSGSRDYADSVKGRFTISRDNARNSLFLQMNSLSNEDTAMYYCAKDKSGYGSYFYGMDVWGQGTTVTVSS

SEQ ID NO: 1123 - skipped

SEQ ID NO: 1124 - protein, 8 aa, synthetic construct
GFSFDNYA

SEQ ID NO: 1125 - skipped

SEQ ID NO: 1126 - protein, 8 aa, synthetic construct
ISWNSGSR

SEQ ID NO: 1127 - skipped

SEQ ID NO: 1128 - protein, 16 aa, synthetic construct
AKDKSGYGSYFYGMDV

SEQ ID NO: 1129 - skipped

SEQ ID NO: 1130 - protein, 108 aa, synthetic construct
EIVLTQSPGTLSLSPGDRATLSCRASQSIRNIYLAWYQQKPGQAPRLLIHGASTRATGIPDRFSGSGSGTDFTLTISRLEPEDFAVYYCQQRVSLPYTFGQGTKLEIK

SEQ ID NO: 1131 - skipped

SEQ ID NO: 1132 - protein, 7 aa, synthetic construct
QSIRNIY

SEQ ID NO: 1133-1135 - skipped (3 entries)

SEQ ID NO: 1136 - protein, 9 aa, synthetic construct
QQRVSLPYT

SEQ ID NO: 1137 - skipped

SEQ ID NO: 1138 - protein, 123 aa, synthetic construct
QVQLVESGGGLVQPGRSLRLSCAASGFSFDDYAMHWVRQAPGQGLEWVSGISWNGGSRDYADSVKGRFTISRDNARNSLFLQMNSLFTEDTALYYCAKDKSGYGSYFYGMDVWGQGTTVTVSS

SEQ ID NO: 1139 - skipped

SEQ ID NO: 1140 - protein, 8 aa, synthetic construct
GFSFDDYA

SEQ ID NO: 1141 - skipped

SEQ ID NO: 1142 - protein, 8 aa, synthetic construct
ISWNGGSR

SEQ ID NO: 1143 - skipped

SEQ ID NO: 1144 - protein, 16 aa, synthetic construct
AKDKSGYGSYFYGMDV

SEQ ID NO: 1145 - skipped

SEQ ID NO: 1146 - protein, 108 aa, synthetic construct
EIVLTQSPGILSLSPGDRATLSCRASQSIRNIYLAWYQQKPGQAPRLLIHGASTRATGIPDRFSGSGSGTDFTLTISRLEPEDFAVYYCQQRVSSPYTFGQGTKLEIK

SEQ ID NO: 1147 - skipped

SEQ ID NO: 1148 - protein, 7 aa, synthetic construct
QSIRNIY

SEQ ID NO: 1149-1151 - skipped (3 entries)

SEQ ID NO: 1152 - protein, 9 aa, synthetic construct
QQRVSSPYT

SEQ ID NO: 1153 - skipped

SEQ ID NO: 1154 - protein, 123 aa, synthetic construct
EVQLVESGGGLVQPGRSLRLSCAASGFTFDDYAMHWVRQAPGKGLEWVSGISWNSGSRDYADSVKGRFTISRDNARNSLFLQMNSLSTEDTALYYCAKDKSGYGSYYYGMDVWGQGTTVTVSS

SEQ ID NO: 1155 - skipped

SEQ ID NO: 1156 - protein, 8 aa, synthetic construct
GFTFDDYA

SEQ ID NO: 1157 - skipped

SEQ ID NO: 1158 - protein, 8 aa, synthetic construct
ISWNSGSR

SEQ ID NO: 1159 - skipped

SEQ ID NO: 1160 - protein, 16 aa, synthetic construct
AKDKSGYGSYYYGMDV

SEQ ID NO: 1161 - skipped

SEQ ID NO: 1162 - protein, 108 aa, synthetic construct
EIVLTQSPGTLSLSPGDRATLSCRASQSIRSIYLAWYQQKPGQAPRLLIHGASTRATGIPDRFSGSGSGTDFTLTISRLEPEDFAVYYCQQRVSSPYTFGQGTKLEIK

SEQ ID NO: 1163 - skipped

SEQ ID NO: 1164 - protein, 7 aa, synthetic construct
QSIRSIY

SEQ ID NO: 1165-1167 - skipped (3 entries)

SEQ ID NO: 1168 - protein, 9 aa, synthetic construct
QQRVSSPYT

SEQ ID NO: 1169 - skipped

SEQ ID NO: 1170 - protein, 123 aa, synthetic construct
EVQLVESGGGLVQPGRSLRLSCVASGFTFADFTMHWVRQAPGKGLEWVSGISWNSNSIDYADSVKGRFTISRDNAKKSLFLQMSSLRAEDTALYYCVKDRSGYSRFYYGMDVWGQGTTVTVSS

SEQ ID NO: 1171 - skipped

SEQ ID NO: 1172 - protein, 8 aa, synthetic construct
GFTFADFT

SEQ ID NO: 1173 - skipped

SEQ ID NO: 1174 - protein, 8 aa, synthetic construct
ISWNSNSI

SEQ ID NO: 1175 - skipped

SEQ ID NO: 1176 - protein, 16 aa, synthetic construct
VKDRSGYSRFYYGMDV

SEQ ID NO: 1177 - skipped

SEQ ID NO: 1178 - protein, 107 aa, synthetic construct
EIVMTQSPATLSVSPGERAILSCRASQNINSNLAWYQQKPGQAPRLLIYGASTRATGVPARFSGSGSGTEFTLTIRSLQSEDFAVYYCQQYYNWPITFGHGTRLEIK

SEQ ID NO: 1179 - skipped

SEQ ID NO: 1180 - protein, 6 aa, synthetic construct
QNINSN

SEQ ID NO: 1181-1183 - skipped (3 entries)

SEQ ID NO: 1184 - protein, 9 aa, synthetic construct
QQYYNWPIT

SEQ ID NO: 1185 - skipped

SEQ ID NO: 1186 - protein, 123 aa, synthetic construct
EVQLVESGGGLVQPGRSLRLSCAASGFTFDDYAMHWVRQAPGKGLEWVSGISWNGGSKDYADSVKGRFTISRDNTRNSLSLQMNSLRIEDTALYYCAKDKSGYGSFYYGLDVWGQGTTVTVSS

SEQ ID NO: 1187 - skipped

SEQ ID NO: 1188 - protein, 8 aa, synthetic construct
GFTFDDYA

SEQ ID NO: 1189 - skipped

SEQ ID NO: 1190 - protein, 8 aa, synthetic construct
ISWNGGSK

SEQ ID NO: 1191 - skipped

SEQ ID NO: 1192 - protein, 16 aa, synthetic construct
AKDKSGYGSFYYGLDV

SEQ ID NO: 1193 - skipped

SEQ ID NO: 1194 - protein, 108 aa, synthetic construct; identical to SEQ ID NO: 1162

SEQ ID NO: 1195 - skipped

SEQ ID NO: 1196 - protein, 7 aa, synthetic construct
QSIRSIY

SEQ ID NO: 1197-1199 - skipped (3 entries)

SEQ ID NO: 1200 - protein, 9 aa, synthetic construct
QQRVSSPYT

SEQ ID NO: 1201 - skipped

SEQ ID NO: 1202 - protein, 123 aa, synthetic construct
EVQLVESGGGLVQPGRSLRLSCAASGFTFDDFTMHWVRQAPGKGLEWVSDISWNSGSIDYADSVKGRFTISRDNARNSLFLQMSSLRTEDTASYYCIKDKSGYGSYNYGLDVWGQGTTVTVSS

SEQ ID NO: 1203 - skipped

SEQ ID NO: 1204 - protein, 8 aa, synthetic construct
GFTFDDFT

SEQ ID NO: 1205 - skipped

SEQ ID NO: 1206 - protein, 8 aa, synthetic construct
ISWNSGSI

SEQ ID NO: 1207 - skipped

SEQ ID NO: 1208 - protein, 16 aa, synthetic construct
IKDKSGYGSYNYGLDV

SEQ ID NO: 1209 - skipped

SEQ ID NO: 1210 - protein, 108 aa, synthetic construct
EIVLTQSPGTLSLSPGDRATLSCRASQSVSSIYLAWYQQKPGQAPRLLIHGASTRATGIPDRFSGSGSGTDFTLTISRLEPEDFALYYCHQRVSSPYTFGQGTKLEIK

SEQ ID NO: 1211 - skipped

SEQ ID NO: 1212 - protein, 7 aa, synthetic construct
QSVSSIY

SEQ ID NO: 1213-1215 - skipped (3 entries)

SEQ ID NO: 1216 - protein, 9 aa, synthetic construct
HQRVSSPYT

SEQ ID NO: 1217 - skipped

SEQ ID NO: 1218 - protein, 123 aa, synthetic construct
EVQLVESGGGLVQPGRSLRLSCAASGFTFDDYAMHWVRQAPGKGLEWVSGISWNSGSKDYADSVKGRFTISRDNTRNSLFLQMNSLRTEDTALYYCAKDKSGYGSFYYGMDVWGQGTTVTVSS

SEQ ID NO: 1219 - skipped

SEQ ID NO: 1220 - protein, 8 aa, synthetic construct
GFTFDDYA

SEQ ID NO: 1221 - skipped

SEQ ID NO: 1222 - protein, 8 aa, synthetic construct
ISWNSGSK

SEQ ID NO: 1223 - skipped

SEQ ID NO: 1224 - protein, 16 aa, synthetic construct
AKDKSGYGSFYYGMDV

SEQ ID NO: 1225 - skipped

SEQ ID NO: 1226 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYSPFGQGTKVEIK

SEQ ID NO: 1227 - skipped

SEQ ID NO: 1228 - protein, 6 aa, synthetic construct
QSISSW

SEQ ID NO: 1229-1231 - skipped (3 entries)

SEQ ID NO: 1232 - protein, 8 aa, synthetic construct
QQYNSYSP

SEQ ID NO: 1233 - skipped

SEQ ID NO: 1234 - protein, 124 aa, synthetic construct
QVQLVESGGGVVQPGRSLRLSCAASGLTFSTYVMHWVRQAPGKGLAWVAVIANDGSNKYYADSVKGRFTISRDNSKNTLYLQMNSLRPEDTAVYFCAKEGGTSGSYYYYGMDVWGQGTTVTVSS

SEQ ID NO: 1235 - skipped

SEQ ID NO: 1236 - protein, 8 aa, synthetic construct
GLTFSTYV

SEQ ID NO: 1237 - skipped

SEQ ID NO: 1238 - protein, 8 aa, synthetic construct
IANDGSNK

SEQ ID NO: 1239 - skipped

SEQ ID NO: 1240 - protein, 17 aa, synthetic construct
AKEGGTSGSYYYYGMDV

SEQ ID NO: 1241 - skipped

SEQ ID NO: 1242 - protein, 114 aa, synthetic construct
DIQMTQSPDSLAVSLGERATINCKSSQSLLFNSINKNYLAWYQQKPGQPPKLLLYWASTRESGIPDRFSGSGSGTDFTLTITSLQAEDVALYYCQQYYSIPWTFGQGTKVEIKR

SEQ ID NO: 1243 - skipped

SEQ ID NO: 1244 - protein, 12 aa, synthetic construct
QSLLFNSINKNY

SEQ ID NO: 1245-1247 - skipped (3 entries)

SEQ ID NO: 1248 - protein, 9 aa, synthetic construct
QQYYSIPWT

SEQ ID NO: 1249 - skipped

SEQ ID NO: 1250 - protein, 118 aa, synthetic construct
QVQLVESGAEVKKPGASVKVSCKTSGYTFTGYYMHWIRQAPGQGLEWMGWINPKSGGTNYAQKFQGRVTMTRDTSISTAYMELSRLRSDDMAVYYCARMGDGAVFDFWGQGTLVTVSS

SEQ ID NO: 1251 - skipped

SEQ ID NO: 1252 - protein, 8 aa, synthetic construct
GYTFTGYY

SEQ ID NO: 1253 - skipped

SEQ ID NO: 1254 - protein, 8 aa, synthetic construct
INPKSGGT

SEQ ID NO: 1255 - skipped

SEQ ID NO: 1256 - protein, 11 aa, synthetic construct
ARMGDGAVFDF

SEQ ID NO: 1257 - skipped

SEQ ID NO: 1258 - protein, 108 aa, synthetic construct
AIQMTQSPSSLSASVGDRVTITCRASQRISSFLNWYQQKPGKAPKLLIYTASNLQNGVPSRFSGSGSGTDFTLTISSLQPEDFATYYCQQSYRTPLTFGGGTKVEIKR

SEQ ID NO: 1259 - skipped

SEQ ID NO: 1260 - protein, 6 aa, synthetic construct
QRISSF

SEQ ID NO: 1261-1263 - skipped (3 entries)

SEQ ID NO: 1264 - protein, 9 aa, synthetic construct
QQSYRTPLT

SEQ ID NO: 1265 - skipped

SEQ ID NO: 1266 - protein, 123 aa, synthetic construct
EVQLVESGGGLVQPGRSLRLSCAASGFTFDDYTMHWVRQAPGKGLEWVSGISWNSGSIGYADSVKGRFTISRDNAKNSLYLQMNSLRAEDTALFYCAKDQSGYGHYYYGMDVWGQGTTVTVSS

SEQ ID NO: 1267 - skipped

SEQ ID NO: 1268 - protein, 8 aa, synthetic construct
GFTFDDYT

SEQ ID NO: 1269 - skipped

SEQ ID NO: 1270 - protein, 8 aa, synthetic construct
ISWNSGSI

SEQ ID NO: 1271 - skipped

SEQ ID NO: 1272 - protein, 16 aa, synthetic construct
AKDQSGYGHYYYGMDV

SEQ ID NO: 1273 - skipped

SEQ ID NO: 1274 - protein, 107 aa, synthetic construct
EIVMTQSPATLSVSPGERATLSCRASQSVSSNLAWYQQKPGQAPRLLIYGASTRATDIPARFSGSGSGTEFTLTISSLQSEDFAVYHCQQYNNWPLTFGGGTKVEIK

SEQ ID NO: 1275 - skipped

SEQ ID NO: 1276 - protein, 6 aa, synthetic construct
QSVSSN

SEQ ID NO: 1277-1279 - skipped (3 entries)

SEQ ID NO: 1280 - protein, 9 aa, synthetic construct
QQYNNWPLT

SEQ ID NO: 1281 - skipped

SEQ ID NO: 1282 - protein, 129 aa, synthetic construct
EVQLVESGGGVVQPGRSLRLSCAASGFTFSYYGMHWVRQAPGKGLEWVAVISFDGKNKYYADSVMGRFTISRDNSKNTLYLQMNSLRAEDSAVFFCARSYDILTGYGAGYSYHYGMDVWGQGTTVTVSS

SEQ ID NO: 1283 - skipped

SEQ ID NO: 1284 - protein, 8 aa, synthetic construct
GFTFSYYG

SEQ ID NO: 1285 - skipped

SEQ ID NO: 1286 - protein, 8 aa, synthetic construct
ISFDGKNK

SEQ ID NO: 1287 - skipped

SEQ ID NO: 1288 - protein, 22 aa, synthetic construct
ARSYDILTGYGAGYSYHYGMDV

SEQ ID NO: 1289 - skipped

SEQ ID NO: 1290 - protein, 108 aa, synthetic construct; identical to SEQ ID NO: 1258

SEQ ID NO: 1291 - skipped

SEQ ID NO: 1292 - protein, 6 aa, synthetic construct
QRISSF

SEQ ID NO: 1293-1295 - skipped (3 entries)

SEQ ID NO: 1296 - protein, 9 aa, synthetic construct
QQSYRTPLT

SEQ ID NO: 1297 - skipped

SEQ ID NO: 1298 - protein, 118 aa, synthetic construct
QVQLVESGAEVKKPGASVKVSCKTSGYTLSGYYMHWMRQAPGQGLEWMGWINPKSGVTNYAQKFQDRVAMTRDTSISTAYMELSRLRSDDTAVYYCARMGDGAVFDFWAQGTLVTVSS

SEQ ID NO: 1299 - skipped

SEQ ID NO: 1300 - protein, 8 aa, synthetic construct
GYTLSGYY

SEQ ID NO: 1301 - skipped

SEQ ID NO: 1302 - protein, 8 aa, synthetic construct
INPKSGVT

SEQ ID NO: 1303 - skipped

SEQ ID NO: 1304 - protein, 11 aa, synthetic construct
ARMGDGAVFDF

SEQ ID NO: 1305 - skipped

SEQ ID NO: 1306 - protein, 108 aa, synthetic construct
DIQLTQSPSSLSASVGDRVTITCRASQRISSFLNWYQQKPGKAPKLLIYTASNLQNGVPSRFSGSGSGTDFTLTISSLQPEDFATYYCQQSYRTPLTFGGGTKLEIKR

SEQ ID NO: 1307 - skipped

SEQ ID NO: 1308 - protein, 6 aa, synthetic construct
QRISSF

SEQ ID NO: 1309-1311 - skipped (3 entries)

SEQ ID NO: 1312 - protein, 9 aa, synthetic construct
QQSYRTPLT

SEQ ID NO: 1313 - skipped

SEQ ID NO: 1314 - protein, 124 aa, synthetic construct
QVQLVESGGGVVQPGRSLRLSCAASGFTFSSYGMHWVRQAPGKGLEWVSTISFDGSNKYYADSVKGRFTISRDNSKNTLYLQMNSLRPEDTAVYYCAKGGGTSGSYFYYGMDVWGQGTTVTVSS

SEQ ID NO: 1315 - skipped

SEQ ID NO: 1316 - protein, 8 aa, synthetic construct
GFTFSSYG

SEQ ID NO: 1317 - skipped

SEQ ID NO: 1318 - protein, 8 aa, synthetic construct
ISFDGSNK

SEQ ID NO: 1319 - skipped

SEQ ID NO: 1320 - protein, 17 aa, synthetic construct
AKGGGTSGSYFYYGMDV

SEQ ID NO: 1321 - skipped

SEQ ID NO: 1322 - protein, 108 aa, synthetic construct
AIQMTQSPSSLSASVGDRVTITCRASQRISSFLNWYQQKPGKAPKLLIYTASNLQNGVPSRFSGSGSGTDFTLTISSLQPEDFATYYCQQSYRTPLTFGGGTKLEIKR

SEQ ID NO: 1323 - skipped

SEQ ID NO: 1324 - protein, 6 aa, synthetic construct
QRISSF

SEQ ID NO: 1325-1327 - skipped (3 entries)

SEQ ID NO: 1328 - protein, 9 aa, synthetic construct
QQSYRTPLT

SEQ ID NO: 1329 - skipped

SEQ ID NO: 1330 - protein, 118 aa, synthetic construct
QVQLVQSGTEVKKPGASMKVSCKASGYTFTGYYIHWVRQAPGQGLEWMGWINPNSDVTKYAQKFQGRVTLTRDTSISAAYIDLSRLRSDDTAIYYCARMGDGAVFDYWGQGTLVTVSS

SEQ ID NO: 1331 - skipped

SEQ ID NO: 1332 - protein, 8 aa, synthetic construct
GYTFTGYY

SEQ ID NO: 1333 - skipped

SEQ ID NO: 1334 - protein, 8 aa, synthetic construct
INPNSDVT

SEQ ID NO: 1335 - skipped

SEQ ID NO: 1336 - protein, 11 aa, synthetic construct
ARMGDGAVFDY

SEQ ID NO: 1337 - skipped

SEQ ID NO: 1338 - protein, 108 aa, synthetic construct
DIQLTQSPSSLSASVGDRVTITCRASQRISSYLNWYQQKPGKAPKVLISVASSLQSGVPSRFSGSGFGTDFTLTISSLQPEDSASYYCQQSYNTPLTFGGGTKLEIKR

SEQ ID NO: 1339 - skipped

SEQ ID NO: 1340 - protein, 6 aa, synthetic construct
QRISSY

SEQ ID NO: 1341-1343 - skipped (3 entries)

SEQ ID NO: 1344 - protein, 9 aa, synthetic construct
QQSYNTPLT

SEQ ID NO: 1345 - skipped

SEQ ID NO: 1346 - protein, 118 aa, synthetic construct
EVQLVESGAEVKKPGASVKVSCKTSGYSFIGYYIHWMRQAPGQGLEWMGWINPKSGVTNYAQRFQGRVTMTRDTSISTAYMELSRLKSDDTAVYFCARMGDGAVFDFWGQGTLVTVSS

SEQ ID NO: 1347 - skipped

SEQ ID NO: 1348 - protein, 8 aa, synthetic construct
GYSFIGYY

SEQ ID NO: 1349 - skipped

SEQ ID NO: 1350 - protein, 8 aa, synthetic construct
INPKSGVT

SEQ ID NO: 1351 - skipped

SEQ ID NO: 1352 - protein, 11 aa, synthetic construct
ARMGDGAVFDF

SEQ ID NO: 1353 - skipped

SEQ ID NO: 1354 - protein, 108 aa, synthetic construct
DIQLTQSPSSLSASVGDRVTITCRASQRISSFLNWYQQKPGKVPKLLIYTASNLQNGVPSRFSGTGSGTDFTLTISSLQPEDFATYYCQQSYRTPLTFGGGTKVDIKR

SEQ ID NO: 1355 - skipped

SEQ ID NO: 1356 - protein, 6 aa, synthetic construct
QRISSF

SEQ ID NO: 1357-1359 - skipped (3 entries)

SEQ ID NO: 1360 - protein, 9 aa, synthetic construct
QQSYRTPLT

SEQ ID NO: 1361 - skipped

SEQ ID NO: 1362 - protein, 118 aa, synthetic construct
EVQLVESGAEVKKPGASVKVSCKTSGYTFTGYYMHWIRQAPGQGLEWMGWINPKSGGTNYAQKFQGRVTMTRDTSISTAYMELSRLRSDDMAVYYCARMGDGAVFDFWGQGTLVTVSS

SEQ ID NO: 1363 - skipped

SEQ ID NO: 1364 - protein, 8 aa, synthetic construct
GYTFTGYY

SEQ ID NO: 1365 - skipped

SEQ ID NO: 1366 - protein, 8 aa, synthetic construct
INPKSGGT

SEQ ID NO: 1367 - skipped

SEQ ID NO: 1368 - protein, 11 aa, synthetic construct
ARMGDGAVFDF

SEQ ID NO: 1369 - skipped

SEQ ID NO: 1370 - protein, 109 aa, synthetic construct
DIVMTQSPSSLSASVGDRVTITCRASQSISSYLNWYQQKPGKAPKLLIYAASSLQSGVPSRFSGSGSGTDFTLTISSLQPEDFATYYCQQSYSTPPLTFGGGTKVDIKR

SEQ ID NO: 1371 - skipped

SEQ ID NO: 1372 - protein, 6 aa, synthetic construct
QSISSY

SEQ ID NO: 1373-1375 - skipped (3 entries)

SEQ ID NO: 1376 - protein, 10 aa, synthetic construct
QQSYSTPPLT

SEQ ID NO: 1377 - skipped

SEQ ID NO: 1378 - protein, 118 aa, synthetic construct
QVQLVQSGTEVKKPGASVKVSCKASGYTFTGYYMHWVRQAPGQGLEWMGWINPNSDVTNYAQKFQGRVTLTRDTSISTAYIDLSRLRSDDTAIYYCARMGDGAVFDYWGQGTLVTVSS

SEQ ID NO: 1379 - skipped

SEQ ID NO: 1380 - protein, 8 aa, synthetic construct
GYTFTGYY

SEQ ID NO: 1381 - skipped

SEQ ID NO: 1382 - protein, 8 aa, synthetic construct
INPNSDVT

SEQ ID NO: 1383 - skipped

SEQ ID NO: 1384 - protein, 11 aa, synthetic construct
ARMGDGAVFDY

SEQ ID NO: 1385 - skipped

SEQ ID NO: 1386 - protein, 108 aa, synthetic construct
DIQMTQSPSSLSASVGDRVTITCRASQRISSYLNWYQQKPGKAPKVLISVASSLQSGVPSRFSGSGSGTDFTLTISSLQPEDFASYYCQQSYNTPLTFGGGTKVEIKR

SEQ ID NO: 1387 - skipped

SEQ ID NO: 1388 - protein, 6 aa, synthetic construct
QRISSY

SEQ ID NO: 1389-1391 - skipped (3 entries)

SEQ ID NO: 1392 - protein, 9 aa, synthetic construct
QQSYNTPLT

SEQ ID NO: 1393 - skipped

SEQ ID NO: 1394 - protein, 127 aa, synthetic construct
QVQLVQSGGGLVKPGGSLRLSCAASGFTFSSYDMSWVRQAPGKGLEWVSYISSSGNTIHYADSVKGRFTISRDNSKNSLYLQMNSLRAEDTAVYYCAKAGPATVTRRYYYYYGLDVWGQGTTVTVSS

SEQ ID NO: 1395 - skipped

SEQ ID NO: 1396 - protein, 8 aa, synthetic construct
GFTFSSYD

SEQ ID NO: 1397 - skipped

SEQ ID NO: 1398 - protein, 8 aa, synthetic construct
ISSSGNTI

SEQ ID NO: 1399 - skipped

SEQ ID NO: 1400 - protein, 20 aa, synthetic construct
AKAGPATVTRRYYYYYGLDV

SEQ ID NO: 1401 - skipped

SEQ ID NO: 1402 - protein, 108 aa, synthetic construct
DIVMTQSPSSLSASVRDRVTITCRASQRISSYLNWFQQKPGKAPKVLIYTASSLQNGVPSRFSGSGSGTDFTLTISSLQPEDFATYYCQQSYRTPLTFGGGTKVEIKR

SEQ ID NO: 1403 - skipped

SEQ ID NO: 1404 - protein, 6 aa, synthetic construct
QRISSY

SEQ ID NO: 1405-1407 - skipped (3 entries)

SEQ ID NO: 1408 - protein, 9 aa, synthetic construct
QQSYRTPLT

SEQ ID NO: 1409 - skipped

SEQ ID NO: 1410 - protein, 118 aa, synthetic construct
QVQLVQSGAEVKKPGASVKVSCKASGYTFIGYYMHWVRQAPGQGLEWMGWINPKSGGTNYAQKFQGRVTMTGDTSISTAYMELSRLRSDDTAVFYCARMGDGAMFDYWGQGTLVTVSS

SEQ ID NO: 1411 - skipped

SEQ ID NO: 1412 - protein, 8 aa, synthetic construct
GYTFIGYY

SEQ ID NO: 1413 - skipped

SEQ ID NO: 1414 - protein, 8 aa, synthetic construct
INPKSGGT

SEQ ID NO: 1415 - skipped

SEQ ID NO: 1416 - protein, 11 aa, synthetic construct
ARMGDGAMFDY

SEQ ID NO: 1417 - skipped

SEQ ID NO: 1418 - protein, 107 aa, synthetic construct
DIQMTQSPSSLSASVGDRVTITCRASQRISSYLNWYQQKAGKAPKVLIYTASSLQSGVPSRFSGSGSGTDFTLTISSLQPEDFATYYCQQSYSTPLTFGGGTKVEIK

SEQ ID NO: 1419 - skipped

SEQ ID NO: 1420 - protein, 6 aa, synthetic construct
QRISSY

SEQ ID NO: 1421-1423 - skipped (3 entries)

SEQ ID NO: 1424 - protein, 9 aa, synthetic construct
QQSYSTPLT

SEQ ID NO: 1425 - skipped

SEQ ID NO: 1426 - protein, 125 aa, synthetic construct
QVQLVQSGGGVVQPGGSLRLSCAASGFTFDDYAMHWVRQAPGKGLEWVSLISGDGGSTYYADSVKGRFTISRDNSKNSLYLQMNSLRTEDTALYYCAKDDSSSSWYYYYYGMDVWGRGTTVTVSS

SEQ ID NO: 1427 - skipped

SEQ ID NO: 1428 - protein, 8 aa, synthetic construct
GFTFDDYA

SEQ ID NO: 1429 - skipped

SEQ ID NO: 1430 - protein, 8 aa, synthetic construct
ISGDGGST

SEQ ID NO: 1431 - skipped

SEQ ID NO: 1432 - protein, 18 aa, synthetic construct
AKDDSSSSWYYYYYGMDV

SEQ ID NO: 1433 - skipped

SEQ ID NO: 1434 - protein, 108 aa, synthetic construct; identical to SEQ ID NO: 1386

SEQ ID NO: 1435 - skipped

SEQ ID NO: 1436 - protein, 6 aa, synthetic construct
QRISSY

SEQ ID NO: 1437-1439 - skipped (3 entries)

SEQ ID NO: 1440 - protein, 9 aa, synthetic construct
QQSYNTPLT

SEQ ID NO: 1441 - skipped

SEQ ID NO: 1442 - protein, 124 aa, synthetic construct
EVQLVESGGGLVQPGRSLRLSCAASGFTFGDYTMHWVRQAPGKGLEWVSDISWNSGSKDYADSVKGRFTISRDNAKNSLYLQMNSLRTEDTAFYYCAKDSRGYGLYYHLGLDVWGQGTTVTVSS

SEQ ID NO: 1443 - skipped

SEQ ID NO: 1444 - protein, 8 aa, synthetic construct
GFTFGDYT

SEQ ID NO: 1445 - skipped

SEQ ID NO: 1446 - protein, 8 aa, synthetic construct
ISWNSGSK

SEQ ID NO: 1447 - skipped

SEQ ID NO: 1448 - protein, 17 aa, synthetic construct
AKDSRGYGLYYHLGLDV

SEQ ID NO: 1449 - skipped

SEQ ID NO: 1450 - protein, 124 aa, synthetic construct
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 1451 - skipped

SEQ ID NO: 1452 - protein, 8 aa, synthetic construct
GFTFADYT

SEQ ID NO: 1453 - skipped

SEQ ID NO: 1454 - protein, 8 aa, synthetic construct
ISWNSGSI

SEQ ID NO: 1455 - skipped

SEQ ID NO: 1456 - protein, 17 aa, synthetic construct
AKDSRGYGHYKYLGLDV

SEQ ID NO: 1457 - skipped

SEQ ID NO: 1458 - protein, 124 aa, synthetic construct
EVQLVESGGGMVQPGRSLRLSCAASGFPFNDYTMHWVRQVPGRGLEWVSDISWNSGSKGYADSVKGRFIISRDNAKNSLYLQMNSLRVEDTALYYCVKDGSGYGRFHYYAMDVWGQGTTVTVSS

SEQ ID NO: 1459 - skipped

SEQ ID NO: 1460 - protein, 8 aa, synthetic construct
GFPFNDYT

SEQ ID NO: 1461 - skipped

SEQ ID NO: 1462 - protein, 8 aa, synthetic construct
ISWNSGSK

SEQ ID NO: 1463 - skipped

SEQ ID NO: 1464 - protein, 17 aa, synthetic construct
VKDGSGYGRFHYYAMDV

SEQ ID NO: 1465 - skipped

SEQ ID NO: 1466 - protein, 124 aa, synthetic construct
EVQLVESGGGLVQPGRSLRLSCAASGFTFADYTMHWVRQVPGKGLEWVSDISWNSGSKDYADSVKGRFTISRDNAKNFLYLQMNSLRAEDTALYYCVKYGSGYGKFYFYAMDVWGQGTTVTVSS

SEQ ID NO: 1467 - skipped

SEQ ID NO: 1468 - protein, 8 aa, synthetic construct
GFTFADYT

SEQ ID NO: 1469 - skipped

SEQ ID NO: 1470 - protein, 8 aa, synthetic construct
ISWNSGSK

SEQ ID NO: 1471 - skipped

SEQ ID NO: 1472 - protein, 17 aa, synthetic construct
VKYGSGYGKFYFYAMDV

SEQ ID NO: 1473 - skipped

SEQ ID NO: 1474 - protein, 124 aa, synthetic construct
EVQLVQSGGGLVQPGRSLRLSCAASGFTFADYTMHWVRQTPGKGLEWVSGISWNSGSKDYADSVKGRFTISRDNAKNSLYLQMNSLRTEDTALYYCAKYGSGYGRYFFYAMDVWGQGTTVTVSS

SEQ ID NO: 1475 - skipped

SEQ ID NO: 1476 - protein, 8 aa, synthetic construct
GFTFADYT

SEQ ID NO: 1477 - skipped

SEQ ID NO: 1478 - protein, 8 aa, synthetic construct
ISWNSGSK

SEQ ID NO: 1479 - skipped

SEQ ID NO: 1480 - protein, 17 aa, synthetic construct
AKYGSGYGRYFFYAMDV

SEQ ID NO: 1481 - skipped

SEQ ID NO: 1482 - protein, 124 aa, synthetic construct
EVQLVESRGALVQPGRSLRLSCAASGFAFNDYTMHWVRQAPGKGLEWVSDISWNSNSKDYADSVKGRFTISRDNAKNSLYLQMNSLRAEDTALYYCVKDGSGYGKFSLYALDVWGQGTTVTVSS

SEQ ID NO: 1483 - skipped

SEQ ID NO: 1484 - protein, 8 aa, synthetic construct
GFAFNDYT

SEQ ID NO: 1485 - skipped

SEQ ID NO: 1486 - protein, 8 aa, synthetic construct
ISWNSNSK

SEQ ID NO: 1487 - skipped

SEQ ID NO: 1488 - protein, 17 aa, synthetic construct
VKDGSGYGKFSLYALDV

SEQ ID NO: 1489 - skipped

SEQ ID NO: 1490 - protein, 124 aa, synthetic construct
QVQLVESGGGLVHPGRSLRLSCAASGFKFDDYTMHWVRQAPGKGLEWVSDISWNSGSKGYADSVKGRFTISRDNAKDSLYLQMDSLRAADTAFYYCAKDKSGYGHFYYYAMDVWGQGTTVTVSS

SEQ ID NO: 1491 - skipped

SEQ ID NO: 1492 - protein, 8 aa, synthetic construct
GFKFDDYT

SEQ ID NO: 1493 - skipped

SEQ ID NO: 1494 - protein, 8 aa, synthetic construct
ISWNSGSK

SEQ ID NO: 1495 - skipped

SEQ ID NO: 1496 - protein, 17 aa, synthetic construct
AKDKSGYGHFYYYAMDV

SEQ ID NO: 1497 - skipped

SEQ ID NO: 1498 - protein, 124 aa, synthetic construct
EVQLVESGGGLVHPGRSLRLSCTASGFKFADYTMHWVRQAPGKGLEWVSDISWNSGSKGYADSVKGRFTISRDNDKNSLYLQMNSLRGEDTALYYCAKDGSGYGRFHFYAIDVWGQGTTVTVSS

SEQ ID NO: 1499 - skipped

SEQ ID NO: 1500 - protein, 8 aa, synthetic construct
GFKFADYT

SEQ ID NO: 1501 - skipped

SEQ ID NO: 1502 - protein, 8 aa, synthetic construct
ISWNSGSK

SEQ ID NO: 1503 - skipped

SEQ ID NO: 1504 - protein, 17 aa, synthetic construct
AKDGSGYGRFHFYAIDV

SEQ ID NO: 1505 - skipped

SEQ ID NO: 1506 - protein, 124 aa, synthetic construct
EVQLVESGGGLVQPGRSLRLSCVASGFTFDDYSMHWVRQAPGKGLEWVSGISWNSGSKDYADSVKGRFTISRDNAKNSLYLQMNSLRAEDTALYYCAKYGSGYGKFYHYGLDVWGQGTTVTVSS

SEQ ID NO: 1507 - skipped

SEQ ID NO: 1508 - protein, 8 aa, synthetic construct
GFTFDDYS

SEQ ID NO: 1509 - skipped

SEQ ID NO: 1510 - protein, 8 aa, synthetic construct
ISWNSGSK

SEQ ID NO: 1511 - skipped

SEQ ID NO: 1512 - protein, 17 aa, synthetic construct
AKYGSGYGKFYHYGLDV

SEQ ID NO: 1513 - skipped

SEQ ID NO: 1514 - protein, 124 aa, synthetic construct
QVQLVESGGGLVQPGRSLRLSCAASGFTFADYTMHWVRQAPGKGLEWVSDISWNSGSMGYADSVKGRFTISRDNAKKSLYLQMNSLRTEDTALYYCAKDGSGYGKYFFYAMDVWGQGTTVTVSS

SEQ ID NO: 1515 - skipped

SEQ ID NO: 1516 - protein, 8 aa, synthetic construct
GFTFADYT

SEQ ID NO: 1517 - skipped

SEQ ID NO: 1518 - protein, 8 aa, synthetic construct
ISWNSGSM

SEQ ID NO: 1519 - skipped

SEQ ID NO: 1520 - protein, 17 aa, synthetic construct
AKDGSGYGKYFFYAMDV

SEQ ID NO: 1521 - skipped

SEQ ID NO: 1522 - protein, 124 aa, synthetic construct
EVQLVQSGGGLVQPGRSLRLSCAASGFTFSDYTMHWVRQGPGKGLEWVSDISWNSGSKGYTDSVKGRFTISRDNAKKSLYLQMNSLRAEDTALYYCVKDGSGYGKYHFYAMDVWGQGTLVTVSS

SEQ ID NO: 1523 - skipped

SEQ ID NO: 1524 - protein, 8 aa, synthetic construct
GFTFSDYT

SEQ ID NO: 1525 - skipped

SEQ ID NO: 1526 - protein, 8 aa, synthetic construct
ISWNSGSK

SEQ ID NO: 1527 - skipped

SEQ ID NO: 1528 - protein, 17 aa, synthetic construct
VKDGSGYGKYHFYAMDV

SEQ ID NO: 1529 - skipped

SEQ ID NO: 1530 - protein, 124 aa, synthetic construct
EVQLVESGGGLVHPGRSLRLSCAASGFTFDDYTMHWVRQAPGKGLEWVSDISWNSGSRGYADSVKGRFTISRDNAENSLYLQMNSLRAEDTALYYCAKDKSGYGHYYYYAMDVWGQGTTVTVSS

SEQ ID NO: 1531 - skipped

SEQ ID NO: 1532 - protein, 8 aa, synthetic construct
GFTFDDYT

SEQ ID NO: 1533 - skipped

SEQ ID NO: 1534 - protein, 8 aa, synthetic construct
ISWNSGSR

SEQ ID NO: 1535 - skipped

SEQ ID NO: 1536 - protein, 17 aa, synthetic construct
AKDKSGYGHYYYYAMDV

SEQ ID NO: 1537 - skipped

SEQ ID NO: 1538 - protein, 124 aa, synthetic construct
EVQLVESGGGLVQPGGSLRLSCEASGFTFADYTLHWVRQAPGKGLEWVSDISWNSGTRGYADSVRGRFTISRDNAKKSLYLQMNSLRSEDTALYYCVKDGSGYGRYNFYAMDVWGQGTTVTVSS

SEQ ID NO: 1539 - skipped

SEQ ID NO: 1540 - protein, 8 aa, synthetic construct
GFTFADYT

SEQ ID NO: 1541 - skipped

SEQ ID NO: 1542 - protein, 8 aa, synthetic construct
ISWNSGTR

SEQ ID NO: 1543 - skipped

SEQ ID NO: 1544 - protein, 17 aa, synthetic construct
VKDGSGYGRYNFYAMDV

SEQ ID NO: 1545 - skipped

SEQ ID NO: 1546 - protein, 123 aa, synthetic construct
EVQLVESGGGLVQPGRSLRLSCAASGFTFADYTMHWVRQGPGKGLEWVSDIGWNSNTIGYADSVKGRFAISRDNAKNSLYLQMNSLRPEDTALYYCVKDKSGYGKFQYGLDVWGQGTTVTVSS

SEQ ID NO: 1547 - skipped

SEQ ID NO: 1548 - protein, 8 aa, synthetic construct
GFTFADYT

SEQ ID NO: 1549 - skipped

SEQ ID NO: 1550 - protein, 8 aa, synthetic construct
IGWNSNTI

SEQ ID NO: 1551 - skipped

SEQ ID NO: 1552 - protein, 16 aa, synthetic construct
VKDKSGYGKFQYGLDV

SEQ ID NO: 1553 - skipped

SEQ ID NO: 1554 - protein, 123 aa, synthetic construct
EVQLVESGGGLVQPGRSLRLSCAASGFTFDDYTMHWVRQGPGKGLEWVSDIGWNSNSIGYADSVKGRFTISRDNAKNSLYLQMNSLRPEDTALYFCVKDKSGYGKFFIGLDVWGQGTMVTVSS

SEQ ID NO: 1555 - skipped

SEQ ID NO: 1556 - protein, 8 aa, synthetic construct
GFTFDDYT

SEQ ID NO: 1557 - skipped

SEQ ID NO: 1558 - protein, 8 aa, synthetic construct
IGWNSNSI

SEQ ID NO: 1559 - skipped

SEQ ID NO: 1560 - protein, 16 aa, synthetic construct
VKDKSGYGKFFIGLDV

SEQ ID NO: 1561 - skipped

SEQ ID NO: 1562 - protein, 123 aa, synthetic construct
QVQLVESGGGLVQPGRSLRLSCAASGFTFDDYTMHWVRQGPGKGLEWVSDIGWNSNTKGYADSVKGRFTISRDNAKNSLYLQMNSLRPEDTALYFCVKDKSGYGKFFIGLDVWGQGTMVTVSS

SEQ ID NO: 1563 - skipped

SEQ ID NO: 1564 - protein, 8 aa, synthetic construct
GFTFDDYT

SEQ ID NO: 1565 - skipped

SEQ ID NO: 1566 - protein, 8 aa, synthetic construct
IGWNSNTK

SEQ ID NO: 1567 - skipped

SEQ ID NO: 1568 - protein, 16 aa, synthetic construct
VKDKSGYGKFFIGLDV

SEQ ID NO: 1569 - skipped

SEQ ID NO: 1570 - protein, 123 aa, synthetic construct
EVQLVESGGGLVQPGRSLRLSCAASGFTFADYTMHWVRQGPGTGLEWVSDIGWSGGSLGYADSVKGRFTISRDNAKNSLYLEMNNLRPEDTALYYCVKDKSGYGKFQYGLDVWGQGTTVTVSS

SEQ ID NO: 1571 - skipped

SEQ ID NO: 1572 - protein, 8 aa, synthetic construct
GFTFADYT

SEQ ID NO: 1573 - skipped

SEQ ID NO: 1574 - protein, 8 aa, synthetic construct
IGWSGGSL

SEQ ID NO: 1575 - skipped

SEQ ID NO: 1576 - protein, 16 aa, synthetic construct
VKDKSGYGKFQYGLDV

SEQ ID NO: 1577 - skipped

SEQ ID NO: 1578 - protein, 123 aa, synthetic construct
EVQLVESGGGVVQPGRSLRLSCAASGFKFDGYTMHWVRQGPGKGLEWVSDIGWNSNTIGYADSVKGRFTISRDNAKNSLYLQMNSLRPEDTALYFCVKDKSGYGKFFIGLDVWGQGTMVTVSS

SEQ ID NO: 1579 - skipped

SEQ ID NO: 1580 - protein, 8 aa, synthetic construct
GFKFDGYT

SEQ ID NO: 1581 - skipped

SEQ ID NO: 1582 - protein, 8 aa, synthetic construct
IGWNSNTI

SEQ ID NO: 1583 - skipped

SEQ ID NO: 1584 - protein, 16 aa, synthetic construct
VKDKSGYGKFFIGLDV

SEQ ID NO: 1585 - skipped

SEQ ID NO: 1586 - protein, 123 aa, synthetic construct
QVQLVESGGGLVQPGRSLRLSCAASGFTFDDYTMHWVRQGPGKGLEWVSDIGWNSNTIGYADSVKGRFTISRDNARNSLYLQMNSLRPEDTALYYCVKDKSGYGKFQYGLDVWGQGTTVTVSS

SEQ ID NO: 1587 - skipped

SEQ ID NO: 1588 - protein, 8 aa, synthetic construct
GFTFDDYT

SEQ ID NO: 1589 - skipped

SEQ ID NO: 1590 - protein, 8 aa, synthetic construct
IGWNSNTI

SEQ ID NO: 1591 - skipped

SEQ ID NO: 1592 - protein, 16 aa, synthetic construct
VKDKSGYGKFQYGLDV

SEQ ID NO: 1593 - skipped

SEQ ID NO: 1594 - protein, 123 aa, synthetic construct
QVQLVESGGGLVQPGRSLRLSCAASGFKFDDYTMHWVRQGPGKGLEWVSDISWSGGSIDYTDSVKGRFSISRDNAKNSLYLQMNSLRVEDTALYYCVKDKSGYGKYSYGLDVWGQGTTVTVSS

SEQ ID NO: 1595 - skipped

SEQ ID NO: 1596 - protein, 8 aa, synthetic construct
GFKFDDYT

SEQ ID NO: 1597 - skipped

SEQ ID NO: 1598 - protein, 8 aa, synthetic construct
ISWSGGSI

SEQ ID NO: 1599 - skipped

SEQ ID NO: 1600 - protein, 16 aa, synthetic construct
VKDKSGYGKYSYGLDV

SEQ ID NO: 1601 - skipped

SEQ ID NO: 1602 - protein, 119 aa, synthetic construct
EVQLVESGGGLVKPGGSLRISCAASGFSFINAWMNWVRQAPGKGLEWVGRIKSISDGGTTDYAASVKGRFTISREDSKNMLFLEMNSLKTEDTAVFYCTTEVARTPNYWGRGTLVTVSS

SEQ ID NO: 1603 - skipped

SEQ ID NO: 1604 - protein, 8 aa, synthetic construct
GFSFINAW

SEQ ID NO: 1605 - skipped

SEQ ID NO: 1606 - protein, 10 aa, synthetic construct
IKSISDGGTT

SEQ ID NO: 1607 - skipped

SEQ ID NO: 1608 - protein, 10 aa, synthetic construct
TTEVARTPNY

SEQ ID NO: 1609 - skipped

SEQ ID NO: 1610 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 1611 - skipped

SEQ ID NO: 1612 - protein, 8 aa, synthetic construct
GFTFDDYS

SEQ ID NO: 1613 - skipped

SEQ ID NO: 1614 - protein, 8 aa, synthetic construct
ISWNSGSK

SEQ ID NO: 1615 - skipped

SEQ ID NO: 1616 - protein, 17 aa, synthetic construct
AKYGSGYGKFYHYGLDV

SEQ ID NO: 1617 - skipped

SEQ ID NO: 1618 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGYFYYYGMDVWGQGTTVTVSS

SEQ ID NO: 1619 - skipped

SEQ ID NO: 1620 - protein, 8 aa, synthetic construct
GFTFDDYS

SEQ ID NO: 1621 - skipped

SEQ ID NO: 1622 - protein, 8 aa, synthetic construct
ISWNSGSI

SEQ ID NO: 1623 - skipped

SEQ ID NO: 1624 - protein, 17 aa, synthetic construct
AKDGSGYGYFYYYGMDV

SEQ ID NO: 1625 - skipped

SEQ ID NO: 1626 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 1627 - skipped

SEQ ID NO: 1628 - protein, 8 aa, synthetic construct
GFTFDDYS

SEQ ID NO: 1629 - skipped

SEQ ID NO: 1630 - protein, 8 aa, synthetic construct
ISWNSGSI

SEQ ID NO: 1631 - skipped

SEQ ID NO: 1632 - protein, 17 aa, synthetic construct
AKDGSGYGKFYYYGMDV

SEQ ID NO: 1633 - skipped

SEQ ID NO: 1634 - protein, 108 aa, synthetic construct; identical to SEQ ID NO: 394

SEQ ID NO: 1635 - skipped

SEQ ID NO: 1636 - protein, 6 aa, synthetic construct
QSISSY

SEQ ID NO: 1637-1639 - skipped (3 entries)

SEQ ID NO: 1640 - protein, 10 aa, synthetic construct
QQSYSTPPIT

SEQ ID NO: 1641 - skipped

SEQ ID NO: 1642 - protein, 123 aa, synthetic construct; identical to SEQ ID NO: 514

SEQ ID NO: 1643 - skipped

SEQ ID NO: 1644 - protein, 8 aa, synthetic construct
GFTFDDYT

SEQ ID NO: 1645 - skipped

SEQ ID NO: 1646 - protein, 8 aa, synthetic construct
ISWNSGSI

SEQ ID NO: 1647 - skipped

SEQ ID NO: 1648 - protein, 16 aa, synthetic construct
AKDNSGYGHYYYGMDV

SEQ ID NO: 1649 - skipped

SEQ ID NO: 1650 - protein, 108 aa, synthetic construct
AEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 1651 - skipped

SEQ ID NO: 1652 - protein, 6 aa, synthetic construct
QSVSSN

SEQ ID NO: 1653-1655 - skipped (3 entries)

SEQ ID NO: 1656 - protein, 9 aa, synthetic construct
QHYINWPLT

SEQ ID NO: 1657 - skipped

SEQ ID NO: 1658 - protein, 123 aa, synthetic construct; identical to SEQ ID NO: 402

SEQ ID NO: 1659 - skipped

SEQ ID NO: 1660 - protein, 8 aa, synthetic construct
GFTFDDFT

SEQ ID NO: 1661 - skipped

SEQ ID NO: 1662 - protein, 8 aa, synthetic construct
ISWNSGSI

SEQ ID NO: 1663 - skipped

SEQ ID NO: 1664 - protein, 16 aa, synthetic construct
AKDNSGYGYYYYGMDV

SEQ ID NO: 1665 - skipped

SEQ ID NO: 1666 - protein, 107 aa, synthetic construct; identical to SEQ ID NO: 410

SEQ ID NO: 1667 - skipped

SEQ ID NO: 1668 - protein, 6 aa, synthetic construct
HSVSRN

SEQ ID NO: 1669-1671 - skipped (3 entries)

SEQ ID NO: 1672 - protein, 9 aa, synthetic construct
QQYNNWPLT

SEQ ID NO: 1673 - skipped

SEQ ID NO: 1674 - protein, 123 aa, synthetic construct; identical to SEQ ID NO: 514

SEQ ID NO: 1675 - skipped

SEQ ID NO: 1676 - protein, 8 aa, synthetic construct
GFTFDDYT

SEQ ID NO: 1677 - skipped

SEQ ID NO: 1678 - protein, 8 aa, synthetic construct
ISWNSGSI

SEQ ID NO: 1679 - skipped

SEQ ID NO: 1680 - protein, 16 aa, synthetic construct
AKDNSGYGHYYYGMDV

SEQ ID NO: 1681 - skipped

SEQ ID NO: 1682 - protein, 107 aa, synthetic construct; identical to SEQ ID NO: 522

SEQ ID NO: 1683 - skipped

SEQ ID NO: 1684 - protein, 6 aa, synthetic construct
QSVSSN

SEQ ID NO: 1685-1687 - skipped (3 entries)

SEQ ID NO: 1688 - protein, 9 aa, synthetic construct
QHYINWPLT

SEQ ID NO: 1689 - skipped

SEQ ID NO: 1690 - protein, 118 aa, synthetic construct; identical to SEQ ID NO: 578

SEQ ID NO: 1691 - skipped

SEQ ID NO: 1692 - protein, 8 aa, synthetic construct
GFTFRSYA

SEQ ID NO: 1693 - skipped

SEQ ID NO: 1694 - protein, 8 aa, synthetic construct
VYYDGNNQ

SEQ ID NO: 1695 - skipped

SEQ ID NO: 1696 - protein, 11 aa, synthetic construct
ARGPGYNWLDP

SEQ ID NO: 1697 - skipped

SEQ ID NO: 1698 - protein, 107 aa, synthetic construct; identical to SEQ ID NO: 586

SEQ ID NO: 1699 - skipped

SEQ ID NO: 1700 - protein, 6 aa, synthetic construct
QSVSRN

SEQ ID NO: 1701-1703 - skipped (3 entries)

SEQ ID NO: 1704 - protein, 9 aa, synthetic construct
QQYNNWPLT

SEQ ID NO: 1705 - skipped

SEQ ID NO: 1706 - protein, 118 aa, synthetic construct; identical to SEQ ID NO: 562

SEQ ID NO: 1707 - skipped

SEQ ID NO: 1708 - protein, 8 aa, synthetic construct
GFTFRSYG

SEQ ID NO: 1709 - skipped

SEQ ID NO: 1710 - protein, 8 aa, synthetic construct
IYYDGKNK

SEQ ID NO: 1711 - skipped

SEQ ID NO: 1712 - protein, 11 aa, synthetic construct
ARGPGYNWLDP

SEQ ID NO: 1713 - skipped

SEQ ID NO: 1714 - protein, 107 aa, synthetic construct; identical to SEQ ID NO: 570

SEQ ID NO: 1715 - skipped

SEQ ID NO: 1716 - protein, 6 aa, synthetic construct
QRISSN

SEQ ID NO: 1717-1719 - skipped (3 entries)

SEQ ID NO: 1720 - protein, 9 aa, synthetic construct
QQHHNWPLT

SEQ ID NO: 1721 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 1722 - protein, 8 aa, synthetic construct
GYTFTRYT

SEQ ID NO: 1723 - protein, 8 aa, synthetic construct
INPSRGYT

SEQ ID NO: 1724 - protein, 12 aa, synthetic construct
ARYYDDHYCLDY

SEQ ID NO: 1725 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 1726 - protein, 11 aa, synthetic construct
LSCRASQSVSY

SEQ ID NO: 1727 - skipped

SEQ ID NO: 1728 - protein, 9 aa, synthetic construct
QQWSSNPLT

SEQ ID NO: 1729 - skipped

SEQ ID NO: 1730 - protein, 124 aa, synthetic construct; identical to SEQ ID NO: 1610

SEQ ID NO: 1731 - skipped

SEQ ID NO: 1732 - protein, 8 aa, synthetic construct
GFTFDDYS

SEQ ID NO: 1733 - skipped

SEQ ID NO: 1734 - protein, 8 aa, synthetic construct
ISWNSGSK

SEQ ID NO: 1735 - skipped

SEQ ID NO: 1736 - protein, 17 aa, synthetic construct
AKYGSGYGKFYHYGLDV

SEQ ID NO: 1737 - skipped

SEQ ID NO: 1738 - protein, 124 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDGSGYGYFYYYGMDVWGQGTTVTVSS

SEQ ID NO: 1739 - skipped

SEQ ID NO: 1740 - protein, 8 aa, synthetic construct
GFTFDDYA

SEQ ID NO: 1741 - skipped

SEQ ID NO: 1742 - protein, 8 aa, synthetic construct
ISWNSGSI

SEQ ID NO: 1743 - skipped

SEQ ID NO: 1744 - protein, 17 aa, synthetic construct
AKDGSGYGYFYYYGMDV

SEQ ID NO: 1745 - skipped

SEQ ID NO: 1746 - protein, 124 aa, synthetic construct; identical to SEQ ID NO: 1618

SEQ ID NO: 1747 - skipped

SEQ ID NO: 1748 - protein, 8 aa, synthetic construct
GFTFDDYS

SEQ ID NO: 1749 - skipped

SEQ ID NO: 1750 - protein, 8 aa, synthetic construct
ISWNSGSI

SEQ ID NO: 1751 - skipped

SEQ ID NO: 1752 - protein, 17 aa, synthetic construct
AKDGSGYGYFYYYGMDV

SEQ ID NO: 1753 - skipped

SEQ ID NO: 1754 - protein, 124 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 1755 - skipped

SEQ ID NO: 1756 - protein, 8 aa, synthetic construct
GFTFDDYA

SEQ ID NO: 1757 - skipped

SEQ ID NO: 1758 - protein, 8 aa, synthetic construct
ISWNSGSI

SEQ ID NO: 1759 - skipped

SEQ ID NO: 1760 - protein, 17 aa, synthetic construct
AKDGSGYGKFYYYGMDV

SEQ ID NO: 1761 - skipped

SEQ ID NO: 1762 - protein, 124 aa, synthetic construct; identical to SEQ ID NO: 1626

SEQ ID NO: 1763 - skipped

SEQ ID NO: 1764 - protein, 8 aa, synthetic construct
GFTFDDYS

SEQ ID NO: 1765 - skipped

SEQ ID NO: 1766 - protein, 8 aa, synthetic construct
ISWNSGSI

SEQ ID NO: 1767 - skipped

SEQ ID NO: 1768 - protein, 17 aa, synthetic construct
AKDGSGYGKFYYYGMDV

SEQ ID NO: 1769 - skipped

SEQ ID NO: 1770 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYHYGLDVWGQGTTVTVSS

SEQ ID NO: 1771 - skipped

SEQ ID NO: 1772 - protein, 8 aa, synthetic construct
GFTFDDYS

SEQ ID NO: 1773 - skipped

SEQ ID NO: 1774 - protein, 8 aa, synthetic construct
ISWNSGSI

SEQ ID NO: 1775 - skipped

SEQ ID NO: 1776 - protein, 17 aa, synthetic construct
AKYGSGYGKFYHYGLDV

SEQ ID NO: 1777 - skipped

SEQ ID NO: 1778 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYHYGLDVWGQGTTVTVSS

SEQ ID NO: 1779 - skipped

SEQ ID NO: 1780 - protein, 8 aa, synthetic construct
GFTFDDYS

SEQ ID NO: 1781 - skipped

SEQ ID NO: 1782 - protein, 8 aa, synthetic construct
ISWNSGSK

SEQ ID NO: 1783 - skipped

SEQ ID NO: 1784 - protein, 17 aa, synthetic construct
AKDGSGYGKFYHYGLDV

SEQ ID NO: 1785 - skipped

SEQ ID NO: 1786 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYYYGLDVWGQGTTVTVSS

SEQ ID NO: 1787 - skipped

SEQ ID NO: 1788 - protein, 8 aa, synthetic construct
GFTFDDYS

SEQ ID NO: 1789 - skipped

SEQ ID NO: 1790 - protein, 8 aa, synthetic construct
ISWNSGSK

SEQ ID NO: 1791 - skipped

SEQ ID NO: 1792 - protein, 17 aa, synthetic construct
AKYGSGYGKFYYYGLDV

SEQ ID NO: 1793 - skipped

SEQ ID NO: 1794 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGMDVWGQGTTVTVSS

SEQ ID NO: 1795 - skipped

SEQ ID NO: 1796 - protein, 8 aa, synthetic construct
GFTFDDYS

SEQ ID NO: 1797 - skipped

SEQ ID NO: 1798 - protein, 8 aa, synthetic construct
ISWNSGSK

SEQ ID NO: 1799 - skipped

SEQ ID NO: 1800 - protein, 17 aa, synthetic construct
AKYGSGYGKFYHYGMDV

SEQ ID NO: 1801 - skipped

SEQ ID NO: 1802 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYHYGLDVWGQGTTVTVSS

SEQ ID NO: 1803 - skipped

SEQ ID NO: 1804 - protein, 8 aa, synthetic construct
GFTFDDYS

SEQ ID NO: 1805 - skipped

SEQ ID NO: 1806 - protein, 8 aa, synthetic construct
ISWNSGSI

SEQ ID NO: 1807 - skipped

SEQ ID NO: 1808 - protein, 17 aa, synthetic construct
AKDGSGYGKFYHYGLDV

SEQ ID NO: 1809 - skipped

SEQ ID NO: 1810 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGLDVWGQGTTVTVSS

SEQ ID NO: 1811 - skipped

SEQ ID NO: 1812 - protein, 8 aa, synthetic construct
GFTFDDYS

SEQ ID NO: 1813 - skipped

SEQ ID NO: 1814 - protein, 8 aa, synthetic construct
ISWNSGSI

SEQ ID NO: 1815 - skipped

SEQ ID NO: 1816 - protein, 17 aa, synthetic construct
AKYGSGYGKFYYYGLDV

SEQ ID NO: 1817 - skipped

SEQ ID NO: 1818 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYHYGMDVWGQGTTVTVSS

SEQ ID NO: 1819 - skipped

SEQ ID NO: 1820 - protein, 8 aa, synthetic construct
GFTFDDYS

SEQ ID NO: 1821 - skipped

SEQ ID NO: 1822 - protein, 8 aa, synthetic construct
ISWNSGSI

SEQ ID NO: 1823 - skipped

SEQ ID NO: 1824 - protein, 17 aa, synthetic construct
AKYGSGYGKFYHYGMDV

SEQ ID NO: 1825 - skipped

SEQ ID NO: 1826 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYYYGLDVWGQGTTVTVSS

SEQ ID NO: 1827 - skipped

SEQ ID NO: 1828 - protein, 8 aa, synthetic construct
GFTFDDYS

SEQ ID NO: 1829 - skipped

SEQ ID NO: 1830 - protein, 8 aa, synthetic construct
ISWNSGSK

SEQ ID NO: 1831 - skipped

SEQ ID NO: 1832 - protein, 17 aa, synthetic construct
AKDGSGYGKFYYYGLDV

SEQ ID NO: 1833 - skipped

SEQ ID NO: 1834 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYHYGMDVWGQGTTVTVSS

SEQ ID NO: 1835 - skipped

SEQ ID NO: 1836 - protein, 8 aa, synthetic construct
GFTFDDYS

SEQ ID NO: 1837 - skipped

SEQ ID NO: 1838 - protein, 8 aa, synthetic construct
ISWNSGSK

SEQ ID NO: 1839 - skipped

SEQ ID NO: 1840 - protein, 17 aa, synthetic construct
AKDGSGYGKFYHYGMDV

SEQ ID NO: 1841 - skipped

SEQ ID NO: 1842 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYYYGMDVWGQGTTVTVSS

SEQ ID NO: 1843 - skipped

SEQ ID NO: 1844 - protein, 8 aa, synthetic construct
GFTFDDYS

SEQ ID NO: 1845 - skipped

SEQ ID NO: 1846 - protein, 8 aa, synthetic construct
ISWNSGSK

SEQ ID NO: 1847 - skipped

SEQ ID NO: 1848 - protein, 17 aa, synthetic construct
AKYGSGYGKFYYYGMDV

SEQ ID NO: 1849 - skipped

SEQ ID NO: 1850 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGLDVWGQGTTVTVSS

SEQ ID NO: 1851 - skipped

SEQ ID NO: 1852 - protein, 8 aa, synthetic construct
GFTFDDYS

SEQ ID NO: 1853 - skipped

SEQ ID NO: 1854 - protein, 8 aa, synthetic construct
ISWNSGSI

SEQ ID NO: 1855 - skipped

SEQ ID NO: 1856 - protein, 17 aa, synthetic construct
AKDGSGYGKFYYYGLDV

SEQ ID NO: 1857 - skipped

SEQ ID NO: 1858 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYHYGMDVWGQGTTVTVSS

SEQ ID NO: 1859 - skipped

SEQ ID NO: 1860 - protein, 8 aa, synthetic construct
GFTFDDYS

SEQ ID NO: 1861 - skipped

SEQ ID NO: 1862 - protein, 8 aa, synthetic construct
ISWNSGSI

SEQ ID NO: 1863 - skipped

SEQ ID NO: 1864 - protein, 17 aa, synthetic construct
AKDGSGYGKFYHYGMDV

SEQ ID NO: 1865 - skipped

SEQ ID NO: 1866 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 1867 - skipped

SEQ ID NO: 1868 - protein, 8 aa, synthetic construct
GFTFDDYS

SEQ ID NO: 1869 - skipped

SEQ ID NO: 1870 - protein, 8 aa, synthetic construct
ISWNSGSI

SEQ ID NO: 1871 - skipped

SEQ ID NO: 1872 - protein, 17 aa, synthetic construct
AKYGSGYGKFYYYGMDV

SEQ ID NO: 1873 - skipped

SEQ ID NO: 1874 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYYYGMDVWGQGTTVTVSS

SEQ ID NO: 1875 - skipped

SEQ ID NO: 1876 - protein, 8 aa, synthetic construct
GFTFDDYS

SEQ ID NO: 1877 - skipped

SEQ ID NO: 1878 - protein, 8 aa, synthetic construct
ISWNSGSK

SEQ ID NO: 1879 - skipped

SEQ ID NO: 1880 - protein, 17 aa, synthetic construct
AKDGSGYGKFYYYGMDV

SEQ ID NO: 1881 - skipped

SEQ ID NO: 1882 - protein, 124 aa, synthetic construct; identical to SEQ ID NO: 1506

SEQ ID NO: 1883 - skipped

SEQ ID NO: 1884 - protein, 8 aa, synthetic construct
GFTFDDYS

SEQ ID NO: 1885 - skipped

SEQ ID NO: 1886 - protein, 8 aa, synthetic construct
ISWNSGSK

SEQ ID NO: 1887 - skipped

SEQ ID NO: 1888 - protein, 17 aa, synthetic construct
AKYGSGYGKFYHYGLDV

SEQ ID NO: 1889 - skipped

SEQ ID NO: 1890 - protein, 108 aa, synthetic construct
DIQMTQSPSSLSASVGDSVTITCRASQSINNYLNWYQQKPGKAPKLLIYTASSLLSGVPSRFSGSGSGTDFTLTISGLHPEDFATYFCQQSFSTPPITFGQGTRLDIK

SEQ ID NO: 1891 - skipped

SEQ ID NO: 1892 - protein, 6 aa, synthetic construct
QSINNY

SEQ ID NO: 1893-1895 - skipped (3 entries)

SEQ ID NO: 1896 - protein, 10 aa, synthetic construct
QQSFSTPPIT

SEQ ID NO: 1897-1935 - skipped (39 entries)

SEQ ID NO: 1936 - protein, 108 aa, synthetic construct; identical to SEQ ID NO: 170

SEQ ID NO: 1937 - skipped

SEQ ID NO: 1938 - protein, 7 aa, synthetic construct
QSVSSSY

SEQ ID NO: 1939-1941 - skipped (3 entries)

SEQ ID NO: 1942 - protein, 9 aa, synthetic construct
QQYGSSPWT

SEQ ID NO: 1943 - skipped

SEQ ID NO: 1944 - protein, 119 aa, synthetic construct
QVQLQQWGAGLLKPSETLSLTCAVYGGSFSGFYWSWIRQSPGKGLEWIGEINRGGSTNSNPSLKSRVTMSLDTSKTQFSLKLRSVTAADTAIYYCARSLELDSDGFDIWGQGTMVTVSS

SEQ ID NO: 1945 - skipped

SEQ ID NO: 1946 - protein, 8 aa, synthetic construct
GGSFSGFY

SEQ ID NO: 1947 - skipped

SEQ ID NO: 1948 - protein, 7 aa, synthetic construct
INRGGST

SEQ ID NO: 1949 - skipped

SEQ ID NO: 1950 - protein, 13 aa, synthetic construct
ARSLELDSDGFDI

SEQ ID NO: 1951 - skipped

SEQ ID NO: 1952 - protein, 108 aa, synthetic construct
ETVLTQSPGTLSLSPGERATLSCRASQSVSSSYVAWYQQKPGQAPRRLIYGASSRATGIPGRFSGSGSGTDFTLTISRLEPEDFAVYYCQQYGSSPWTFGQGTKVEIK

SEQ ID NO: 1953 - skipped

SEQ ID NO: 1954 - protein, 7 aa, synthetic construct
QSVSSSY

SEQ ID NO: 1955-1957 - skipped (3 entries)

SEQ ID NO: 1958 - protein, 9 aa, synthetic construct
QQYGSSPWT

SEQ ID NO: 1959 - protein, 113 aa, Homo sapiens
GVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSV

SEQ ID NO: 1960 - protein, 11 aa, synthetic construct
VSGWRLFKKIS